SEQUENCE LISTING (202 sequences)

SEQ ID NO: 1 - DNA, 420 bp, Homo sapiens
  misc_feature (83)..(83): n is a, c, g, or t
cactttgcaactccctgggtaagagggacgacacctctggtttttcaataccaattacatggaacttttctgtaatgggtacnaatgaagaagtttctaaaaacacacacaaagcacattgggccaactatttagtaagcccggatagacttattgccaaaaacaaaaaatagctttcaaaagaaatttaagttctatgagaaattccttagtcatggtgttgcgtaaatcatattttagctgcacggcattaccccacacagggtggcagaacttgaagggttactgacgtgtaaatgctggtatttgatttcctgtgtgtgttgccctggcattaagggcattttacccttgcagttttactaaaacactgaaaaatattccaagcttcatattaaccctacctgtcaacgtaacgat

SEQ ID NO: 2 - DNA, 437 bp, Homo sapiens
cctacccacctcaaaatgtctgtactgcaagagggccctgggcctctgctttccatattcacgtttggccagagttgtagtcccaaagaagagcatgggtggcagatggtagggaattgaactggcctgtgcaatgggcatggagcacaaggggtcacagcatgcctcctgccttaccgtggcagtacggagacagtccagaacatggtcttcttgccacggggtgttgttgtctctggtggtgctgcatgtctgtggctcacctttattcttgaaactgaggtttacctggatctggctactgaggctagagcccacagcagaatggggttgggcctgtggccccccaaactagggggtgtgggttcatcacagtgttgccttttgtctcctaaagatagggatctacttttgaagggaattgttcctcccaaata

SEQ ID NO: 3 - DNA, 161 bp, Homo sapiens
agagctgatcacaagcacaaatctttcccactagccatttaataagttaaaaaaagatacaaaaacaaaaacctactagtcttgaacaaactgtcatacgtatgggacctacacttaatctatatgctttacactagctttctgcatttaataggttagaa

SEQ ID NO: 4 - DNA, 475 bp, Homo sapiens
ggtgatgggttgtgttatgcttgtattgaatgctgtcttgacatctcttgccttgtcctccggtatgttctaaagctgtgtctgagatctggatctgcccatcactttggcctagggacagggctaattaatttgctttatacattttcttttactttccttttttcctttctggaggcatcacatgctggtgctgtgtctttatgaatgttttaaccattttcatggtggaagaattttatatttatgcagttgtacaattttatttttttctgcaagaaaaagtgtaatgtatgaaataaaccaaagtcacttgtttgaaaataaatctttattttgaactttataaaagcaatgcagtaccccatagactggtgttaaatgttgtctacagtgcaaaatccatgttctaacatatgtaataattgccaggagtacagtgctcttgttgatcttgtattcagtcaggttaaaa

SEQ ID NO: 5 - DNA, 477 bp, Homo sapiens
ggtgaaatttctaactgttctctgttcccggaaccgaaatcacctgttgcatgtgtttgatgaatacaaaaggatatcacagaaggatattgaacagagtattaaatctgaaacatctggtagctttgaagatgctctgctggctatagtaaagtgcatgaggaacaaatctgcatattttgctgaaaagctctataaatcgatgaagggcttgggcaccgatgataacaccctcatcagagtgatggtttctcgagcagaaattgacatgttggatatccgggcacacttcaagagactctatggaaagtctctgtactcgttcatcaagggtgacacatctggagactacaggaaagtactgcttgttctctgtggaggagatgattaaaataaaaatcccagaaggacaggaggattctcaacactttgaatttttttaacttcatttttctacactgctattatcattatctc

SEQ ID NO: 6 - DNA, 420 bp, Homo sapiens
ccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgag

SEQ ID NO: 7 - DNA, 493 bp, Homo sapiens
ccaccttcacctcggagggacggagaaagaagtggagacagtcctttcccaccattcctgcctttaagccaaagaaacaagctgtgcaggcatggtcccttaaggcacagtgggagctgagctggaaggggccacgtggatgggcaaagcttgtcaaagatgccccctccaggagagagccaggatgcccagatgaactgactgaaggaaaagcaagaaacagtttcttgcttggaagccaggtacaggagaggcagcatgcttgggctgacccagcatctcccagcaagacctcatctgtggagctgccacagagaagtttgtagccaggtactgcattctctcccatcctggggcagcactccccagagctgtgccagcaggggggctgtgccaacctgttcttagagtgtagctgtaagggcagtgcccatgtgtacattctgcctagagtgtagcctaaagggcagggcccacgtgtatagtatctgta

SEQ ID NO: 8 - DNA, 522 bp, Homo sapiens
tcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgt

SEQ ID NO: 9 - DNA, 444 bp, Homo sapiens
gacaaaccatttccaacagcaacacagccactaaaacacaaaaagggggattgggcggaaagtgagagccagcagcaaaaactacattttgcaacttgttggtgtggatctattggctgatctatgcctttcaactagaaaattctaatgattggcaagtcacgttgttttcaggtccagagtagtttctttctgtctgctttaaatggaaacagactcataccacacttacaattaaggtcaagcccagaaagtgataagtgcagggaggaaaagtgcaagtccattatgtaatagtgacagcaaaggcccaggggagaggcattgccttctctgcccacagtctttccgtgtgattgtctttgaatctgaatcagccagtctcagatgccccaaagtttcggttcctatgagcccggggcatgatctgatccccaagacatg

SEQ ID NO: 10 - DNA, 335 bp, Homo sapiens
taacacttggctcttggtacctgtgggttagcatcaagttctccccagggtagaattcaatcagagctccagtttgcatttggatgtgtaaattacagtaatcccatttcccaaacctaaaatctgtttttctcatcagactctgagtaactggttgctgtgtcataacttcatagatgcaggaggctcaggtgatctgtttgaggagagcaccctaggcagcctgcagggaataacatactggccgttctgacctgttgccagcagatacacaggacatggatgaaattcccgtttcctctagtttcttcctgtagtactcctcttttagatcc

SEQ ID NO: 11 - DNA, 525 bp, Homo sapiens
gaggatggcacaagcgattcacgtaggatctgcccctgtgaccaaaacacctcccattgggccccacttccaacactggtgatcacatttcaacatgaggtttagggaaacaaatgcctaaactacagcactgtacataaactaacaggaaatgctgcttttgatcctcaaagaagtgatatagccaaaattgtaatttaagaagcctttgtcagtatagcaagatgttaactatagaatcaatctaggagtattcactgtaaaattcaacttttctgtatgtttgaacattttcacaatctcataggagtttttaaaaagaagagaaagaagatatactttgctttggagaaatctactttttgacttacatgggtttgctgtaattaagtgcccaatattgaaaggctgcaagtactttgtaatcactctttggcatgggtaaataagcatggtaacttatattgaaatatagtgctcttgctttggataactgtaaagggacccatgctgatagactggaaa

SEQ ID NO: 12 - DNA, 445 bp, Homo sapiens
aagttcattcttaagcttgctttttttgagactggtgtttgttagacagccacagtcctgtctgggttagggtcttccacatttgaggatccttcctatctctccatgggactagactgctttgttattctatttattttttaatttttttcgagacaggatctcactctgttgcccaggatggagtgcagtggtgagatcacggctcattgcagcctcgacctcccaggtgatcctcccacctcagcttccagattagctggtgctataggcatgcaccaccacgtccatctaaatttctttattatttgtagagatgaggtcttgccatgttacccaggctggtctcaactcctgggctcaagcgatcctcctgcctcagtctctcaaagtgctgggattacaggtgtgagccactgtgcccagcctaattgcagtaagacaa

SEQ ID NO: 13 - DNA, 526 bp, Homo sapiens
tgcctctcgcgcatggaggacgagaacaaccggctgcggctggagagcaagcggctgggtggcgacgacgcgcgtgtgcgggagctggagctggagctggaccggctgcgcgccgagaacctccagctgctgaccgagaacgaactgcaccggcagcaggagcgagcgccgctttccaagtttggagactagactgaaacttttttgggggagggggcaaaggggactttttacagtgatggaatgtaacattatatacatgtgtatataagacagtggacctttttatgacacataatcagaagagaaatccccctggctttggttggtttcgtaaatttagctatatgtagcttgcgtgctttctcctgttcttttaattatgtgaaactgaagagttgcttttcttgttttcctttttagaagtttttttccttaatgtgaaagtaatttgaccaagttataatgcatttttgtttttaacaaatcccctccttaaacggagctataaggtggccaaatctga

SEQ ID NO: 14 - DNA, 480 bp, Homo sapiens
acctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcaggggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggagaggcatgctgcggaggtgcgcaggaacaaggaactccaggttgaactgtctggctgaagcaagggagggtctggcacgcc

SEQ ID NO: 15 - DNA, 508 bp, Homo sapiens
  misc_feature (121)..(121): n is a, c, g, or t
accaatcacgcctacagtgctttgaaggtttcctctcctaggctagtttcaaacaggccctaaacaagtctgctgctgccctctcatcagacctccgcaccctcaccccaccatcacttanactactttaatccagttccttcaaagtgatacccccacaggtaagccctcagcatcctgaatacatcatccgcagcctgggaaccttctccctcgtacagcacaggaacctgacacatagtaggcacacagtaaacgtttgtgaatgaatgggagtcatccagtcctgactcttctgtctcttgaggtcccttgaatcttccgcttcctccccaccgatttcagcgtgtccacatcacagctccctccagaagctgcaagagcttcttagcagttcctggtctgaaccctctcccagtcctcatcttccaccctaaaactagagtgatcttcctaaaacttcacttaacccctcagctatgaaaaggcttccaggagtttccatgaa

SEQ ID NO: 16 - DNA, 526 bp, Homo sapiens
gtccacattcctgcaagcattgattgagacatttgcacaatctaaaatgtaagcaaagtagtcattaaaaatacaccctctacttgggctttatactgcatacaaatttactcatgagccttcctttgaggaaggatgtggatctccaaataaagatttagtgtttattttgagctctgcatcttaacaagatgatctgaacacctctcctttgtatcaataaatagccctgttattctgaagtgagaggaccaagtatagtaaaatgctgacatctaaaactaaataaatagaaaacaccaggccagaactatagtcatactcacacaaagggagaaatttaaactcgaaccaagcaaaaggcttcacggaaatagcatggaaaaacaatgcttccagtggccacttcctaaggaggaacaaccccgtctgatctcagaattggcaccacgtgagcttgctaagtgataatatctgtttctactacggatttaggcaacaggacctgtacattgtcacattgcat

SEQ ID NO: 17 - DNA, 456 bp, Homo sapiens
cacaaaggataccagggccctacggaaggctctgacccatctggaaatgcggcgagctgctcgccgacccaacttgcccctgaaggtgaagccaacgctgattgcagtgcggccccctgtccctctacctgcaccctcacatcctgccagcaccaatgagcctattgtcctggaggactgagcacctgtggggaagggaggtgggctgagaggtagagggtggatgcccagggcacccaaacctcccttccctttcgtgtcgaagggagtgaggagtgaattaaggaagagagcaagtgagtgtgtgtccctggaggggttgggcgccctctggtgttaccacctcgagacttgtctcatgcctccatgcttgccgatggaggacagactgcaggaacttggcccatgtgggaacctagcctgttttggggggtaggacccacagatgtcttggac

SEQ ID NO: 18 - DNA, 475 bp, Homo sapiens
gaaattctttcccagtctgtcgatttatgcctcagccacttgcctgtgctacaattcattgtgttacctgtagattcaggtaatacaaaccatatataatcatcaagtaatacaaactaatttagtaatagcctgggttaagtattattagggccctgtgtctgcatgtagaaaaaaaaattcacatgatgcacttcaaattcaaataaaaatccttttggcatgttcccatttttgcttagctcaattagtgtggctaaccaagagataactgtaaatgtgacattgatttgctcttactacagctacagtgattgggggaggaaaagtcccaacccaatgggctcaaacttctaaggggtactcctctcatccccttatccttctccctcgacattttctccctctttcttcccatgaccccaaagccaagggcaacagatcagtaaagaacgtggtcagagtagaacccctg

SEQ ID NO: 19 - DNA, 466 bp, Homo sapiens
gaatatcacagcttaccttgggaatactactgacaatttctttaaaatttccaacctgaagatgggtcataattacacgttcaccgtccaagcaagatgcctttttggcaaccagatctgtggggagcctgccatcctgctgtacgatgagctggggtctggtgcagatgcatctgcaacgcaggctgccagatctacggatgttgctgctgtggtggtgcccatcttattcctgatactgctgagcctgggggtggggtttgccatcctgtacacgaagcaccggaggctgcagagcagcttcaccgccttcgccaacagccactacagctccaggctggggtccgcaatcttctcctctggggatgacctgggggaagatgatgaagatgcccctatgataactggattttcagatgacgtccccatggtgatagcctgaaagagctttcctcactagaaacca

SEQ ID NO: 20 - DNA, 432 bp, Homo sapiens
  misc_feature (307)..(307): n is a, c, g, or t
gagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataatngtggagaggagagaatgagagagtgtggaaaaaaaaagaataatgacccggcccccgccctctgcccccagctgctcctcgcagttcggttaattggttaatcacttaacctgcttttgtcactc

SEQ ID NO: 21 - DNA, 530 bp, Homo sapiens
aagcggcgcaaccaggagatgcagcagaagttggtggagctgtcggctgagaacgagaagctgcaccagcgcgtggagcagctcacgcgggacctggccggcctccggcagttcttcaagcagctgcccagcccgcccttcctgccggccgccgggacagcagactgccggtaacgcgcggccggggcgggagagactcagcaacgacccatacctcagacccgacggcccggagcggagcgcgccctgccctggcgcagccagagccgccgggtgcccgctgcagtttcttgggacataggagcgcaaagaagctacagcctggacttaccaccactaaactgcgagagaagctaaacgtgtttattttcccttaaattatttttgtaatggtagctttttctacatcttactcctgttgatgcagctaaggtacatttgtaaaaagaaaaaaaaccagacttttcagacaaaccctttgtattgtagataagaggaaaagactgagcatgctcacttttttatattaa

SEQ ID NO: 22 - DNA, 544 bp, Homo sapiens
tgttccggaaggacatggcctccaactacaaggagctgggcttccagggctaggcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtgcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggca

SEQ ID NO: 23 - DNA, 516 bp, Homo sapiens
ctgtggtgcatggcagcggaggccctgagccgagggtgggccctgtggcaggccctgcttgccctgctctgctggctctggcatgggctggctcaccctgccagctggctacagcccctgggcccgccagccaccccgcctggctcaccaccctgcagtttgctcctggacagcagcctctccagcaactgggatgacgacagcctagggccttcactctcccctgaggctgtcctggcccggactgtggggagcacctccaccccccggagcaggtgcacacccagggatgccctggacctaagtgacatcaactcagagcctcctcggggctccttcccctcctttgagcctcggaacctcctcagcctgtttgaggacaccctagacccaacctgagccccagactctgcctctgcacttttaaccttttatcctgtgtctctcccgtcgcccttgaaagctggggcccctcgggaactcccatggtcttctctgcctggccgtgtctaataa

SEQ ID NO: 24 - DNA, 488 bp, Homo sapiens
gaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtcca

SEQ ID NO: 25 - DNA, 361 bp, Homo sapiens
ttcaagaaccggtttccaaagacagtcttctaattcctcattagtaataagtaaaatgtttattgttgtagctctggtatataatccattcctcttaaaatataagacctctggcatgaatatttcatatctataaaatgacagatcccaccaggaaggaagctgttgctttctttgaggtgatttttttcctttgctccctgttgctgaaaccatacagcttcataaataattttgcttgctgaaggaagaaaaagtgtttttcataaacccattatccaggactgtttatagctgttggaaggactaggtcttccctagcccccccagtgtgcaagggcagtgaagacttgattgtaca

SEQ ID NO: 26 - DNA, 302 bp, Homo sapiens
cctccctatcgtctgaacagttgtcttcctcagcctcctcccgcccccaccttgggaatgtaaatacaccgtgactttgaaagtttgtacccctgtccttccctttacgccactagtgtgtaggcagatgtctgagtccctaggtggtttctaggattgatagcaattagctttgatgaacccatcccaggaaaaataaaaacagacaaaaaaaaaggaaagattggttctcccagcactgctcagcagccacagcctccctgtatgcctgtgcttggtctactgataagccctctacaaaa

SEQ ID NO: 27 - DNA, 385 bp, Homo sapiens
ttccttttgtagattcccagtttattttctaagactgcaaagatcactttgtcaccagccctgggacctgagaccaagggggtgtcttgtgggcagtgagggggtgaggagaggctggcatgaggttcagtcattccagtgagctccaaagaggggccacctgttctcaaaagcatgttggggaccaggaggtaaaactggccatttatggtgaacctgtgtcttggagctgacttactaagtggaatgagccgaggatttgaatatcagttctaaccttgatagaagaaccttgggttacatgtggttcacattaagaggatagaatcctttggaatcttatggcaaccaaatgtggcttgacgaagtcgtggtttcatctctt

SEQ ID NO: 28 - DNA, 502 bp, Homo sapiens
gcaagcaccccaagttcgaggagatcctcacccgcctgcgtctgcagaagaggggtacaggtgcggtggacacagctgccgtgggctcagtatttgacgtgtccaacgctgatcggctgggctcgtccgaagtagaacaggtgcagctggtggtggatggtgtgaagctcatggtggaaatggagaagaagttggagaaaggccagtccatcgacgacatgatccccgcccagaagtaggcgcctgcccacctgccaccgactgctggaaccccagccagtgggagggcctggcccaccagagtcctgctccctcactcctcgccccgccccctgtcccagagtccacctgggggctctctccacccttctcagagttccagtttcaaccagagttccaaccaatgggctccatcctctggattctggccaatgaaatatctccctggcagggtcctcttcttttcccagagctcctccccaaccaggagctctagttaatg

SEQ ID NO: 29 - DNA, 338 bp, Homo sapiens
tgtttggctgtagcagtgcggccctggctgtgcatggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaaccccatggagctgtcttattgatgctagaagcctcataactgttctacctc

SEQ ID NO: 30 - DNA, 406 bp, Homo sapiens
gataaaacggcaacacagctcacaagaacagactttccagctgctgaagttatggaaacatcaaaacaaagcccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagc

SEQ ID NO: 31 - DNA, 400 bp, Homo sapiens
agagaggtgctattcaagtgattctgaaggcaccccaaggtatatctgtaatttaaagattactgcaaatatctttactttactgtgggtttttagtacatctgttaatttagtgtttctttgtgtgttttgtagactagtgttcttccatccttcaactgagctcaaagtaggttttgttgtaacattgtgattaggatttaaactaattcagagaattgtatcttttactgtacatactgtattctttaagttttaatttgttgtcatactgtctgtgctgatggcttggcttaagattttgatgcataaatgaggtcactgttgatcagtgttgctagtagcttggcagctcttcataaaagcatattgggttggaaaggtgtttgcctatttttca

SEQ ID NO: 32 - DNA, 506 bp, Homo sapiens
aatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcagtagactcctctttgtcacttctctggagatccagcattccttatttggaaatgactttgtttatgtgtctatccctggtaatgatgttgtagtgcagcttaatttcaattcagtctttactttgccactag

SEQ ID NO: 33 - DNA, 427 bp, Homo sapiens
ttccctccacctccaagacaggtggcggccgggcaggcactcttaagcccacctccccctcttgttgccttcgatttcggcaaagcctgggcaggtgccaccgggaaggaatggcatcgagatgctgggcggggacgcggcgtggcgagggggcttgacggcgttggcggggctgggcacaggggcagccgcagggaggcagggatggcaaggcgtgaagccaccctggaaggaactggaccaaggtcttcagaggtgcgacagggtctggaatctgaccttactctagcaggagtttttgtagactctccctgatagtttagtttttgataaagcatgctggtaaaaccactaccctcagagagagccaaaaatacagaagaggcggagagcgcccctccaaccaggctgttattcccctggactc

SEQ ID NO: 34 - DNA, 547 bp, Homo sapiens
gtacatgggactatgcttttctcaaagccccattaactgcttcctataattttgatagtgggaccacatacgtaaaaatctctcatttgtgtggagtcatttctgatttcaggggagatccttgtgtttatcagaaagggcagaagtaggggaagaataatttggtatccttatctagtgtttgattgtcaatgctggagaaaaatatctgtaagagtgtttatacagtacacttcagttatcttgatctccctttcctatatgatgatttgcttaaatatccatattaagtaagtctcaaggtagggtaggcagcctgagagtctagaggcctttagttataaaggaatctagccagtgaacataattcttattactagactgccacaaggaagaaattaacttaccctgtatatcagggtacaaaaaattcagtgatgtgcctaaataagttataaagatttaggccaatcagaagctaacagcagtttcaggtagaggtgcatgcctaatgttagttagtgtagattccatttactgcattctt

SEQ ID NO: 35 - DNA, 457 bp, Homo sapiens
tttcccctagttgacctgtctataagagaattatatatttctaactatataaccctaggaatttagacaacctgaaatttattcacatatatcaaagtgagaaaatgcctcaattcacatagatttcttctctttagtataattgacctactttggtagtggaatagtgaatacttactataatttgacttgaatatgtagctcatcctttacaccaactcctaattttaaataatttctactctgtcttaaatgagaagtacttggttttttttttcttaaatatgtatatgacatttaaatgtaacttattattttttttgagaccgagtcttgctctgttacccaggctggagtgcagtgggtgatcttggctcactgcaagctctgccctccccgggttcgcaccattctcctgcctcagcctcccaattagcttggcctacagtcatctgcc

SEQ ID NO: 36 - DNA, 507 bp, Homo sapiens
ggaaagcaggattccatcgctggaacaattacatgatggactggaaaaatcaatttaacgattacactagcaagaaagaaagttgtgtgggtctctaattaatagatttaccctttatagaacatattttcctttagatcaaggcaaaaatatcaggagcttttttacacacctactaaaaaagttattatgtagctgaaacaaaaatgccagaaggataatattgattcctcacatctttaacttagtattttacctagcatttcaaaacccaaatggctagaacatgtttaattaaatttcacaatataaagttctacagttaattatgtgcatattaaaacaatggcctggttcaatttctttctttccttaataaatttaagttttttccccccaaaattatcagtgctctgcttttagtcacgtgtattttcattaccactcgtaaaaaggtatcttttttaaatgaattaaatattgaaacactgtacaccatagtttaca

SEQ ID NO: 37 - DNA, 550 bp, Homo sapiens
gaggagaacactagacatgccaactcgggagcattctgcctgcctgggaacggggtggacgagggagtgtctgtaaggactcagtgttgactgtaggcgcccctggggtgggtttagcaggctgcagcaggcagaggaggagtacccccctgagagcatgtgggggaaggccttgctgtcatgtgaatccctcaatacccctagtatctggctgggttttcaggggctttggaagctctgttgcaggtgtccgggggtctaggactttagggatctgggatctggggaaggaccaacccatgccctgccaagcctggagcccctgtgttggggggcaaggtgggggagcctggagcccctgtgtgggagggcgaggcgggggagcctggagcccctgtgtgggagggcgaggcgggggatcctggagcccctgtgtcggggggcgagggaggggaggtggccgtcggttgaccttctgaacatgagtgtcaactccaggacttgcttccaagcccttccctctgttggaaattgggtgtgccctggctcc

SEQ ID NO: 38 - DNA, 421 bp, Homo sapiens
tgcttccagccttcgtaattagacttcaccctgagtacacacacaatcactgccactctcactatagacaaaccacactccctcctctgtcacccagtcactgccatctcaacacacatccccaccctgtgtacacacaatctctgttattcatactctcactccttatgcgcactctcaacagggcatgtagtctgcactcaagcatgccatcccagcctcaccctgcattttattcggctcatcccattttccctgaacattttcgctgaactagggccctggcaggatgctgggactgtgcaaggaggtaggacctatgcccacggagctaagagacaggaacacaggctcatctcccgcactaaccaacccctgggatggctcacagcctgctcccagtgctgtgtcatgacctgaa

SEQ ID NO: 39 - DNA, 501 bp, Homo sapiens
atgcttgcccaacacactgtgaaatagttaccaaaatttgtacaaatgcagcatcttcattctttctgagaagacaagatggttttctttacatgaacaaatgaacaaaagagatcctagatccataacgtagctaaggcatctaagagtttgctgttgataatcttgctgaccaaaaactactggagagtaacacaggttatatgccatcacaaatacaatgctcatgaagaactgatttgtagagtcaatgaacctgtgtccagaattttaataggctctctattggaaggagaaagaatttcaagttaacagtatctaactttatcatagttgatgttagtaaattttaaaaaatgattttatatgtatgacaaaaatctttgtaaaatgcgcaagtgcaataatttaaagaggtcttaactttgcatttataaattataaatattgtacatgtgtgtaattttttcatgtattcatttgcagtctttgtatttaaaa

SEQ ID NO: 40 - DNA, 530 bp, Homo sapiens
tttccattcccaatctagtgctagatgtataaatctttcttttgattcttcctaacaaaatattttctgggttaaaaccccagccaactcattgggttgtagccaaaggttcactctcaagaagctttaatatttaaataaaatcatattgaatgtttccaacctggagtataatattcagatataaaacagttttgtcagtctttcttagtgcctgtgtggatttttgtgaaaatgtcaaagagaaaacttatatactatttcccttgaaattttaaactatattttctttacaggtatttataatataccaatgcttttatcaaacagaattttaaagagcataataaattatattaaagaaccaaaagttttcctgagaataagaaagtttcacccaataaaatatttttgaaaggcatgttcctctgtcaatgaaaaaaagtacatgtatgtgttgtgatattaaaagtgacatttgtctaatagcctaatacaacatgtagctgagtttaacatgtgtggtcttg

SEQ ID NO: 41 - DNA, 471 bp, Homo sapiens
gaacctaggagagtcaacatctggaggattttagtctttcttacacatatgtgtgattttaaacgaatattctcagaccacaggaaactcttcatccccctgttgtttaccagtaacagtatatcacagacctttccaaatgtttgtatatgtaatcagatgtacatttatattgaaaaacaaatgagatggacttaaagagcacatcctgataaatactttctctctcacctgtactatatttctattagactaaagttatgtgattttttttttacattttttcagatgactagcaattttgatagtttataagataatgcaaagaactttctctgacaaactaactgcagtaacagaaacctttcttttcagttactctttttcaagaatgaaagattattatacaaaaaattgtatactacttgatggaaccaactttgtacatcttggccatgtcactggtcattg

SEQ ID NO: 42 - DNA, 416 bp, Homo sapiens
catgctaggctttctcagtggggaaaaaaatggctggatagaactgggacaaacacagacccatctttaggggtctggattttgtaggtccgactacacagcagtgttaactcatttctcatgccattagctctctacaaaataaagcaaagtagttctagtgtggtcgttataaaccaatattgtgaaaaatagcaactattcatttgttcacaacatgcgtatttatagagtagttaggtaccatttgtaaggtaaatcctttaaaattctataatacatactaaaatagtggttattggtctgatatatgctgctcttggttctataaactagataaaagcagtgctttgtgaaatgcagtgttctctcttaacgccactggtgataggaagtagttcccttcagttcaaatc

SEQ ID NO: 43 - DNA, 471 bp, Homo sapiens
  misc_feature (200)..(200): n is a, c, g, or t
ttcctcccctgtagggtttggacagacccacccccagccttgcccagctttcaaaggacaaaagggagcatcccccacctactctcaggtttttgaggaaacaaagatttgtggtaactgaaggtgttgggtcagtggccaggtgccgacactgagctgtgacccagaggggacgctgaggaagtgggcgtgagtggacntgtcaggtggttaccaggcactggttgttgatggtcggtggttgggtgtgggcagtcatcagtcatcaggtgtgctcaggggacaatctcccctcaaccgcacatgtgccactgttcagcggagctgactggtttcncctggtagagggnccggctgtttcctgacagatgcctggtgagcaggggaagcaggacccagtggtcancaggtgtctttaactgtcattgtgtgtggaatgtcgcagactcctccacgtggcgggaatgagct

SEQ ID NO: 44 - DNA, 489 bp, Homo sapiens
gcaccacgacgatgacgttcacttgttttgtgtttttcgatctcttcaacgccttgacctgccgctctcagaccaagctgatatttgagatcggctttctcaggaaccacatgttcctctactccgtcctggggtccatcctggggcagctggcggtcatttacatccccccgctgcagagggtcttccagacggagaacctgggagcgcttgatttgctgtttttaactggattggcctcatccgtcttcattttgtcagagctcctcaaactatgtgaaaaatactgttgcagccccaagagagtccagatgcaccctgaagatgtgtagtggaccgcactccgcggcaccttccctaatcatctcgatctggttgtgactgtggcccctgccgtgtctcctcgtcaggggagacttttaggaggccgcagccttccatcaccggatcagtttttcctcttaggaaagctgcaggaacctcgtgggc

SEQ ID NO: 45 - DNA, 546 bp, Homo sapiens
  misc_feature (76)..(76): n is a, c, g, or t
gtggctttcctaggaatgggtcgtacaaagctaagtggtaatgatgctatttggggaaaggtcttttttgcttaantttgttttttaaaactctgatgattncttgagcaacaggcaggttatctgcctggttgaattctggttgaaccgtgtattctaatatttctggttaagtggtgactgggtaaggaaaccacttggggtagcagttcaacaattcacttacgaatgtttataagctttccatttcctaggtaattttttaaaagccagtcaaaacaaaaactttactgaaaatggacagaaataggaaatggactttttccttactgtctatacctcctgaaccttggtattgtaaagatctggggacctctgggtctgttctgaccattccctagtctccatggccaagcactcaaggattgatggacaccacacaccagctatattcatttgccaagatcaacagctccttctccaaacaactcaagcccccaattccnatcgcattcnnttngggtgagatgcaactaacagcccctt

SEQ ID NO: 46 - DNA, 520 bp, Homo sapiens
  misc_feature (48)..(48): n is a, c, g, or t
gcaggctagatccgaggtggcagctccagcccccgggctcgccccctngcgggcgtgccccgcgcgccccgggcggccgaaggccgggccgccccgtcccgccccgtagttgctctttcggtagtggcgatgcgccctgcatgtctcctcacccgtggatcgtgacgactcgaaataacagaaacaaagtcaataaagtgaaaataaataaaaatccttgaacaaatccgaaaaggcttggagtcctcgcccagatctctctcccctgcgagccctttttatttgagaaggaaaaagagaaaagagaatcgtttaagggaacccggcgcccagccaggctccagtggcccgaacggggcggcgagggcggcgagggcgccgaggtccggcccatcccagtcctgtggggctggccgggcagagaccccggacccaggcccaggcctaacctgctaaatgtccccggacggttctggtctcctcggccactttcagtgcgtcggttcgttttgattctttt

SEQ ID NO: 47 - DNA, 545 bp, Homo sapiens
tgcagttttgcatgtaatcggttatacctttattggacttttatagacattttttatttgcatgaaaaaaactcactaaatttacatcactaaacaaaggttaacccttgtgtgaaatgaaggaactgtcaataattgacagccaactaatacagtaaactgttatactagttttgagctttagacctcagccttttgtgtggaagaagtcacagctttcttaggctttaaaggaaaagaaggaaggacttaaatagcttttcttcctaccgggattacctatgtttttccttgcttgcaatctcatctgattttgctagaaatcacaaccatattgtttatgcatattgcatgagtattaccaagaaaaaaatctttaaaagttgtgatgtgacatgatataaaggatctctttatgttaaatgtctttccatgtacctctggtgtgtcagggattttgtgcctcaaaaaatgtttccaaggttgtgtgtttatactgtgtattttttttaaattcacggtgaacagcacttttattatttcca

SEQ ID NO: 48 - DNA, 539 bp, Homo sapiens
aggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctcc

SEQ ID NO: 49 - DNA, 542 bp, Homo sapiens
gtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgt

SEQ ID NO: 50 - DNA, 329 bp, Homo sapiens
aaagcccaacatcccatggctgtttctcacagatcccaaattggccatggaagtttattttggcccttgtagtccctaccagtttaggctggtgggcccagggcagtggccaggagccagaaatgccatgctgacccagtgggaccggtcgttgaaacccatgcagacacgagtggtcgggagacttcagaagccttgcttctttttccattggctgaagctctttgcaattcctattctgttaatcgctgttttccttgtgttgacctaatcatcattttctctaggatttctgaaagttactgacaatacccagacaggggctttgc

SEQ ID NO: 51 - DNA, 438 bp, Homo sapiens
taattacgtctgaggctggaagctgggaaacccaataaatgaactcctttagtttattacaacaagaagacgttgtgatacaagagattcctttcttcttgtgacaaaacatctttcaaaacttaccttgtcaagtcaaaatttgttttagtacctgtttaaccattagaaatatttcatgtcaaggaggaaaacattagggaaaacaaaaatgatataaagccatatgaggttatattgaaatgtattgagcttatattgaaatttattgttccaattcacaggttacatgaaaaaaaatttactaagcttaactacatgtcacacattgtacatggaaacaagaacattaagaagtccgactgacagtatcagtactgttttgcaaatactcagcatactttggatccatttcatgcaggattgtgttgttttaac

SEQ ID NO: 52 - DNA, 427 bp, Homo sapiens
agcagtggaggagcacacggacctttccccagagcccccagcatcccttgctcacacctgcagtagcggtgctgtccaggtggcttacagatgaacccaactgtggagatgatgcagttggcccaacctcactgacggtgaaaaaatgtttgccagggtccagaaactttttttggtttatttctcatacagtgtattggcaactttggcacaccagaatttgtaaactccaccagtcctactttagtgagataaaaagcacactcttaatcttcttccttgttgctttcaagtagttagagttgagctgttaaggacagaataaaatcatagttgaggacagcaggttttagttgaattgaaaatttgactgctctgccccctagaatgtgtgtattttaagcatatgtagctaatctcttgtgtt

SEQ ID NO: 53 - DNA, 486 bp, Homo sapiens
ttcagcttcatttgtgtcaatgggcaatgacaggtaaattaagacatgcactatgaggaataattatttatttaataacaattgtttggggttgaaaattcaaaaagtgtttatttttcatattgtgccaatatgtattgtaaacatgtgttttaattccaatatgatgactcccttaaaatagaaataagtggttatttctcaacaaagcacagtgttaaatgaaattgtaaaacctgtcaatgatacagtccctaaagaaaaaaaatcattgctttgaagcagttgtgtcagctactgcggaaaaggaaggaaactcctgacagtcttgtgcttttcctatttgttttcatggtgaaaatgtactgagattttggtattacactgtatttgtatctctgaagcatgtttcatgttttgtgactatatagagatgtttttaaaagtttcaatgtgattctaatgtcttcatttcattgtatgatg

SEQ ID NO: 54 - DNA, 520 bp, Homo sapiens
ctgtctgacacggactgcaataccagaaagttctgcctccagccccgcgatgagaagccgttctgtgctacatgtcgtgggttgcggaggaggtgccagcgagatgccatgtgctgccctgggacactctgtgtgaacgatgtttgtactacgatggaagatgcaaccccaatattagaaaggcagcttgatgagcaagatggcacacatgcagaaggaacaactgggcacccagtccaggaaaaccaacccaaaaggaagccaagtattaagaaatcacaaggcaggaagggacaagagggagaaagttgtctgagaacttttgactgtggccctggactttgctgtgctcgtcatttttggacgaaaatttgtaagccagtccttttggagggacaggtctgctccagaagagggcataaagacactgctcaagctccagaaatcttccagcgttgcgactgtggccctggactactgtgtcgaagccaattgaccagcaatcggcagcatgctcgat

SEQ ID NO: 55 - DNA, 526 bp, Homo sapiens
gccctcttcctttaggcatgtgagaaaatcagcctagcagtttaaaccccactttcctccacttagcaccataggcaagggggcagatcccagagcccctctcaccccccccaccacaggcctgctccttccttagccttggctaagatggtccttctgtgtcttgcaaagactccccaagtggacagggagcccctgggagggcagccagtgagggtggggtgggactgaagcgttgtgtgcaaatccagcttccatcccctccccaacctggcaggattctccatgtgtaaacttcacccccaggacccaggatcttctcctttctgggcatccctttgtgggtgggcagagccctgacccacagctgtgttactgcttggagaagcatatgtaggggcataccctgtggtgttgtgctgtgtctggctgtgggataaatgtgtgtgggaatattgaaacatcgcctaggaattgtggtttgtatataaccctctaagcccctatcccttgtcgatgacagtca

SEQ ID NO: 56 - DNA, 419 bp, Homo sapiens
accaggagtgtcagcttttagaaggatcatggtcatgtgagcttctggtcaccggaagccagaaatactcagctgccatgttgatccacaaaggtgggaggatgtggggaagggggaaagcggtgaggacgcagagtgcaggctgtggcctcggcatcccgcaggaggtccctagaacatgccgtttcatgtcacctgctacagctctcccccagctagtatgatgatccgttttacaaatgcagaaatgatcttaatattcatgaccactggccaggcgaggtggctcacacctgtaatcccagcactttgggaggccaaggcgggtggatcacaaggtcaagagttcgagaccagcctgaccaacgtggtgaaaccccgtctctactaaaaatagaagcattagccgagcctggtgg

SEQ ID NO: 57 - DNA, 390 bp, Homo sapiens
gcgcagagtagctgcttcctggacgtgcgcgcccaggccagtgctgtgagcaggcggggaggaggctgccggaggagcctgagcctggcaggttcccctgccctgaggctgtgagcagctagtggtggcttctcctgcctttttcagggaactgggaaacttaggggactgagctggggagggaggcaggtgggtggtaagagggaaactctggagagcctgcacccaggtactgagtggggagtgtacagaccctgccttgggggttctgggaatgatgcaactggttttactagtgtgcaagtgtgttcatccccaagttctcttttgtcctcacatgcagagttgtgcatgcccctgagtgtgaacaggtttgcctacgttggtgca

SEQ ID NO: 58 - DNA, 504 bp, Homo sapiens
tggtttattgccgtgtgctatgcctttgtgttctcagctctgattgagtttgccacagtaaactatttcactaagagaggttatgcatgggatggcaaaagtgtggttccagaaaagccaaagaaagtaaaggatcctcttattaagaaaaacaacacttacgctccaacagcaaccagctacacccctaatttggccaggggcgacccgggcttagccaccattgctaaaagtgcaaccatagaacctaaagaggtcaagcccgaaacaaaaccaccagaacccaagaaaacctttaacagtgtcagcaaaattgaccgactgtcaagaatagccttcccgctgctatttggaatctttaacttagtctactgggctacgtatttaaacagagagcctcagctaaaagcccccacaccacatcaatagatcttttactcacattctgttgttcagttcctctgcactgggaatttatttatgttctcaacgcagtaattccca

SEQ ID NO: 59 - DNA, 385 bp, Homo sapiens
tagaagtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctccggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatctt

SEQ ID NO: 60 - DNA, 499 bp, Homo sapiens
aagcttcacttcaacttcactacttctgtagtctcatcttgagtaaaagagaacccagccaactatgaagttccttgtctttgccttcatcttggctctcatggtttccatgattggagctgattcatctgaagagaaatttttgcgtagaattggaagattcggttatgggtatggcccttatcagccagttccagaacaaccactatacccacaaccataccaaccacaataccaacaatataccttttaatatcatcagtaactgcaggacatgattattgaggcttgattggcaaatacgacttctacatccatattctcatctttcataccatatcacactactaccactttttgaagaatcatcaaagagcaatgcaaatgaaaaacactataatttactgtatactctttgtttcaggatacttgccttttcaattgtcacttgatgatataattgcaatttaaactgttaagctgtgttcagtactgtttc

SEQ ID NO: 61 - DNA, 464 bp, Homo sapiens
ggtttgttaccatcctttaatcataactaaaacattgaaaacagaacaaatgagaaaagaaaaaaaacctgccgattaacaatgacgaaaatcatgcatgatctgaaaggtgtggaaagaaacacaattaggtctcactctggttaggcattatttatttaattatgttgtatatcattgtttgcagggcaacattctatgcattgaactgagcactaactgggctagcttctggtagacgtttgtggctagtgcgattcacagtctactgcctgttccactgaaacattttgtcatattcttgtattcaaagaaaaaaggaaaaaaagattattgtaaatattttatttaatgcacacattcacacagtggtaacagactgccagtgttcatcctgaaatgtctcacggattgatctacctgtccatgtatgtctgctgagctttctccttggttatgttttt

SEQ ID NO: 62 - DNA, 506 bp, Homo sapiens
taaagagctcatttttcaggtccgccacacctatgaaattcccctggtgctggtgggtaacaaaattgatctggaacagttccgccaggtttctacagaagaaggcttgagtcttgcccaagaatataattgtggtttttttgagacctctgcagccctcagattctgtattgatgatgcttttcatggcttagtgagggaaattcgcaagaaggagtccatgccatccttgatggaaaagaaactgaagagaaaagacagcctgtggaagaagctcaaaggttctttgaagaagaagagagaaaatatgacatgatatctttgcttttgagttcctcacgctctctgaattttattagttggacaattccatatgtagcattctgcttcaatattatctctctatgtgtctctctctctttaaatatctgcctgtaggtaaaagcaagctctgcatatctgtacctcttgagatagttttgttttgcctttaacagttggatgga

SEQ ID NO: 63 - DNA, 436 bp, Homo sapiens
gaggggtcaccgtgcaggatggaaatttctccttttctctggagtcagtgaagaagctcaaagacctccaggagccccaggagcccagggttgggaaactcaggaactttgcacccatccctggtgaacctgtggttcccatcctctgtagcaacccgaactttccagaagaactcaagcctctctgcaaggagcccaatgcccaggagatacttcagaggctggaggaaatcgctgaggacccgggcacatgtgaaatctgtgcctacgctgcctgtaccggatgctaggggggcttgcccactgcctgcctcccctccgcagcagggaagctcttttctcctgcagaaagggccacccatgatactccactcccagcagctcaacctaccctggtccagtcgggaggagcagcccggggaggaactgggtgact

SEQ ID NO: 64 - DNA, 429 bp, Homo sapiens
ctccccccgagagaaggctgcaaagctgggaagcccagggtgtgctcctcccgcccttttggacccccgggcttgcaccggctgcactctgagaaccagctgcgcgcggagcggtgcaatgcagcacccaccctgcgagcctggcaattgcttgtcattaaaagaaaaaaaaattacggagggctccgggggtgtgtgttggggaggggagaccgatgcttctaacccagcccccgctttgactgcgtgttgtgcagctgagcgcgaggccaacgttgagcaaggccttgcagggaggttgctcctgtgtaattacgaaagaaggctagtccgaaggtgcaaaatagcagggagaggacgcgcccccttaggaacaagacctctggatgtttccagtttcaaattgaaagaagaggggcgccccccttg

SEQ ID NO: 65 - DNA, 513 bp, Homo sapiens
acagcagcagttatggccggagcgaccgctactcgaggggccgacaccgggtgggcagaccagatcgtgggctctctctgtccatggaaaggggctgccctccccagcgtgattcttacagccggtcaggctgcagggtgcccaggggcggaggccgtctaggaggccgcttggagagaggaggaggccggagcagatactaagcaggaacagacttgggaccaaaaatcccttttcaacgaaactaacaaaaagaagaacctgttgtatggtaactacccaaggactagtacaaggaagagttgtttttaccttttaagaatttcctgttaagatcgtctccatttttatgcttttgggagaaaaaacttaaaattcgtttagtttagttttggaattgttaacgtttctttcaacaagctcctgttaaaagtatatgaacctgagtactagtcttcttacatttacaagtagaaattcgattaatggcttcttcccttgtaaattttcttg

SEQ ID NO: 66 - DNA, 551 bp, Homo sapiens
cagccagagcattggactgatccagcatttgagaactcatgttagagagaaaccttttacatgcaaagactgtggaaaagcgtttttccagattagacaccttaggcaacatgagattattcatactggtgtgaaaccctatatttgtaatgtatgtagtaaaaccttcagccatagtacatacctaactcaacaccagagaactcatactggagaaagaccatataaatgtaaggaatgtgggaaagcctttagccagagaatacatctttctatccatcagagagtccatactggagtaaaaccttatgaatgcagtcattgtgggaaagcctttaggcatgattcatcctttgctaaacatcagagaattcatactggagaaaaaccttatgattgtaatgagtgtggaaaagccttcagctgtagttcatcccttattagacactgcaaaacacatttaagaaataccttcagcaatgttgtgtgaaatatactaaacatcaaagaatctatgttggagcacaagattctaaatcagtggttccctg

SEQ ID NO: 67 - DNA, 316 bp, Homo sapiens
gagtcactccaggaaagagctgatgaggctacaacccagaagcagtctggggaagacaaccaggaccttgctatctcctttgcaggaaatggactctctgctcttagaacctcaggttctcaggcaagagccacctgctattgccgaaccggccgttgtgctacccgtgagtccctctccggggtgtgtgaaatcagtggccgcctctacagactctgctgtcgctgagcttcctagatagaaaccaaagcagtgcaagattcagttcaaggtcctgaaaaaagaaaaacattttactctgtgtaccttgtgtctt

SEQ ID NO: 68 - DNA, 510 bp, Homo sapiens
gtgacgctcaatctacagtttattcatatattcaagaccatgtatgtgtatctatagccactggttcctccatgagatcagatggaacagacaatgcctatgtggctgatggcaccatgtgtggtccagaaatgtactgtgtaaataaaacctgcagaaaagttcatttaatgggatataactgtaatgccaccacaaaatgcaaagggaaagggatatgtaataattttggtaattgtcaatgcttccctggacatagacctccagattgtaaattccagtttggttccccagggggtagtattgatgatggaaattttcagaaatctggtgacttttatactgaaaaaggctacaatacacactggaacaactggtttattctgagtttctgcatttttctgccgtttttcatagttttcaccactgtgatctttaaaagaaatgaaataagtaaatcatgtaacagagagaatgcagagtataatcgtaattcatccgttgtatcag

SEQ ID NO: 69 - DNA, 344 bp, Homo sapiens
gagccactccaagctgaggatgatccactgcaggcaaaagcttatgaggctgatgcccaggagcagcgtggggcaaatgaccaggactttgccgtctcctttgcagaggatgcaagctcaagtcttagagctttgggctcaacaagggctttcacttgccattgcagaaggtcctgttattcaacagaatattcctatgggacctgcactgtcatgggtattaaccacagattctgctgcctctgagggatgagaacagagagaaatatattcataatttactttatgacctagaaggaaactgtcgtgtgtcccatacattgccatcaactttgtttcctcat

SEQ ID NO: 70 - DNA, 479 bp, Homo sapiens
gctggaggtgacgctactgagaactttgaggatgtcgggcactctacagatgccagggaaatgtccaaaacattcatcattggggagctccatccagatgacagaccaaagttaaacaagcctccagaaccttaaaggcggtgtttcaaggaaactcttatcactactattgattctagttccagttggtggaccaactgggtgatccctgccatctctgcagtggccgtcgccttgatgtatcgcctatacatggcagaggactgaacacctcctcagaagtcagcgcaggaagagcctgctttggacacgggagaaaagaagccattgctaactacttcaactgacagaaaccttcacttgaaaacaatgattttaatatatctctttctttttcttccgacattagaaacaaaacaaaaagaactgtcctttctgcgctcaaatttttcgagtgtgcctttttattcatctacttt

SEQ ID NO: 71 - DNA, 541 bp, Homo sapiens
gagctcaagccagcatagctccaccaagtgatctactgttccaaatctctataaccacctgcttcccactcagcctgcaatagtgtttcccactctctgcttggcatcaatagatgcataagggtcaaccacatttttcctcaagttccctggagaagaagctgaactcctggtttctccatccccatgaccttcccagggccatggaggtcctgctgctggtctgggatgatgatgcccctggaaaccttcctgcaatggccccttactttggacagcaacccctgagcccaagccagttttggccttcacagcctggccggttcccactctggcccatctcccattcttactgggagttggagatttgaagccagtcatctcagcactgtctgaggagggcagagccatgggttctgtgctggagggtgcacggccaagatctccagactgctggttcccagggaaccctccctacatctgggcttcagatcctgactcccttctgtcccctaattccctgagctgtagatcctctggt

SEQ ID NO: 72 - DNA, 547 bp, Homo sapiens
cgcacccgcatcacaggggaggaggtggaggtgcaggactccgtgcccgcagactccggcctctatgcttgcgtaaccagcagcccctcgggcagtgacaccacctacttctccgtcaatgtttcagcttgcccagatctccaggaggctaagtggtgctcggccagcttccactccatcactcccttgccatttggacttggtactcggcttagtgattagaggccctgaacaggtggtggtatccctgctctgctggagaggaacccagatgctctcccctcctcggaggatgatgatgatgatgatgactcctcttcagaggagaaagaaacagataacaccaaaccaaaccccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctgga

SEQ ID NO: 73 - DNA, 407 bp, Homo sapiens
ctgccctgtacatgctagttcaacagaaaggaatggcctttcaccttctcctggtggcaggcaagcagatgtcctctgcggagataccgccagctccccaggacgcagactgactcctgtttgctcgctggaccaaccccaggcagaaggtggaaggtgggaacagaggtttagctgcaggacatgtattcccattgcaccgagacctaactgccgctcagagtgtagaccgagatggtgcagatgcctgcagtgccattaaaatgtgggtgaaggtgacatcaggattatgtgccccaggccgggctcagtggctcacacctgtaatcccagcactttgggaggccaaggtgggcggatcacctgaggtcaggagtttgcgacaagcctgccaacaagctgaaacc

SEQ ID NO: 74 - DNA, 533 bp, Homo sapiens
gaaatctctgatataagctgggtgtggtggctcgtgcctgtagtctcagctgctgggcaactgcagaccagcctgggcaacatagtaagaccctgtctcaaaaaaataatctctggtacaatggtcatgttccaaagttccttacttgggcctcttgagtgcagtggctcacacctggaatcccagtgctttgagaggctgaggaggcaggaggttcacttgtgcccaggaatttgaggctgcagtgagctatgattgtgccactgcactccagcctgggtgacagagcaagactgtgctctcttaaaaataagaaagagcctcttcatcttcaaaaggactacatctgaagtttccccagaaggacaaatgtctacttagaccttataaatttccaaaataagagagtcagagccagaggtggcttgtaagttgacttctgttgagatctgaccacatttgatctcttgttttaattttccaactaactgaacttggaagaaaacccaaaccaagttttaatctgatgccta

SEQ ID NO: 75 - DNA, 564 bp, Homo sapiens
ccatgagcaacttccagagctggacaacttgggcctggatagcttttccagtggacctggggaagaggctttgttgcagatgagatcaaacatcatctatgactccactgcccgaatcagaaggaacgccaaaggaaactactgtaagaggaccccgctctacatcgacttcaaggagattgggtgggactcctggatcatcgctccgcctggatacgaagcctatgaatgccgtggtgtttgtaactaccccctggcagagcatctcacacccacaaagcatgcaattatccaggccttggtccacctcaagaattcccagaaagcttccaaagcctgctgtgtgcccacaaagctagagcccatctccatcctctatttagacaaaggcgtcgtcacctacaagtttaaatacgaaggcatggccgtctccgaatgtggctgtagatagaagaagagtcctatggcttatttaataactgtaaatgtgtatatttggtgttcctatttaatgagattatttaataagggtgtacagtaatagaggcttgctgccttcaggaa

SEQ ID NO: 76 - DNA, 533 bp, Homo sapiens
atgatctgcatgtttctggtggcatggtccccttattccatcgtgtgcttatgggcttcttttggtgacccaaagaagattcctccccccatggccatcatagctccactgtttgcaaaatcttctacattctataacccctgcatttatgtggttgctaataaaaagtttcggagggcaatgcttgccatgttcaaatgtcagactcaccaaacaatgcctgtgacaagtattttacccatggatgtatctcaaaacccattggcttctggaagaatctgaaataagagaaaaggacacgctatcaaaacactttagttttttgacaatgcttttcttttaaatatgagcccatttagatcaagtgcagacatggatcattgtcctatgagagtgtaagctcctcaagcacagctcgtgcttccgtttgtgcactctggctgctgtagtgtatgcttctctgtgtcctgatatatcaacttattgctcatctcctttgatgaattaggcatcagaggttaaggtcccctttc

SEQ ID NO: 77 - DNA, 510 bp, Homo sapiens
gaacaggagagttcccaggccagtacggaagaatgtgagaaaaataagcaggacacaattacaactaaaaaatatatctaagcatttgcaaaggcgacaataaattattgacgcttaacctttccagtttataagactggaatataatttcaaaccacacattagtacttatgttgcacaatgagaaaagaaattagtttcaaatttacctcagcgtttgtgtatcgggcaaaaatcgttttgcccgattccgtattggtatacttttgcttcagttgcatatcttaaaactaaatgtaatttattaactaatcaagaaaaacatctttggctgagctcggtggctcatgcctgtaatcccaacactttgagaagctgaggtgggaggagtgcttgaggccaggagttcaagaccagcctgggcaacatagggagacccccatctttacgaagaaaaaaaaaaaggggaaaagaaaatcttttaaatctttggatttgatcactacaagt

SEQ ID NO: 78 - DNA, 531 bp, Homo sapiens
ccgagccgagcttactgtgagtgtggagatgttatcccaccatgtaaagtcgcctgcgcaggggagggctgcccatctccccaacccagtcacagagagataggaaacggcatttgagtgggtgtccagggccccgtagagagacatttaagatggtgtatgacagagcattggccttgaccaaatgttaaatcctctgtgtgtatttcataagttattacaggtataaaagtgatgacctatcatgaggaaatgaaagtggctgatttgctggtaggattttgtacagtttagagaagcgattatttattgtgaaactgttctccactccaactcctttatgtggatctgttcaaagtagtcactgtatatacgtatagagaggtagataggtaggtagattttaaattgcattctgaatacaaactcatactccttagagcttgaattacatttttaaaatgcatatgtgctgtttggcaccgtggcaagatggtatcagagagaaacccatcaattgctcaaatactc

SEQ ID NO: 79 - DNA, 522 bp, Homo sapiens
ttgtggctacaaaggatgggctgaagctggggtctggaccttcaatcaaagccttagatgggagatctcaagtttcaatatcatgttttggcaaaacattcgatgctcccacatccttacctaaagctaccagaaaggctttgggaactgtcaacagagctacagaaaagtcagtaaagaccaatggacccctcaaacaaaaacagccaagcttttctgccaaaaagatgactgagaagactgttaaagcaaaaaactctgttcctgcctcagatgatggctatccagaaatagaaaaattatttcccttcaatcctctaggcttcgagagttttgacctgcctgaagagcaccagattgcacatctccccttgagtgaagtgcctctcatgatacttgatgaggagagagagcttgaaaagctgtttcagctgggccccccttcacctttgaagatgccctctccaccatggaaatccaatctgttgcagtctcctttaagcattctgttgaccctggatg

SEQ ID NO: 80 - DNA, 541 bp, Homo sapiens
ggtttaaggatcagtcctctgcagtttcgctaaggccccctttgtgtgcatgggtcagtcaccatatgttccccccagagaatgtgtctatatcctccttctaacagcaccttccccctgcagctactcttcagatctggctctctgtaccctaaaacctagtatctttttctcttctatggaaaatccgaaggtctaaacttgacttttttgaggtcttctcaacttgactacagttgtgctcataattgtccttgcctttccagcttaattattttaaggaacaaatgaaaactctgggctgggtggagtggctcatacctgtaatcccagcactttgggaggctacggtgggcagatcatctgaggccaggagttcgagacctgcctggccaacatggcaacaccccgtctctaataaaaatataaaaattagcctggcatggtagcatgcgcctatagtcccagctgctcaggaggctgaggcatgagaatcgcttgaacctaggaggtggaggttgcattcaactgagatcatacc

SEQ ID NO: 81 - DNA, 454 bp, Homo sapiens
actggtctatctctatcctgacattcccaaggaggaggcattcggaaagtattgtcggccagagagccaggagcatcctgaagctgacccaggcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagc

SEQ ID NO: 82 - DNA, 533 bp, Homo sapiens
ttgacagctctttaagcccacatgcagcagtgggtcagataaccctgtggcagtgacacgggcaaattggcatttgaataaagccctgggaccacctcaacatgcgtagcctcttgtcttaaatgtactccccatggcagcatggaggaggcaagacctgtgggtcaattttgaactggccttactttgatttttaaaacaagagactcagggaaagtactaaaccaaaatctctgattttactttgcgttttctgtagtttttgttttactgagatgcttttgtaaaggaaaataatactgtgacagtttagtaattctacagattcttaatatttctccatcatggccttttacttcacaattttctgaagtctgaattcaattacaattttttttttttaccaatttaatctcaaatgttgtttaactgctttaaattcatatacgtagagtattataaactgcagagatgaaaaatgtgttttcacgggatttatattgtgaactaaactaagcctactttttgtgact

SEQ ID NO: 83 - DNA, 483 bp, Homo sapiens
gagacttctcacttctggttggaggtttcacatatggctcaactcaagtcattaatctctttttaatttttactcttgaattccttaaacttcgctcattatgaaatgttttaaaattatgacaaaaattactctgtctaaccacttgccttgtctgctaccagtttgttaaaaattattccccccaaccagtaattccaccagtactacttgatttgtgttatatttcctatgtacatgtacagcctttgttttgcttgcttgtctatttttactttcccttttttgggtcaaatttttcttttgctttgtttgaagaaggaatatacagaagtaaaatcttgtcttctctgctgattctttaattaatatgagccggatactttccactgtcttcttggcactttcaggatttcttaatgctgatatatggactcttagaatggaatttttgaagaaaaatctcaaagcctgtatcgttct

SEQ ID NO: 84 - DNA, 529 bp, Homo sapiens
ataggttacccttgaaattcattagtttgtcataaagttttaggaaaggtaggacccggaaagaagttctaattagttgtctaaatatttttcagtgagccaagaaattcaccatgaaaaaacaagaataacaaatagaagggaagagataggatgggaaagctaacaaattaaagttttggcaaaaaggaatatatgtaaatagctaattatttacttttgtgcttactttatttagattatttctatcagttacaatctttttctagttaagtgtacctaatttatggaatgggtgctatcctgtttatgtgtgtcttggtttttcttggctacagaaaaactgttgcagggcaacactagtttgatatttgatttactctccaatgagactcaatggctgggccgtggtagactcatagttcctcttgttctttattaaattcatcctgctaattagatttctagtgacttgtaacatgtagtttacactgaattgcaattacagatgcatacaactactatacta

SEQ ID NO: 85 - DNA, 525 bp, Homo sapiens
ataacagcatatgcatttccccaccgcgttgtgtctgcagcttctttgccaatatagtaatgcttttagtagagtactagatagtatcagttttggattcttattgttatcacctatgtacaatggaaagggattttaagcacaaacctgctgctcatctaacgttggtacataatctcaaatcaaaagttatctgtgactattatatagggatcacaaaagtgtcacatattagaatgctgacctttcatatggattattgtgagtcatcagagtttattataacttattgttcatattcatttctaagttaatttaagtaatcatttattaagacagaattttgtataaactatttattgtgctctctgtggaactgaagtttgatttatttttgtactacacggcatgggtttgttgacactttaattttgctataaatgtgtggaatcacaagttgctgtgatacttcatttttaaattgtgaactttgtacaaattttgtcatgctggatgttaacacat

SEQ ID NO: 86 - DNA, 432 bp, Homo sapiens
tcatgtcttattcttccctgtgaaaccaggattaatcgtggactcctggcagcttaacctagctcagttgcagtgctaagcatgccccgcccccattcagtgatacctgtttgggaagtatatacttccccaaaagtactcttggccctaagttttaggaactttccccgacctggatcccttgtcatacctgtgttactgtttaaagcacacccacccaacttacaagatcttaggctgctgtggtggtgaagcaccttgagtctgctgatattcgggagaacaaggatctgcagtttccccttttctcccctctgaagagtggttcttatgtgcaatctgcagtaaccttgaactccagagctgcactatagaggagaatgcatgccactatgacagcagtatgccaagctttgtgttcatctcctaata

SEQ ID NO: 87 - DNA, 185 bp, Homo sapiens
atttcgttttgcttttggttgcctgaatgttgtcaccaagtgaaaaaattatttaactatatgtaaaatttctcttttaaaaaaaagttttactgatgttaaacgttctcagtgccaatgtcagactgtgctcctccctctcctgaacctctaccctcaccctgagctgtcttgttgaaaacagt

SEQ ID NO: 88 - DNA, 361 bp, Homo sapiens
tattctcgactgtaatggcattgcagtagggccaaaacaagtccaagcttcttaaaatgattggtggttaatttttcaaagcagaaattttaagccaaaaacaaacgaaaggaaagcggggaggggaaaacagaccctcccactggtgccgttgctgcgttctttcaatgctgactggactgtgtttttcctatgcagtgtcagctcctctgtctggttgtttacctgttcctgttcgtgcttgtaatgctcacttatgttttctctgtataacttgtgattccagggctgtttgtcaacagtatacaaaagaattgtgcctctcccaagtccagtgtgactttatcttctgggtggtttg

SEQ ID NO: 89 - DNA, 552 bp, Homo sapiens
gaaatcagcgaggctcaagttccaagcaaaccattccaaaatgtggaattctgtgacttcagtaggcatgaacctgatggggaagcatttgaagacaaagatttggaaggcagaattgaaactgataccaaggttttggagatactatatgagtttcctagagtttttagttctgtcatgaaacctgagaatatgattgtaccaataaaactaagctctgattctgaaattgtacaacaaagcatgcaaacatcagatggaatattgaatcccagcagcggaggcatcaccactacttctgttcctggaagtccagatggtgtctttgatcaaacttgcgtagattttgaagttgagagtgtaggtggtatagccaatagtacaggtttcatcttagatcaaaagatacagattccattcctgcaactatgggtcacatctctctgtcagagagcacaaatgacactgttagtccagtaatgattagagaatgtgagaagaatgacagcactgctgatgagttacatgtaaagcacgaacctcctgatacag

SEQ ID NO: 90 - DNA, 419 bp, Homo sapiens
gggctcaaagcattaatccagttactgaaaagagaatacaagtggagcaaacaagagatgaagatcttgatacagactcattggactgaatttcccccttccccccatgatggaagaatgttcagattctaaattgaggacttcattattaatggcattactgtgttatgattaacaaatttcttgtaaggtacacactacatactaaggtcggccatcattccgtttttttttttttttttttttttaaccaagcttaaaatgaagcttaaaatgaagctttgtgtttgaaagtaataacaagctcagacgaagatggtggttgtacattattcatctagaaaatataaaaattcattttgttttgaagctagttattaaactggaatagcagttatatccctgagaatggggccctt

SEQ ID NO: 91 - DNA, 394 bp, Homo sapiens
gctgctgttttcttctaactgcagggaaaatgctgtctaaaagaaaataataaatttgtatctgctgagttctcttagcataaggcaccaacaaaacaaccttcaggaagggagaagaaaccatcctcccactcatccttcagaggatttagataaagtgaagggaagaatcgttctccagctccttcggaatttacgccggcatcagggcaggcttgttactgctggatccattgtctgctcaaggttacttattccactaagacgtacatcctaccacggaccacggctttgtagctagccaggctctgagtgtgtgtgtagatgaaccatttctctctccagtaaatgaatgacagtctttctagggctcttgtcttctgctgggaggcag

SEQ ID NO: 92 - DNA, 417 bp, Homo sapiens
agtcactctcccagatggacggactctgtttcctggccaaggcaacaattcctacgtgttccctggagttgctcttggggtggtggcctgcggactgagacacatcgatgataaggtcttcctcaccactgctgaggtcatatctcagcaagtgtcagataaacacctgcaagaaggccggctctatcctcctttgaataccattcgagacgtttcgttgaaaattgcagtaaagattgtgcaagatgcatacaaagaaaagatggccactgtttatcctgaaccccaaaacaaagaagaatttgtctcctcccagatgtacagcactaattatgaccagatcctacctgattgttatccgtggcctgcagaagtccagaaaatacagaccaaagtcaaccagtaacgcaacagcta

SEQ ID NO: 93 - DNA, 454 bp, Homo sapiens
gttccacttctctaggtagacaattaagttgtcacaaactgtgtgaatgtatttgtagtttgttccaaagtaaatctatttctatattgtggtgtcaaagtagagtttaaaaattaaacaaaaaagacattgctccttttaaaagtcctttcttaagtttagaatacctctctaagaattcgtgacaaaaggctatgttctaatcaataaggaaaagcttaaaattgttataaatacttcccttacttttaatatagtgtgcaaagcaaactttattttcacttcagactagtaggactgaatagtgccaaattgcccctgaatcataaaaggttctttggggtgcagtaaaaaggacaaagtaaatataaaatatatgttgacaataaaaactcttgcctttttcatagtattagaaaaaaatttctaatttacctatagcaacatttcaaat

SEQ ID NO: 94 - DNA, 435 bp, Homo sapiens
gcatttgaaactgagcactaaactgggctagctttctggtagaccgttttgtggctagtgcgatttcacagtctactgcctgtttccactgaaaacatttttgtcatattcttgtattcaaagaaaacaggaaaaaagttattgtaaatattttatttaatgcacacattcacacagtggtaacagactgccagtgttcatcctgaaatgtctcacggattgatctacctgtctatgtatgtctgctgagctttctccttggttatgttttttctcttttacctttctcctcccttacttctatcagaaccaattctatgcgccaaatacaacagggggatgtgtcccagtacacttacaaaataaaacataactgaaagaagagcagttttatgatttgggtgcgtttttgtgtttatactgggccaggtcctg

SEQ ID NO: 95 - DNA, 352 bp, Homo sapiens
ggcagccttccttgtgatcaaaaaaggtaatcccagaaacgtacccgttcactcgtgggtcttaaaatggtttcatatctctattgtgactaattttctctcggtctactgccttttcaatcaggaatagatttgccatgaagccagtgaagtttttaagtgtctaggcttctcattagtgccaactctcctagacctggtgcctgttttttttccaagttttgtttctacttctatccattttttaaattaaactttttattttgaaataattatcacactcacaagctgtgggaagaaataatagagatcctgtgtctctttcatccagttttcctcaagggtaacatct

SEQ ID NO: 96 - DNA, 521 bp, Homo sapiens
tgctcaacgtgcactacagaaccccgacgacacacacaatgccctcatgggtgaagactgtattcttgaacctgctccccagggtcatgttcatgaccaggccaacaagcaacgagggcaacgctcagaagccgaggcccctctacggtgccgagctctcaaatctgaattgcttcagccgcgcagagtccaaaggctgcaaggagggctacccctgccaggacgggatgtgtggttactgccaccaccgcaggataaaaatctccaatttcagtgctaacctcacgagaagctctagttctgaatctgttgatgctgtgctgtccctctctgctttgtcaccagaaatcaaagaagccatccaaagtgtcaagtatattgctgaaaatatgaaagcacaaaatgaagccaaagaggaacaaaaagcccaagagatccaacaattgaaacgaaaagaaaagtccacagaaacatccgatcaagaacctgggctatgaatttccaatcttcaacaacctgtt

SEQ ID NO: 97 - DNA, 469 bp, Homo sapiens
cagcgctgccagcaggcatacatgcagtacatccaccaccgcttgattcacctgactcctgcggactacgacgactttgtgaatgcgatccggagtgcccgcagcgccttctgcctgacgcccatgggcatgatgcagttcaacgacatcctacagaacctcaagcgcagcaaacagaccaaggagctgtggcagcgggtctcactcgagatggccaccttctccccctgagtctttcacccttagggtcctatacagggacccaggcctgtggctatgggggcccctcacacagggggagtgaaacttggctggacagatcatcctcactcagttccctggtagcacagactgacagctgctcttgggctatagcttggggccaagatgtctcacaccctagaagcctagggctgggggagacagccctgtctgggagggggcgttgggtggcctctggtatttattt

SEQ ID NO: 98 - DNA, 426 bp, Homo sapiens
gtcactcatttccttgaacagcacccccctttatactagcagccatttgtgccattgcctgtgccctagggtttgtggggagagagcgagggatcactgagcagttttcccagagctccatgggaaggcaagctctccctcccaatgggagccccactgtcactaactgtaaactcaggctcaggcttcaactgcctacccccatcctcatatttctgtctgtcccagcacctcaggagcattctcattgtggccggctaactccgcctggatgtgaacaggcaagcacagtgggaaatgagtcacgtacttgtattgcacagtggacacctctagaggtccattggtttaaagggatagggaaggaggagggatgagaccatcaccccctcccagaagtaaatctagtatctgagttttctttat

SEQ ID NO: 99 - DNA, 404 bp, Homo sapiens
  misc_feature (374)..(374): n is a, c, g, or t
caagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagntgccgcaccagcccgaacaccattgaggga

SEQ ID NO: 100 - DNA, 376 bp, Homo sapiens
tttccccttggaagacactattgatctcaacctgctgacttttcctaatgcttacctgaaggaacccatcctggctagaaagggtgatggtactggaccggtattcaaccttgagttttcaagctgccaaacaggtcttaagggaggtgcttatatcccaccaacactctcccagctcccatgtccccaagacctctggagtttcctcttgaatgtacatgaaccactgtaatagcattagacttttaattgagtgtgcaatcgttttccatggagtttggtccgttcattattttttagttaactacacttcttgatattcaaatgttctattaaaaaaactgagtatgaagaaaaacactttactactgcagaa

SEQ ID NO: 101 - DNA, 476 bp, Homo sapiens
  misc_feature (89)..(89): n is a, c, g, or t
tcccccatttacaatccttcatgtattacatagaaggattgcttttttaaaaatatactgcgggttggaaagggatatttaatctttgngaaactattttagaaaatatgtttgtagaacaattatttttgaaaaagatttaaagcaataacaagaaggaaggcgagaggagcagaacattttggtctagggtggtttctttttaaaccattttttcttgttaatttacagttaaacctaggggacaatccggattggccctcccccttttgtaaataacccaggaaatgtaataaattcattatcttagggtgatctgccctgccaatcagactttggggagatggcgatttgattacagacgttcgggggggtggggggcttgcagtttgttttggagataatacagtttcctgctatctgccgctcctatctagaggcaacacttaagcagtaattgctgttgcttgttgtca

SEQ ID NO: 102 - DNA, 330 bp, Homo sapiens
agcctgaaacaggaactcacatgagactcagggccaccaggaaatgcttaaaatacatactctttcccaaaagcaaatctataattctgtttcaattttatgaatatatgaatagacaaaatgaatcgaattacataactatgtcattcattaaatggcaacaatgctgacagcaagcagtagatcctctgattccaattaccatttgttttttacccaattctatttgctagaggtagtaagtactctggcactcataaatcacatgatgataaaaaggaacatgaggccgggtatggtggctcacaactgtaatccccataccttggg

SEQ ID NO: 103 - DNA, 550 bp, Homo sapiens
  misc_feature (276)..(276): n is a, c, g, or t
tatgggtcagttacagcagccctcacctcaaagggctggcctgcttctcagcctacattcatttgcaagcttcaatctctggaccatctggtgttcacaggtgttagagggttaggggttaggggctagttttggatttgattcataggtaggagggcttagattttaaggcacttctgaaagtcaatccctggacaaggcagtcatcacataagaacagctaccttctccacttggtggcacaagaggtagggaggggagtatgggttcatttgncttcgcattatgcaaggtgaaaccgtttgttttccctctccattttccctaactaaatgaaaaggacacattctgaaatcccttttgttggagaataagtcagtctgaggggaaatgggaggccagagatgagaaccctttgaaaagattgtaaaatactgattttcattctttcaagcttatttgtaaatacctatttgaatgctgtgtatttgtacaggaatttgagcaaaaaatgtatagagtgtgatgtccaattggtattcagcactat

SEQ ID NO: 104 - DNA, 555 bp, Homo sapiens
  misc_feature (45)..(50): n is a, c, g, or t
gagcttcgttgatggtcttttctgtactggaggcctcctgaggcnnnnnnagccccaggacccattaagccacccccgtgttcctgccgtcagtgccaactnnnnnatgtggaagcatctacccgttcactccagtcccaccccacgcctgactcccctctggaaactgcaggccagatggttgctgccacaacttgtgtaccttcagggatggggctcttactccctcctgaggccagctgctctaatatcgatggtcctgcttgccagagagttcctctacccagcaaaaatgagtgtctcagaagtgtgctcctctggcctcagttctcctcttttggaacaacataaaacaaatttaattttctacgcctctggggatatctgctcagccaatggaaaatctgggttcaaccagcccctgccatttcttaagactttctgctccactcacaggatcctgagctgcacttacctgtgagagtcttcaaacttttaaaccttgccagtcaggacttttgctattgcaaatagaaaacccaactcaacctgctt

SEQ ID NO: 105 - DNA, 408 bp, Homo sapiens
ctgcctggttaccgtggcgatgtgcttaatgcagcgttgaaaatacagaatactgactcctctgtccctcctggccccggactccctccctccctcccttcctcttctggagcgtgaaatgagattggtcaagataaaaaaggaaaagattcggttatttttttaagagtgtggataatggggcctctcaatcaaaatcccagtctccagtcggttccccccattccccttccaacccctccaccttcccctgccgcctgcttagaggaggaggaagaaacataaagcacaaggcttttctcttaattatgaatcattccctgagggcaggcccagggcaaggggttcctggggcccagagtctgacctgtgaggtagctagaaggcttgagcctctcatcaaagtcc

SEQ ID NO: 106 - DNA, 418 bp, Homo sapiens
ctttgcaggactttagcgttttctccacagattcctgcctgcagctttcagatgcagtttcacccagtttgccaggttccctcgacagtcccgtagatatttcagctgacagcttagacttttttacagacacactcaccacaatcgacttgcagcatctgaattactaaaaacattaaagcaaaacaaagcatcaccaaacaaaaactcctttgaccaggtggttttgccttcttttatttgggagtttattttttattttcttcttgacctaccccttccctcctttaagtgttgaggattttctgtttagtgattccctgacccagtttcaaacagagccatcttttacagattattttggagttttagttgttttaaacctaactcaacaaccctttatgtgattcctgagagc

SEQ ID NO: 107 - DNA, 521 bp, Homo sapiens
  misc_feature (172)..(172): n is a, c, g, or t
gtcaccctgaggaaggttcattgccattgtcatcaccatggaaacaacgttcctctccacctgcattatgtactacatgacaggcatcaatctggggaaataataaaattatcacctttgtcagaccataagagtttctccaaaagtggtcagtttggctgggcaatatttnctctcatctaacaaacacaatccattgtcatgaaattacccttaggatgagtcttctttaatcaatcatatattgggcggaaaaaacaccagctttgacccgaagtagttgaagagctacttcattcttttctgaagttgtgtgttgctgctagaaatagtcatttgtgaattatccaaattgtttaaattcacaattgaattagttttttcttcctttttgcttgaagcaaacagttgacaatttttaaccttttcattttatgtttttgtactctgcagactgaaaagacaaagtttatcttggccttactgtataaaggtgtgctgtgtccaccgttgtgtacaga

SEQ ID NO: 108 - DNA, 526 bp, Homo sapiens
  misc_feature (84)..(84): n is a, c, g, or t
gaggtctggcactagtagcacaacctaaggtggcattacagatctttgagcgagccacagcaacttttctgccaagtcagcttnagttnagacttcagtgaatcaggntattgctatcctaatgtatgtctctatgagtgtatntagccacanantctgcccttggttgantttctgactcattgcttgcttgcttgtttccttgctttggaaaactatnnaagattgctaaaaaataccactgcaaagtgatggaaaagggtggagaacaggggagtagccaggctggatggctcaaatataaatgaatgaggaattctttatgaagtatcagtcagattttatgattaagtgatgtaatataggaattatgtaaaagggaagaatgtctgatactgatctattagagaggtactttagaggcttcttgattggcataaagttcctaaggttatagattttccccccttttggctgtatagcaaagtgttttaatccacggttgtgccttattgttccattaaaa

SEQ ID NO: 109 - DNA, 479 bp, Homo sapiens
  misc_feature (424)..(425): n is a, c, g, or t
caatgggaggggtcggagctcttccttcccctctgtggagtcacttttgtattctttttaaccagatttcttaaaatgttgttgttttgtgaatcctgacattggttcttacttttgtatgctgcctcctctgtgccctcccagacgctgactgggaaacacaagaagtacaaccaacaggaaccagcgccaagggcaggcagcggcctccttgctcccctcccttactcctccctctgctgcctcctccccccaccaagtttcagggccctggattgttcccagttcccattgtggtcccttcagagctcctttccaacagcatctctctgtcgaagaaagaagctctgtcaagttagagagagacaatgtgtaggaaatgttcttttttaaaaaaaaataacaaaaacaaaacaaaactatnnannntgtgattgttttccttgttaatctgctccaaccacctgaacatctaagta

SEQ ID NO: 110 - DNA, 554 bp, Homo sapiens
  misc_feature (266)..(266): n is a, c, g, or t
gagacgggagtttaccccgatcacagaaaccataccaactgaaagacaaatcagcatcttgctggacgacccctcacagagctcctagatccttgaagtgtgaacttcagcagctgagagagatggggtctcactatgttgcccaggctggtcttgaactcctggactcaagcaatcctctcacctcagcctcccaaagtgctgggattacagattttataaatattgttgatctttttgaaaaaccaactgttggcttcattttntttattgtgtaatactaccttagaggacagcagttcctaatacctacttttattatgagtctctgccatttataaagaactgtggacagcacagggaatgggggaagaaaactctggtgcagcttgaatcttggtagcaaaacagtgacttcatcagaaaattttgtcactctctattagatataatggagtttgaccatttggaatttggaatttttcaaatgaatatgacaaaaatttaaaaaactcttgtattactatgtgataacacagatctttacaacttta

SEQ ID NO: 111 - DNA, 446 bp, Homo sapiens
  misc_feature (47)..(47): n is a, c, g, or t
aagccttcaccagatggtcaagcagatgctggtgccatgcccttgancntcncnccaccatcccccacctagccactatatgggttgttagatattttgaccacctcctcttcnctcactccactattcaactcactgcatcatcaatgtacttattacaaacctgtcacaagccaggtcttatgctaggtgctcctctcaacaggttcttgagctggcaggggagagagagacattcaaacaccaaggattaatataccattacaggtttaaagacagaggcctataagggtcccctggcagtgccatggaggtagggcatggtcggctgtacctgtagaggtgtctaaagggaggcttgcaagctgccccttgaaggacgagcagaaaattgtacatgaggacaagtaggaaaggaattccaggaggagggatcagcatgtgca

SEQ ID NO: 112 - DNA, 371 bp, Homo sapiens
  misc_feature (68)..(69): n is a, c, g, or t
ggactaaatcgagccttattatacatcagcagtctcacactggagaaagtccttttaagttaaggganngnnnnnnannntnnancaaatgtaatactggtcagcgccaaaaaactcacactggagaaaggtcttatgagtgtggtgaatccagcaaagtgtttaaatacaactccagcctcattaaacatcagataattcatactggaaaaaggccttagtggagtgaatgcaggaaagtcaccaaaactgtcacctcattcagcaccaaaaggttcacatcggaccaagaacctattaatatatgtaaatctaatgttgaaagagttcagatggaaatctgcgaggatttcctgctgggaactacatta

SEQ ID NO: 113 - DNA, 533 bp, Homo sapiens
  misc_feature (167)..(167): n is a, c, g, or t
aattgggcaggctcttgggaagtagaaagttctggtgtttttgctggtgaaggttttgactgtggagctcttctaacacccatatcagtgtctgtttctctgcatgtggctgctgccctgttggtggagctctgggggcagagaccaggccgccgtccagtggcgcnccgtgcgcaccagctgcctgctgtttacacccaggtgcgccgagtctctttcatacagcacagcaaatgataatagctagtgacaatgtgtttcctgtgcactcgtgaaaatgcagggaggacaactgcatgcttagatctgtttcttttttcagacattcaaatgttctaatatctgaagctaacattttgtaggatataggatgctgattatgtgaacaattagtcattggttttctgtactgctatgaatatgtctgatttcaagttttggtcaaatatctaaaatgcaaggtgaaagtgcctttgtctctatgcttctaaaatcgctcatgcttagttgtggtatggatgtcttccgcagtg

SEQ ID NO: 114 - DNA, 544 bp, Homo sapiens
  misc_feature (358)..(358): n is a, c, g, or t
cttggtaagccttgcctgtagcggctccgctgccgagtgctttgacaccaggcgctcccagagctctgcccccactgccaagcggcagctgctccggagggcacggggggctggatttggctgtggcttctccagctctgcacaagagccccccttccctggccctgctgcagcatgactgcctcctggctcgtgtcacccactctgtctctgtctctcttcatacgtttccagctgagctgggatccatagtctgtttccctctccacgaccaatctatttatcttctctggaacttcttgtaatgccgggagtgcagagcttacaagttggggcaggaagctttagaagcccaggnagccctgagaggctctttccttgtaagtgggtctctccccaggagcctcttggaatatttagcagggacttttacccatgctgggtctagagaccctcccgcccctctgtttcctgccctcctacttagactgggatctggtttccctcagctggttcccttgctagcgtgtgactctgtgtgtct

SEQ ID NO: 115 - DNA, 436 bp, Homo sapiens
  misc_feature (55)..(55): n is a, c, g, or t
gttcacagcagtgggtaggcccagcagtggttcttgacatcacacgatgaggcgngcatctcccgtcatccagggagaccagaggacccttgtctcactcccagttggctnttagtcacagccccgctttgtctttgacatggacgtttgtgatgatcacgttcctcccgctccccgtgtntgaagagtgctccctgactggctgccgtctcctccctgtcgggtctggctgggttctccanagggagtgctgcggaggggacacagcanaggccccatgctcgtgatgtatgttgcagatcattttcccccattctgtccttttttgttaaattgtggtaaaaagcacataacataaactgtaccnccttaaccatttgaaagtatatatcccagactgtcttttatctttagacttcacttgtggtttgttgcc

SEQ ID NO: 116 - DNA, 276 bp, Homo sapiens
tcccctggaagttgtcctttctgatcctctcttcttttcccatttacaaatgatttcgtgactgtagtttttgttcaccttctgtgcatctggcctgggggctgttagctcagaggagaggagcaaacaggaaaatgacttctgttctgtccccgctgttttgggggaagtctctcccactttgggatcctgctgaagctaggttcatgaggtcggaaatccccaccacatttgcctagactttgggcacaggagttcttagtccaccaaatcaga

SEQ ID NO: 117 - DNA, 331 bp, Homo sapiens
cattttctctaactttatctcctatgcatttccttatgtgtcctgtacagcagtatattccaaaatccccagtggatgtctgaaaaccacatatagtaccaaactgtatatatgctatgttttgtttcatacatacctataataaagtttaatttatgaattaggcacaataagagataagcaggctggacgtgctggctcacgcctgtaatcccagcactttgggaggctgaggcgggtggattgctttagcccaggagtttaagaccagcctggccaacatggcaaaaccccgtctctataaaaaatgtggaaattaatcaggtgtggt

SEQ ID NO: 118 - DNA, 482 bp, Homo sapiens
gagatgaccgaaaacttcaacccctgcagtcagcaatggtcaacagaaagggcccaattctccacgacaatgcatgatcgcacattacacaactaaagcttcaaaagttgaactaactgggctacgaagttttgcctcatccaccatattcacctgacctcccgccaaccgactaccacttcttcaatcatctcgacaactttttgcaaggaaaacacttccacaaccagtagaatgcaaaaagtgctttccaagagttcactgaatcctgaagcacggatttttatgctacaggaataaacaaacttatttttcattggtaaaaatgtgttgattgtaatggatcctattttgattaatgaagatgtgtttgagcctagttataatgatttaaaattcacgatccaaaaccgcaattacttttgcatcagcctaatatgaggaagtaatagttgaacagaataattctttcctggaagtct

SEQ ID NO: 119 - DNA, 455 bp, Homo sapiens
ttggtttggtctggtttggctacctgattcctgctgtctttttctacgccaggtgaagaggcactttcaagatccttctctgagacctgcaccaataagactataccaatgttcagttgaaacatcaggtataagtttagcggaaacgaaagtacaacctgctttgaaataaattccaaggacagattgtcattaacgaaatagaaagtggactatgcccctcatgctgccagcgcctggtatgatgcggcgtgacacgcagcgcttgcggcagtacaatgcccccaatcacccgccccgccccgacgcgccgcccactcacggcaaagagagccacctagtgagggattattctcatttccgcggtggggttctgcttttctttctaccatgagcgcccaaggatagacactcctactacctattacctcaaatagcctacatttctttccgaa

SEQ ID NO: 120 - DNA, 544 bp, Homo sapiens
  misc_feature (150)..(150): n is a, c, g, or t
agaacactgagcgaggctctgtagatggatgtaataaaaatctataaaacaatgtgtttaaacctaagaattctactgctttccaattccttccctctgctccttttcctaacctcctgcttctccagcccttccctctgtccctttcanccctcaggccctcctctccccttagtccccaccaccctgtcacttctaaattgtggctctagcattgtcccattacctgctangtgactgttctctccacagtggtcctgctcctgtgagtcagagtgtgtcatttcctcacctaaaacactccagtggctccacctcggtcttgtgaagcttctagaatgtcaggcacgtgagcatatgagggcatacctggttcatcttaggcactaaattnnnntttgttgactgaatgaatgaaatatgaatgtattaaattgcatcacagaaagttataaaatgtaaaacactgaaaaattaagaaatattttatnttatgtaactagtgtgcatatcaattcattccgagtctgttgagcctgtgtat

SEQ ID NO: 121 - DNA, 338 bp, Homo sapiens
  misc_feature (193)..(194): n is a, c, g, or t
aatgattcaactcatgtgatccagtgttacattcagtgtggtaatgaagaacagtcaaaacaggcttttgaagaattgggagataatttggttgaattaagtaaagccaaatactccagaaatattttaaagaaatgtctcacgttgtgaacatgtaccctagaacttaaagtataataaaaaaaaaaaaaannggaaagtatcttgcacaagctcacgtagctggtaagttacatagttgggatctgaattcagttgtggcttcatgcctgagcttttaactactactactaaactgagaaggcacttgcttgagtaaattatgtcatcctcttaat

SEQ ID NO: 122 - DNA, 443 bp, Homo sapiens
  misc_feature (30)..(30): n is a, c, g, or t
gatgtggcatgtgatgacattgcacatggncagttaantgngccaagaagngcagcagtagcagcaacnggagatgcaaagcccaacatgatggggagagaaantnttctttcaatatgtgcttctgtaccaaaagtggaatttcacgagagacatattttggaacatttttccttttgtgtgtgcgtgagtgtttccctgtttccagccaagggtattgtgagtttctcctgggcctccttcagaatctgggtgctctggaaagcagtgttttggcaacatggggaaagtatggcagtgtgggagggtcagctgggtctgggtttgaatattgcatttgaatattttaccagcattgatgtcggataaattatttagtccctgtaagcctcagttttntcttnttctacatacacataatatatttgactctttgttgtgat

SEQ ID NO: 123 - DNA, 510 bp, Homo sapiens
  misc_feature (135)..(135): n is a, c, g, or t
tttcctaactttctgatcccttggaggtgataatcaaatattctagtctgaggcattgggatacatggtgctaggttctgagactctgcgtcaggcctgaaccctgcattttgtggaggtgggtgggagaatgtncccctggggaacatgcctagacacgggggacaacagttgccctcatggggaggtacctgtttactcgctgttatgggaccgctttcacaaaaccactgcaggtgagtgagttcctgctgaatatcaggcctggtgtctctagactcattattncccccacccaacccctatgttagttcatctcgagccacatttttattgccataatccaggcctggacaggccaagatcttttaacaattttaattactgaaaataataactgcattttttttnaaagcccaacttttnggtanagtcagcccaaaatacagtctttgtgttgccatctgggaactggatttggaattgttcttccatgagactgcagagcag

SEQ ID NO: 124 - DNA, 447 bp, Homo sapiens
ccacctcctccaggaaagccagaaagaccacccccacaaggaggtaaccagtcccaaggtcccccacctcatccaggaaagccagaaggaccacccccacaggaaggaaacaagtcccgaagtgcccgatctcctccaggaaagccacaaggaccaccccaacaagaaggcaacaagcctcaaggtcccccacctcctggaaagccacaaggcccacccccagcaggaggcaatccccagcagcctcaggcacctcctgctggaaagccccaggggccacctccacctcctcaagggggcaggccacccagacctgcccagggacaacagcctccccagtaatctaggattcaatgacaggaagtgaataagaagatatcagtgaattcaaataattcaattgctacaaatgccgtgacattggaacaaggtcatcatagctctaac

SEQ ID NO: 125 - DNA, 562 bp, Homo sapiens
gtttgatgtctattatctcacttcatcctcaccaggaccccatccgagccttaatttcagttgacagtaactattggatccccaggaatatgtttgcatatttggggagaaaatactattggaggggaacagaaatgctactaagggtctcactgtgtcacccaggctggagtccatcaaagctcactgcagccttaaccttctgtgctcaagggatcctcccacttaagcctcctgagtagctggaactacaggcatatgccaccgagcctggctaatctttgatttttttgtacagattgtgtctccttatgttgctcaggctggactcaaacttctggtctcaagcgatctttccatcttagcttcccaaattgttggaattatggacatgagccagtgtgcttggcctgattttttttttttttttaatgagaaaaacgttccttaagaaaagtttcattgtaagacgaggacttgctatgttgccagtttggtcttgaactcggtctcaagtgattctcctgccttgggttcccaaagcgtttgggccggcagatgt

SEQ ID NO: 126 - DNA, 484 bp, Homo sapiens
ctgaattggaacacaccagcactgtggtggaggtctgtgaggcaattgcgtcagttcaggcagaagcaaatacagtttggactgaggcatcacaatctgcagaaatctctgaagaacctgcggaatggtcaagcaactatttctaccctacttatgatgaaaatgaagaagaaaataggcccctcatgagacctgtgtcggagatggctctcctatattgatgaagctactatgtcaaatggcaagtagctctttcctgcctgcttctcagctcatttggaaaaatactgcgcaaaagacattgagctcaaatgatgcagatgttgttttcaggttaatggacacgcaaagaaaccacagcacatacttcttttctttcatttaataaagcttttaattatggtacgctgtctttttaaaatcatgtatttaatgtgtcagatattgtgcttgaaagattctcatctcagaatacttttggact

SEQ ID NO: 127 - DNA, 544 bp, Homo sapiens
  misc_feature (257)..(257): n is a, c, g, or t
gagtgtcttgactattctggctctttgtattttcatgtaaggtttttctcccatataagttttaaaatcagcttgtcaattccaacaacaatgatgcacttgatagtttgggaatttattatagctatcaatcagttttgggaaaattgacgtctttacaatattgagttttctgattcatgaacatggtttacctctcttcccatgggggtctcctttaaggtttaccaataggattttatatttggggccattgnggtcttgcttatcttaagtnnnnnnnnnnnnnnnaaatctcttgaccncatgatctgcccgccttgtcctcccaaagtgctgggattacaggcgtgagccaccgcacctggcctgcaatacagtattgttaaccgtcttcaccatgttgtacgttagagctccagaaattatttancatgcataactgaaactttatactctttgaacaccacctccccatttccctctcccggcagccatttgtgcctctcggttctctttattagcttccattttgtgggtcagt

SEQ ID NO: 128 - DNA, 522 bp, Homo sapiens
tacgtcaaagaccgctgctgcagtagctgcccagtcaggaatacttgataggacaatttctgtaattatgaagaatcaaacaccaacaaagaagtatgatggctacacatcatgtccactggtgaccggctacaaccgtgtgattcttgctgagtttgactacaaagcagagccgctagaaaccttcccctttgatcaaagcaaagagcgcctttccatgtatctcatgaaagctgacctgatgcctttcctgtattggaatatgatgctaaggggttactggggaggaccagcgtttctgcgcaagttgtttcatctaggtatgagttaaggatggctcagcacttgctcatcttggatggcttctgggccaaaactgcagtcactgaatgaccaagagcagcacgaaggacttggaacctatccttgtaaagagttccttgatgggtaatggtgaccaaatgcctcccttttcagtacctttgaacagcaaccatgtgggctactcatgatgggcttgat

SEQ ID NO: 129 - DNA, 544 bp, Homo sapiens
ttggatgccctaactgctgatgtgaaggagaaaatgtataacgtcttgttgtttgttgatggagggtggatggtggatgttagagaggatgccaaagaagaccatgaaagaacacatcaaatggtcttactgagaaagctttgtctgccaatgttgtgttttctgcttcatacgatattgcacagtactggtcagtatcaggaatgcctacagttagcagatatggtatcctctgagcgccacaaactgtacctggtattttctaaggaagagctaaggaagttgctgcagaagctcagagagtcctctctaatgctcctagaccagggacttgacccattagggtatgaaattcagttatagtttaatctttgtaatctcactaattttcatgataaatgaagtttttaataaaatatacttgttattagtaattttttcttttgcattaccatgtaaaatttagacatttgaattttgtacttttcagaatattatcgtgacactttcaacatgtagggatatcagcgtttctctgtgtgct

SEQ ID NO: 130 - DNA, 436 bp, Homo sapiens
aggtcacagtatcctcgtttgaaagataattaagatcccccgtggagaaagcagtgacacattcacacagctgttccctcgcatgttatttcatgaacatgacctgttttcgtgcactagacacacagagtggaacagccgtatgcttaaagtacatgggccagtgggactggaagtgacctgtacaagtgatgcagaaaggagggtttcaaagaaaaaggattttgtttaaaatactttaaaaatgttatttcctgcatcccttggctgtgatgcccctctcccgatttcccaggggctctgggagggacccttctaagaagattgggcagttgggtttctggcttgagatgaatccaagcagcagaatgagccaggagtagcaggagatgggcaaagaaaactggggtgcactcagctctcacaggggtaatca

SEQ ID NO: 131 - DNA, 402 bp, Homo sapiens
gcacctcggagttgcagctgtgacactcataggttactcccaggagtgtgctgagcagaaggcaagctcttgctggatgaaacccctccaggtggggttggggagacttgatattcacatccaacagtttgaaaagggagagctcaattcccagcgtcaccccatggcttgtgttgcctgctacgcattgacttggatctccaggagtcccctgcacataccttctccatcgtgtcagctgtgtttctcttgattccgtgacacccggtttattagttcaaaagtgtgacaccttttctgggcaaggaacagcccctttaaggagcaaatcacttctgtcacagttattatggtaatatgaggcaatctgattagcttcacagactgagtctccacaacacc

SEQ ID NO: 132 - DNA, 390 bp, Homo sapiens
tcaagtgagtgagttcccctctacttttagccttccacccaaactggaagcctctaggtgctatcaattatttatatccatcgtttacatccatgaaattggctgaataattactcctctgcctggcgtagacatgtgctttgggaaaaaaacgagtttataatcctataatgaagaatactggcacaggcaatgctcactcgaaaacttcaagtaatttctagttggttttggaatgcttgataaagttcctttacagctttattttcctgatttgttttggtttagatcaaagttcaaattaattttaacttagctaatgaactcatcaccaggacagttggagggggtaggccgaggttaaatggtccacgtttcaaaaatgttaat

SEQ ID NO: 133 - DNA, 503 bp, Homo sapiens
cttttgttcttgctgggttatttattttgattttagcattaaatgtcatctcaggatatctctaaaaggggttgtttaattcctaattgtatagaaagctagtttggtgaattgtattggttaattgactgtttaaggccttaacaggtgaatctagagcctacttttattttggttaaagaaaaagaaaatatcaataattcaattttgtgtcttttctcaatttattagcaaacacaagacattttatgtattatttcgatttacttcctaattataaaagctgcttttttgcagaacattccttgaaaatataaggttttgaaaagacataattttacttgaatctttgtggggtacaggttgatctttatattttactggttgttttaaaaattctagaaaagagatttctaggcctcatgtataaccagggttttgaggataaagaactgtatttttagaactatctcatcatagcatatctgctttggaataactat

SEQ ID NO: 134 - DNA, 346 bp, Homo sapiens
ttaccctcgtggctaagcaagtgtctgcaggagcagagatggctggaaggggcctctgcacacggaagatggcttgttcagcccattcacctcctgaggatgtgggcagtctcctccaagaacacatggagctgcttcctgatcccaagcaggtcattgccactggaaggacatggccccggtgatccatgcttcatgcccacccagaaacacacccctcagtgtgtgcctcagtttactttggagatcagttgtcgtttttagtgctcctttaggcttactaaaacagttttggaaacaaagctattttgaagtattcaagcagaggaattccctaacactgacc

SEQ ID NO: 135 - DNA, 506 bp, Homo sapiens
gaccattttgcgagtgtagccctgtttcactcggatcaggttggcacggccgcctgcgtgtctgtccacctcatccctccgtgtatctgagggagtaaaggtgaggtctttattgcttcactgcctaattttctcacccacattcgctgaagcgatggagagtcgggggccagtagccagccaaccccgtggggaccggggttgtctgtcatttatgtggctggaaagcacccaaagtggtggtcaggagggtcgctgctgtggaaggggtctccgttcttggtgctgtatttgaaacgggtgtagagagaagcttgtgtttttgtttgtaatggggagaagcgtggccaggcagtggcacgtggcatcgcatggtgggctcggcagcaccttgcctgtgtttctgtgagggaggctgctttctgtgaaatttctttatatttttctatttttagtactgtatggatgttactgagcactacacatgatccttctgtgcttgcttg

SEQ ID NO: 136 - DNA, 378 bp, Homo sapiens
aaggaaggccagagagccgcgcagttctctgcaggtgcagatgcaggcagtggaggtggcctgagcaggcagaaggacaccaagcgccctatgttgcttgtcattcatgacgtggtcttggagcttctgactagttcagactgccacgccaaccccagaaaataccccacatgccagaaaagtgaagtcctaggtgtttccatctatgtttcaatctgtccatctaccaggcctcgcgataaaaacaaaacaaaaaaacgctgccaggttttagaagcagttctggtctcaaaaccatcaggatcctgccaccagggttcttttgaaatagtaccacatgtaaaagggaatttggctttcacttcatctaatcactga

SEQ ID NO: 137 - DNA, 562 bp, Homo sapiens
tcccggctacatgggagcgcggtgtgagttcccagtgcaccccgacggcgcaagcgccttgcccgcggccccgccgggcctcaggcccggggaccctcagcgctaccttttgcctccggctctgggactgctcgtggccgcgggcgtggccggcgctgcgctcttgctggtccacgtgcgccgccgtggccactcccaggatgctgggtctcgcttgctggctgggaccccggagccgtcagtccacgcactcccggatgcactcaacaacctaaggacgcaggagggttccggggatggtccgagctcgtccgtagattggaatcgccctgaagatgtagaccctcaagggatttatgtcatatctgctccttccatctacgctcgggaggtagcgacgccccttttccccccgctacacactgggcgcgctgggcagaggcagcacctgctttttccctacccttcctcgattctgtccgtgaaatgaattgggtagagtctctggaaggttttaagcccattttcagttctaacttactttcatcctattttgcatccc

SEQ ID NO: 138 - DNA, 528 bp, Homo sapiens
tgaagaaaaccttcattacccgcttctgcttattttgaccaaacatggatagaagattaagcttctcaaagacgaagaaacgtatcaagtgcatagggaatatttttacaaaaacggaaatctgtaaggggtataatcgcctgcctgcgccctttgcagcatttcacgtgtgggctatggactccacctgtcctcacccacgttattccccagctgccctctccagctccctccccgcctctttttacactctgcttgttgctcgtcctgccctaaacctttgtttgtctttaaatgtgtataagctgcctgtctgtgacttgaatttgactggtgaacaaactaaatatttttccctgtaattgagacagaatttcttttgatgatacccatccctccttcattttttttttttttttggtctttgttctgttttggtggtggtagtttttaatcagtaaacccagcaaatatcatgattctttcctggttagaaaaataaataaagtgtatctttttatctccctc

SEQ ID NO: 139 - DNA, 371 bp, Homo sapiens
tattcacaagttttggagggctttttgttcctctgatagacatgactgacttttagctgtcataatgtattaacctaacagatgaaatatgttaaatatgtggttgctctttatccctttgtacaagcattaaaaaaactgctgttttataagaagactttttgttgtactatgtgcatgcatactacctatttctaaactttgccatattgaggcctttataaactattgatttatgtaatactagtgcaattttgcttgaacaatgttatgcatatcataaactttttcaggttcttgtttaagtacattttttaaattgaacagtatttttcattttggttataatatagtcattttgcctatgtttc

SEQ ID NO: 140 - DNA, 324 bp, Homo sapiens
ctcagcccctgtcaacagtggggaccccaccaccaccatcctggagtgattccaactcaactcaaaggacacccagagctgccatctggtatctgccagtttttccaaatgacctgtaccctacccagtaccctgctccccctttcccataattcatgacatcaaaacaccagcttttcaccttttccttgagactcaggaggaccaaagcagcagccttttgctttttcttttttcttccctccccttatcaagggttgaaggaagggagccatccttactgttcagagacagcaactccctcccgtaactcaggctgagaag

SEQ ID NO: 141 - DNA, 339 bp, Homo sapiens
gtttctgtgattcaggatcctcttgggagagtatattcaataaaagcccggaggtggtgactcctttgcagctccagtgttgccagcgcctagtggagctttgtaaacagtgcctgctagtggtttacaaatatgcaactgacaaaagaggatcactttcaggcattggtcctgactggggtaattccaggtatttactaccagggagcacccaattcttcttgagaacaccaacctacaacttgaagtacaattcacctggaatgactcgctccaatgttttgtttacatccagatatggccatctgtgaaacagaagggaagatcgccattggttat

SEQ ID NO: 142 - DNA, 414 bp, Homo sapiens
ggaggtcccaaatatgtggtctatcaccactgaattcatgtaatagataagaaaaaaattagaggtggatgtcttgttttgtgtcatgaattactaaaatctcttagtagttgtggtatatttttgagtaaaattaccatttccagatttgagtttgaagggcttttatagttgtattttcctcctcactgttaataatcataatcctttttcagtattttagtggccttgaacaactggtttatctacaatctcaaatcctaagtgtataattatgtgcaatgttcaatacctcatataatacttgctcaacagtatagtggtaccaatggcattaagatggtgtttttgttctacatatttttcaataatttattctttctaatgttgaaattatatcaggctttaccggtt

SEQ ID NO: 143 - DNA, 524 bp, Homo sapiens
gaagttgcaacattcgtttgataggaattccagaaaaggagagttatgagaatagggcagaggacataattaaagaaataattgatgaaaactttgcagaactaaagaaaggttcaagtcttgagattgtcagtgcttgtcgagtacctagtaaaattgatgaaaagagactgactcctagacacatcttggtgaaattttggaattctagtgataaagagaaaataataagggcttctagagagagaagagaaattacctaccaaggaacaagaatcaggttgacagcagacttatcactggacacactggatgctagaagtaaatggagcaatgtcttcaaagttctgctggaaaaaggctttaatcctagaatcctatatccagccaaaatggcatttgattttaggggcaaaacaaaggtatttcttagtattgaagaatttagagattatgttttgcatatgcccaccttgagagaattactggggaataatataccttagcacgccagggtgactaca

SEQ ID NO: 144 - DNA, 487 bp, Homo sapiens
gttatacagatgccatgctccacaccacgagcagtgtacaaatctggctgcccgtttactttctgagcaagcactggagtccactccgacctttttctttgaacatgcatgctgctggaatatgtataaatcagaactagcagaagtagcagagtgatgggagcaaaataggcactgaattcgtcaactcttttttgtgagcctacttgtgaatattacctcagatacctgttgtcactcttcacaggttatttaagttcttgaagctgggaggaaaaagatggagtagcttggaaagattccagcactgagccgtgagccggtcatgagccacgataaaaaatgccagtttggcaaactcagcactcctgttccctgctcaggtatatgcgatctctactgagaagcaagcacaaaagtagaccaaagtattaatgagtatttcctttctccataagtgcaggactgttactcactactaaactct

SEQ ID NO: 145 - DNA, 547 bp, Homo sapiens
gaacgtcgtatgagatcctacaatggaagaataaaatcacctcattcttcatttcagatctgaacattagcagtgatctagatttttttttttttaaacaaaattaagtgtgcttagagtcatccctctacatgggctgtggctgtcagcccataggtttgtcagtttcacatcaaaactgtgggtataaactgttgaaaccaatcacattaaaatatttagctgggcacagtggtgtgcatctgtagtcccagctacttgggaggctgaggcaggaggatcgcttaagcacaggagttggaatccagcctgagcaacagagcaaaaccccgtctctaaaatacaaataaaatatttgtgtagtttttgattaaaattgactacagcggtcagtataaaatacatgtcgcttttaaggaagtgctctttatgtatctaacagatggaagtttttgcattggtaagagcatttatatatgctttgtttcagggtttatggatttgtattcatatattgtcaaataggtttcatactctaattttactt

SEQ ID NO: 146 - DNA, 514 bp, Homo sapiens
agattatatccctatcttctttttcatgtaaaccactggtcacaaatgaactgatctctgtatcccattattactataagaggtgggaatcccaaaactgcttagattgcagtacatgagtttacacaaagacttcaacaattgcacatcttcattctcccaactgagtgtagtatgtggagcataaaacagcatattcttagtatttcatgaatatcagatggtctttaaatgtctctttatggatgtattgttcacattatggctttaaaataatgaatatgtaaaagtgaggtagtgaacatcctaaatttctacactggaattactaaataatcttatttcataaaatgggaaatatatgttaaatgacatcactggatgaacttgaagatcttttacttgttaacaaaaaaatactatggacagctttctgattgttggggtaaatagcaaatgttcaaactttgcaggcattttgacattcatcataacaacacaattcctagacatt

SEQ ID NO: 147 - DNA, 478 bp, Homo sapiens
ttaggcagtctgtggtgctcagtcacctctgtcttcgatgagaaacagcagtggaaattctgtgaaacgaatgagtatgggggaaattctctcaggaagccctgcatcttcccctccatctacagaaataatgtggtctctgattgcatggaggatgaaagcaacaagctctggtgcccaaccacagagaacatggataaggatggaaagtggagtttctgtgccgacaccagaatttccgcgttggtccctggctttccttgtcactttccgttcaactataaaaacaagaattattttaactgcactaacaaaggatcaaaggagaaccttgtgtggtgtgcaacttcttacaactacgaccaagaccacacctgggtgtattgctgatgctgaggaaaggagaaatatcttcagaggaagactgccgccatactgaggctgagcacagatttgtctttttcattgcatctgtcaa

SEQ ID NO: 148 - DNA, 426 bp, Homo sapiens
gtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttgaagtgcgatagtgacattatggaagagtcaaaccacaaccattctttcttgtcatagttcccatcataaaataatgacccaagcagacgttcaaa

SEQ ID NO: 149 - DNA, 503 bp, Homo sapiens
tatgcattttttaccacaatttttaaaaagtttgaatagaaatttttaatgtctttgagtggattttgttttttgaacagttggatagacttctgcgtaagaaagctggattgactgttgttccttcatataatgccttgagaaattctgaatatcaaaggcagtttgtttggaagacttctaaagaaactgctccagcttttgcagccaatcaggtagcttaatggatgtaatacatttctgagtaccattatcttatctagtaatgtagatttacatagaattaagagttgaaagaaattaagtacttaagtagcctggaggtaggttctagaaaaccaaaatgagagttttgctaaaatcatcctattacttatgatttatggtagtaatattatactgtcctaggcttctgatgatcattgttgccagatgcagcacatatactaaatatgagacagggtaatgaaaacttggggaactggtaagtttttgcatgctac

SEQ ID NO: 150 - DNA, 541 bp, Homo sapiens
tgacccctttgatattccagcaagtgcagaatggagatgcagacatcaaggtttctttctggcagtgggcccatgaagatggttggccctttgatgggccaggtggtatcttaggccatgcctttttaccaaattctggaaatcctggagttgtccattttgacaagaatgaacactggtcagcttcagacactggatataatctgttcctggttgcaactcatgagattgggcattctttgggcctgcagcactctgggaatcagagctccataatgtaccccacttactggtatcacgaccctagaaccttccagctcagtgccgatgatatccaaaggatccagcatttgtatggagaaaaatgttcatctgacataccttaatgttagcacagaggacttattcaacctgtcctttcagggagtttattggaggatcaaagaactgaaagcactagagcagccttggggactgctaggatgaagccctaaagaatgcaacctagtcaggttagctgaaccgacactcaaaacgctac

SEQ ID NO: 151 - DNA, 511 bp, Homo sapiens
aaggtagaaagccttccgtccagtgtgcgaatctctgtgaacgtgtaagaattcacagtcaggaggactactttgaatgttttcagtgcggcaaagcttttctccagaatgtgcatcttcttcaacatctcaaagcccatgaggcagcaagagtccttcctcctgggttgtcccacagcaagacatacttaattcgttatcagcggaaacatgactacgttggagagagagcctgccagtgttgtgactgtggcagagtcttcagtcggaattcatatctcattcagcattatagaactcacactcaagagaggccttaccagtgtcagctatgtgggaaatgtttcggccgaccctcatacctcactcaacattatcaactccattctcaagagaaaactgttgagtgcgatcactgttgagaaacctttagtcacagcacacacttttctcaacattattggcttcctcctagagtgttgtgagtgtgagaaggcctttcactagcccc

SEQ ID NO: 152 - DNA, 505 bp, Homo sapiens
atgttactacaaacttgattaaacttctggtggaaattccatcacattttatgcaattttcaatttatttctccaatttatttttaatgccacatggacattatattccttaaccattcttttgcatgtgattaacatttgtgaaattaaccacttaagcaagtgtttttgctttgatgaaagaaaaatgtttaaaatcctactggatatgaaactgaaagtaatgttttgtgttttttgtttcaaatgaaagtgtaaattaagaatttgttggcagggcgtggtggctcatgcctgtaatcccagcactttgggaggccgaggtgggcagatcacctgaggtcagcagtccaagaccaccctggccaacatggtgaagtcccgtctctactaaaaatacaaaaatcagctgggcatggtggcgggcacttgtagtcccagctactcaggaggctgaagcaggagaatcacttgaactcaggaggcagaagttgcggttagccga

SEQ ID NO: 153 - DNA, 477 bp, Homo sapiens
cctctctccactctctagaaatattaaggctaggctgctgctgtatgtcagggctagtcccctcttctatgaatccagaataactctgaagaagccgagtaacaggcatgaagtgaagagaaatcgctgtaacaggaagacagcaaagcagatgctaatgaccacactatttaacgaactggaaccaacgagaaaatacggtattactgaagactgcacttccttgaacagagtgctcttctcagcaaatcggaaatgcctacacaaatcgctttacaagaaagactgtttcaaagcagcacctttctcaatgttctcgttcaggtgacaattcttcttggtctcagctccaattttattgtcattttcatcaataaggatacacatctctgccaggagttgaacctgttgcttgtcgaggtggttagtgtttatttcaggcatcattacaaaatgtctgatctgttctagaaccct

SEQ ID NO: 154 - DNA, 332 bp, Homo sapiens
aagtatctccatacaaaatacggttgaattacaaaaagaaaattgtaacattagcatggacaaacctggcaggtactccttaactctcctaagtaataaaaactgtaaaatgcaaataagccttcgatgacatttactaacctttactaaagtatcaatgatgacttggttgtttaaacagctgacatttgggcaatttgagtatgtcaaactcaataatactggttttcatttgcaagatccacttaaaacttaaggaggccaaaaaacatcatttaaaataccctataaattataatcatacatatgatacgaaaaatatcctacttcag

SEQ ID NO: 155 - DNA, 195 bp, Homo sapiens
catacacatacgtattttccgtagtgctctgggtgggggaaaatgtttaaattgtattagcaaatgctaacttacactttatagcatttatcagctgtggcatattacctgtaacatgtttaaattaaggcaaaggcaatcaaaaacctttttgttttgtagcctgcttttgctttcacaatttgtcttacaatt

SEQ ID NO: 156 - DNA, 487 bp, Homo sapiens
gctggccaagactactgggccgtgctttctggaaaaggcatttcagccacgctgatgatcttctccctcttggagttcttcgtagcttgtgccacagcccattttgccaaccaagcaaacaccacaaccaatatgtctgtcctggttattccaaatatgtatgaaagcaaccctgtgacaccagcgtcttcttcagctcctcccagatgcaacaactactcagctaatgcccctaaatagtaaaagaaaaaggggtatcagtctaatctcatggagaaaaactacttgcaaaaacttcttaagaagatgtcttttattgtctacaatgatttctagtctttaaaaactgtgtttgagatttgtttttaggttggtcgctaatgatggctgtatctcccttcactgtctcttcctacattaccactactacatgctggcaaaggtgaaggatcagaggactgaaaaatgattctgcaactctcttaaa

SEQ ID NO: 157 - DNA, 391 bp, Homo sapiens
tgacatgcaccagagggtccacaggggagagcgaccctataattgtaaggaatgtggaaagagctttggctgggcttcatgtcttttgaaacatcagagactccacagtggagaaaagccattgaaatctggagtgtgggaagagatctactcagaattcacagcttcatttacatcagtaagtctatgtgggagaaaagccatataaatgtgagaagtgtgggaagggctttggctgggcctcaactcatctgacccatcaattctccacagcagagaaaaaccattcaaatatgagaactgtgggaagagctttgtacatagatcatatctttttttttttttttgagacagagtctcactctttcacccaagcctgactgcagtggcg

SEQ ID NO: 158 - DNA, 472 bp, Homo sapiens
gaaaagcgccctgtgctgagtaaagcagccagtcttctcttgtcacagtaaaaggctgggagtaaaatttcccataaacacaggggaaacctacatttactcacatgccaaggaaaatggcacggaagacccacgtgtagccacagcagagtctatgcagagggcctgcaaatgcctggggtgcgagtgaatgcctggaggggcggagtttccaagataacagctattgtgttttctttttcacacttcagaagagaatcctaaggactagactccgctcagtgcattcctttttcatacactgatctcaagtacaatcacataattttgaaaatccatgtagtcctccctaaataaaattataaggataggtttctatttccttccgattacctagatacctccgtcttctggaaaaccccaaaaagaccagtagacgaatcaggaaggtcctaggagtgattcctccaat

SEQ ID NO: 159 - DNA, 317 bp, Homo sapiens
tgcccccacagagcaatacactgaagcctaaacatctatctggtgtttttaaaaagttaaaagaaaaatagattttttttcacaaggtgacaatagtgatttttaccatctggatacagcctggtgtaagcagacgtccattaccaccctcacccacattttcaggtgtctacatcagccttagtcattatggatagtaaatcgacctttaagaattcctggggtggactttgcaaacacattctacaacctgatggtttttactgctcaaactgtcaccatcatcttttgcaatgtgttgctcactgttgtcaata

SEQ ID NO: 160 - DNA, 476 bp, Homo sapiens
ggacagtctcagggttctgttctcgccttcacccggaccttcattgctacccctggcagcagttccagtctgtgcatcgtgaatgacgagctgtttgtgagggatgccagcccccaagagactcagagtgccttctccatcccagtgtccacactctcctccagctctgagccctccctctcccaggagcagcaggaaatggtgcaggctttctctgcccagtctgggatgaaactggagtggtctcagaagtgccttcaggacaatgagtggaactacactagagctggccaggccttcactatgctccagaccgagggcaagatccccgcagaggccttcaagcaaatctcctaaaaggagccctccgatgtcttctttgtcttcgttcacatcctctttgtttcctcttttcaccagcctaaggcctggctgaccaggaagccaacgttaacttgcaggccacgtgacataac

SEQ ID NO: 161 - DNA, 528 bp, Homo sapiens
aagtctgcattgaatccgctgatctactactggaggattaagaaattccatgatgcttgcctggacatgatgcctaagtccttcaagtttttgccgcagctccctggtcacacaaagcgacggatacgtcctagtgctgtctatgtgtgtggggaacatcggacggtggtgtgaatattggaactggctgacattttgggtgatgcttgttctttattgacattgaattctctttctcatagcctctccactttatttttttttatagggtttgtgtatgtatgtgtgtgagcagtgtaaagaaagaatggtaattatagttctgttaccaagaataaataataggaaagtgattacaaatattacctccagggttcaatagaaatcctcaatttagggtgaggagacttttttttggttttggggtttttccttgattgattttgttttcatagtgggaatcaggattgtgctttattgagcctgcagttacattgaattgtaggtgtttcgtgtgctgctaaggta

SEQ ID NO: 162 - DNA, 477 bp, Homo sapiens
gggactgtcgatgtagctgataagctagtgacatttggtctggcaaaaaacatcacacctcaaaggcagagtgctttaaatacagaaaagatgtataggacgaattgctgctgcacagagttacagaaacaagttgaaaaacatgaacatattcttctcttcctcttaaacaattcaaccaatcaaaataaatttattgaaatgaaaaaactggtaaaaagttaagtaagttaaatcgtatgttttcgcctcttctgtgatcaccaataggacatcttcaggcatattggcaggatagagctaatggagtgaaacctattgtaaggctgtactttcgtgatttaatgacctgaggtttggtcataatgcttctgctgtttttgtaggtttatctgatcgttttcctttgctactgctaatggaactgaacccccaggggtattccagttgtaatagcctttccttactgttgtttgg

SEQ ID NO: 163 - DNA, 435 bp, Homo sapiens
gttgagttgaaattctgccgcttactcaatggccttgggtgatgatgctgtaccctaattctaaaggaagcaatgaacccccttttcagctaccttactgataagcacttatgttctgccttctgctatcctgatggttcgggttgtctgtcttactatctacttcttgagtagagagaccacattaaatttattgctgtatctcacagggcatcttgctagtgtgcacaggctcgcctccctacctctgccccgatggtgtgaaggggagagggcgaggttccttagtggcagggctttgctgttcttcactctcagccccctgaaagcagttcttcctgcctctgagcctgtctttccttctgctgttaacttctttcctacttttcttgcatccctctcccttccttttcctgccgtctttcttgtagacat

SEQ ID NO: 164 - DNA, 264 bp, Homo sapiens
aaaaggactaactcacatggctgcagtaagtgctggctgttagctggaagcacaaccaaggctgttaacaggtgtgccttggttctcttccatatggcttctcttttgttttcagtactctgcagtttaattatgatgcatgcaggtgtgaatttctgtttattctgcttgggatgtgttttccttctgggatctgtgaatcggtttctcattatttttgtaaaacctgaagccagttatctcttaaaataccagctctccttg

SEQ ID NO: 165 - DNA, 523 bp, Homo sapiens
ctggacttcttggatgagctcaccctgaaccgcccaggcggtctgctcttggtgttcagaatcacatcaatgcgaacgtcacagcgccttcgagggcgcagattttaactgccacgtatttttaagttgtacttttctgtggaggaaattgtgccttttgaaacgacgttttgtgtgtgtatttcacgttagcatttcattgcataggcaaaacactagtcacaattgggtagatgtgacatccatatacttgtttacattttatctgttctcatgtcaaagactactccttgccccattgaatatatagtggtagcaggtgtacaaattggtcaagttgcaattatttatgagagaataatgataaatgtaaaatatctaaagcatgaatctaagagcacgcaatatataattttaaagaaaatattctatttggtagaatacaaatgtggtgtgtgttgttttataatgactgctgtacagtgggtatagtattttggttttggttccagattgtgcaatc

SEQ ID NO: 166 - DNA, 518 bp, Homo sapiens
gtgaagacatcaagagctcgaagtgtaaattacccgaacaagaatcactaccaaatgataacaaagacattttacaacggcttgatccttcttcattctcaactaagcattctatgcctgtaccaagcatggtgccatcctacatggcaatgactactgctgccaaaaggaaacggaaattaacaagttctacatcaaacagttcgttaactgcagacgtaaattctggatttgccaaacgtgttcgacaagataattcaagtgagaagcacttacaagaaaacaaaccaacaatggaacataaaagaaacatctgtaaaataaatccaagcatggttagaaaatttggaagaaatatttcaaaaggaaatctaagataaatcacttcaaaaccaagcaaaatgaagttgatcaaatctgcttttcaaagtttatcaataccctttcaaaaatatatttaaaatctttgaaagaagacccatcttaaagctaagtttacccaagtactttcagcaagc

SEQ ID NO: 167 - DNA, 177 bp, Homo sapiens
cgggagcctgtctcagaactatcagtacgaggtgtgcctggcaggaggctcagggacgaatgagttccagttcctgaaaccagtattacctaatattcagggccattcttttgggccagaaatggaacaaaactctaactttaggaatggctttggtttcagccttcagttaaagta

SEQ ID NO: 168 - DNA, 576 bp, Homo sapiens
gaactccaccataaagcaactgctggcattttgctggtcagttcctgctcttttttcttttggtttagttctatctgaggccgatgtttccggtatgcagagctataagatacttgttgcttgcttcaatttctgtgcccttactttcaacaaattctgggggacaatattgttcactacatgtttctttacccctggctccatcatggttggtatttatggcaaaatctttatcgtttccaaacagcatgctcgagtcatcagccatgtgcctgaaaacacaaagggggcagtgaaaaaacacctatccaagaaaaaggacaggaaagcagcgaagacactgggtatagtaatgggggtgtttctggcttgctggttgccttgttttcttgctgttctgattgacccatacctagactactccactcccatactaatattggatcttttagtgtggctccggtacttcaactctacttgcaaccctcttattcatggcttttttaatccatggtttcagaaagcattcaagtacatagtgtcaggaaaaatatttagctcccattcagaaactgc

SEQ ID NO: 169 - DNA, 526 bp, Homo sapiens
cacatctggacccatcagtgactgcctgccatagcctgagagtgtcttggggagaccttgcagagggggagaattgttccttctgctttcctaggggactcttgagcttagaaactcatcgtacacttgaccttgagccttctatttgcctcatctataacatgaagtgctagcatcagatatttgagagctcttagctctgtacccgggtgcctggtttttggggagtcatccgcagagtcactcacccactgtgtttctggtgccaaggctcttgagggccccactctcatccctcctttccctaccagggactcggaggaaggcataggagatatttccaggcttacgaccctgggctcacgggtacctatttatatgctcagtgcagagcactgtggatgtgccaggaggggtagccctgttcaagagcaatttctgccctttgtaaattatttaagaaacctgctttgtcattttattagaaagaaaccagcgtgtgactttcctagataacactgctttc

SEQ ID NO: 170 - DNA, 447 bp, Homo sapiens
ccgccaggagagcgtgcagctcgaagagaactgcctgtgccgcttccactggtgctgcgtagtacagtgccaccgttgccgtgtgcgcaaggagctcagcctctgcctgtgacccgccgcccggccgctagactgacttcgcgcagcggtggctcgcacctgtgggacctcagggcaccggcaccgggcgcctctcgccgctcgagcccagcctctccctgccaaagcccaactcccagggctctggaaatggtgaggcgaggggcttgagaggaacgcccacccacgaaggcccagggcgccagacggccccgaaaaggcgctcggggagcgtttaaaggacactgtacaggccctccctccccttggcctctaggaggaaacagttttttagactggaaaaaagccagtctaaaggcctctggatactgggctccccagaactgc

SEQ ID NO: 171 - DNA, 394 bp, Homo sapiens
gcgatgcagaaatgaaccaccggagttcaatgcgagttcttggggatgttgtcaggagacctcccattcataggagaagtttcagtctagaaggcttgacaggaggagctggtgtcggaaacaagccatcctcatctctagaagtaagctctgcaaatgccgaagagctcagacacccattcagtggtgaggaacgggttgactctttggtgtcactttcagaagaggatctggagtcagaccagagagaacataggatgtttgatcagcagatatgtcacagatctaagcagcagggatttaattactgtacatcagccatttcctctccattgacaaaatccatctcattaatgacaatcagccatcctggattggacaattcacggccctt

SEQ ID NO: 172 - DNA, 480 bp, Homo sapiens
  misc_feature (57)..(57): n is a, c, g, or t
gtaggctcagcgatagtggtcctcttacagagaaacggggagcaggacgacgggggngctggggntggcgggggagggtgcccacaaaaagaatcaggacttgtactgggaaaaaaacccctaaattaattatatttcttggacattccctttcctaacatcctgaggcttaaaaccctgatgcaaacttctcctttcagtggttggagaaattggccgagttcaaccattcactgcaatgcctattccaaactttaaatctatctattgcaaaacctgaaggactgtagttagcggggatgatgttaagtgtggccaagcgcacggcggcaagttttcaagcactgagtttctattccaagatcatagacttactaaagagagtgacaaatgcttccttaatgtcttctataccagaatgtaaatatttttgtgttttgtgttaatttgttagaattctaacacactatatacttccaa

SEQ ID NO: 173 - DNA, 24 bp, Homo sapiens
agtcactcacccactgtgtttctg

SEQ ID NO: 174 - DNA, 22 bp, Homo sapiens
ctgtgttctgcatggtttggat

SEQ ID NO: 175 - DNA, 20 bp, Homo sapiens
atttgagtgggtgtccaggg

SEQ ID NO: 176 - DNA, 20 bp, Homo sapiens
aaaggaccgcatcagtgagc

SEQ ID NO: 177 - DNA, 21 bp, Homo sapiens
aagaagattgggcagttgggt

SEQ ID NO: 178 - DNA, 22 bp, Homo sapiens
aagataaacagccccaggaacc

SEQ ID NO: 179 - DNA, 24 bp, Homo sapiens
gtaggaaaaatgcaagccatctct

SEQ ID NO: 180 - DNA, 23 bp, Homo sapiens
aaaggaaagattggttctcccag

SEQ ID NO: 181 - DNA, 20 bp, Homo sapiens
tcacagctccctccagaagc

SEQ ID NO: 182 - DNA, 23 bp, Homo sapiens
caggcttttgagctgatcttgaa

SEQ ID NO: 183 - DNA, 23 bp, Homo sapiens
gccaacagtacaatagcccacaa

SEQ ID NO: 184 - DNA, 25 bp, Homo sapiens
agttggaaatgtggagtattttgga

SEQ ID NO: 185 - DNA, 20 bp, Homo sapiens
ctgaccgagaacgaactgca

SEQ ID NO: 186 - DNA, 21 bp, Homo sapiens
agcgattcacgtaggatctgc

SEQ ID NO: 187 - DNA, 20 bp, Homo sapiens
tgccccttaatgccattgaa

SEQ ID NO: 188 - DNA, 22 bp, Homo sapiens
ggtagggaaaggagggatgaga

SEQ ID NO: 189 - DNA, 21 bp, Homo sapiens
ggttggaagaagttcggttgg

SEQ ID NO: 190 - DNA, 20 bp, Homo sapiens
ggtcaaggccaatgctctgt

SEQ ID NO: 191 - DNA, 18 bp, Homo sapiens
agcagttggcgtgcttgg

SEQ ID NO: 192 - DNA, 22 bp, Homo sapiens
tcctgctactcctggctcattc

SEQ ID NO: 193 - DNA, 22 bp, Homo sapiens
ccactgaggagctgtctgcttt

SEQ ID NO: 194 - DNA, 24 bp, Homo sapiens
catgattagtactgctagcggacc

SEQ ID NO: 195 - DNA, 24 bp, Homo sapiens
agtagaccaagcacaggcatacag

SEQ ID NO: 196 - DNA, 21 bp, Homo sapiens
gatgaggactgggagagggtt

SEQ ID NO: 197 - DNA, 23 bp, Homo sapiens
tttggagaagctaaagttcgtgg

SEQ ID NO: 198 - DNA, 24 bp, Homo sapiens
ccacgacctacaatgatgatatcg

SEQ ID NO: 199 - DNA, 27 bp, Homo sapiens
catagtacggataatactgcagaggaa

SEQ ID NO: 200 - DNA, 18 bp, Homo sapiens
agtcccctttgccccctc

SEQ ID NO: 201 - DNA, 21 bp, Homo sapiens
atcaccagtgttggaagtggg

SEQ ID NO: 202 - DNA, 19 bp, Homo sapiens
ttttgccatggacaatgca

(19) United States
(12) Patent Application Publication
(10) Pub. No.: US 2010/0184063 A1
(43) Pub. Date: Jul. 22, 2010

(54) PROGNOSTIC AND PREDICTIVE GENE SIGNATURE FOR NON-SMALL CELL LUNG CANCER AND ADJUVANT CHEMOTHERAPY

(76) Inventors: Ming-Sound Tsao, Toronto (CA); Frances A. Shepherd, Toronto (CA); Igor Jurisica, Toronto (CA); Sandy D. Der, Toronto (CA); Chang-Qi Zhu, Thornhill (CA); Dan Strumpf, Toronto (CA); Lesley Seymour, Kingston (CA); Keyue Ding, Kingston (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/684,370

(22) Filed: Jan. 8, 2010

Related U.S. Application Data
(63) Continuation-in-part of application No. 12/465,954, filed on May 14, 2009.
(60) Provisional application No. 61/071,728, filed on May 14, 2008.

(51) Int. Cl. C12Q 1/68

(52) U.S. Cl. 435/6

(57) ABSTRACT

The application provides methods of prognosing and classifying lung cancer patients into poor survival groups or good survival groups and for determining the benefit of adjuvant chemotherapy by way of a multigene signature. The application also includes kits and computer products for use in the methods of the application.

### CROSS-REFERENCE TO RELATED APPLICATION

This application is a continuation-in-part of U.S. utility application Ser. No. 12/465,954 filed 14 May 2009 (pending), which claims benefit under 35 U.S.C. §119(e) to U.S. Provisional Application Ser. No. 61/071728, filed 14 May 2008 (now abandoned), incorporated herein by reference in its entirety.

### FIELD

The application relates to compositions and methods for prognosing and classifying non-small cell lung cancer and for determining the benefit of adjuvant chemotherapy.

### BACKGROUND OF THE INVENTION

In North America, lung cancer is the leading cancer in males and the leading cause of cancer deaths in both males and females¹. Non-small cell lung cancer (NSCLC) represents 80% of all lung cancers and has an overall 5-year survival rate of only 16%¹. Tumor stage is the primary determinant for treatment selection for NSCLC patients. Recent clinical trials have led to the adoption of adjuvant cisplatin-based chemotherapy in early stage NSCLC patients (Stages IB-IIIA). The 5-year survival advantage conferred by adjuvant chemotherapy in recent trials are 4% in the International Adjuvant Lung Trial (IALT) involving 1,867 Stage I-III patients², 15% in the National Cancer Institute of Canada Clinical Trials Group (NCIC CTG) BR.10 Trial involving 483 Stage IB-II patients³, and 9% in the Adjuvant Navelbine International Trialist Association (ANITA) trial involving 840 Stage IB-IIIA patients⁴. Pre-planned stratification analysis in the later two trials showed no significant survival benefit for Stage IB patients^{3, 4}. This was also demonstrated in the Cancer and Leukemia Group (CALGB) Trial 9633 that tested the benefit of chemotherapy on 344 Stage IB patients receiving carboplatin and paclitaxel or observation⁵. Although initially presented in 2004 as a positive trial, recent survival analyses show no significant survival advantage with chemotherapy for either disease-free survival (HR=0.80, p=0.065) or overall survival (HR=0.83, p=0.12)⁵. In an attempt to draw an overall conclusion regarding the effectiveness of adjuvant cisplatin-based chemotherapy, the Lung Adjuvant Cisplatin Evaluation (LACE) meta-analysis⁶ was conducted which synthesized information from the 5 largest published, cisplatin-based trials that did not administer concurrent thoracic radiation [Adjuvant Lung Project Italy (ALPI)⁷, Big Lung Trial (BLT)⁸, IALT², BR.10³, and ANITA⁹]. The study found a 5.3% absolute survival advantage at 5-year (HR=0.89, 95%CI 0.82-0.96, p=0.004). However, stratified analysis by stage showed that the Stage IB patients did not benefit significantly from cisplatin treatment (HR=0.92, 95%CI 0.78-1.10). Moreover, a detriment for chemotherapy was suggested in Stage IA patients (HR=1.41, 95%CI 0.96-2.09)⁶. Therefore, the current standard of treatment for patients with Stage I NSCLC remains surgical resection alone. However, 30 to 40 percent of these Stage I patients are expected to relapse after the initial surgery^{10, 11}, indicating that a subgroup of these patients might benefit from adjuvant chemotherapy.

The lack of consistent prognostic molecular markers for early stage NSCLC patients led to attempts to identify novel gene expression signatures using genome wide microarray platforms. Such multi-gene signatures might be stronger than individual genes to predict poor prognosis and poor prognostic patients could potentially benefit from adjuvant therapies. Previous microarray studies have identified prognostic signatures that demonstrated minimal overlaps in the gene sets¹²⁻²⁰. While only one of the early studies involved secondary signature validation in independent datasets¹², all recently reported signatures were tested for validation^{13-16, 20}. Nevertheless, lack of direct overlaps between signatures remains. One of the potential confounding factors is that signatures were derived from patients operated at single institutions, which may introduce biases.

### SUMMARY OF THE INVENTION

As discussed in the Background section, certain patients suffering from NSCLC benefit from adjuvant chemotherapy. Attempts to identify systematically patient subpopulations in which adjuvant therapy would lead to increased survival or improve patient prognosis have generally failed. Efforts to assemble prognostic molecular markers have yielded various non-overlapping gene sets but have fallen short of establishing a gene signature with a minimal set of genes that is predictive regardless of the form of NSCLC (e.g., adenocarcinoma or squamous cell carcinoma) or stage, and serves as a reliable classifier for adjuvant therapy benefit.

As will be discussed in more detail below, Applicants have identified from historical patient data a minimal set of fifteen genes whose expression levels, either alone or in combination with that of one to 3 additional genes, is prognostic of survival outcome and diagnostic of adjuvant therapy benefit. The fifteen genes are provided in Table 4. Optional additional genes may be selected from those provided in Table 3. The prognostic and diagnostic value of the gene sets identified by Applicants was verified by validation against independent data sets, as set forth in the Examples below. The present disclosure provides methods and kits useful for obtaining and utilizing expression information for the fifteen, and optionally one to 3 additional genes, to obtain prognostic and diagnostic information for patient with NSCLC.

The methods of the present disclosure generally involve obtaining from a patient relative expression data, at the DNA, messenger RNA (mRNA), or protein level, for each of the fifteen, and optional additional, genes, processing the data and comparing the resulting information to one or more reference values. Relative expression levels are expression data normalized according to techniques known to those skilled in the art. Expression data may be normalized with respect to one or more genes with invariant expression, such as “housekeeping” genes. In some embodiments, expression data may be processed using standard techniques, such as transformation to a z-score, and/or software tools, such as RMAexpress v0.3.

In one aspect, a multi-gene signature is provided for prognosing or classifying patients with lung cancer. In some embodiments, a fifteen-gene signature is provided, comprising reference values for each of fifteen different genes based on relative expression data for each gene from a historical data set with a known outcome, such as good or poor survival, and/or known treatment, such as adjuvant chemotherapy. In one embodiment, four reference values are provided for each of the fifteen genes listed in Table 4. In one embodiment, the reference values for each of the fifteen genes are principal component values set forth in Table 10.

In some embodiments, a sixteen-, seventeen-, or eighteen-gene signature comprises reference values for each of sixteen, seventeen, or eighteen different genes based on relative expression data for each gene from a historical data set with a known outcome and/or known treatment. In some embodiments, reference values are provided for one, two, three genes in addition to those listed in Table 4, and the genes are selected from those listed in Table 3. In some embodiments, a single reference value for each gene is provided.

In one aspect, relative expression data from a patient are combined with the gene-specific reference values on a gene-by-gene basis for each of the fifteen, and optional additional, genes, to generate a test value which allows prognosis or therapy recommendation. In some embodiments, relative expression data are subjected to an algorithm that yields a single test value, or combined score, which is then compared to a control value obtained from the historical expression data for a patient or pool of patients. In some embodiments, the control value is a numerical threshold for predicting outcomes, for example good and poor outcome, or making therapy recommendations, for example adjuvant therapy in addition to surgical resection or surgical resection alone. In some embodiments, a test value or combined score greater than the control value is predictive, for example, of high risk (poor outcome) or benefit from adjuvant therapy, whereas a combined score falling below the control value is predictive, for example, of low risk (good outcome) or lack of benefit from adjuvant therapy.

In one embodiment, the combined score is calculated from relative expression data multiplied by reference values, determined from historical data, for each gene. Accordingly, the combined score may be calculated using the algorithm of Formula I below:

Combined score=0.557×PC1+0.328×PC2+0.43×PC3+0.335×PC4

where PC1 is the sum of the relative expression level for each gene in a multi-gene signature multiplied by a first principal component for each gene in the multi-gene signature, PC2 is the sum of the relative expression level for each gene multiplied by a second principal component for each gene, PC3 is the sum of the relative expression level for each gene multiplied by a third principal component for each gene, and PC4 is the sum of the relative expression level for each gene multiplied by a fourth principal component for each gene. In some embodiments, the combined score is referred to as a risk score. A risk score for a subject can be calculated by applying Formula I to relative expression data from a test sample obtained from the subject.

In some embodiments, PC1 is the sum of the relative expression level for each gene provided in Table 4 multiplied by a first principal component for each gene, respectively, as set forth in Table 10; PC2 is the sum of the relative expression level for each gene provided in Table 4 multiplied by a second principal component for each gene, respectively, as set forth in Table 10; PC3 is the sum of the relative expression level for each gene provided in Table 4 multiplied by a third principal component for each gene, respectively, as set forth in Table 10; and PC4 is the sum of the relative expression level for each gene provided in Table 4 multiplied by a fourth principal component for each gene, respectively, as set forth in Table 10.1

The present inventors have identified a gene signature that is prognostic for survival as well as predictive for benefit from adjuvant chemotherapy.

Accordingly in one embodiment, the application provides a method of prognosing or classifying a subject with non-small cell lung cancer comprising the steps:

a. determining the expression of fifteen biomarkers in a test sample from the subject, wherein the biomarkers correspond to genes in Table 4, and

b. comparing the expression of the fifteen biomarkers in the test sample with expression of the fifteen biomarkers in a control sample,

wherein a difference or a similarity in the expression of the fifteen biomarkers between the control and the test sample is used to prognose or classify the subject with NSCLC into a poor survival group or a good survival group.

In an aspect, the application provides a method of predicting prognosis in a subject with non-small cell lung cancer comprising the steps:

a. obtaining a subject biomarker expression profile in a sample of the subject;

b. obtaining a biomarker reference expression profile associated with a prognosis, wherein the subject biomarker expression profile and the biomarker reference expression profile each have fifteen values, each value representing the expression level of a biomarker, wherein each biomarker corresponds to one gene in Table 4; and

c. selecting the biomarker reference expression profile most similar to the subject biomarker expression profile, to thereby predict a prognosis for the subject.

In another aspect, the prognoses and classifying methods of the application can be used to select treatment. For example, the methods can be used to select or identify subjects who might benefit from adjuvant chemotherapy. Accordingly, in one embodiment, the application provides a method of selecting a therapy for a subject with NSCLC, comprising the steps:

a. classifying the subject with NSCLC into a poor survival group or a good survival group according to the method of the application; and

b. selecting adjuvant chemotherapy for the poor survival group or no adjuvant chemotherapy for the good survival group.

In another embodiment, the application provides a method of selecting a therapy for a subject with NSCLC, comprising the steps:

a. determining the expression of fifteen biomarkers in a test sample from the subject, wherein the fifteen biomarkers correspond to the fifteen genes in Table 4;

b. comparing the expression of the fifteen biomarkers in the test sample with the fifteen biomarkers in a control sample;

c. classifying the subject in a poor survival group or a good survival group,

wherein a difference or a similarity in the expression of the fifteen biomarkers between the control sample and the test sample is used to classify the subject into a poor survival group or a good survival group;

d. selecting adjuvant chemotherapy if the subject is classified in the poor survival group and selecting no adjuvant chemotherapy if the subject is classified in the good survival group.

Another aspect of the application provides compositions useful for use with the methods described herein.

The application also provides for kits used to prognose or classify a subject with NSCLC into a good survival group or a poor survival group or for selecting therapy for a subject with NSCLC that includes detection agents that can detect the expression products of the biomarkers.

The present disclosure provides probes for detecting the biomarkers described herein. Exemplary probes include mRNA oligonucleotides, cDNA oligonucleotides, and PCR primers. The probes are capable of detecting or hybridizing to, each of the at least 15, and optionally 16, 17, or 18 biomarkers described herein.

In one aspect, the present disclosure provides kits useful for carrying out the diagnostic and prognostic tests described herein. The kits generally comprise reagents and compositions for obtaining relative expression data for the fifteen, and optional additional, genes described in Tables 3 and 4. The kits typically comprise probes for detecting the at least 15 biomarkers described herein. The present disclosure also provides antibodies capable of specifically binding to the protein products of the biomarkers described herein. As will be recognized by the skilled artisans, the contents of the kits will depend upon the means used to obtain the relative expression information.

Kits may comprise a labeled compound or agent capable of detecting protein product(s) or nucleic acid sequence(s) in a sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

In some embodiments, the kits are oligonucleotide-based kits, which may comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. Kits may also comprise, e.g., a buffering agent, a preservative, or a protein stabilizing agent. The kits can further comprise components necessary for detecting the detectable label (e.g., an enzyme or a substrate). The kits can also contain a control sample or a series of control samples which can be assayed and compared to the test sample. Each component of a kit can be enclosed within an individual container and all of the various containers can be within a single package, along with instructions for interpreting the results of the assays performed using the kit.

In some embodiments, the kits are antibody-based kits, which may comprise, for example: (1) a first antibody (e.g., attached to a solid support) which binds to a marker protein; and, optionally, (2) a second, different antibody which binds to either the protein or the first antibody and is conjugated to a detectable label.

A further aspect provides computer implemented products, computer readable mediums and computer systems that are useful for the methods described herein.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the derivation and testing of the prognostic signature;

FIG. 2 shows the survival outcome based on the 15-gene signature in training and test sets;

FIG. 3 shows a comparison of chemotherapy vs. observation in low and high risk patients with microarray data;

FIG. 4 shows a consort diagram for microarray study of BR. 10 patients;

FIG. 5 shows the effect of adjuvant chemotherapy in microarray profiled patients;

FIG. 6 shows the effect of microarray batch processing at 2 different times. The samples were profiled in 2 batches at 2 times (January 2004 and June 2005). Unsupervised clustering shows that the expression patterns of these two batches differed significantly with samples arrayed on January 2004 aggregated in cluster 1 (93%) and samples arrayed on June 2005 in cluster 2 (73%);

FIG. 7 provides graphs of percent survival over time of Stage IB-II patients who received no adjuvant therapy, classified into either a low risk or a high risk group based on a 15-gene signature prognostic for overall survival. The prognostic signature was validated in 4 separate datasets as depicted in FIGS. 7A-D. DCC: Director's Challenge Consortium adenocarcinoma dataset (FIG. 7A); NLCI: Netherlands Cancer Institute (FIG. 7B); Duke: Duke University (FIG. 7C); UM-SQ: University of Michigan squamous cancer dataset (FIG. 7D); HR: unadjusted hazard ratio; and

FIG. 8 shows validation of the 15-gene prognostic signature on overall survival of patients with different stages of NSCLC in a cohort of 183 patients from Princess Margaret Hospital/University Health Network who received no adjuvant therapy. FIG. 8A. Stage I and II; FIG. 8B. Stage I; FIG. 8C. Stage IB and II; FIG. 8D. Stage II. HR: unadjusted hazard ratio.

### DETAILED DESCRIPTION OF THE INVENTION

The application relates to 15 biomarkers that form a 15-gene signature, and provides methods, compositions, computer implemented products, detection agents and kits for prognosing or classifying a subject with non-small cell lung cancer (NSCLC) and for determining the benefit of adjuvant chemotherapy.

The term “biomarker” as used herein refers to a gene that is differentially expressed in individuals with non-small cell lung cancer (NSCLC) according to prognosis and is predictive of different survival outcomes and of the benefit of adjuvant chemotherapy. In some embodiments, a 15-gene signature comprises 15 biomarker genes listed in Table 4. Optional additional biomarkers for a 16-, 17-, or 18-gene signature may be selected from the genes listed in Table 3.

Accordingly, one aspect of the invention is a method of prognosing or classifying a subject with non-small cell lung cancer, comprising the steps:

a. determining the expression of fifteen biomarkers in a test sample from the subject, wherein the biomarkers correspond to genes in Table 4, and

b. comparing the expression of the fifteen biomarkers in the test sample with expression of the fifteen biomarkers in a control sample,

wherein a difference or a similarity in the expression of the fifteen biomarkers between the control and the test sample is used to prognose or classify the subject with NSCLC into a poor survival group or a good survival group.

In another aspect, the application provides a method of predicting prognosis in a subject with non-small cell lung cancer (NSCLC) comprising the steps:

a. obtaining a subject biomarker expression profile in a sample of the subject;

b. obtaining a biomarker reference expression profile associated with a prognosis, wherein the subject biomarker expression profile and the biomarker reference expression profile each have fifteen values, each value representing the expression level of a biomarker, wherein each biomarker corresponds to a gene in Table 4; and

c. selecting the biomarker reference expression profile most similar to the subject biomarker expression profile, to thereby predict a prognosis for the subject.

The term “reference expression profile” as used herein refers to the expression of the 15 biomarkers or genes listed in Table 4 associated with a clinical outcome in a NSCLC patient. The reference expression profile comprises 15 values, each value representing the expression level of a biomarker, wherein each biomarker corresponds to one gene in Table 4. The reference expression profile is identified using one or more samples comprising tumor wherein the expression is similar between related samples defining an outcome class or group such as poor survival or good survival and is different to unrelated samples defining a different outcome class such that the reference expression profile is associated with a particular clinical outcome. The reference expression profile is accordingly a reference profile of the expression of the 15 genes in Table 4, to which the subject expression levels of the corresponding genes in a patient sample are compared in methods for determining or predicting clinical outcome.

As used herein, the term “control” refers to a specific value or dataset that can be used to prognose or classify the value, e.g., expression level or reference expression profile obtained from the test sample associated with an outcome class. In one embodiment, a dataset may be obtained from samples from a group of subjects known to have NSCLC and good survival outcome or known to have NSCLC and have poor survival outcome or known to have NSCLC and have benefited from adjuvant chemotherapy or known to have NSCLC and not have benefited from adjuvant chemotherapy. The expression data of the biomarkers in the dataset can be used to create a “control value” that is used in testing samples from new patients. A control value is obtained from the historical expression data for a patient or pool of patients with a known outcome. In some embodiments, the control value is a numerical threshold for predicting outcomes, for example good and poor outcome, or making therapy recommendations, for example adjuvant therapy in addition to surgical resection or surgical resection alone.

In some embodiments, the “control” is a predetermined value for the set of 15 biomarkers obtained from NSCLC patients whose biomarker expression values and survival times are known. Alternatively, the “control” is a predetermined reference profile for the set of fifteen biomarkers obtained from NSCLC patients whose survival times are known. Using values from known samples allows one to develop an algorithm for classifying new patient samples into good and poor survival groups as described in the Example.

Accordingly, in one embodiment, the control is a sample from a subject known to have NSCLC and good survival outcome. In another embodiment, the control is a sample from a subject known to have NSCLC and poor survival outcome.

A person skilled in the art will appreciate that the comparison between the expression of the biomarkers in the test sample and the expression of the biomarkers in the control will depend on the control used. For example, if the control is from a subject known to have NSCLC and poor survival, and there is a difference in expression of the biomarkers between the control and test sample, then the subject can be prognosed or classified in a good survival group. If the control is from a subject known to have NSCLC and good survival, and there is a difference in expression of the biomarkers between the control and test sample, then the subject can be prognosed or classified in a poor survival group. For example, if the control is from a subject known to have NSCLC and good survival, and there is a similarity in expression of the biomarkers between the control and test sample, then the subject can be prognosed or classified in a good survival group. For example, if the control is from a subject known to have NSCLC and poor survival, and there is a similarity in expression of the biomarkers between the control and test sample, then the subject can be prognosed or classified in a poor survival group.

As used herein, a “reference value” refers to a gene-specific coefficient derived from historical expression data. The multi-gene signatures of the present disclosure comprise gene-specific reference values. In some embodiments, the multi-gene signature comprises one reference value for each gene in the signature. In some embodiments, the multi-gene signature comprises four reference values for each gene in the signature. In some embodiments, the reference values are the first four components derived from principal component analysis for each gene in the signature.

The term “differentially expressed” or “differential expression” as used herein refers to a difference in the level of expression of the biomarkers that can be assayed by measuring the level of expression of the products of the biomarkers, such as the difference in level of messenger RNA transcript expressed or proteins expressed of the biomarkers. In a preferred embodiment, the difference is statistically significant. The term “difference in the level of expression” refers to an increase or decrease in the measurable expression level of a given biomarker as measured by the amount of messenger RNA transcript and/or the amount of protein in a sample as compared with the measurable expression level of a given biomarker in a control. In one embodiment, the differential expression can be compared using the ratio of the level of expression of a given biomarker or biomarkers as compared with the expression level of the given biomarker or biomarkers of a control, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment the differential expression is measured using p-value. For instance, when using p-value, a biomarker is identified as being differentially expressed as between a first sample and a second sample when the p-value is less than 0.1, preferably less than 0.05, more preferably less than 0.01, even more preferably less than 0.005, the most preferably less than 0.001.

The term “similarity in expression” as used herein means that there is no or little difference in the level of expression of the biomarkers between the test sample and the control or reference profile. For example, similarity can refer to a fold difference compared to a control. In a preferred embodiment, there is no statistically significant difference in the level of expression of the biomarkers.

The term “most similar” in the context of a reference profile refers to a reference profile that is associated with a clinical outcome that shows the greatest number of identities and/or degree of changes with the subject profile.

The term “prognosis” as used herein refers to a clinical outcome group such as a poor survival group or a good survival group associated with a disease subtype which is reflected by a reference profile such as a biomarker reference expression profile or reflected by an expression level of the fifteen biomarkers disclosed herein. The prognosis provides an indication of disease progression and includes an indication of likelihood of death due to lung cancer. In one embodiment the clinical outcome class includes a good survival group and a poor survival group.

The term “prognosing or classifying” as used herein means predicting or identifying the clinical outcome group that a subject belongs to according to the subject's similarity to a reference profile or biomarker expression level associated with the prognosis. For example, prognosing or classifying comprises a method or process of determining whether an individual with NSCLC has a good or poor survival outcome, or grouping an individual with NSCLC into a good survival group or a poor survival group.

The term “good survival” as used herein refers to an increased chance of survival as compared to patients in the “poor survival” group. For example, the biomarkers of the application can prognose or classify patients into a “good survival group.” These patients are at a lower risk of death after surgery.

The term “poor survival” as used herein refers to an increased risk of death as compared to patients in the “good survival” group. For example, biomarkers or genes of the application can prognose or classify patients into a “poor survival group.” These patients are at greater risk of death from surgery.

Accordingly, in one embodiment, the biomarker reference expression profile comprises a poor survival group. In another embodiment, the biomarker reference expression profile comprises a good survival group.

The term “subject” as used herein refers to any member of the animal kingdom, preferably a human being that has NSCLC or that is suspected of having NSCLC.

NSCLC patients are classified into stages, which are used to determine therapy. Staging classification testing may include any or all of history, physical examination, routine laboratory evaluations, chest x-rays, and chest computed tomography scans or positron emission tomography scans with infusion of contrast materials. For example, Stage I includes cancer in the lung, but has not spread to adjacent lymph nodes or outside the chest. Stage I is divided into two categories based on the size of the tumor (IA and IB). Stage II includes cancer located in the lung and proximal lymph nodes. Stage II is divided into 2 categories based on the size of tumor and nodal status (IIA and IIB). Stage III includes cancer located in the lung and the lymph nodes. Stage III is divided into 2 categories based on the size of tumor and nodal status (IIIA and IIIB). Stage IV includes cancer that has metastasized to distant locations. The term “early stage NSCLC” includes patients with Stage I to IIIA NSCLC. These patients are treated primarily by complete surgical resection.

In an aspect, a multi-gene signature is prognostic of patient outcome and/or response to adjuvant chemotherapy. The present disclosure provides a prognostic signature that is a stage-independent classifier. In some embodiments, a minimal signature for 15 genes is provided. In one embodiment, the signature comprises reference values for each of the 15 genes listed in Table 4. In some embodiments, the 15-gene signature is associated with the early stages of NSCLC. Accordingly, in one embodiment, the multi-gene signature is an independent prognostic factor for a subject with stage I NSCLC. In another embodiment, the multi-gene signature is an independent prognostic factor for a subject with Stage II NSCLC. In some embodiments, a 16-, 17-, 18-gene signature is prognostic of patient outcome and/or response to adjuvant chemotherapy. In some embodiments, the signature comprises reference values for one, two or three genes selected from those listed in Table 3, in addition to reference values for each of the 15 genes listed in Table 4. In some embodiments, the additional one, two, or three genes are selected from RGS4, UGT2B4, and MCF2 listed in Table 3.

In some embodiments, the multi-gene signature comprises four coefficients, or reference values, for each gene in the signature. In one embodiment, the four coefficients are the first four principal components derived from principal component analysis described in Example 1 below. In one embodiment, the 15-gene signature comprises the principal component values listed in Table 10 below. In some embodiments, a 16-, 17-, 18-gene signature comprises coefficients for a sixteenth, seventeenth, and eighteenth gene, respectively, derived from principal component analysis as described in Example 1 below. In some embodiments, the coefficients for a sixteenth, seventeenth, and eighteenth gene, respectively, are the first four principal components derived according to Example 1. In some embodiments, the additional one, two, or three genes are selected from RGS4, UGT2B4, and MCF2 listed in Table 3.

The term “test sample” as used herein refers to any cancer-affected fluid, cell or tissue sample from a subject which can be assayed for biomarker expression products and/or a reference expression profile, e.g., genes differentially expressed in subjects with NSCLC according to survival outcome.

The phrase “determining the expression of biomarkers” as used herein refers to determining or quantifying RNA or proteins expressed by the biomarkers. The term “RNA” includes mRNA transcripts, and/or specific spliced variants of mRNA. The terms “RNA product of the biomarker,” “biomarker RNA,” or “target RNA” as used herein refers to RNA transcripts transcribed from the biomarkers and/or specific spliced variants. In the case of “protein,” it refers to proteins translated from the RNA transcripts transcribed from the biomarkers. The term “protein product of the biomarker” or “biomarker protein” refers to proteins translated from RNA products of the biomarkers.

A person skilled in the art will appreciate that a number of methods can be used to detect or quantify the level of RNA products of the biomarkers within a sample, including arrays, such as microarrays, RT-PCR (including quantitative PCR), nuclease protection assays and Northern blot analyses. Any analytical procedure capable of permitting specific and quantifiable (or semi-quantifiable) detection of the 15 and, optionally, additional biomarkers may be used in the methods herein presented, such as the microarray methods set forth herein, and methods known to those skilled in the art.

Accordingly, in one embodiment, the biomarker expression levels are determined using arrays, optionally microarrays, RT-PCR, optionally quantitative RT-PCR, nuclease protection assays or Northern blot analyses.

In some embodiments, the biomarker expression levels are determined by using an array. cDNA microarrays consist of multiple (usually thousands) of different cDNA probes spotted (usually using a robotic spotting device) onto known locations on a solid support, such as a glass microscope slide. Microarrays for use in the methods described herein comprise a solid substrate onto which the probes are covalently or non-covalently attached. The cDNAs are typically obtained by PCR amplification of plasmid library inserts using primers complementary to the vector backbone portion of the plasmid or to the gene itself for genes where sequence is known. PCR products suitable for production of microarrays are typically between 0.5 and 2.5 kB in length. In a typical microarray experiment, RNA (either total RNA or poly A RNA) is isolated from cells or tissues of interest and is reverse transcribed to yield cDNA. Labeling is usually performed during reverse transcription by incorporating a labeled nucleotide in the reaction mixture. A microarray is then hybridized with labeled RNA, and relative expression levels calculated based on the relative concentrations of cDNA molecules that hybridized to the cDNAs represented on the microarray. Microarray analysis can be performed by commercially available equipment, following manufactuer's protocols, such as by using Affymetrix GeneChip technology, Agilent Technologies cDNA microarrays, Illumina Whole-Genome DASL array assays, or any other comparable microarray technology.

In some embodiments, probes capable of hybridizing to one or more biomarker RNAs or cDNAs are attached to the substrate at a defined location (“addressable array”). Probes can be attached to the substrate in a wide variety of ways, as will be appreciated by those in the art. In some embodiments, the probes are synthesized first and subsequently attached to the substrate. In other embodiments, the probes are synthesized on the substrate. In some embodiments, probes are synthesized on the substrate surface using techniques such as photopolymerization and photolithography.

In some embodiments, microarrays are utilized in a RNA-primed, Array-based Klenow Enzyme (“RAKE”) assay. See Nelson, P. T. et al. (2004) Nature Methods 1(2):1-7; Nelson, P. T. et al. (2006) RNA 12(2):1-5, each of which is incorporated herein by reference in its entirety. In these embodiments, total RNA is isolated from a sample. Optionally, small RNAs can be further purified from the total RNA sample. The RNA sample is then hybridized to DNA probes immobilized at the 5′-end on an addressable array. The DNA probes comprise a base sequence that is complementary to a target RNA of interest, such as one or more biomarker RNAs capable of specifically hybridizing to a nucleic acid comprising a sequence that is identically present in one of the genes listed in Table 4 under standard hybridization conditions.

In some embodiments, the addressable array comprises DNA probes for no more than the 15 genes listed in Table 4. In some embodiments, the addressable array comprises DNA probes for each of the 15 genes listed in Table 4 and optionally, no more than one, two, or three additional genes selected from those listed in Table 3. In one embodiment, the addressable array comprises DNA probes for each of the 15 genes listed in Table 4 and DNA probes for one, two, or all three of RGS4, UGT2B4, and MCF2 listed in Table 3.

In some embodiments, quantitation of biomarker RNA expression levels requires assumptions to be made about the total RNA per cell and the extent of sample loss during sample preparation. In some embodiments, the addressable array comprises DNA probes for each of the 15 genes listed in Table 4 and, optionally, one, two, three, or four housekeeping genes. In one embodiment, the addressable array comprises DNA probes for each of the 15 genes listed in Table 4, one, two, three, or four housekeeping genes, and, additionally, no more than one, two, three or four additional genes selected from those listed in Table 3.

In some embodiments, expression data are pre-processed to correct for variations in sample preparation or other non-experimental variables affecting expression measurements. For example, background adjustment, quantile adjustment, and summarization may be performed on microarray data, using standard software programs such as RMAexpress v0.3, followed by centering of the data to the mean and scaling to the standard deviation.

After the sample is hybridized to the array, it is exposed to exonuclease I to digest any unhybridized probes. The Klenow fragment of DNA polymerase I is then applied along with biotinylated dATP, allowing the hybridized biomarker RNAs to act as primers for the enzyme with the DNA probe as template. The slide is then washed and a streptavidin-conjugated fluorophore is applied to detect and quantitate the spots on the array containing hybridized and Klenow-extended biomarker RNAs from the sample.

In some embodiments, the RNA sample is reverse transcribed using a biotin/poly-dA random octamer primer. The RNA template is digested and the biotin-containing cDNA is hybridized to an addressable microarray with bound probes that permit specific detection of biomarker RNAs. In typical embodiments, the microarray includes at least one probe comprising at least 8, at least 9, at least 10, at least 11, at least 12, at least 13, at least 14, at least 15, at least 16, at least 17, at least 18, at least 19, even at least 20, 21, 22, 23, or 24 contiguous nucleotides identically present in each of the genes listed in Table 4. After hybridization of the cDNA to the microarray, the microarray is exposed to a streptavidin-bound detectable marker, such as a fluorescent dye, and the bound cDNA is detected. See Liu C. G. et al. (2008) Methods 44:22-30, which is incorporated herein by reference in its entirety.

In one embodiment, the array is a U133A chip from Affymetrix. In another embodiment, a plurality of nucleic acid probes that are complementary or hybridizable to an expression product of the genes listed in Table 4 are used on the array. In a particular embodiment, the probe target sequences are listed in Table 9. In some embodiments, the probe target sequences are selected from SEQ ID NO: 3, 11-15, 22, 26, 35, 49, 78, 85, 130, 133, and 169. In one embodiment, fifteen probes are used, each probe hybridizable to a different target sequence selected from SEQ ID NO: 3, 11-15, 22, 26, 35, 49, 78, 85, 130, 133, and 169. In some embodiments, a plurality of nucleic acid probes that are complementary or hybridizable to an expression product of some or all the genes listed in Table 3 are used on the array. In some embodiments, the probe target sequences are selected from those listed in Table 11. In some embodiments, the probe target sequences are selected from SEQ ID NO: 1-172.

The term “nucleic acid” includes DNA and RNA and can be either double stranded or single stranded.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “probe” as used herein refers to a nucleic acid sequence that will hybridize to a nucleic acid target sequence. In one example, the probe hybridizes to an RNA product of the biomarker or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In some embodiments, compositions are provided that comprise at least one biomarker or target RNA-specific probe. The term “target RNA-specific probe” encompasses probes that have a region of contiguous nucleotides having a sequence that is either (i) identically present in one of the genes listed in Tables 3 or 4, or (ii) complementary to the sequence of a region of contiguous nucleotides found in one of the genes listed in Tables 3 or 4, where “region” can comprise the full length sequence of any one of the genes listed in Tables 3 or 4, a complementary sequence of the full length sequence of any one of the genes listed in Tables 3 or 4, or a subsequence thereof.

In some embodiments, target RNA-specific probes consist of deoxyribonucleotides. In other embodiments, target RNA-specific probes consist of both deoxyribonucleotides and nucleotide analogs. In some embodiments, biomarker RNA-specific probes comprise at least one nucleotide analog which increases the hybridization binding energy. In some embodiments, a target RNA-specific probe in the compositions described herein binds to one biomarker RNA in the sample.

In some embodiments, more than one probe specific for a single biomarker RNA is present in the compositions, the probes capable of binding to overlapping or spatially separated regions of the biomarker RNA.

It will be understood that in some embodiments in which the compositions described herein are designed to hybridize to cDNAs reverse transcribed from biomarker RNAs, the composition comprises at least one target RNA-specific probe comprising a sequence that is identically present in a biomarker RNA (or a subsequence thereof).

In some embodiments, a biomarker RNA is capable of specifically hybridizing to at least one probe comprising a base sequence that is identically present in one of the genes listed in Table 4. In some embodiments, a biomarker RNA is capable of specifically hybridizing to at least one nucleic acid probe comprising a sequence that is identically present in one of the genes listed in Table 3. In some embodiments, a target RNA is capable of specifically hybridizing to at least one nucleic acid probe, and comprises a sequence that is identical to a sequence selected from SEQ ID NO: 1-172, or a sequence listed in Table 11. In some embodiments, a target RNA is capable of specifically hybridizing to at least one nucleic acid probe, and comprises a sequence that is identical to a sequence listed in Table 9. In some embodiments, a target RNA is capable of specifically hybridizing to at least one nucleic acid probe, and comprises a sequence that is identical to a sequence selected from SEQ ID NO: 3, 11-15, 22, 26, 35, 49, 78, 85, 130, 133, and 169. In some embodiments, a biomarker RNA is capable of specifically hybridizing to at least one probe comprising a base sequence that is identically present in one of the genes listed in Table 4.

In some embodiments, the composition comprises a plurality of target or biomarker RNA-specific probes each comprising a region of contiguous nucleotides comprising a base sequence that is identically present in one or more of the genes listed in Table 4, or in a subsequence thereof In some embodiments, the composition comprises a plurality of target or biomarker RNA-specific probes each comprising a region of contiguous nucleotides comprising a base sequence that is complementary to a sequence listed in Table 9. In some embodiments, the composition comprises a plurality of target RNA-specific probes each comprising a region of contiguous nucleotides comprising a base sequence that is complementary to a sequence selected from SEQ ID NO: 3, 11-15, 22, 26, 35, 49, 78, 85, 130, 133, and 169.

As used herein, the terms “complementary” or “partially complementary” to a biomarker or target RNA (or target region thereof), and the percentage of “complementarity” of the probe sequence to that of the biomarker RNA sequence is the percentage “identity” to the reverse complement of the sequence of the biomarker RNA. In determining the degree of “complementarity” between probes used in the compositions described herein (or regions thereof) and a biomarker RNA, such as those disclosed herein, the degree of “complementarity” is expressed as the percentage identity between the sequence of the probe (or region thereof) and the reverse complement of the sequence of the biomarker RNA that best aligns therewith. The percentage is calculated by counting the number of aligned bases that are identical as between the 2 sequences, dividing by the total number of contiguous nucleotides in the probe, and multiplying by 100.

In some embodiments, the microarray comprises probes comprising a region with a base sequence that is fully complementary to a target region of a biomarker RNA. In other embodiments, the microarray comprises probes comprising a region with a base sequence that comprises one or more base mismatches when compared to the sequence of the best-aligned target region of a biomarker RNA.

As noted above, a “region” of a probe or biomarker RNA, as used herein, may comprise or consist of 8, 9, 10, 11, 12, 13, 14, 15, 16, 17, 18, 19, 20, 21, 22, 23, 24, 25, 26, 27, 28, 29 or more contiguous nucleotides from a particular gene or a complementary sequence thereof. In some embodiments, the region is of the same length as the probe or the biomarker RNA. In other embodiments, the region is shorter than the length of the probe or the biomarker RNA.

In some embodiments, the microarray comprises fifteen probes each comprising a region of at least 10 contiguous nucleotides, such as at least 11 contiguous nucleotides, such as at least 13 contiguous nucleotides, such as at least 14 contiguous nucleotides, such as at least 15 contiguous nucleotides, such as at least 16 contiguous nucleotides, such as at least 17 contiguous nucleotides, such as at least 18 contiguous nucleotides, such as at least 19 contiguous nucleotides, such as at least 20 contiguous nucleotides, such as at least 21 contiguous nucleotides, such as at least 22 contiguous nucleotides, such as at least 23 contiguous nucleotides, such as at least 24 contiguous nucleotides, such as at least 25 contiguous nucleotides with a base sequence that is identically present in one of the genes listed in Table 4.

In some embodiments, the microarray component comprises fifteen probes each comprising a region with a base sequence that is identically present in each of the genes listed in Table 4. In some embodiments, the microarray comprises sixteen, seventeen, eighteen probes, each of which comprises a region with a base sequence that is identically present in each of the genes listed in Table 4 and, optionally, one, two, or three of the genes listed in Table 3. In one embodiment, the one, two, or three genes from Table 3 are selected from RGS4, UGT2B4, and MCF2.

In another embodiment, the biomarker expression levels are determined by using quantitative RT-PCR. RT-PCR is one of the most sensitive, flexible, and quantitative methods for measuring expression levels. The first step is the isolation of mRNA from a target sample. The starting material is typically total RNA isolated from human tumors or tumor cell lines. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Numerous RNA isolation kits are commercially available.

In some embodiments, the primers used for quantitative RT-PCR comprise a forward and reverse primer for each gene listed in Table 4. In one embodiment, the primers used for quantitative RT-PCR are listed in Table 7. In one embodiment, primers comprising sequences identical to the sequences of SEQ ID NO: 173-202 are used for quantitative RT-PCR, wherein primers with sequences identifical to SEQ ID NO: 173-187 are forward primers and primers with sequences identifical to SEQ ID NO: 188-202 are reverse primers.

In some embodiments the analytical method used for detecting at least one biomarker RNA in the methods set forth herein includes real-time quantitative RT-PCR. See Chen, C. et al. (2005) Nucl. Acids Res. 33:e179, which is incorporated herein by reference in its entirety. Although PCR can use a variety of thermostable DNA-dependent DNA polymerases, it typically employs the Taq DNA polymerase, which has a 5′-3′ nuclease activity but lacks a 3′-5′ proofreading endonuclease activity. In some embodiments, RT-PCR is done using a TaqMan® assay sold by Applied Biosystems, Inc. In a first step, total RNA is isolated from the sample. In some embodiments, the assay can be used to analyze about 10 ng of total RNA input sample, such as about 9 ng of input sample, such as about 8 ng of input sample, such as about 7 ng of input sample, such as about 6 ng of input sample, such as about 5 ng of input sample, such as about 4 ng of input sample, such as about 3 ng of input sample, such as about 2 ng of input sample, and even as little as about 1 ng of input sample containing RNA.

The TaqMan® assay utilizes a stem-loop primer that is specifically complementary to the 3′-end of a biomarker RNA. The step of hybridizing the stem-loop primer to the biomarker RNA is followed by reverse transcription of the biomarker RNA template, resulting in extension of the 3′ end of the primer. The result of the reverse transcription step is a chimeric (DNA) amplicon with the step-loop primer sequence at the 5′ end of the amplicon and the cDNA of the biomarker RNA at the 3′ end. Quantitation of the biomarker RNA is achieved by RT-PCR using a universal reverse primer comprising a sequence that is complementary to a sequence at the 5′ end of all stem-loop biomarker RNA primers, a biomarker RNA-specific forward primer, and a biomarker RNA sequence-specific TaqMan® probe.

The assay uses fluorescence resonance energy transfer (“FRET”) to detect and quantitate the synthesized PCR product. Typically, the TaqMan® probe comprises a fluorescent dye molecule coupled to the 5′-end and a quencher molecule coupled to the 3′-end, such that the dye and the quencher are in close proximity, allowing the quencher to suppress the fluorescence signal of the dye via FRET. When the polymerase replicates the chimeric amplicon template to which the TaqMan® probe is bound, the 5′-nuclease of the polymerase cleaves the probe, decoupling the dye and the quencher so that FRET is abolished and a fluorescence signal is generated. Fluorescence increases with each RT-PCR cycle proportionally to the amount of probe that is cleaved.

In some embodiments, quantitation of the results of RT-PCR assays is done by constructing a standard curve from a nucleic acid of known concentration and then extrapolating quantitative information for biomarker RNAs of unknown concentration. In some embodiments, the nucleic acid used for generating a standard curve is an RNA of known concentration. In some embodiments, the nucleic acid used for generating a standard curve is a purified double-stranded plasmid DNA or a single-stranded DNA generated in vitro.

In some embodiments, where the amplification efficiencies of the biomarker nucleic acids and the endogenous reference are approximately equal, quantitation is accomplished by the comparative Cₜ (cycle threshold, e.g., the number of PCR cycles required for the fluorescence signal to rise above background) method. Cₜ values are inversely proportional to the amount of nucleic acid target in a sample. In some embodiments, Cₜ values of the target RNA of interest can be compared with a control or calibrator, such as RNA from normal tissue. In some embodiments, the Cₜ values of the calibrator and the target RNA samples of interest are normalized to an appropriate endogenous housekeeping gene (see above).

In addition to the TaqMan® assays, other RT-PCR chemistries useful for detecting and quantitating PCR products in the methods presented herein include, but are not limited to, Molecular Beacons, Scorpion probes and SYBR Green detection.

In some embodiments, Molecular Beacons can be used to detect and quantitate PCR products. Like TaqMan® probes, Molecular Beacons use FRET to detect and quantitate a PCR product via a probe comprising a fluorescent dye and a quencher attached at the ends of the probe. Unlike TaqMan® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target, the stem-loop structure is abolished so that the dye and the quencher become separated in space and the dye fluoresces. Molecular Beacons are available, e.g., from Gene Link™ (see http://www.genelink.com/newsite/products/mbintro.asp).

In some embodiments, Scorpion probes can be used as both sequence-specific primers and for PCR product detection and quantitation. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to the 5′-end of the Scorpion probe, and a quencher is attached to the 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g., Premier Biosoft International (see http://www.premierbiosoft.com/tech_notes/Scorpion.html).

In some embodiments, RT-PCR detection is performed specifically to detect and quantify the expression of a single biomarker RNA. The biomarker RNA, in typical embodiments, is selected from a biomarker RNA capable of specifically hybridizing to a nucleic acid comprising a sequence that is identically present in one of the genes set forth in Table 4. In some embodiments, the biomarker RNA specifically hybridizes to a nucleic acid comprising a sequence that is identically present in at least one of the genes in Table 3.

In various other embodiments, RT-PCR detection is utilized to detect, in a single multiplex reaction, each of 15, each of 16, each of 17, even each of 18 biomarker RNAs. The biomarker RNAs, in some embodiments, are capable of specifically hybridizing to a nucleic acid comprising a sequence that is identically present in one of the fifteen genes listed in Table 4 and optionally one, two, or three additional genes listed in Table 3.

In some multiplex embodiments, a plurality of probes, such as TaqMan probes, each specific for a different RNA target, is used. In typical embodiments, each target RNA-specific probe is spectrally distinguishable from the other probes used in the same multiplex reaction.

In some embodiments, quantitation of RT-PCR products is accomplished using a dye that binds to double-stranded DNA products, such as SYBR Green. In some embodiments, the assay is the QuantiTect SYBR Green PCR assay from Qiagen. In this assay, total RNA is first isolated from a sample. Total RNA is subsequently poly-adenylated at the 3′-end and reverse transcribed using a universal primer with poly-dT at the 5′-end. In some embodiments, a single reverse transcription reaction is sufficient to assay multiple biomarker RNAs. RT-PCR is then accomplished using biomarker RNA-specific primers and an miScript Universal Primer, which comprises a poly-dT sequence at the 5′-end. SYBR Green dye binds non-specifically to double-stranded DNA and upon excitation, emits light. In some embodiments, buffer conditions that promote highly-specific annealing of primers to the PCR template (e.g., available in the QuantiTect SYBR Green PCR Kit from Qiagen) can be used to avoid the formation of non-specific DNA duplexes and primer dimers that will bind SYBR Green and negatively affect quantitation. Thus, as PCR product accumulates, the signal from SYBR green increases, allowing quantitation of specific products.

RT-PCR is performed using any RT-PCR instrumentation available in the art. Typically, instrumentation used in real-time RT-PCR data collection and analysis comprises a thermal cycler, optics for fluorescence excitation and emission collection, and optionally a computer and data acquisition and analysis software.

In some embodiments, the method of detectably quantifying one or more biomarker RNAs includes the steps of: (a) isolating total RNA; (b) reverse transcribing a biomarker RNA to produce a cDNA that is complementary to the biomarker RNA; (c) amplifying the cDNA from step (b); and (d) detecting the amount of a biomarker RNA with RT-PCR.

As described above, in some embodiments, the RT-PCR detection is performed using a FRET probe, which includes, but is not limited to, a TaqMan® probe, a Molecular beacon probe and a Scorpion probe. In some embodiments, the RT-PCR detection and quantification is performed with a TaqMan® probe, i.e., a linear probe that typically has a fluorescent dye covalently bound at one end of the DNA and a quencher molecule covalently bound at the other end of the DNA. The FRET probe comprises a base sequence that is complementary to a region of the cDNA such that, when the FRET probe is hybridized to the cDNA, the dye fluorescence is quenched, and when the probe is digested during amplification of the cDNA, the dye is released from the probe and produces a fluorescence signal. In such embodiments, the amount of biomarker RNA in the sample is proportional to the amount of fluorescence measured during cDNA amplification.

The TaqMan® probe typically comprises a region of contiguous nucleotides comprising a base sequence that is complementary to a region of a biomarker RNA or its complementary cDNA that is reverse transcribed from the biomarker RNA template (i.e., the sequence of the probe region is complementary to or identically present in the biomarker RNA to be detected) such that the probe is specifically hybridizable to the resulting PCR amplicon. In some embodiments, the probe comprises a region of at least 6 contiguous nucleotides having a base sequence that is fully complementary to or identically present in a region of a cDNA that has been reverse transcribed from a biomarker RNA template, such as comprising a region of at least 8 contiguous nucleotides, or comprising a region of at least 10 contiguous nucleotides, or comprising a region of at least 12 contiguous nucleotides, or comprising a region of at least 14 contiguous nucleotides, or even comprising a region of at least 16 contiguous nucleotides having a base sequence that is complementary to or identically present in a region of a cDNA reverse transcribed from a biomarker RNA to be detected.

Preferably, the region of the cDNA that has a sequence that is complementary to the TaqMan® probe sequence is at or near the center of the cDNA molecule. In some embodiments, there are independently at least 2 nucleotides, such as at least 3 nucleotides, such as at least 4 nucleotides, such as at least 5 nucleotides of the cDNA at the 5′-end and at the 3′-end of the region of complementarity.

In typical embodiments, all biomarker RNAs are detected in a single multiplex reaction. In these embodiments, each TaqMan® probe that is targeted to a unique cDNA is spectrally distinguishable when released from the probe. Thus, each biomarker RNA is detected by a unique fluorescence signal.

In some embodiments, expression levels may be represented by gene transcript numbers per nanogram of cDNA. To control for variability in cDNA quantity, integrity and the overall transcriptional efficiency of individual primers, RT-PCR data can be subjected to standardization and normalization against one or more housekeeping genes as has been previously described. See, e.g., Rubie et al., Mol. Cell. Probes 19(2):101-9 (2005).

Appropriate genes for normalization in the methods described herein include those as to which the quantity of the product does not vary between between different cell types, cell lines or under different growth and sample preparation conditions. In some embodiments, endogenous housekeeping genes useful as normalization controls in the methods described herein include, but are not limited to, ACTB, BAT1, B2M, TBP, U6 snRNA, RNU44, RNU 48, and U47. In typical embodiments, the at least one endogenous housekeeping gene for use in normalizing the measured quantity of RNA is selected from ACTB, BAT1, B2M, TBP, U6 snRNA, U6 snRNA, RNU44, RNU 48, and U47. In some embodiments, normalization to the geometric mean of two, three, four or more housekeeping genes is performed. In some embodiments, one housekeeping gene is used for normalization. In some embodiments, two, three, four or more housekeeping genes are used for normalization.

In some embodiments, labels that can be used on the FRET probes include colorimetric and fluorescent labels such as Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides useful in the preparation of RT-PCR probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Examples of fluorescently labeled deoxyribonucleotides useful in the preparation of RT-PCR probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. Fluorescently labeled nucleotides are commercially available and can be purchased from, e.g., Invitrogen.

In some embodiments, dyes and other moieties, such as quenchers, are introduced into nucleic acids used in the methods described herein, such as FRET probes, via modified nucleotides. A “modified nucleotide” refers to a nucleotide that has been chemically modified, but still functions as a nucleotide. In some embodiments, the modified nucleotide has a chemical moiety, such as a dye or quencher, covalently attached, and can be introduced into an oligonucleotide, for example, by way of solid phase synthesis of the oligonucleotide. In other embodiments, the modified nucleotide includes one or more reactive groups that can react with a dye or quencher before, during, or after incorporation of the modified nucleotide into the nucleic acid. In specific embodiments, the modified nucleotide is an amine-modified nucleotide, i.e., a nucleotide that has been modified to have a reactive amine group. In some embodiments, the modified nucleotide comprises a modified base moiety, such as uridine, adenosine, guanosine, and/or cytosine. In specific embodiments, the amine-modified nucleotide is selected from 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP. In some embodiments, nucleotides with different nucleobase moieties are similarly modified, for example, 5-(3-aminoallyl)-GTP instead of 5-(3-aminoallyl)-UTP. Many amine modified nucleotides are commercially available from, e.g., Applied Biosystems, Sigma, Jena Bioscience and TriLink.

In some embodiments, the methods of detecting at least one biomarker RNA described herein employ one or more modified oligonucleotides, such as oligonucleotides comprising one or more affinity-enhancing nucleotides. Modified oligonucleotides useful in the methods described herein include primers for reverse transcription, PCR amplification primers, and probes. In some embodiments, the incorporation of affinity-enhancing nucleotides increases the binding affinity and specificity of an oligonucleotide for its target nucleic acid as compared to oligonucleotides that contain only deoxyribonucleotides, and allows for the use of shorter oligonucleotides or for shorter regions of complementarity between the oligonucleotide and the target nucleic acid.

In some embodiments, affinity-enhancing nucleotides include nucleotides comprising one or more base modifications, sugar modifications and/or backbone modifications.

In some embodiments, modified bases for use in affinity-enhancing nucleotides include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine.

In some embodiments, affinity-enhancing modifications include nucleotides having modified sugars such as 2′-substituted sugars, such as 2′-O-alkyl-ribose sugars, 2′-amino-deoxyribose sugars, 2′-fluoro- deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing modifications include backbone modifications such as the use of peptide nucleic acids (e.g., an oligomer including nucleobases linked together by an amino acid backbone). Other backbone modifications include phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

In some embodiments, the oligomer includes at least one affinity-enhancing nucleotide that has a modified base, at least nucleotide (which may be the same nucleotide) that has a modified sugar and at least one internucleotide linkage that is non-naturally occurring.

In some embodiments, the affinity-enhancing nucleotide contains a locked nucleic acid (“LNA”) sugar, which is a bicyclic sugar. In some embodiments, an oligonucleotide for use in the methods described herein comprises one or more nucleotides having an LNA sugar. In some embodiments, the oligonucleotide contains one or more regions consisting of nucleotides with LNA sugars. In other embodiments, the oligonucleotide contains nucleotides with LNA sugars interspersed with deoxyribonucleotides. See, e.g., Frieden, M. et al. (2008) Curr. Pharm. Des. 14(11):1138-1142.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g., in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art. In one embodiment, primer sets for the 15 genes are those listed in Table 7.

In addition, a person skilled in the art will appreciate that a number of methods can be used to determine the amount of a protein product of the biomarker of the invention, including immunoassays such as Western blots, ELISA, and immunoprecipitation followed by SDS-PAGE and immunocytochemistry.

Accordingly, in another embodiment, an antibody is used to detect the polypeptide products of the fifteen biomarkers listed in Table 4. In another embodiment, the sample comprises a tissue sample. In a further embodiment, the tissue sample is suitable for immunohistochemistry.

The term “antibody” as used herein is intended to include monoclonal antibodies, polyclonal antibodies, and chimeric antibodies. The antibody may be from recombinant sources and/or produced in transgenic animals. The term “antibody fragment”” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Conventional techniques of molecular biology, microbiology and recombinant DNA techniques are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

For example, antibodies having specificity for a specific protein, such as the protein product of a biomarker, may be prepared by conventional methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

In some embodiments, recombinant antibodies are provided that specifically bind protein products of the fifteen genes listed in Table 4, and optionally expression products of one or more genes listed in Table 3. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine monoclonal antibody (mAb) and a human immunoglobulin constant region. (See, e.g., Cabilly et al., U.S. Pat. No. 4,816,567; and Boss et al., U.S. Pat. No. 4,816,397, which are incorporated herein by reference in their entirety.) Single-chain antibodies have an antigen binding site and consist of single polypeptides. They can be produced by techniques known in the art, for example using methods described in Ladner et al., U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88. Multi-specific antibodies are antibody molecules having at least two antigen-binding sites that specifically bind different antigens. Such molecules can be produced by techniques known in the art, for example using methods described in Segal, U.S. Pat. No. 4,676,980 (the disclosure of which is incorporated herein by reference in its entirety); Holliger et al., (1993) Proc. Natl. Acad. Sci. USA 90:6444-6448; Whitlow et al., (1994) Protein Eng 7:1017-1026 and U.S. Pat. No. 6,121,424.

Monoclonal antibodies directed against any of the expression products of the genes listed in Table 4 and, optionally, against expression products of one or more genes listed in Table 3, can be identified and isolated by screening a recombinant combinatorial immunoglobulin library (e.g., an antibody phage display library) with the polypeptide(s) of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

In some embodiments, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a protein product. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies such as Abgenix, Inc. (Fremont, Calif.), can be engaged to provide human antibodies directed against a selected antigen using technology similar to that described above.

Antibodies may be isolated after production (e.g., from the blood or serum of the subject) or synthesis and further purified by well-known techniques. For example, IgG antibodies can be purified using protein A chromatography. Antibodies specific for a protein can be selected or (e.g., partially purified) or purified by, e.g., affinity chromatography. For example, a recombinantly expressed and purified (or partially purified) expression product may be produced, and covalently or non-covalently coupled to a solid support such as, for example, a chromatography column. The column can then be used to affinity purify antibodies specific for the protein products of the genes listed in Tables 3 and 4 from a sample containing antibodies directed against a large number of different epitopes, thereby generating a substantially purified antibody composition, i.e., one that is substantially free of contaminating antibodies. By a substantially purified antibody composition it is meant, in this context, that the antibody sample contains at most only 30% (by dry weight) of contaminating antibodies directed against epitopes other than those of the protein products of the genes listed in Tables 3 and 4, and preferably at most 20%, yet more preferably at most 10%, and most preferably at most 5% (by dry weight) of the sample is contaminating antibodies. A purified antibody composition means that at least 99% of the antibodies in the composition are directed against the desired protein.

In some embodiments, substantially purified antibodies may specifically bind to a signal peptide, a secreted sequence, an extracellular domain, a transmembrane or a cytoplasmic domain or cytoplasmic membrane of a protein product of one of the genes listed in Tables 3 and 4. In an embodiment, substantially purified antibodies specifically bind to a secreted sequence or an extracellular domain of the amino acid sequences of a protein product of one of the genes listed in Tables 3 and 4.

In some embodiments, antibodies directed against a protein product of one of the genes listed in Tables 3 and 4 can be used to detect the protein products or fragment thereof (e.g., in a cellular lysate or cell supernatant) in order to evaluate the level and pattern of expression of the protein. Detection can be facilitated by the use of an antibody derivative, which comprises an antibody coupled to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

A variety of techniques can be employed to measure expression levels of each of the fifteen, and optional additional, genes given a sample that contains protein products that bind to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of the fifteen, and optional additional products of the genes listed in Tables 4 and 3.

In one embodiment, antibodies, or antibody fragments or derivatives, can be used in methods such as Western blots or immunofluorescence techniques to detect the expressed proteins. In some embodiments, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of the prognostic markers. In some embodiments, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art (Ausubel, supra, unit 10.1-10.6). A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

Numerous labels are available which can be generally grouped into the following categories:

a. Radioisotopes, such as .sup.36S, .sup.14C, .sup.125I, .sup.3H, and .sup.131I. The antibody variant can be labeled with the radioisotope using the techniques described in Current Protocols in Immunology, Vol. 1-2, Coligen et al., Ed., Wiley-Interscience, New York, Pubs. (1991) for example and radioactivity can be measured using scintillation counting.

b. Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques disclosed in Current Protocols in Immunology, supra, for example. Fluorescence can be quantified using a fluorimeter;

c. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

In some embodiments, a detection label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some embodiments, the antibody need not be labeled, and the presence thereof can be detected using a labeled antibody, which binds to the antibody.

The 15-gene signature described herein can be used to select treatment for NCSLC patients. As explained herein, the biomarkers can classify patients with NSCLC into a poor survival group or a good survival group and into groups that might benefit from adjuvant chemotherapy or not.

Accordingly, in one embodiment, the application provides a method of selecting a therapy for a subject with NSCLC, comprising the steps:

a. classifying the subject with NSCLC into a poor survival group or a good survival group according to the methods described herein; and

b. selecting adjuvant chemotherapy for the subject classified as being in the poor survival group or no adjuvant chemotherapy for the subject classified as being in the good survival group.

In another embodiment, the application provides a method of selecting a therapy for a subject with NSCLC, comprising the steps:

a. determining the expression of fifteen biomarkers in a test sample from the subject, wherein the fifteen biomarkers correspond to the fifteen genes in Table 4;

b. comparing the expression of the fifteen biomarkers in the test sample with the fifteen biomarkers in a control sample;

c. classifying the subject in a poor survival group or a good survival group,

wherein a difference or a similarity in the expression of the fifteen biomarkers between the control sample and the test sample is used to classify the subject into a poor survival group or a good survival group; and

d. selecting adjuvant chemotherapy if the subject is classified in the poor survival group and selecting no adjuvant chemotherapy if the subject is classified in the good survival group.

The term “adjuvant chemotherapy” as used herein means treatment of cancer with chemotherapeutic agents after surgery where all detectable disease has been removed, but where there still remains a risk of small amounts of remaining cancer. Typical chemotherapeutic agents include cisplatin, carboplatin, vinorelbine, gemcitabine, doccetaxel, paclitaxel and navelbine.

In another aspect, the application provides compositions useful in detecting changes in the expression levels of the 15 genes listed in Table 4. Accordingly in one embodiment, the application provides a composition comprising a plurality of isolated nucleic acid sequences wherein each isolated nucleic acid sequence hybridizes to:

a. a RNA product of one of the 15 genes listed in Table 4; and/or

b. a nucleic acid complementary to a),

wherein the composition is used to measure the level of RNA expression of the 15 genes. In a particular embodiment, the plurality of isolated nucleic acid sequences comprise isolated nucleic acids hybridizable to the 15 probe target sequences as set out in Table 9. In one embodiment, the plurality of isolated nucleic acid sequences comprise isolated nucleic acids hybridizable to SEQ ID NO: 3, 11-15, 22, 26, 35, 49, 78, 85, 130, 133, and 169.

In another embodiment, the application provides a composition comprising 15 forward and 15 reverse primers for amplifying a region of each gene listed in Table 4. In particular embodiment, the 30 primers are as set out in Table 7. In one embodiment, the 30 primers each comprise a sequence that is identical to the sequence of one of SEQ ID NO: 173-202.

In a further aspect, the application also provides an array that is useful in detecting the expression levels of the 15 genes set out in Table 4. Accordingly, in one embodiment, the application provides an array comprising for each gene shown in Table 4 one or more nucleic acid probes complementary and hybridizable to an expression product of the gene. In a particular embodiment, the array comprises the nucleic acid probes hybridizable to the probe target sequences listed in Table 9. In one embodiment, the array comprises the nucleic acid probes hybridizable to sequences identical to each of SEQ ID NO: 3, 11-15, 22, 26, 35, 49, 78, 85, 130, 133, and 169.

In yet another aspect, the application also provides for kits used to prognose or classify a subject with NSCLC into a good survival group or a poor survival group or to select a therapy for a subject with NSCLC that includes detection agents that can detect the expression products of the biomarkers. Accordingly, in one embodiment, the application provides a kit to prognose or classify a subject with early stage NSCLC comprising detection agents that can detect the expression products of 15 biomarkers, wherein the 15 biomarkers comprise 15 genes in Table 4. In another embodiment, kits for classifying a subject comprise detection agents that can detect the expression of 16, 17, or 18 biomarkers, wherein 15 biomarkers comprise the 15 genes in Table 4, and the additional biomarkers are selected from the genes listed in Table 3. In one embodiment, the additional sixteenth, seventeenth, and eighteenth biomarkers may be selected from RGS4, UGT2B4, and MCF2 listed in Table 3.

In one embodiment, the application provides a kit to select a therapy for a subject with NSCLC, comprising detection agents that can detect the expression products of 15 biomarkers, wherein the 15 biomarkers comprise 15 genes in Table 4. In some embodiments, kits for selecting therapy for a subject comprise detection agents that can detect the expression of 16, 17, or 18 biomarkers, wherein 15 biomarkers comprise the 15 genes in Table 4, and the additional biomarkers are selected from the genes listed in Table 3. In one embodiment, the additional sixteenth, seventeenth, and eighteenth biomarkers may be selected from RGS4, UGT2B4, and MCF2 listed in Table 3.

The materials and methods of the present disclosure are ideally suited for preparation of kits produced in accordance with well known procedures. In some embodiments, kits comprise agents (like the polynucleotides and/or antibodies described herein as non-limiting examples) for the detection of expression of the disclosed sequences, such as for example, SEQ ID NO: 3, 11-15, 22, 26, 35, 49, 78, 85, 130, 133, and 169, the target sequences listed in Table 9, or the target sequences listed in Table 11. Kits, may comprise containers, each with one or more of the various reagents (sometimes in concentrated form), for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes (e.g., appropriate length poly(T) or random primers linked to a promoter reactive with the RNA polymerase). A set of instructions will also typically be included.

In some embodiments, a kit may comprise a plurality of reagents, each of which is capable of binding specifically with a target nucleic acid or protein. Suitable reagents for binding with a target protein include antibodies, antibody derivatives, antibody fragments, and the like. Suitable reagents for binding with a target nucleic acid (e.g., a genomic DNA, an mRNA, a spliced mRNA, a cDNA, or the like) include complementary nucleic acids. For example, nucleic acid reagents may include oligonucleotides (labeled or non-labeled) fixed to a substrate, labeled oligonucleotides not bound with a substrate, pairs of PCR primers, molecular beacon probes, and the like.

In some embodiments, kits may comprise additional components useful for detecting gene expression levels. By way of example, kits may comprise fluids (e.g., SSC buffer) suitable for annealing complementary nucleic acids or for binding an antibody with a protein with which it specifically binds, one or more sample compartments, a material which provides instruction for detecting expression levels, and the like.

In some embodiments, kits for use in the RT-PCR methods described herein comprise one or more target RNA-specific FRET probes and one or more primers for reverse transcription of target RNAs or amplification of cDNA reverse transcribed therefrom.

In some embodiments, one or more of the primers is “linear”. A “linear” primer refers to an oligonucleotide that is a single stranded molecule, and typically does not comprise a short region of, for example, at least 3, 4 or 5 contiguous nucleotides, which are complementary to another region within the same oligonucleotide such that the primer forms an internal duplex. In some embodiments, the primers for use in reverse transcription comprise a region of at least 4, such as at least 5, such as at least 6, such as at least 7 or more contiguous nucleotides at the 3′-end that has a base sequence that is complementary to region of at least 4, such as at least 5, such as at least 6, such as at least 7 or more contiguous nucleotides at the 5′-end of a target RNA.

In some embodiments, the kit further comprises one or more pairs of linear primers (a “forward primer” and a “reverse primer”) for amplification of a cDNA reverse transcribed from a target RNA. Accordingly, in some embodiments, the forward primer comprises a region of at least 4, such as at least 5, such as at least 6, such as at least 7, such as at least 8, such as at least 9, such as at least 10 contiguous nucleotides having a base sequence that is complementary to the base sequence of a region of at least 4, such as at least 5, such as at least 6, such as at least 7, such as at least 8, such as at least 9, such as at least 10 contiguous nucleotides at the 5′-end of a target RNA. Furthermore, in some embodiments, the reverse primer comprises a region of at least 4, such as at least 5, such as at least 6, such as at least 7, such as at least 8, such as at least 9, such as at least 10 contiguous nucleotides having a base sequence that is complementary to the base sequence of a region of at least 4, such as at least 5, such as at least 6, such as at least 7, such as at least 8, such as at least 9, such as at least 10 contiguous nucleotides at the 3′-end of a target RNA.

In some embodiments, the kit comprises at least a first set of primers for amplification of a cDNA that is reverse transcribed from a target RNA capable of specifically hybridizing to a nucleic acid comprising a sequence identically present in one of the genes listed in Table 4. In some embodiments, the kit comprises at least fifteen sets of primers, each of which is for amplification of a different target RNA capable of specifically hybridizing to a nucleic acid comprising a sequence identically present in a different gene listed in Table 4. In one embodiment, the kit comprises fifteen forward and fifteen reverse primers described in Table 7, comprising sequences identical to SEQ ID NOs 173-202. In some embodiments, the kit comprises one, two, or three more sets of primers, in addition to the fifteen sets of primers, each of the additional sets being for amplification of a different target RNA capable of specifically hybridizing to a nucleic acid comprising a sequence identically present in a different gene listed in Table 3. In some embodiments, the kit comprises one, two, or three more sets of primers, in addition to the fifteen sets of primers, each of the additional sets being for amplification of a different target RNA capable of specifically hybridizing to a nucleic acid comprising a sequence identically present in RGS4, UGT2B4, or MCF2 listed in Table 3. In some embodiments, the kit comprises at least one set of primers that is capable of amplifying more than one cDNA reverse transcribed from a target RNA in a sample.

In some embodiments, probes and/or primers for use in the compositions described herein comprise deoxyribonucleotides. In some embodiments, probes and/or primers for use in the compositions described herein comprise deoxyribonucleotides and one or more nucleotide analogs, such as LNA analogs or other duplex-stabilizing nucleotide analogs described above. In some embodiments, probes and/or primers for use in the compositions described herein comprise all nucleotide analogs. In some embodiments, the probes and/or primers comprise one or more duplex-stabilizing nucleotide analogs, such as LNA analogs, in the region of complementarity.

In some embodiments, the compositions described herein also comprise probes, and in the case of RT-PCR, primers, that are specific to one or more housekeeping genes for use in normalizing the quantities of target RNAs. Such probes (and primers) include those that are specific for one or more products of housekeeping genes selected from ACTB, BAT1, B2M, TBP, U6 snRNA, RNU44, RNU 48, and U47.

In some embodiments, the kits for use in real time RT-PCR methods described herein further comprise reagents for use in the reverse transcription and amplification reactions. In some embodiments, the kits comprise enzymes such as reverse transcriptase, and a heat stable DNA polymerase, such as Taq polymerase. In some embodiments, the kits further comprise deoxyribonucleotide triphosphates (dNTP) for use in reverse transcription and amplification. In further embodiments, the kits comprise buffers optimized for specific hybridization of the probes and primers.

In some embodiments, kits are provided containing antibodies to each of the protein products of the genes listed in Table 4, conjugated to a detectable substance, and instructions for use. In some embodiments, the kits comprise antibodies to one, two, or three protein products of the genes listed in Table 3, in addition to antibodies to each of the protein products of the genes listed in Table 4. In some embodiments, the kit comprises antibodies to the protein product of one, two, or all three of RGS4, UGT2B4, or MCF2 listed in Table 3, in addition to antibodies to each of the protein products of the genes listed in Table 4. Kits may comprise an antibody, an antibody derivative, or an antibody fragment, which binds specifically with a marker protein, or a fragment of the protein. Such kits may also comprise a plurality of antibodies, antibody derivatives, or antibody fragments wherein the plurality of such antibody agents binds specifically with a marker protein, or a fragment of the protein.

In some embodiments, kits may comprise antibodies such as a labeled or labelable antibody and a compound or agent for detecting protein in a biological sample; means for determining the amount of protein in the sample; means for comparing the amount of protein in the sample with a standard; and instructions for use. Such kits can be supplied to detect a single protein or epitope or can be configured to detect one of a multitude of epitopes, such as in an antibody detection array. Arrays are described in detail herein for nucleic acid arrays and similar methods have been developed for antibody arrays.

A person skilled in the art will appreciate that a number of detection agents can be used to determine the expression of the biomarkers. For example, to detect RNA products of the biomarkers, probes, primers, complementary nucleotide sequences or nucleotide sequences that hybridize to the RNA products can be used. To detect protein products of the biomarkers, ligands or antibodies that specifically bind to the protein products can be used.

Accordingly, in one embodiment, the detection agents are probes that hybridize to the 15 biomarkers. In a particular embodiment, the probe target sequences are as set out in Table 9. In one embodiment, the probe target sequences are identical to SEQ ID NO: 3, 11-15, 22, 26, 35, 49, 78, 85, 130, 133, and 169. In another embodiment, the detection agents are forward and reverse primers that amplify a region of each of the 15 genes listed in Table 4. In a particular embodiment, the primers are as set out in Table 7. In one embodiment, the primers comprise the polynucleotide sequences of SEQ ID NO: 173-202.

A person skilled in the art will appreciate that the detection agents can be labeled.

The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The kit can also include a control or reference standard and/or instructions for use thereof. In addition, the kit can include ancillary agents such as vessels for storing or transporting the detection agents and/or buffers or stabilizers.

In some aspects, a multi-gene signature is provided for prognosis or classifying patients with lung cancer. In some embodiments, a fifteen-gene signature is provided, comprising reference values for each of the fifteen genes based on relative expression data from a historical data set with a known outcome, such as good or poor survival, and/or known treatment, such as adjuvant chemotherapy. In one embodiment, four reference values are provided for each of the fifteen genes listed in Table 4. In one embodiment, the reference values for each of the fifteen genes are principal component values set forth in Table 10.

In one aspect, relative expression data from a patient are combined with the gene-specific reference values on a gene-by-gene basis for each of the fifteen, and, optionally, additional genes, to generate a test value which allows prognosis or therapy recommendation. In some embodiments, relative expression data are subjected to an algorithm that yields a single test value, or combined score, which is then compared to a control value obtained from the historical expression data for a patient or pool of patients.

In some embodiments, the control value is a numerical threshold for predicting outcomes, for example good and poor outcome, or making therapy recommendations for a subject, for example adjuvant chemotherapy in addition to surgical resection or surgical resection alone. In some embodiments, a test value or combined score greater than the control value is predictive, for example, of a poor outcome or benefit from adjuvant chemotherapy, whereas a combined score falling below the control value is predictive, for example, of a good outcome or lack of benefit from adjuvant chemotherapy for a subject.

In some embodiments, a method for prognosing or classifying a subject with NSCLC comprises:

a. measuring expression levels of at least 15 biomarkers from Table 4, and optionally, an additional one, two, or three biomarkers from Table 3 in a test sample,

b. calculating a combined score or test value for the subject from the expression levels of the, and,

c. comparing the combined score to a control value,

wherein a combined score greater than the control value is used to classify a subject into a high risk or poor survival group and a combined score lower than the control value is used to classify a subject into a lower risk or good survival group.

In one embodiment, the combined score is calculated from relative expression data multiplied by reference values, determined from historical data, for each gene. Accordingly, the combined score may be calculated using Formula I below:

Combined score=0.557×PC1+0.328×PC2+0.43×PC3+0.335×PC4

where PC1 is the sum of the relative expression level for each gene in a multi-gene signature multiplied by a first principal component for each gene in the multi-gene signature, PC2 is the sum of the relative expression level for each gene multiplied by a second principal component for each gene, PC3 is the sum of the relative expression level for each gene multiplied by a third principal component for each gene, and PC4 is the sum of the relative expression level for each gene multiplied by a fourth principal component for each gene. In some embodiments, the combined score is referred to as a risk score. A risk score for a subject can be calculated by applying Formula I to relative expression data from a test sample obtained from the subject.

In some embodiments, PC1 is the sum of the relative expression level for each gene provided in Table 4 multiplied by a first principal component for each gene, respectively, as set forth in Table 10; PC2 is the sum of the relative expression level for each gene provided in Table 4 multiplied by a second principal component for each gene, respectively, as set forth in Table 10; PC3 is the sum of the relative expression level for each gene provided in Table 4 multiplied by a third principal component for each gene, respectively, as set forth in Table 10; and PC4 is the sum of the relative expression level for each gene provided in Table 4 multiplied by a fourth principal component for each gene, respectively, as set forth in Table 10.

In one embodiment, the control value is equal to −0.1. A subject with a risk score of more than −0.1 is classified as high risk (poor prognosis). A patient with a risk score of less than −0.1 is classified as lower risk (good prognosis). In some embodiments, adjuvant chemotherapy is recommended for a subject with a risk score of more than −0.1 and not recommended for a subject with a risk score of less than −0.1.

In a further aspect, the application provides computer programs and computer implemented products for carrying out the methods described herein. Accordingly, in one embodiment, the application provides a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

In another embodiment, the application provides a computer implemented product for predicting a prognosis or classifying a subject with NSCLC comprising:

a. a means for receiving values corresponding to a subject expression profile in a subject sample; and

b. a database comprising a reference expression profile associated with a prognosis, wherein the subject biomarker expression profile and the biomarker reference profile each has fifteen values, each value representing the expression level of a biomarker, wherein each biomarker corresponds to one gene in Table 4;

wherein the computer implemented product selects the biomarker reference expression profile most similar to the subject biomarker expression profile, to thereby predict a prognosis or classify the subject.

In yet another embodiment, the application provides a computer implemented product for determining therapy for a subject with NSCLC comprising:

a. a means for receiving values corresponding to a subject expression profile in a subject sample; and

b. a database comprising a reference expression profile associated with a therapy, wherein the subject biomarker expression profile and the biomarker reference profile each has fifteen values, each value representing the expression level of a biomarker, wherein each biomarker corresponds to one gene in Table 4;

wherein the computer implemented product selects the biomarker reference expression profile most similar to the subject biomarker expression profile, to thereby predict the therapy.

Another aspect relates to computer readable mediums such as CD-ROMs. In one embodiment, the application provides computer readable medium having stored thereon a data structure for storing a computer implemented product described herein.

In one embodiment, the data structure is capable of configuring a computer to respond to queries based on records belonging to the data structure, each of the records comprising:

a. a value that identifies a biomarker reference expression profile of the 15 genes in Table 4;

b. a value that identifies the probability of a prognosis associated with the biomarker reference expression profile.

In another aspect, the application provides a computer system comprising

a. a database including records comprising a biomarker reference expression profile of fifteen genes in Table 4 associated with a prognosis or therapy;

b. a user interface capable of receiving a selection of gene expression levels of the 15 genes in Table 4 for use in comparing to the biomarker reference expression profile in the database; and

c. an output that displays a prediction of prognosis or therapy according to the biomarker reference expression profile most similar to the expression levels of the fifteen genes.

In some embodiments, the application provides a computer implemented product comprising

a. a means for receiving values corresponding to relative expression levels in a subject, of at least 15 biomarkers comprising the fifteen genes in Table 4, and optionally, additional one, two, or three genes selected from the genes listed in Table 3;

b. an algorithm for calculating a combined scire based on the relative expression levels of the at least 15 biomarkers;

c. an output that displays the combined score; and, optionally,

d. an output that displays a prognosis or therapy recommendation based on the combined score.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

The following non-limiting example is illustrative of the present invention:

### Example 1

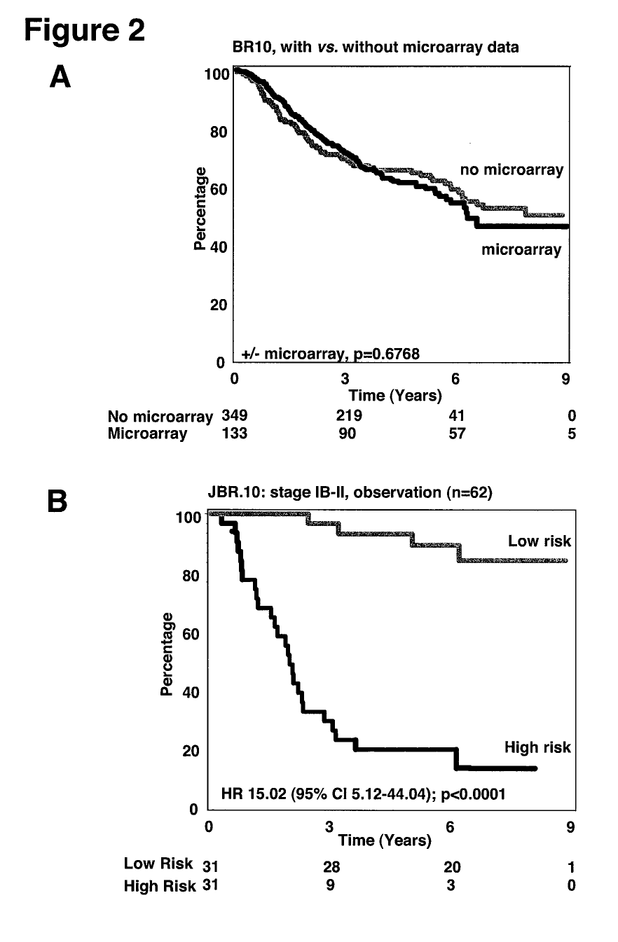

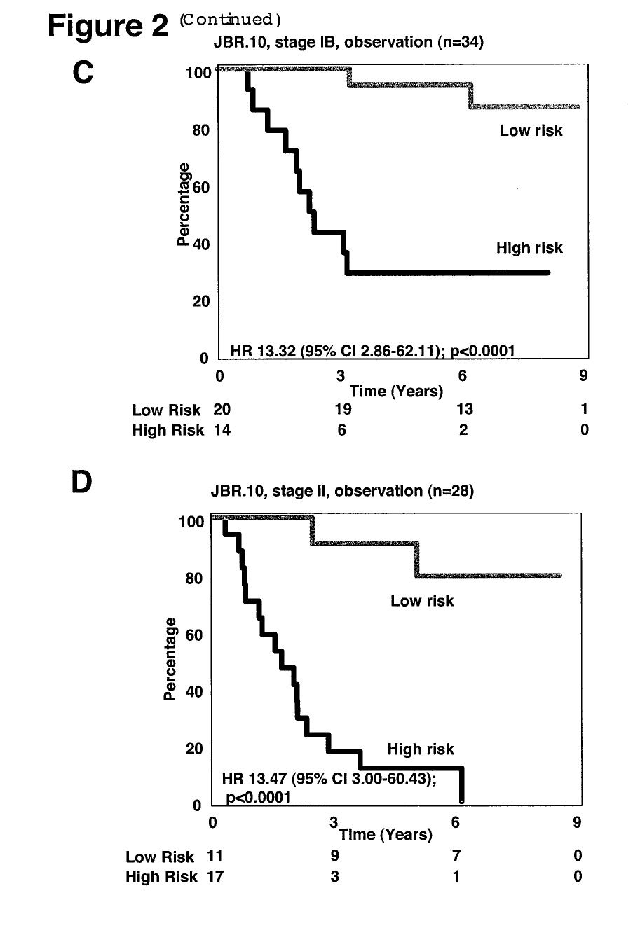

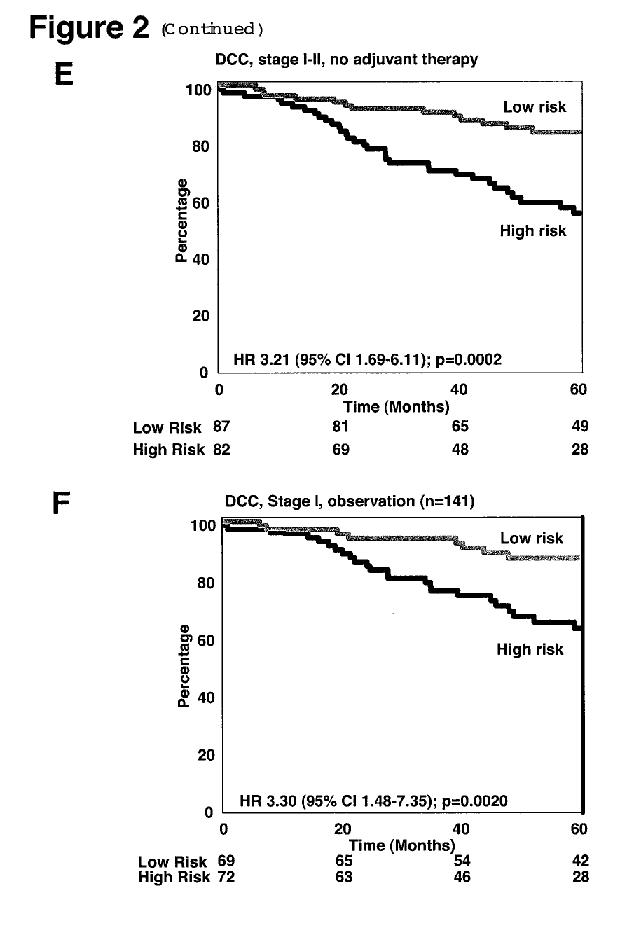

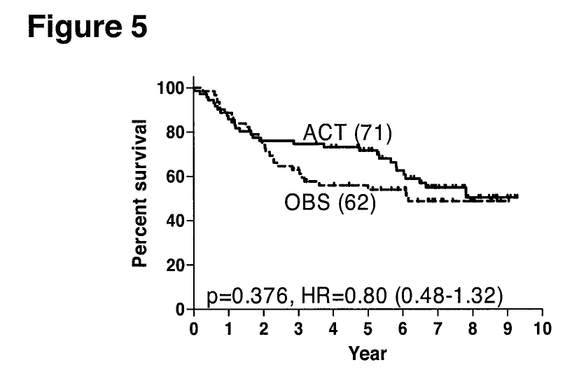

Table 1 compared the demographic features of 133 patients with microarray profiling to 349 without the profiling. Stage IB patients had more representation in the observation cohort (55% vs. 42%, p=0.01), but all other factors were similarly distributed. There was no significant difference in the overall survivals of patients with or without gene profiling (FIG. 2A). For these 133 patients, adjuvant chemotherapy reduced the death rate by 20% (HR 0.80, 95% CI 0.48-1.32, p=0.38; FIG. 5).

A. Prognostic Gene Expression Signature in JBR.10 Patients

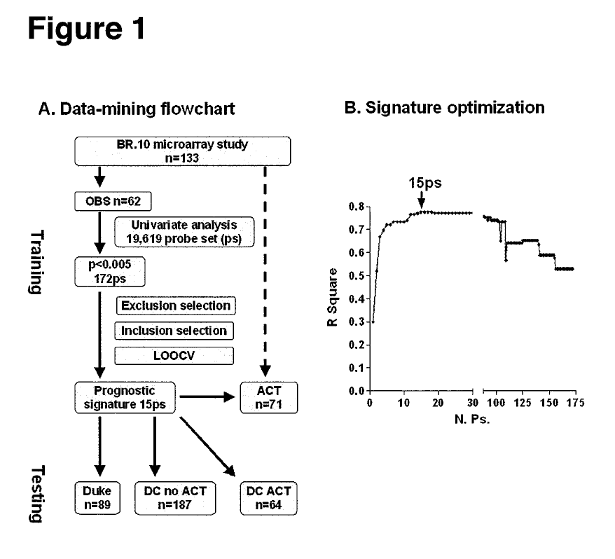

Using a p>0.005 as cut-off, 172 of 19,619 probe sets were significantly associated with prognosis in 62 observation patients (FIG. 1A and Table 3). Using a method that was designed to identify the minimum expression gene set that can distinguish most patients with poor and good survival outcomes, a 15-gene prognostic signature was identified (FIG. 1A and Table 4). This signature was able to separate the 62 non-adjuvant treated patients into 31 low-risk and 31 high-risk patients for death (HR 15.020, 95% CI 5.12-44.04, p<0.0001; FIG. 2B). Furthermore, stratified analysis showed that the signature was also highly prognostic in 34 Stage IB patients (HR 13.32, 95% CI 2.86-62.11, p<0.0001, FIG. 2C) and 28 Stage II patients (HR 13.47, 95% CI 3.0-60.43, p<0.0001, FIG. 2D). Multivariate analysis adjusting for tumor stage, age, gender and histology showed that the prognostic signature was an independent prognostic marker (HR 18.0, 95% CI 5.8-56.1; p<0.0001, Table 2). This did not differ following additional adjustment for surgical procedure and tumor size.

B. Validation of General Applicability of Prognostic Signature (Summary)

Applying the risk score algorithm (equation) established from the 62 BR.10 observation patients, the 15-gene signature was demonstrated to be an independent prognostic marker among all 169 DCC patients (HR 2.9, 95% CI 1.5-5.6, p=0.002; Table 2). Subgroup analyses also showed significant results among patients from DCC-UM (HR 1.5, 95% CI 0.54-4.31, p=0.4; Table 2) and HLM (HR 1.2, 95% CI 0.43-3.6, p=0.7; Table 2). The signature was also prognostic among UM-SQ patients (HR 2.3, 95% CI 1.1-4.7, p=0.026; Table 2), and in the Duke's patients (HR 1.5, 95% CI 0.81-2.89, p=0.19; Table 2).

The prognostic value of the signature was tested in Stage I patients of the DCC (n=141) patients and was able to identify patients with significantly different survival outcome (Table 8).

C. Prediction of Chemotherapy Benefit

When tested on the microarray data of 71 JBR.10 patients who received adjuvant chemotherapy, the 15-gene signature was not prognostic (HR 1.5, 95% CI 0.7-3.3, p=0.28, Table 2). The signature was also not prognostic when applied separately to Stage IB and Stage II patients (Table 2). Among the DCC patients, 41 were identified as having received adjuvant chemotherapy with or without radiotherapy. The 15-gene signature was also not prognostic for these 41 patients (HR 1.1, 95% CI 0.5-2.5, p=0.8) (Table 2).

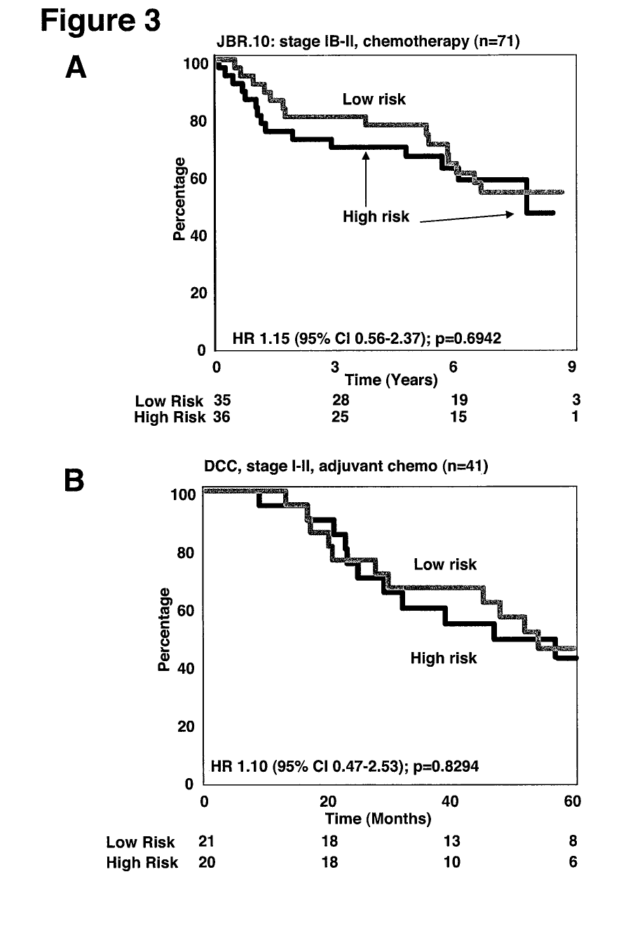

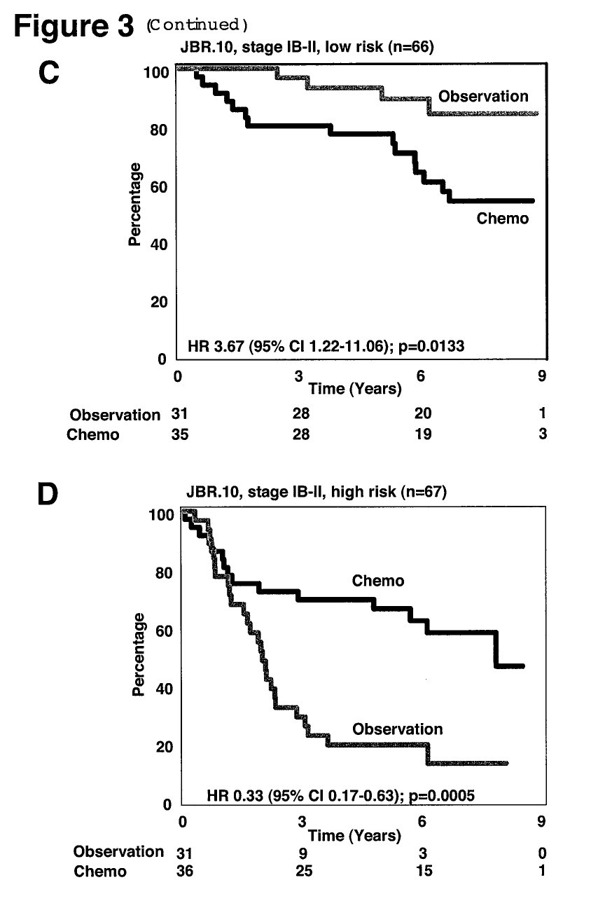

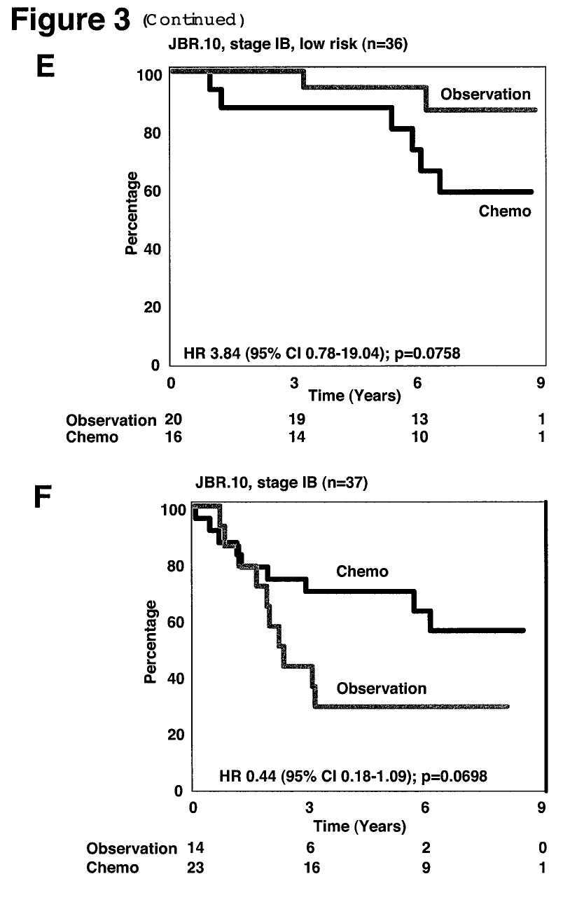

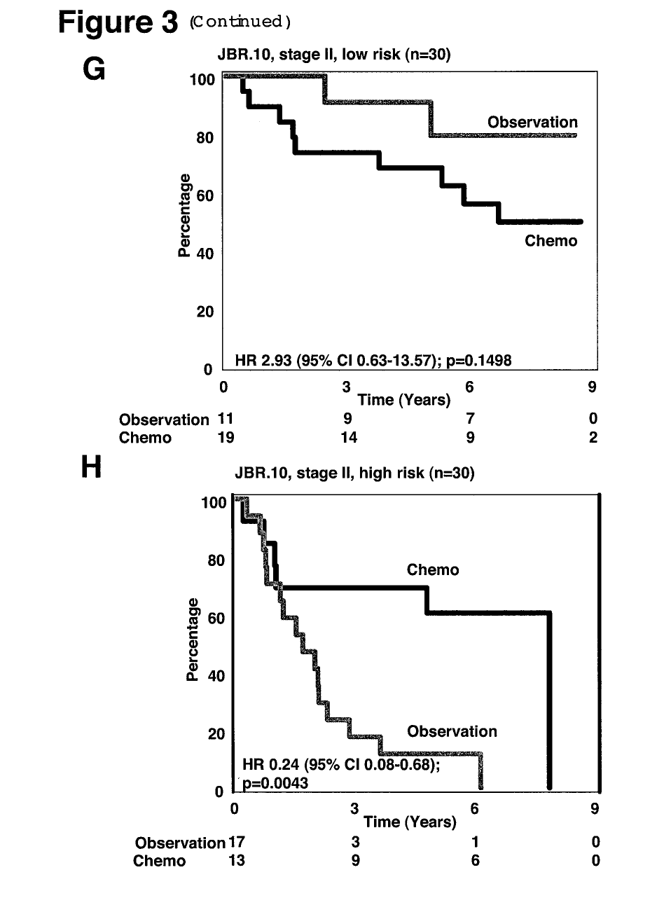

Stratified analysis showed that in JBR.10 patients with microarray data, only patients classified to the high-risk group derived benefit from the adjuvant chemotherapy (FIGS. 3C and 3D). High-risk patients showed 67% improved survival when treated by adjuvant chemotherapy compared to observation (HR=0.33, 95%CI 0.17-0.63, p=0.0005, FIG. 3D), while those assigned to the low risk group did not benefit (FIG. 3C). These results were reproduced when applied separately to both the Stage IB (FIGS. 3E and 3F) and Stage II (FIGS. 3G and 3H) patients.

Multivariate analysis showed that the decrease of survival associated with adjuvant chemotherapy was independent of the stage (HR=2.26, 95% CI 1.03-4.96, p=0.04). A Cox regression model with chemotherapy received and risk group indicator and their interaction term as independent covariates were performed to fit the overall survival data on the 133 patients with microarray data. This analysis revealed that the interaction term is highly significant (p=0.0003) with the high-risk group deriving significantly greater benefit from adjuvant chemotherapy.

D. The Initial Study Population

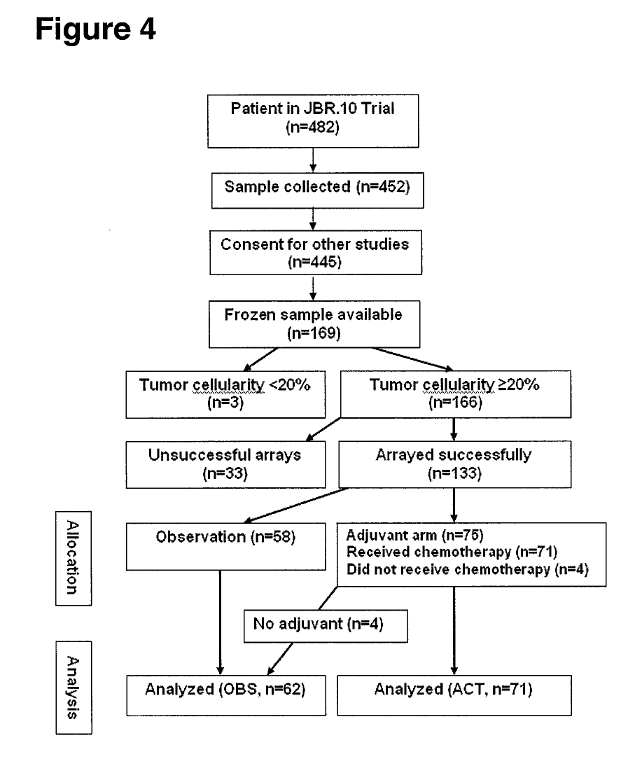

The initial study population comprised a subset of the patients randomized in the JBR.10 trial. There were 169 frozen tumor samples collected from patients who had their surgery at one of the BR.10 Canadian Centres have consented to the use of their samples for “future” studies in addition to RAS mutation analysis. The samples were harvested using a standardized protocol that was agreed upon during trial protocol development by designated pathologists from each participating centre. All tumors and corresponding normal lung tissue were collected as soon as or within 30 min after resection, and were snap-frozen in liquid nitrogen. For each frozen tissue fragment, a 1 mm cross-section slice was fixed in 10% buffered formalin and submitted for paraffin embedding. Histological evaluation of the HE stained sections revealed 166 samples that contained ≧20% tumor cellularity. Among the latter, gene expression profiling was completed successfully in samples from 133 patients. These included 58 patients randomized to the observation (OBS) arm and 75 to the adjuvant chemotherapy (ACT) arm. However, 4 ACT patients refused chemotherapy, and for the purpose of this analysis, they were assigned to the OBS arm. Therefore, the final distribution included 62 OBS patients and 71 ACT patients (FIGS. 1 and 4).

E. Microarray Data Analysis

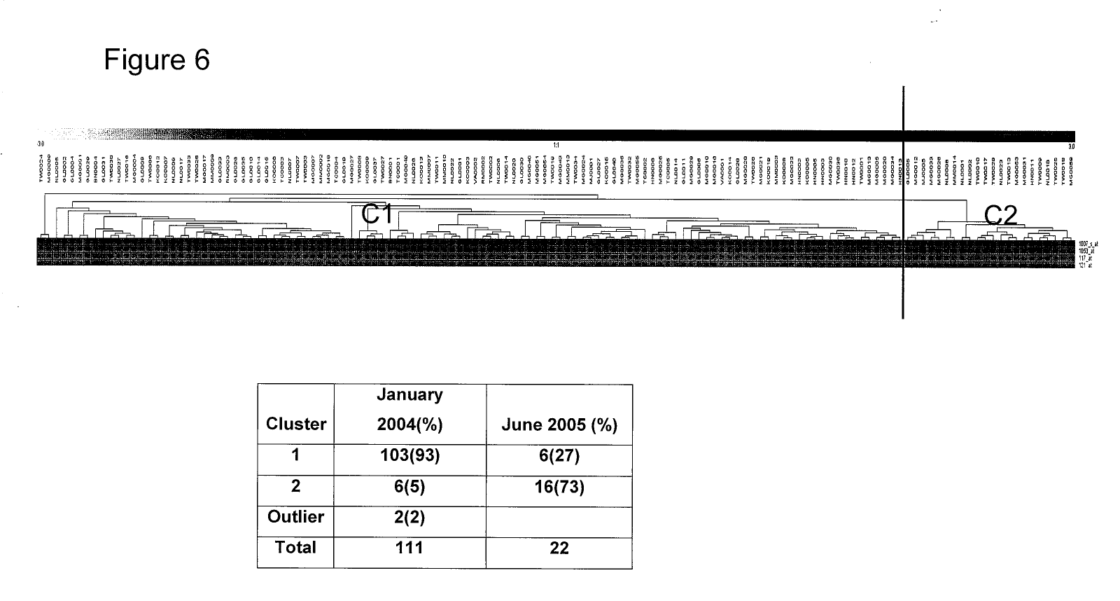

The raw microarray data from Affymetrix U133A (Affymetrix, Santa Clara, Calif.) were pre-processed using RMAexpress v0.32, then were twice log2 transformed since the distribution of additional log2 transformed data appeared more normal. Probe sets were annotated using NetAffx v4.2 annotation tool and only grade A level probe sets 3 (NA24) were included for further analysis. Affymetrix U133A chip contains 22,215 probe sets (19,619 probe sets with grade A annotation). Since the microarray hybridizations were performed in two batches at two separate occasions (January 2004, and June 2005), and unsupervised clustering showed that a batch difference was significant (FIG. 6), a distance-weighted discrimination (DWD) algorithm (https://genome.unc.edu/pubsup/dwd/index.html) was applied to homogenize the two batches. The DWD algorithm first finds a hyperplane that separates the two batches and adjusts the data by projecting the different batches on the DWD plane, finds the batch mean, and then subtracts out the DWD plane multiplied by this mean. In addition, the data were Z score transformed which made the validation across different datasets possible.

F. Univariate Analysis

The association of the expression of the individual probe set with overall survival (date of randomization to date of last follow up or death) was evaluated by Cox proportional hazards regression. The expression data for 62 patients in observation arm revealed 1312 probe sets that were associated with overall survival at p<0.05. Using a more stringent selection criteria of p<0.005, 172 probe sets with grade A annotation were prognostic.

G. Gene Set Signature Selection

To generate the gene expression signature, an exclusion selection procedure was firstly applied and followed by an inclusion process. The MAximizing R Square Algorithm (MARSA) included 3 sequential steps: a) probe set pre-selection; b) signature optimization; and c) leave-one-out-cross-validation. First, the candidate probe sets were pre-selected by their associations with survival at p<0.005 level. To remove the cross platform variation, expression data was z score transformed and risk score (z score weighted by the coefficient of the univariate Cox regression) was used to synthesize the information of the probe set combination. The candidate probe sets were then subjected to an exclusion followed by an inclusion selection procedure. For the preselected 172 probe sets, the exclusion procedure excluded one probe at a time, summed up the risk score of the remaining 171 probes, the calculated the R square (R², Goodness-of-fit) of the Cox model^{5,6}. Risk score was dichotomized by an outcome-orientated optimization of cutoff macro based on log-rank statistics (http://ndc.mayo.edu/mayo/research/biostat/sasmacros.cfm) before being introduced to the Cox proportional hazards model. A probe set was excluded if its exclusion resulted in obtaining the largest R². The procedure was repeated until there was only one probe set left. An inclusion procedure was followed using the probe set left by the exclusion procedure as the starting probe set. It included one probe set at a time, summed up the risk score of the included probe sets and risk score was dichotomized and R² was calculated. The probe set was included if its inclusion resulted in obtaining the largest R². The exclusion procedure produced a largest R square of 0.67 by a minimal 7 probe combination and the inclusion procedure generated a largest R2 of 0.78 by a minimal 15 probe combination (FIG. 1B), therefore, the 15 gene combination (Table 4) was selected as a candidate signature. Finally, the 15-gene signature (Table 4) was established after passing the internal validation by leave-one-out-cross-validation (LOOCV) and external validation on other datasets (listed below). All statistical analyses were performed using SAS v9.1 (SAS Institute, CA). The risk score was calculated as Table 4.

H. Prognostic Modeling by Principal Component Analysis of Signature Genes

Principal components analysis (PCA) (based on correlation matrix) was carried out to synthesize the information across the chosen gene probe sets and reduce the number of covariates in building the prognostic model. The eigenvalue of greater than or equal to 1 was used as cutoff point in determining how many proponents to include in the model, and those significantly correlated to disease-specific survival (DSS) were included in the final multivariable model. The PCA analysis was done based on all 133 patients with microarray data. When correlated to the DSS based on the 62 observation patients, the first 4 principal components were found to satisfy the criteria and were included in the prognostic model. Table 10 lists the four principal components for each of the 15 genes in the 15-gene signature. The same analysis can be applied to derive principal component coefficients for additional genes selected from the 172 genes listed in Table 3, such as for example, RGS4, UGT2B4, and/or MCF2. Furthermore, one of skill will appreciate from the above description how to obtain the first four principal component coefficients for any of the genes listed in Table 3.

To determine the gene signature prognostic group, multivariate Cox regression model with the first 4 principal components were fitted to the disease specific survival of the 62 observation patients. The linear prognostic scores were calculated by the sum of the multiplication of the estimated coefficient from Cox model and the corresponding principal component value. Using the prognostic score, patients were divided into low and high risk group based on the median of the prognostic score, i.e., those with prognostic score less than the median as low risk group, while those with score no less than the median as high risk group. For the 62 observation patients with microarray data, 31 patients were classified in each group. Applying the same rule to the 73 chemo-treated patients, 36 patients were classified in low risk group and 37 patients in high-risk group.

I. Validation of General Applicability of Prognostic Signature

Validation of the 15-gene signature was carried out on Stage I-II cases from Duke, UM-SQ, and DCC who did not receive adjuvant chemotherapy. When the risk score was dichotomized using the cutoff determined from the BR.10 training set, the 15-gene signature was able to separate 38 cases of low risk from 47 cases of high risk (log rank p=0.226) of NSCLC in the Duke dataset. Multivariate analysis (adjusted for stage, histology and patients' age and gender) showed that the 15-gene signature was an independent prognostic factor (HR=1.5, 95%CI 0.81-2.89, p=0.19, Table 2). UM-SQ contains squamous cell carcinoma only and the cases have the worst survival rate. However, the 15-gene signature was still able to separate 50 cases of low risk from 56 cases with high risk (log rank p=0.0447) and this separation was independent of stage and patients' age and gender (HR=2.3, 95% CI 1.1-4.7 p=0.026, Table 2). The DCC dataset contained only adenocarcinoma cases. Applying the 15-gene signature on DCC Stage I and II, was able to separate 87 low risk cases from the 82 high risk cases (log rank p=0.0002, FIG. 2E). Multivariate analysis (adjusted for stage and patients' age and gender) showed that the prognostic value of the 15-gene signature was independent prognostic factor (HR=2.9, 95%CI 1.5-5.6, p=0.002, Table 2). There were 67 Stage IB-II cases without chemotherapy in MI, the 15-gene signature was able to separate 44 low risk cases from the 23 high risk cases (log rank p=0.013). Multivariate analysis (adjusted for stage and patients' age and gender) showed that the prognostic value of the 15-gene signature was independent prognostic factor (HR=1.5, 95%CI 0.54-4.31, p=0.4, Table 2). Cases from MSKCC had a significantly better 5-year overall survival compared to other datasets. However, the 15-gene signature was able to separate 32 cases of low risk from 32 cases of high risk in MSKCC (log rank p=0.16). Multivariate analysis (adjusted for stage) revealed that the 15-gene signature was an independent prognostic factor. Validation of the 15-gene signature on HLM revealed that the 15-gene signature was able to separate 26 cases of low risk from 24 cases of high risk (log rank p=0.0084). Multivariate analysis (adjusted for stage) showed that there was a trend to separation by the 15-gene signature (HR=1.2, 95%CI 0.43-3.6, p=0.7). These validation data confirm that the 15-gene signature is a strong prognostic signature and its power of predicting the outcome of NSCLC is independent of and superior to that of stage.

J. The Benefit of Chemotherapy was Limited to High Risk Patients

A total of 30 deaths were observed in the ACT. Six of them were due to other malignancies. The 15-gene signature was unable to separate the good/bad outcome patients (p=0.83, data not shown) in the ACT. However, stratified analysis showed that only patients with high risk derived benefit from adjuvant chemotherapy (FIG. 3D). Upon receiving adjuvant chemotherapy, the survival rate of the 36 high-risk patients was significantly improved (HR=0.33, 95%CI 0.17-0.63, p=0.0005, FIG. 3D). On the other hand, the application of chemotherapy on low risk patients resulted in a decrease in survival rate (HR=3.67, 95%CI 1.22-11.06, p=0.0133, FIG. 3C). Death was evenly distributed between the low and high risk groups in the ACT arm (15 deaths in low and high risk group, respectively). Each of these two groups contained 3 deaths that were not due to lung cancer. Stratification by risk group and stage showed that the survival rate of high risk patients from both Stage IB and Stage II was significantly improved by chemotherapy (FIGS. 3F and H). Moreover, for low risk patients of Stage II, chemotherapy was associated with significantly decreased survival (FIGS. 3E and G). A Cox regression model with chemotherapy received and risk group indicator and their interaction term as independent covariates was performed to fit the overall survival data on the 133 patients with microarray data. This analysis revealed that the interaction term is highly significant (p=0.0002) with the high-risk group deriving significantly greater benefit from adjuvant chemotherapy.

Gene expression signature is thought to represent the altered key pathways in carcinogenesis and thus is able to predict patients' outcome. However, being able to faithfully represent the altered key pathways, the signature must be generated from genome-wide gene expression data. The present study used all information generated by Affymetrix U133A chip on NSCLC samples from a randomized clinical trial to derive a 15-gene signature. The 15-gene signature was able to identify 50% (31/62) Stage IB-II NSCLC patients had relative good outcome. Multivariate analysis indicated that the 15-gene signature was an independent prognostic factor. Moreover, its independent prognostic effect had been in silico validated on 169 adenocarcinomas without adjuvant chemo- or radio-therapy from DCC and 85 NSCLC from Duke and 106 squamous cell carcinomas of the lung from the University of Michigan (UM-SQ). Importantly, the 15-gene signature was able to predict the response to adjuvant chemotherapy with high-risk patients across the stages being benefited from adjuvant chemotherapy. This finding was also validated on DCC dataset.

Adjuvant chemotherapy for completely resected early stage NSCLC was a research question until the results of a series of positive trials^{2, 4}, including BR.10³, were published. However, whether chemotherapy played a beneficial role in Stage IB remained to be clarified²⁻⁶. The present study showed that the Stage IB patients were potentially able to be separated into low (49.3%, 36/73) and high (50.7%, 37/73) risk groups using the 15-gene signature. Upon administering the adjuvant chemotherapy to Stage IB patients, the survival rate of patients with high risk was significantly improved (p=0.0698, FIG. 3F) whereas patients with low risk did not experience a benefit in survival (p=0.0758, FIG. 3E). Therefore the effect of chemotherapy on Stage IB NSCLC was neutralized and thus gave an incorrect impression that no beneficial effect was existed³. Based on the evidence provided here and from the meta-analysis⁶, it may be concluded that 50.7% (37/73) Stage IB NSCLC patients have the potential to benefit from adjuvant chemotherapy.

Another significance of the present study was that the signature was able to identify a subgroup (50%, 30/60) of patients from Stage II who did not benefit from adjuvant chemotherapy (p=0.1498, FIG. 3G). In current practice, adjuvant chemotherapy is recommended for all patients. However, the 15-gene signature suggests that about a half of the Stage II patients may not benefit from adjuvant chemotherapy.

The gene ontology analysis showed that in the 15-gene signature, 4 genes (FOSL2, HEXIM1, IKBKAP, MYT1L, and ZNF236) were involved in the regulation of transcription. EDN3 and STMN2 played a role in signal transduction. Transformed 3T3 cell double minute 2 (MDM2), an E3 ubiquitin ligase, which targets p53 protein for degradation, plays a key role in cell cycle and apoptosis. Dworakowska D. et al.²⁴ reported that overexpression of MDM2 protein was correlated with low apoptotic index, which was associated with poorer survival. Myoglobin (MB) palyed a role in response to hypoxia and Uridine monophosphate synthetase (UMPS) participated in the ‘de novo’ pyrimidine base biosynthetic process, however, none of them has not been explored in lung cancer. The L1 cell adhesion molecule (L1CAM) involved in cell adhesion whose overexpression was associated with tumor metastasis and poor prognosis²⁵⁻²⁸. ATPase, Na+/K+ transporting, beta 1 polypeptide (ATP1B1) was involved in ion transport which was reported recently to be able to discriminate the serous low malignant potential and invasive epithelial ovarian tumors²⁹. These findings indicated that cellular transcription, cell cycle and apoptosis, cell adhesion and response to hypoxia were important for lung cancer progression.

The range of expression levels of members of the 15-gene signature was broad, from very low expression level such as MDM2 and ZNF236 to fairly high expression such as TRIM14 or very high expression such as ATP1B1 (Table 4). Least variable gene (<5%), such as UMPS (Table 4), was also a member of the signature. These data suggested that it may not be a good practice to exclude low expressed and least variable probe set in the data pre-selection process in an arbitrary way. The signature generated using the present strategy performed better than that of Raponi et al.'s method of using the top 50 genes. There are only 3 genes (IKBKAP, L1CAM, and FAM64A) whose significance in association with survival is in the top 50 genes (Table 4).

K. Patients and Samples

Included in the JBR.10 protocol was the collection of snap-frozen or formalin-fixed paraffin embedded tumor samples for KRAS mutation analysis and tissue banking for future laboratory studies3. Altogether 445 of 482 randomized patients consented to banking. Snap-frozen tissues were collected from 169 Canadian patients (FIG. 4). Histological evaluation of the HE section from the snap-frozen tumor samples revealed 166 that contained an estimated >20% tumor cellularity; gene expression profiling was completed in 133 of these patient samples, using the U133A oligonucleotide microarrays (Affymetrix, Santa Clara, Calif.). Profiling was not completed in 33 patient samples. Of 133 patients with microarray profiles, 62 did not received post-operative adjuvant chemotherapy and were group as observation patients, while 71 patients were received chemotherapy. University Health Network Research Ethics Board approved the study protocol.

L. RNA Isolation and Microarray Profiling

Total RNA was isolated from frozen tumor samples after homogenization in guanidium isothiocyanate solution and acid phenol-chloroform extraction. The quality of isolated RNA was assessed initially by gel electrophoresis, followed by the Agilent Bioanalyzer. Ten micrograms of total RNA was processed, labeled, and hybridized to Affymetrix's HG-U133A GeneChips. Microarray hybridization was performed at the Center for Cancer Genome Discovery of Dana Farber Cancer Institute.

M. Microarray Data Analysis and Gene Annotation

The raw microarray data were pre-processed using RMAexpress v0.3²². Probe sets were annotated using NetAffx v4.2 annotation tool and only grade A level probe sets²³ (NA22) were included for further analysis. Because the microarray profiling was done in two separate batches at different times and unsupervised heuristic K-means clustering identified a systematic difference between the two batches (FIG. 6), the distance-weighted discrimination (DWD) method (https://genome.unc.edu/pubsup/dwd/index.html) was used to adjust the difference. The DWD method first finds a separating hyperplane between the two batches and adjusts the data by projecting the different batches on the DWD plane, discover the batch mean, and then subtracts out the DWD plane multiplied by this mean. The data were then transformed to Z score by centering to its mean and scaling to its standard deviation. This transformation was necessary for validation on different datasets in which different expression ranges are likely to exist, and for validation on different platforms, such as qPCR where the data scale is different.

N. Derivation of Signature

The pre-selected probe sets by univariate analysis at p<0.005 were selected by an exclusion procedure. The exclusion selection excluded one probe set at a time based on the resultant R square (R², Goodness-of-fit^{15, 16}) of the Cox model. It kept repeating until there was only one probe set left. The procedure was repeated until there was only one probe set left. An inclusion procedure was followed using the probe set left by the exclusion procedure as the starting probe set. It included one probe set at a time based on the resultant R² of the Cox model. Finally, the R² was plotted against the probe set and a set of minimum number of probe sets yet having the largest R² was chosen as candidate signature. Gene signature was established after passing the internal validation by leave-one-out-cross-validation (LOOCV) and external validation on other datasets (listed below). All statistical analyses were performed using SAS v9.1 (SAS Institute, CA).

O. Validation in Separate Microarray Datasets

The prognostic value of this 15-gene signature was tested on separate microarray datasets. Three represented subsets of microarray data from the NCI Director's Challenge Consortium (DCC) for the Molecular Classification of Lung Adenocarcinoma (Nature Medicine, in review/in press). In total, the Consortium analyzed the profiles of 442 tumors, including 177 from University of Michigan (UM), 79 from H. L. Moffitt Cancer Centre (HLM), 104 from Memorial Sloan-Kettering Cancer Centre (MSK), and 82 from our group. As 39 of the latter tumors overlap with samples used in this study, only data from the first 3 groups were used for validation. In addition, patients who were noted as either unknown or having received adjuvant chemotherapy and/or radiotherapy were excluded. Therefore, the DCC dataset used in this validation study included only 169 patients: 67 from UM, 46 from HLM, 56 from MSK. Two additional published microarray datasets were also used for validation: the Duke's University dataset of 85 non-small cell lung cancer patients (Potti et al, NEJM), and the University of Michigan dataset of 106 squamous cell carcinomas patients (UM-SQ) (Raponi et al). Raw data of these microarray studies were downloaded and RMA pre-processed. The expression levels were Z score transformed after double log2 transformation. Risk score was the Z score weighted by the coefficient of the Cox model from the OBS. Demographic data of the DCC cohort was listed in Table 5.

P. Statistical Analysis

Risk score was the product of coefficient of Cox proportional model and the standardized expression level. The univariate association of the expression of the individual probe set with overall survival (date of randomization to date of last followup or death) was evaluated by Cox proportional hazards regression. A stringent p<0.005 was set as a selection criteria in order to minimize the possibility of false-positive results.

### Example 2

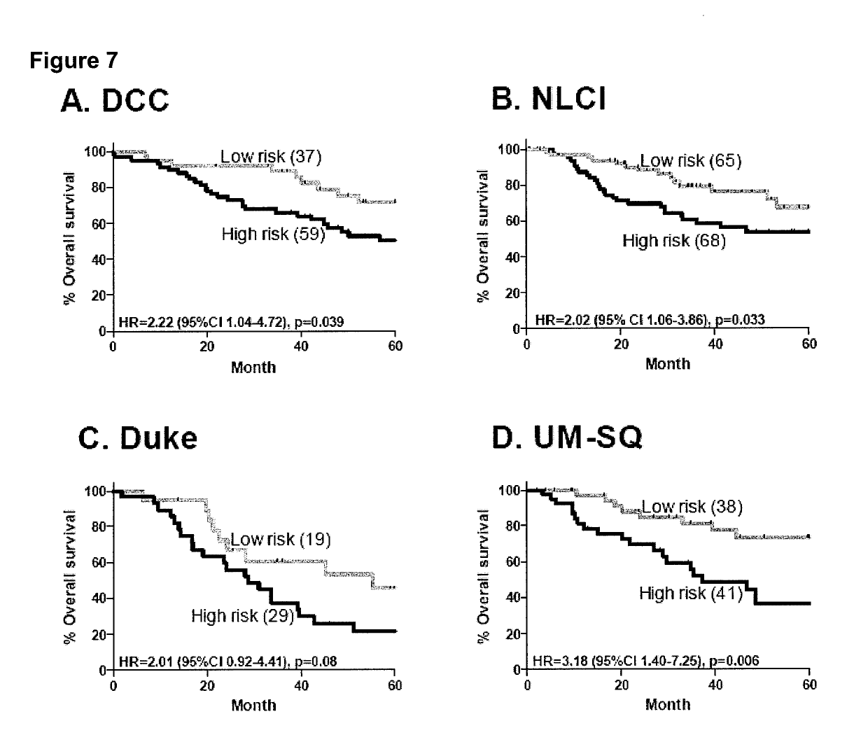

The 15-gene signature was additionally tested for its prognostic significance in a subset of Stage IB and II patient samples from four independent published microarray datasets, three as described previously (DCC, UM-SQ and Duke), and the fourth from the Netherlands Cancer Institute (NLCI). These datasets, comprised of resected Stage IB-II NSCLC patients who had not received any type of adjuvant therapy (total n=356; Table 13). As described, the risk score was the expression level weighted by the coefficients of the four PCs derived from the training set. When the risk score was dichotomized at −0.1, the 15-gene signature classified into low and high risk groups, respectively, 37 and 59 of 96 ADC patients from DCC (p=0.039, FIG. 7A); 65 and 68 of 133 NSCLC patients from NLCI (p=0.033, FIG. 7B); 19 and 29 of 48 NSCLC patients from Duke University (p=0.08, FIG. 7C); and 38 and 41of 79 SQCC patients from UM-SQ (p=0.006, FIG. 7D). Multivariate analysis demonstrated that the signature was an independent prognostic factor in these four validation datasets after adjusting for other potentially prognostic clinical factors (DCC: HR 2.26, CI 1.02-4.97, p=0.044; NLCI: HR 2.27, CI 1.18-4.35, p=0.014; Duke: HR 1.96, CI 0.9-4.4, p=0.11; UM-SQ: HR 3.57, 95% CI 1.48-8.58, p=0.005, Table 12). The insignificant p-value in the Duke dataset might be due to its small sample size (n=48). HR compares the overall survival of the high-risk (poor prognosis) patient group to that of the low-risk (good prognosis) group, after adjustment for tumor histologic subtype, stage, age and sex. The model was not adjusted for histology for UM-SQ. Since the NLCI dataset did not contain information on sex this covariate was not included in the model.

To reflect the JBR.10 population, validation was restricted to Stage IB-II patients who received neither adjuvant chemotherapy nor radiotherapy. The 15-gene signature was tested in four independent microarray datasets including a subset of the National Cancer Institute Director's Challenge Consortium (DCC) for the Molecular Classification of Lung Adenocarcinoma 15. The Consortium profiled 442 lung adenocarcinomas, including 177 from University of Michigan (UM), 79 from H. L. Moffitt Cancer Center (HLM), 104 from Memorial Sloan-Kettering Cancer Center (MSK), and 39 samples from the CAN/DF cohort, excluding 43 samples from JBR.10 that were part of the training set. Therefore, the DCC validation dataset included 96 patients (27 UM, 38 HLM, 31 MSK). The three additional microarray datasets included 89 NSCLC patients without adjuvant therapy from Duke University (Duke, 48 Stage IB-II) 8, 129 squamous cell carcinoma patients without adjuvant therapy from the University of Michigan (UM-SQ, 79 Stage IB-II) 9, and 172 NSCLC patients without adjuvant therapy from the Netherlands Cancer Institute (NLCI, 133 Stage IB-II) 18. Probe sets matching from Affymetrix U133A to the Agilent 44K platform used in the NLCI study was based on Unigene ID mapping obtained from NetAffx annotation (NA22), and annotation provided by Roepman et al (http://research.agendia.com/), respectively. Expression level was averaged if multiple matching probe sets were found in the NLCI data.

### Example 3

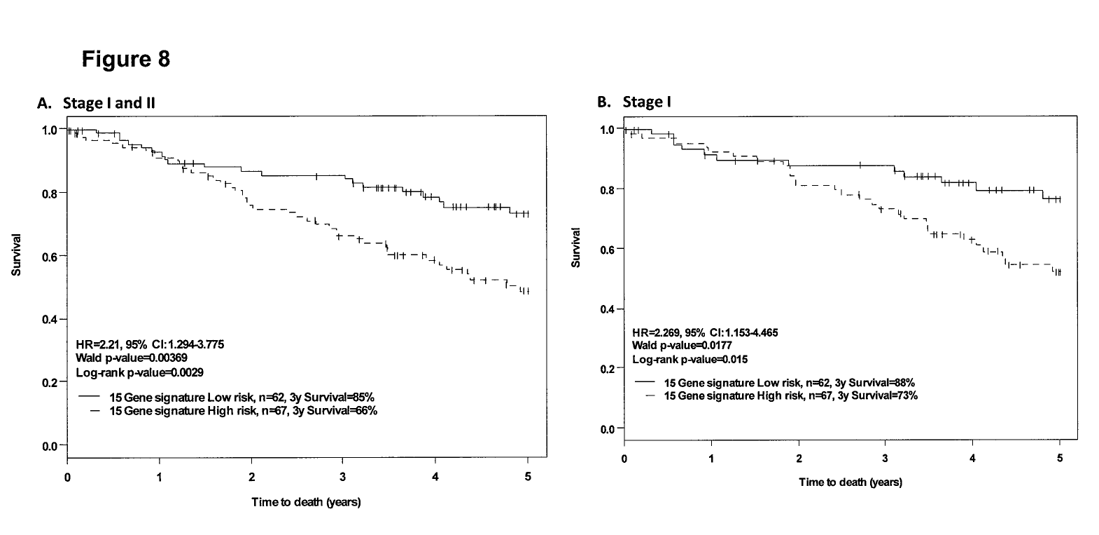

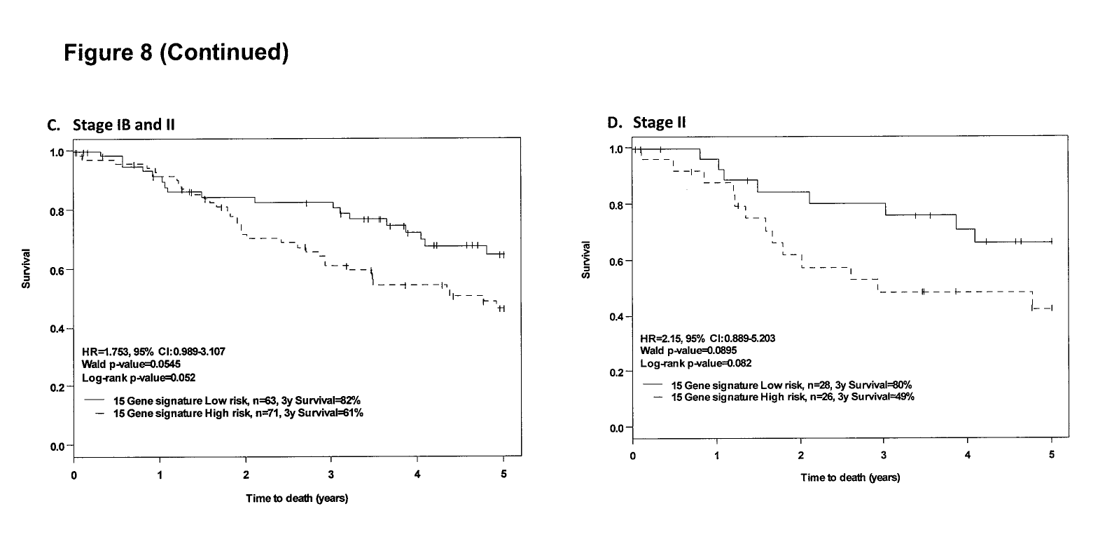

Validation was performed in a fifth patient cohort comprising 183 frozen tumor samples from patients with early stage NSCLC (Stage I/II) treated at Princess Margaret Hospital/University Health Network (2000-2005) with the clinical and demographic profile outlined in Table 14. Whole genome, microarray-based profiling was performed on these samples and patients were segregated into high and low risk groups using the expression values of the 15-gene signature derived from the arrays. The survival of these two risk groups was significantly different (n=183, HR =2.21, 95% CI: 1.28-3.81, p-value=0.0045, FIG. 8A). Subset analysis using this signature to segregate patients into good and poor prognosis subgroups within Stage I (n=129, HR 2.30, 95% CI 1.15-4.59, p-value=0.019, FIG. 8B), Stages IB and II (n=134, HR=1.753, p-value=0.052, FIG. 8C) and Stage II (n=54, HR 2.075, 95% CI 0.83- 5.17, p-value=0.12, FIG. 8D) indicated that the signature is also an independent prognostic factor for at least Stage I NSCLC. The magnitude of the difference for Stage II was similar to Stage I. Provided a larger sample size, statistical significance of the signature as an independent prognostic factor is likely for Stage II NSCLC. Again, the signature was tested using a Cox proportional hazards model while controlling for clinical factors, age, sex, and histology.

For the whole genome expression analysis of the cohort of 183 UHN patients, total RNA was isolated from frozen tumors by homogenization in guanidium isothiocyanate solution and acid phenol-chloroform extraction, purified by RNeasy mini kit and checked by Agilent Bioanalyzer for quality. Total RNA was processed, labeled, and hybridized to Affymetrix HG-U133 Plus 2.0 GeneChips. Raw microarray data were pre-processed using RMAexpress v0.3 16. Expression data for the 15 genes were extracted and validation was performed as described for the above datasets.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Baseline factors of BR.10 patients
with and without microarray profiles

| | | Microarray | | No microarray | | |
|---|---|---|---|---|---|---|
| | All | profiled | | profiled | | |
| | Patients | (n = 133) | | (n = 349) | | |
| Factor | (n = 482) | n | % | n | % | P value |
| Treatment received | | | | | | |
| ACT | 231 | 71 | 53% | 160 | 46% | 0.14 |
| OBS | 251 | 62 | 47% | 189 | 54% | |
| Age | | | | | | |
| <65 | 324 | 87 | 65% | 237 | 68% | 0.6 |
| ≧65 | 158 | 46 | 35% | 112 | 32% | |
| Gender | | | | | | |
| Male | 314 | 91 | 68% | 223 | 64% | 0.35 |
| Female | 168 | 42 | 32% | 126 | 36% | |
| Performance Status | | | | | | |
| 0 | 236 | 67 | 50% | 169 | 49% | 0.72 |
| 1 | 245 | 66 | 50% | 179 | 51% | |
| Stage of Disease | | | | | | |
| IB | 219 | 73 | 55% | 146 | 42% | 0.01 |
| II | 263 | 60 | 45% | 203 | 58% | |
| Surgery | | | | | | |
| Pneumonectomy | 113 | 33 | 25% | 80 | 23% | 0.66 |
| Other Resection | 369 | 100 | 75% | 269 | 77% | |
| Pathologic type | | | | | | |
| Adenocarcinoma | 256 | 71 | 53% | 185 | 53% | 0.56 |
| Squamous | 179 | 52 | 39% | 127 | 36% | |
| Other | 47 | 10 | 8% | 37 | 11% | |
| Ras Mutation Status | | | | | | |
| Present | 117 | 28 | 21% | 89 | 26% | 0.12* |
| Absent | 333 | 105 | 79% | 228 | 65% | |
| Unknown | 32 | 0 | 0% | 32 | 9% | |

*P-value: Without include those missing or unknown.

TABLE 2
Comparison of 5-yr Survival (multivariate) of High and Low Risk
Groups in Untreated Patients and Patients who Received
Adjuvant Chemotherapy

| | | | |
|---|---|---|---|
| n | HR* | 95% CI | p value |

Observation/untreated Patients

| | | | | |
|---|---|---|---|---|
| JBR.10 (randomized with | 62 | 18.0 | 5.8-56.1 | <0.0001 |
| microarray) | | | | |
| Stage IB | 34 | 29.9 | 4.5-197.4 | 0.0004 |
| Stage II | 28 | 16.4 | 3.0-88.1 | 0.001 |
| DCC (no adjuvant therapy) | 169 | 2.9 | 1.5-5.6 | 0.002 |
| UM | 67 | 1.5 | 0.54-4.31 | 0.4 |
| HLM | 46 | 1.2 | 0.43-3.60 | 0.7 |
| MSK | 56 | NA** | NA | |
| Duke | 85 | 1.5 | 0.81-2.89 | 0.19 |
| UM-Squamous | 106 | 2.3 | 1.1-4.7 | 0.026 |

Patients Treated With Adjuvant Chemotherapy

| | | | | |
|---|---|---|---|---|
| BR.10 (randomized with | 71 | 1.5 | 0.7-3.3 | 0.28 |
| microarray) | | | | |
| BR.10 Stage I | 39 | 1.7 | 0.5-5.6 | 0.36 |
| BR.10 Stage II | 32 | 1.2 | 0.4-3.6 | 0.8 |
| DCC (not randomized) | 41 | 1.1 | 0.5-2.5 | 0.8 |

n: number of patients;
HR: hazard ratio;
CI: confidence interval.
*HR compares the survival of the poor prognostic group to that of the good prognostic group as determined by the 15-gene signature with the adjustment of stage and patients' age and gender. For BR.10, and Duke, the effect of histology was also adjusted.
**All events were in high risk group and female patients.

TABLE 3
172 U133A probe sets that were prognostic at p < 0.005 for the 62 BR.10 observation arm patients

| | Representative | | | | | | | |
|---|---|---|---|---|---|---|---|---|
| Probe Set ID | Public ID | UniGene ID | Gene Symbol | Coefficients | HR | HRL | HRH | p value |
| 200878_at | AF052094 | Hs.468410 | EPAS1 | −0.58 | 0.56 | 0.37 | 0.84 | 0.0048 |
| 201228_s_at | NM_006321 | Hs.31387 | ARIH2 | 0.47 | 1.60 | 1.17 | 2.18 | 0.0029 |
| 201242_s_at | BC000006 | Hs.291196 | ATP1B1 | −0.69 | 0.50 | 0.35 | 0.71 | 0.0001 |
| 201243_s_at | NM_001677 | Hs.291196 | ATP1B1 | −0.54 | 0.58 | 0.41 | 0.83 | 0.0028 |
| 201301_s_at | NM_001153 | Hs.422986 | ANXA4 | −0.55 | 0.58 | 0.40 | 0.83 | 0.0028 |
| 201502_s_at | NM_020529 | Hs.81328 | NFKBIA | −0.62 | 0.54 | 0.36 | 0.79 | 0.0016 |
| 202023_at | NM_004428 | Hs.516664 | EFNA1 | −0.67 | 0.51 | 0.35 | 0.76 | 0.0009 |
| 202035_s_at | AF017987 | Hs.213424 | SFRP1 | 0.69 | 1.99 | 1.39 | 2.86 | 0.0002 |
| 202036_s_at | AF017987 | Hs.213424 | SFRP1 | 0.84 | 2.31 | 1.56 | 3.44 | 0.0000 |
| 202037_s_at | AF017987 | Hs.213424 | SFRP1 | 0.74 | 2.09 | 1.43 | 3.07 | 0.0002 |
| 202490_at | AF153419 | Hs.494738 | IKBKAP | 0.42 | 1.53 | 1.17 | 1.99 | 0.0018 |
| 202707_at | NM_000373 | Hs.2057 | UMPS | 0.60 | 1.81 | 1.24 | 2.66 | 0.0023 |
| 202814_s_at | NM_006460 | Hs.15299 | HEXIM1 | 0.59 | 1.80 | 1.20 | 2.70 | 0.0045 |
| 203001_s_at | NM_007029 | Hs.521651 | STMN2 | 0.55 | 1.73 | 1.21 | 2.47 | 0.0027 |
| 203147_s_at | NM_014788 | Hs.575631 | TRIM14 | −0.56 | 0.57 | 0.39 | 0.82 | 0.0028 |
| 203438_at | AI435828 | Hs.233160 | STC2 | 0.67 | 1.96 | 1.29 | 2.96 | 0.0015 |
| 203444_s_at | NM_004739 | Hs.173043 | MTA2 | 0.38 | 1.46 | 1.12 | 1.89 | 0.0046 |
| 203475_at | NM_000103 | Hs.511367 | CYP19A1 | 0.56 | 1.76 | 1.23 | 2.52 | 0.0021 |
| 203509_at | NM_003105 | Hs.368592 | SORL1 | −0.58 | 0.56 | 0.39 | 0.81 | 0.0020 |
| 203928_x_at | AI870749 | Hs.101174 | MAPT | 0.44 | 1.55 | 1.15 | 2.10 | 0.0044 |
| 203973_s_at | M83667 | Hs.440829 | CEBPD | −0.61 | 0.54 | 0.38 | 0.77 | 0.0005 |
| 204179_at | NM_005368 | Hs.517586 | MB | 0.47 | 1.60 | 1.16 | 2.22 | 0.0044 |
| 204267_x_at | NM_004203 | Hs.77783 | PKMYT1 | 0.63 | 1.87 | 1.28 | 2.73 | 0.0011 |
| 204338_s_at | AL514445 | Hs.386726 | RGS4 | 0.57 | 1.77 | 1.23 | 2.53 | 0.0021 |
| 204531_s_at | NM_007295 | Hs.194143 | BRCA1 | 0.60 | 1.82 | 1.21 | 2.75 | 0.0043 |
| 204584_at | AI653981 | Hs.522818 | L1CAM | 0.56 | 1.75 | 1.30 | 2.35 | 0.0002 |
| 204684_at | NM_002522 | Hs.645265 | NPTX1 | 0.48 | 1.61 | 1.18 | 2.19 | 0.0024 |
| 204810_s_at | NM_001824 | Hs.334347 | CKM | 0.46 | 1.58 | 1.20 | 2.09 | 0.0012 |
| 204817_at | NM_012291 | — | ESPL1 | 0.53 | 1.70 | 1.24 | 2.34 | 0.0010 |
| 204933_s_at | BF433902 | Hs.81791 | TNFRSF11B | 0.51 | 1.67 | 1.27 | 2.20 | 0.0003 |
| 204953_at | NM_014841 | Hs.368046 | SNAP91 | 0.59 | 1.81 | 1.31 | 2.49 | 0.0003 |
| 205046_at | NM_001813 | Hs.75573 | CENPE | 0.62 | 1.86 | 1.28 | 2.70 | 0.0012 |
| 205189_s_at | NM_000136 | Hs.494529 | FANCC | 0.53 | 1.70 | 1.21 | 2.40 | 0.0023 |
| 205217_at | NM_004085 | Hs.447877 | TIMM8A | 0.64 | 1.90 | 1.26 | 2.85 | 0.0020 |
| 205386_s_at | NM_002392 | Hs.567303 | MDM2 | 0.49 | 1.63 | 1.19 | 2.23 | 0.0025 |
| 205433_at | NM_000055 | Hs.420483 | BCHE | 0.58 | 1.79 | 1.23 | 2.62 | 0.0024 |
| 205481_at | NM_000674 | Hs.77867 | ADORA1 | 0.49 | 1.63 | 1.20 | 2.23 | 0.0020 |
| 205491_s_at | NM_024009 | Hs.522561 | GJB3 | 0.46 | 1.58 | 1.18 | 2.11 | 0.0021 |
| 205501_at | AI143879 | Hs.348762 | — | 0.40 | 1.49 | 1.13 | 1.97 | 0.0043 |
| 205825_at | NM_000439 | Hs.78977 | PCSK1 | 0.59 | 1.81 | 1.24 | 2.65 | 0.0023 |
| 205893_at | NM_014932 | Hs.478289 | NLGN1 | 0.40 | 1.49 | 1.13 | 1.97 | 0.0048 |
| 205938_at | NM_014906 | Hs.245044 | PPM1E | 0.52 | 1.68 | 1.22 | 2.31 | 0.0013 |
| 205946_at | NM_003382 | Hs.490817 | VIPR2 | 0.50 | 1.65 | 1.17 | 2.33 | 0.0043 |
| 206043_s_at | NM_014861 | Hs.6168 | ATP2C2 | −0.55 | 0.57 | 0.39 | 0.84 | 0.0044 |
| 206096_at | AI809774 | Hs.288658 | ZNF35 | 0.55 | 1.73 | 1.20 | 2.49 | 0.0034 |
| 206228_at | AW769732 | Hs.155644 | PAX2 | 0.50 | 1.65 | 1.27 | 2.15 | 0.0002 |
| 206232_s_at | NM_004775 | Hs.591063 | B4GALT6 | 0.44 | 1.56 | 1.17 | 2.07 | 0.0021 |
| 206401_s_at | J03778 | Hs.101174 | MAPT | 0.39 | 1.48 | 1.13 | 1.94 | 0.0049 |
| 206426_at | NM_005511 | Hs.154069 | MLANA | 0.63 | 1.87 | 1.26 | 2.77 | 0.0018 |
| 206496_at | NM_006894 | Hs.445350 | FMO3 | 0.53 | 1.70 | 1.22 | 2.37 | 0.0018 |
| 206505_at | NM_021139 | Hs.285887 | UGT2B4 | 0.61 | 1.84 | 1.26 | 2.69 | 0.0017 |
| 206524_at | NM_003181 | Hs.389457 | T | 0.78 | 2.18 | 1.35 | 3.53 | 0.0015 |
| 206552_s_at | NM_003182 | Hs.2563 | TAC1 | 0.97 | 2.63 | 1.53 | 4.53 | 0.0005 |
| 206619_at | NM_014420 | Hs.159311 | DKK4 | 0.54 | 1.72 | 1.20 | 2.45 | 0.0029 |
| 206622_at | NM_007117 | Hs.182231 | TRH | 0.53 | 1.70 | 1.23 | 2.37 | 0.0015 |
| 206661_at | NM_025104 | Hs.369998 | DBF4B | 0.55 | 1.73 | 1.27 | 2.36 | 0.0005 |
| 206672_at | NM_000486 | Hs.130730 | AQP2 | 0.37 | 1.45 | 1.13 | 1.84 | 0.0030 |
| 206678_at | NM_000806 | Hs.175934 | GABRA1 | 0.39 | 1.48 | 1.16 | 1.89 | 0.0014 |
| 206799_at | NM_006551 | Hs.204096 | SCGB1D2 | 0.41 | 1.51 | 1.15 | 1.99 | 0.0032 |
| 206835_at | NM_003154 | Hs.250959 | STATH | 0.46 | 1.59 | 1.16 | 2.18 | 0.0042 |
| 206940_s_at | NM_006237 | Hs.493062 | POU4F1 | 0.54 | 1.72 | 1.23 | 2.40 | 0.0017 |
| 206984_s_at | NM_002930 | Hs.464985 | RIT2 | 0.47 | 1.59 | 1.16 | 2.20 | 0.0045 |
| 207003_at | NM_002098 | Hs.778 | GUCA2A | 0.62 | 1.85 | 1.23 | 2.79 | 0.0032 |
| 207028_at | NM_006316 | Hs.651453 | MYCNOS | 0.48 | 1.61 | 1.19 | 2.18 | 0.0020 |
| 207208_at | NM_014469 | Hs.121605 | HNRNPG-T | 0.51 | 1.66 | 1.23 | 2.26 | 0.0010 |
| 207219_at | NM_023070 | Hs.133034 | ZNF643 | 0.60 | 1.82 | 1.27 | 2.60 | 0.0011 |
| 207529_at | NM_021010 | — | DEFA5 | 0.65 | 1.91 | 1.38 | 2.64 | 0.0001 |
| 207597_at | NM_014237 | Hs.127930 | ADAM18 | 0.63 | 1.87 | 1.36 | 2.58 | 0.0001 |
| 207814_at | NM_001926 | Hs.711 | DEFA6 | 0.61 | 1.85 | 1.21 | 2.81 | 0.0041 |
| 207843_x_at | NM_001914 | Hs.465413 | CYB5A | −0.55 | 0.58 | 0.39 | 0.84 | 0.0047 |
| 207878_at | NM_015848 | — | KRT76 | 0.41 | 1.51 | 1.17 | 1.95 | 0.0017 |
| 207937_x_at | NM_023110 | Hs.264887 | FGFR1 | 0.43 | 1.54 | 1.14 | 2.08 | 0.0045 |
| 208157_at | NM_009586 | Hs.146186 | SIM2 | 0.45 | 1.56 | 1.19 | 2.05 | 0.0013 |
| 208233_at | NM_013317 | Hs.468675 | PDPN | 0.54 | 1.72 | 1.18 | 2.49 | 0.0043 |
| 208292_at | NM_014482 | Hs.158317 | BMP10 | 0.44 | 1.55 | 1.17 | 2.05 | 0.0025 |
| 208314_at | NM_006583 | Hs.352262 | RRH | 0.56 | 1.75 | 1.19 | 2.58 | 0.0044 |
| 208368_s_at | NM_000059 | Hs.34012 | BRCA2 | 0.62 | 1.86 | 1.26 | 2.73 | 0.0018 |
| 208399_s_at | NM_000114 | Hs.1408 | EDN3 | 0.48 | 1.61 | 1.18 | 2.20 | 0.0028 |
| 208511_at | NM_021000 | Hs.647156 | PTTG3 | 0.49 | 1.63 | 1.17 | 2.29 | 0.0043 |
| 208684_at | U24105 | Hs.162121 | COPA | −0.52 | 0.59 | 0.41 | 0.85 | 0.0041 |
| 208992_s_at | BC000627 | Hs.463059 | STAT3 | −0.67 | 0.51 | 0.34 | 0.77 | 0.0012 |
| 209434_s_at | U00238 | — | PPAT | 0.43 | 1.54 | 1.15 | 2.06 | 0.0033 |
| 209839_at | AL136712 | Hs.584880 | DNM3 | 0.54 | 1.72 | 1.18 | 2.50 | 0.0049 |
| 209859_at | AF220036 | Hs.368928 | TRIM9 | 0.45 | 1.57 | 1.16 | 2.12 | 0.0032 |
| 210016_at | BF223003 | Hs.434418 | MYT1L | 0.60 | 1.82 | 1.31 | 2.52 | 0.0003 |
| 210247_at | AW139618 | Hs.445503 | SYN2 | 0.64 | 1.89 | 1.30 | 2.75 | 0.0008 |
| 210302_s_at | AF262032 | Hs.584852 | MAB21L2 | 0.59 | 1.81 | 1.34 | 2.44 | 0.0001 |
| 210315_at | AF077737 | Hs.445503 | SYN2 | 0.66 | 1.94 | 1.31 | 2.87 | 0.0009 |
| 210455_at | AF050198 | Hs.419800 | C10orf28 | 0.57 | 1.76 | 1.24 | 2.50 | 0.0015 |
| 210758_at | AF098482 | Hs.493516 | PSIP1 | 0.42 | 1.52 | 1.17 | 1.97 | 0.0015 |
| 210918_at | AF130075 | — | — | 0.46 | 1.59 | 1.24 | 2.04 | 0.0003 |
| 211204_at | L34035 | Hs.21160 | ME1 | 0.54 | 1.72 | 1.26 | 2.33 | 0.0006 |
| 211264_at | M81882 | Hs.231829 | GAD2 | 0.53 | 1.71 | 1.19 | 2.44 | 0.0034 |
| 211341_at | L20433 | Hs.493062 | POU4F1 | 0.57 | 1.77 | 1.21 | 2.58 | 0.0031 |
| 211516_at | M96651 | Hs.68876 | IL5RA | 0.60 | 1.82 | 1.26 | 2.62 | 0.0013 |
| 211772_x_at | BC006114 | Hs.89605 | CHRNA3 | 0.52 | 1.69 | 1.22 | 2.33 | 0.0014 |
| 212359_s_at | W89120 | Hs.65135 | KIAA0913 | −0.53 | 0.59 | 0.42 | 0.82 | 0.0019 |
| 212528_at | AI348009 | Hs.633087 | — | −0.79 | 0.45 | 0.29 | 0.70 | 0.0004 |
| 212531_at | NM_005564 | Hs.204238 | LCN2 | −0.57 | 0.56 | 0.38 | 0.84 | 0.0049 |
| 213197_at | AB006627 | Hs.495897 | ASTN1 | 0.66 | 1.93 | 1.36 | 2.74 | 0.0002 |
| 213260_at | AU145890 | Hs.599993 | — | 0.51 | 1.67 | 1.18 | 2.35 | 0.0036 |
| 213458_at | AB023191 | — | KIAA0974 | 0.43 | 1.54 | 1.19 | 1.99 | 0.0010 |
| 213482_at | BF593175 | Hs.476284 | DOCK3 | 0.53 | 1.70 | 1.19 | 2.42 | 0.0032 |
| 213603_s_at | BE138888 | Hs.517601 | RAC2 | −0.62 | 0.54 | 0.37 | 0.79 | 0.0017 |
| 213917_at | BE465829 | Hs.469728 | PAX8 | 0.52 | 1.69 | 1.21 | 2.36 | 0.0022 |
| 214457_at | NM_006735 | Hs.592177 | HOXA2 | 0.72 | 2.06 | 1.40 | 3.03 | 0.0002 |
| 214608_s_at | AJ000098 | Hs.491997 | EYA1 | 0.55 | 1.73 | 1.24 | 2.42 | 0.0013 |
| 214665_s_at | AK000095 | Hs.406234 | CHP | −0.52 | 0.59 | 0.43 | 0.82 | 0.0014 |
| 214822_at | AF131833 | Hs.495918 | FAM5B | 0.54 | 1.72 | 1.23 | 2.41 | 0.0017 |
| 215102_at | AK026768 | Hs.633705 | DPY19L1P1 | 0.49 | 1.64 | 1.22 | 2.20 | 0.0011 |
| 215180_at | AL109703 | Hs.651358 | — | 0.43 | 1.54 | 1.16 | 2.06 | 0.0029 |
| 215289_at | BE892698 | — | ZNF749 | 0.46 | 1.58 | 1.19 | 2.09 | 0.0017 |
| 215356_at | AK023134 | Hs.646351 | ECAT8 | 0.46 | 1.58 | 1.15 | 2.17 | 0.0048 |
| 215476_at | AF052103 | Hs.159157 | — | 0.49 | 1.63 | 1.21 | 2.21 | 0.0016 |
| 215705_at | BC000750 | — | PPP5C | 0.52 | 1.68 | 1.22 | 2.32 | 0.0016 |
| 215715_at | BC000563 | Hs.78036 | SLC6A2 | 0.75 | 2.12 | 1.37 | 3.29 | 0.0008 |
| 215850_s_at | AK022209 | Hs.651219 | NDUFA5 | 0.48 | 1.62 | 1.18 | 2.23 | 0.0030 |
| 215944_at | U80773 | — | — | 0.49 | 1.64 | 1.20 | 2.24 | 0.0019 |
| 215953_at | AL050020 | Hs.127384 | DKFZP564C196 | 0.47 | 1.59 | 1.16 | 2.19 | 0.0038 |
| 215973_at | AF036973 | — | HCG4P6 | 0.55 | 1.74 | 1.30 | 2.32 | 0.0002 |
| 216050_at | AK024584 | Hs.406847 | — | 0.44 | 1.55 | 1.15 | 2.08 | 0.0035 |
| 216066_at | AK024328 | Hs.429294 | ABCA1 | 0.50 | 1.65 | 1.22 | 2.22 | 0.0010 |
| 216240_at | M34428 | Hs.133107 | PVT1 | 0.46 | 1.58 | 1.15 | 2.18 | 0.0046 |
| 216881_x_at | X07882 | Hs.528651 | PRB4 | 0.41 | 1.51 | 1.14 | 1.99 | 0.0042 |
| 216989_at | L13779 | Hs.121494 | SPAM1 | 0.46 | 1.58 | 1.15 | 2.16 | 0.0044 |
| 217004_s_at | X13230 | Hs.387262 | MCF2 | 0.39 | 1.48 | 1.14 | 1.91 | 0.0032 |
| 217253_at | L37198 | Hs.632861 | — | 0.51 | 1.66 | 1.17 | 2.35 | 0.0041 |
| 217995_at | NM_021199 | Hs.511251 | SQRDL | −0.82 | 0.44 | 0.29 | 0.66 | 0.0001 |
| 218768_at | NM_020401 | Hs.524574 | NUP107 | 0.63 | 1.88 | 1.31 | 2.70 | 0.0006 |
| 218881_s_at | NM_024530 | Hs.220971 | FOSL2 | −0.52 | 0.60 | 0.42 | 0.85 | 0.0044 |
| 218980_at | NM_025135 | Hs.436636 | FHOD3 | 0.63 | 1.88 | 1.29 | 2.74 | 0.0011 |
| 219000_s_at | NM_024094 | Hs.315167 | DCC1 | 1.06 | 2.90 | 1.89 | 4.44 | 0.0000 |
| 219171_s_at | NM_007345 | Hs.189826 | ZNF236 | 0.56 | 1.76 | 1.20 | 2.56 | 0.0035 |
| 219182_at | NM_024533 | Hs.156784 | FLJ22167 | 0.48 | 1.62 | 1.18 | 2.22 | 0.0027 |
| 219425_at | NM_014351 | Hs.189810 | SULT4A1 | 0.74 | 2.11 | 1.41 | 3.14 | 0.0003 |
| 219520_s_at | NM_018458 | Hs.527524 | WWC3 | −0.49 | 0.61 | 0.44 | 0.84 | 0.0029 |
| 219537_x_at | NM_016941 | Hs.127792 | DLL3 | 0.55 | 1.73 | 1.23 | 2.44 | 0.0018 |
| 219617_at | NM_024766 | Hs.468349 | C2orf34 | 0.53 | 1.70 | 1.19 | 2.43 | 0.0035 |
| 219643_at | NM_018557 | Hs.470117 | LRP1B | 0.55 | 1.73 | 1.30 | 2.30 | 0.0001 |
| 219704_at | NM_015982 | Hs.567494 | YBX2 | 0.75 | 2.12 | 1.42 | 3.16 | 0.0002 |
| 219882_at | NM_024686 | Hs.445826 | TTLL7 | 0.51 | 1.66 | 1.18 | 2.35 | 0.0038 |
| 219937_at | NM_013381 | Hs.199814 | TRHDE | 0.54 | 1.71 | 1.23 | 2.38 | 0.0015 |
| 219955_at | NM_019079 | Hs.562195 | L1TD1 | 0.60 | 1.82 | 1.25 | 2.65 | 0.0018 |
| 220029_at | NM_017770 | Hs.408557 | ELOVL2 | 0.52 | 1.68 | 1.18 | 2.40 | 0.0038 |
| 220076_at | NM_019847 | Hs.156727 | ANKH | 0.77 | 2.17 | 1.53 | 3.07 | 0.0000 |
| 220294_at | NM_014379 | Hs.13285 | KCNV1 | 0.45 | 1.56 | 1.16 | 2.11 | 0.0036 |
| 220366_at | NM_022142 | Hs.104894 | ELSPBP1 | 0.53 | 1.69 | 1.19 | 2.41 | 0.0034 |
| 220394_at | NM_019851 | Hs.199905 | FGF20 | 0.61 | 1.84 | 1.30 | 2.60 | 0.0006 |
| 220397_at | NM_020128 | Hs.591036 | MDM1 | 0.41 | 1.51 | 1.17 | 1.95 | 0.0015 |
| 220541_at | NM_021801 | Hs.204732 | MMP26 | 0.50 | 1.64 | 1.24 | 2.18 | 0.0006 |
| 220653_at | NM_015363 | — | ZIM2 | 0.60 | 1.83 | 1.33 | 2.53 | 0.0002 |
| 220700_at | NM_018543 | Hs.188495 | WDR37 | 0.59 | 1.80 | 1.22 | 2.66 | 0.0029 |
| 220703_at | NM_018470 | Hs.644603 | C10orf110 | 0.59 | 1.80 | 1.26 | 2.58 | 0.0012 |
| 220771_at | NM_016181 | Hs.633593 | LOC51152 | 0.60 | 1.81 | 1.23 | 2.67 | 0.0025 |
| 220817_at | NM_016179 | Hs.262960 | TRPC4 | 0.47 | 1.60 | 1.19 | 2.14 | 0.0019 |
| 220834_at | NM_017716 | Hs.272789 | MS4A12 | 0.52 | 1.68 | 1.27 | 2.22 | 0.0003 |
| 220847_x_at | NM_013359 | Hs.631598 | ZNF221 | 0.50 | 1.65 | 1.19 | 2.28 | 0.0025 |
| 220852_at | NM_014099 | Hs.621386 | PRO1768 | 0.48 | 1.62 | 1.19 | 2.20 | 0.0022 |
| 220970_s_at | NM_030977 | Hs.406714 | KRTAP2-4/ | 0.49 | 1.64 | 1.16 | 2.31 | 0.0050 |
| | | | LOC644350 | | | | | |
| 220981_x_at | NM_022053 | Hs.648337 | NXF2 | 0.45 | 1.56 | 1.19 | 2.05 | 0.0014 |
| 220993_s_at | NM_030784 | Hs.632612 | GPR63 | 0.38 | 1.46 | 1.13 | 1.88 | 0.0041 |
| 221018_s_at | NM_031278 | Hs.333132 | TDRD1 | 0.81 | 2.25 | 1.51 | 3.37 | 0.0001 |
| 221077_at | NM_018076 | Hs.127530 | ARMC4 | 0.56 | 1.76 | 1.25 | 2.47 | 0.0013 |
| 221137_at | AF118071 | — | — | 0.46 | 1.59 | 1.15 | 2.20 | 0.0049 |
| 221168_at | NM_021620 | Hs.287386 | PRDM13 | 0.68 | 1.96 | 1.33 | 2.91 | 0.0007 |
| 221258_s_at | NM_031217 | Hs.301052 | KIF18A | 0.62 | 1.86 | 1.34 | 2.58 | 0.0002 |
| 221319_at | NM_019120 | Hs.287793 | PCDHB8 | 0.40 | 1.49 | 1.14 | 1.96 | 0.0041 |
| 221393_at | NM_014627 | — | TAAR3 | 0.50 | 1.64 | 1.17 | 2.31 | 0.0043 |
| 221591_s_at | BC005004 | Hs.592116 | FAM64A | 0.72 | 2.05 | 1.38 | 3.05 | 0.0004 |
| 221609_s_at | AY009401 | Hs.29764 | WNT6 | 0.40 | 1.50 | 1.15 | 1.95 | 0.0028 |
| 221718_s_at | M90360 | Hs.459211 | AKAP13 | −0.64 | 0.53 | 0.36 | 0.78 | 0.0013 |
| 221950_at | AI478455 | Hs.202095 | EMX2 | 0.67 | 1.96 | 1.41 | 2.72 | 0.0001 |

TABLE 4
Features Of 15 Probe Sets In The Gene Signature

| | | | | | Rank of | | Rank of | | Rank of | |
|---|---|---|---|---|---|---|---|---|---|---|
| | Gene | | Entrez | | expression | | variation | | significant | |
| Probe Set | Symbol | Gene Title | Gene ID | Coef.* | [n = 19619 (%)] | | [n = 19619 (%)] | | [n = 172 (%)] | |
| 201243_s_at | ATP1B1 | ATPase, Na+/K+ transporting, beta 1 | 481 | −0.54 | 517 | (2.6) | 2224 | (11.3) | 111 | (64.5) |
| | | polypeptide | | | | | | | | |
| 203147_s_at | TRIM14 | Tripartite motif-containing 14 | 8518 | −0.56 | 3532 | (18.0) | 9499 | (48.4) | 112 | (65.1) |
| 221591_s_at | FAM64A | Family with sequence similarity 64, | 7372 | 0.72 | 6171 | (31.5) | 6108 | (31.1) | 29 | (16.9) |
| | | member A | | | | | | | | |
| 218881_s_at | FOSL2 | FOS-like antigen 2 | 10614 | −0.52 | 6526 | (33.3) | 12445 | (63.4) | 155 | (90.1) |
| 202814_s_at | HEXIM1 | Hexamethylene bis-acetamide inducible 1 | 11075 | 0.59 | 7415 | (37.8) | 9026 | (46.0) | 161 | (93.6) |
| 204179_at | MB | myoglobin | 9830 | 0.47 | 7703 | (39.3) | 7942 | (40.5) | 156 | (90.7) |
| 204584_at | L1CAM | L1 cell adhesion molecule | 4151 | 0.56 | 9327 | (47.5) | 3329 | (17.0) | 17 | (9.9) |
| 202707_at | UMPS | Uridine monophosphate synthetase | 3897 | 0.60 | 12311 | (62.8) | 18737 | (95.5) | 101 | (58.7) |
| 208399_s_at | EDN3 | Endothelin 3 | 4193 | 0.48 | 16344 | (83.3) | 8234 | (42.0) | 110 | (64.0) |
| 203001_s_at | STMN2 | Stathmin-like 2 | 2315 | 0.55 | 16948 | (86.4) | 5690 | (29.0) | 109 | (63.4) |
| 210016_at | MYT1L | Myelin transcription factor 1-like | 1908 | 0.60 | 17902 | (91.2) | 18637 | (95.0) | 27 | (15.7) |
| 202490_at | IKBKAP | Inhibitor of kappa light polypeptide gene | 23040 | 0.42 | 18769 | (95.7) | 10412 | (53.1) | 84 | (48.8) |
| | | enhancer in B-cells, kinase complex- | | | | | | | | |
| | | associated protein | | | | | | | | |
| 206426_at | MLANA | Melan-A | 2355 | 0.63 | 19159 | (97.7) | 17172 | (87.5) | 81 | (47.1) |
| 205386_s_at | MDM2 | Mdm2, transformed 3T3 cell double | 7776 | 0.49 | 19251 | (98.1) | 14275 | (72.8) | 104 | (60.5) |
| | | minute 2 | | | | | | | | |
| 219171_s_at | ZNF236 | Zinc finger protein 236 | 54478 | 0.56 | 19383 | (98.8) | 17046 | (86.9) | 132 | (76.7) |

*Coefficient of the Cox model

TABLE 5
Demographic Distributions of Patients In Validation Sets

| | | | | | | |
|---|---|---|---|---|---|---|
| | DCC, All | DCC, UM | DCC, HLM | DCC, MSK | Duke | UM-SQ |
| Clinical Factors | n = 360 (%) | n = 177 (%) | n = 79 (%) | n = 104 (%) | n = 89 (%) | n = 129 (%) |

Pathology Type

| | | | | | | |
|---|---|---|---|---|---|---|
| Adeno | 360 (100) | 177 (100) | 79 (100) | 104 (100) | 43 (48) | 0 |
| Non-Adeno | 0 (0) | 0 (0) | 0 (0) | 0 (0) | 46 (52) | 129 (100) |

Disease stage

| | | | | | | |
|---|---|---|---|---|---|---|
| I | 220 (61) | 116 (66) | 41 (52) | 63 (61) | 67 (75) | 73 (57) |
| II | 69 (19) | 29 (16) | 20 (25) | 20 (19) | 18 (20) | 33 (25) |
| III | 69 (19) | 32 (18) | 16 (20) | 21 (20) | 3 (3) | 23 (18) |
| IV | 0 (0) | 0 (0) | 0 (0) | 0 (0) | 1 (2) | 0 (0) |
| Unknown | 2 (1) | 0 (0) | 2 (3) | 0 (0) | 0 (0) | 0 (0) |

Adjuvant chemotherapy

| | | | | | | |
|---|---|---|---|---|---|---|
| No | 210 (58) | 76 (43) | 61 (77) | 73 (70) | 89 (100) | NS |
| Yes | 64 (18) | 17 (10) | 16 (20) | 31 (30) | 0 (0) | NS |
| Unknown | 86 (24) | 84 (47) | 2 (3) | 0 (0) | 0 (0) | NS |

Adjuvant radiotherapy

| | | | | | | |
|---|---|---|---|---|---|---|
| No | 209 (58) | 76 (43) | 57 (72) | 76 (73) | 89 (100) | NS |
| Yes | 64 (18) | 17 (10) | 19 (24) | 28 (27) | 0 (0) | NS |
| Unknown | 87 (24) | 84 (47) | 3 (4) | 0 (0) | 0 (0) | NS |

Age (year)

| | | | | | | |
|---|---|---|---|---|---|---|
| <65 | 163 (45) | 87 (49) | 17 (34) | 49 (47) | 33 (37) | 52 (40) |
| ≧65 | 197 (55) | 90 (51) | 25 (66) | 55 (53) | 56 (63) | 77 (60) |

Gender

| | | | | | | |
|---|---|---|---|---|---|---|
| Male | 177 (49) | 100 (56) | 40 (51) | 37 (36) | 54 (61) | 82 (64) |
| Female | 183 (51) | 77 (44) | 39 (49) | 67 (64) | 35 (39) | 47 (36) |

DCC: Directors' Challenge Consortium;
UM: University of Michigan;
HLM: H. Lee Moffitt Cancer Center;
MSK: Memorial Sloan-Kettering Cancer Center;
NS: Not specified

TABLE 6
Adjuvant therapies in the Director's Challenge Consortium
(DCC) Patients

| | Adjuvant | | | |
|---|---|---|---|---|
| | radiotherapy | | | |
| Adjuvant Chemotherapy | No | Yes | Unknown | Total |
| All | | | | |
| No | 190 | 20 | 0 | 210 |
| Yes | 19 | 44 | 1 | 64 |
| Unknown | 0 | 0 | 86 | 86 |
| University of Michigan (UM) | | | | |
| No | 76 | 0 | 0 | 76 |
| Yes | 0 | 17 | 0 | 17 |
| Unknown | 0 | 0 | 84 | 84 |
| H. Lee Moffitt (HLM) | | | | |
| No | 51 | 10 | 0 | 61 |
| Yes | 6 | 9 | 1 | 16 |
| Unknown | 0 | 0 | 2 | 2 |
| Memorial Sloan-Kettering (MSK) | | | | |
| No | 63 | 10 | 0 | 73 |
| Yes | 13 | 18 | 0 | 31 |
| Unknown | 0 | 0 | 0 | 0 |

TABLE 7
Primers for qPCR Validation

| | SEQ | | | | | |
|---|---|---|---|---|---|---|
| Q. | ID | | SEQ | | Amplicon | |
| ene | NO | Forward | ID NO | Reverse | Length | Tm |
| FAM64A | 173 | AGTCACTCACCCACTGTGTTTCTG | 188 | GGTAGGGAAAGGAGGGATGAGA | 71 | 83 |
| MB | 174 | CTGTGTTCTGCATGGTTTGGAT | 189 | GGTTGGAAGAAGTTCGGTTGG | 71 | 76 |
| EDN3 | 175 | ATTTGAGTGGGTGTCCAGGG | 190 | GGTCAAGGCCAATGCTCTGT | 71 | 80 |
| ZNF236 | 176 | AAAGGACCGCATCAGTGAGC | 191 | AGCAGTTGGCGTGCTTGG | 71 | 85 |
| FOSL2 | 177 | AAGAAGATTGGGCAGTTGGGT | 192 | TCCTGCTACTCCTGGCTCATTC | 71 | 80 |
| MYT1L | 178 | AAGATAAACAGCCCCAGGAACC | 193 | CCACTGAGGAGCTGTCTGCTTT | 72 | 81 |
| MLANA | 179 | GTAGGAAAAATGCAAGCCATCTCT | 194 | CATGATTAGTACTGCTAGCGGACC | 77 | 74 |
| L1CAM | 180 | AAAGGAAAGATTGGTTCTCCCAG | 195 | AGTAGACCAAGCACAGGCATACAG | 71 | 81 |
| TRIM14 | 181 | TCACAGCTCCCTCCAGAAGC | 196 | GATGAGGACTGGGAGAGGGTT | 71 | 82 |
| STMN2 | 182 | CAGGCTTTTGAGCTGATCTTGAA | 197 | TTTGGAGAAGCTAAAGTTCGTGG | 71 | 79 |
| UMPS | 183 | GCCAACAGTACAATAGCCCACAA | 198 | CCACGACCTACAATGATGATATCG | 70 | 78 |
| ATP1B1 | 184 | AGTTGGAAATGTGGAGTATTTTGGA | 199 | CATAGTACGGATAATACTGCAGAGGAA | 71 | 78 |
| HEXIM1 | 185 | CTGACCGAGAACGAACTGCA | 200 | AGTCCCCTTTGCCCCCTC | 99 | 83 |
| IKBKAP | 186 | AGCGATTCACGTAGGATCTGC | 201 | ATCACCAGTGTTGGAAGTGGG | 71 | 82 |
| MDM2 | 187 | TGCCCCTTAATGCCATTGAA | 202 | TTTTGCCATGGACAATGCA | 75 | 77 |

TABLE 8
Risk Group Based on 15-Gene Signature in Stage I Patients

| | n | HR | 95% CI | p value |
|---|---|---|---|---|
| BR.10 | 34 | 13.3 | 2.9-62.1 | <0.0001 |
| Observation arm | | | | |
| DCC | 141 | 3.3 | 1.5-7.4 | 0.002 |
| No adjuvant therapy | | | | |
| UM | 57 | 1.9 | 0.6-6.1 | 0.28 |
| HLM | 37 | 2.5 | 0.9-6.9 | 0.07 |
| MSK | 47 | NA | NA | 0.05 |
| Duke | 67 | 1.06 | 0.5-2.2 | 0.88 |
| UM-SQ | 73 | 1.4 | 0.6-3.1 | 0.44 |

n: number of patients;
HR: hazard ratio;
CI: confidence interval
* HR and CI cannot be calculated as no death occurred in the good prognosis group, p value the score test.

TABLE 9
Probe set target sequences of the 15-gene signature

| | | |
|---|---|---|
| SEQ ID | Probe | |
| NO: | Set ID | Target sequence |

| | | |
|---|---|---|
| 35 | 205386_S_AT | tttcccctagttgacctgtctataagagaattatatatttctaactatataaccctaggaatttagacaacctgaaattt |
| | | attcacatatatcaaagtgagaaaatgcctcaattcacatagatttcttctctttagtataattgacctactttggtagt |
| | | ggaatagtgaatacttactataatttgacttgaatatgtagctcatcctttacaccaactcctaattttaaataatttcta |
| | | ctctgtcttaaatgagaagtacttggttttttttttcttaaatatgtatatgacatttaaatgtaacttattattttttttgaga |
| | | ccgagtcttgctctgttacccaggctggagtgcagtgggtgatcttggctcactgcaagctctgccctccccggg |
| | | ttcgcaccattctcctgcctcagcctcccaattagcttggcctacagtcatctgcc |
| 78 | 208399_S_AT | ccgagccgagcttactgtgagtgtggagatgttatcccaccatgtaaagtcgcctgcgcaggggagggctgcc |
| | | catctccccaacccagtcacagagagataggaaacggcatttgagtgggtgtccagggccccgtagagagac |
| | | atttaagatggtgtatgacagagcattggccttgaccaaatgttaaatcctctgtgtgtatttcataagttattacagg |
| | | tataaaagtgatgacctatcatgaggaaatgaaagtggctgatttgctggtaggattttgtacagtttagagaagc |
| | | gattatttattgtgaaactgttctccactccaactcctttatgtggatctgttcaaagtagtcactgtatatacgtataga |
| | | gaggtagataggtaggtagattttaaattgcattctgaatacaaactcatactccttagagcttgaattacatttttaa |
| | | aatgcatatgtgctgtttggcaccgtggcaagatggtatcagagagaaacccatcaattgctcaaatactc |
| 4 | 201243_S_AT | ggtgatgggttgtgttatgcttgtattgaatgctgtcttgacatctcttgccttgtcctccggtatgttctaaagctgtgt |
| | | ctgagatctggatctgcccatcactttggcctagggacagggctaattaatttgctttatacattttcttttactttccttt |
| | | tttcctttctggaggcatcacatgctggtgctgtgtctttatgaatgttttaaccattttcatggtggaagaattttatatt |
| | | tatgcagttgtacaattttatttttttctgcaagaaaaagtgtaatgtatgaaataaaccaaagtcacttgtttgaaaat |
| | | aaatctttattttgaactttataaaagcaatgcagtaccccatagactggtgttaaatgttgtctacagtgcaaaatcc |
| | | atgttctaacatatgtaataattgccaggagtacagtgctcttgttgatcttgtattcagtcaggttaaaa |
| 22 | 204179_AT | tgttccggaaggacatggcctccaactacaaggagctgggcttccagggctaggcccctgccgc |
| | | tcccacccccacccatctgggccccgggttcaagagagagcggggtctgatctcgtgtagccata |
| | | tagagtttgcttctgagtgtctgctttgtttagtagaggtgggcaggaggagctgaggggctggggct |
| | | ggggtgttgaagttggctttgcatgcccagcgatgcgcctccctgtgggatgtcatcaccctggga |
| | | accgggagtgcccttggctcactgtgttctgcatggtttggatctgaattaattgtcctttcttctaaatc |
| | | ccaaccgaacttcttccaacctccaaactggctgtaaccccaaatccaagccattaactacacct |
| | | gacagtagcaattgtctgattaatcactggccccttgaagacagcagaatgtccctttgcaatgag |
| | | gaggagatctgggctgggcgggccagctggggaagcatttgactatctggaacttgtgtgtgcctc |
| | | ctcaggtatggca |
| 169 | 221591_S_AT | cacatctggacccatcagtgactgcctgccatagcctgagagtgtcttggggagaccttgcagagggggagaa |
| | | ttgttccttctgctttcctaggggactcttgagcttagaaactcatcgtacacttgaccttgagccttctatttgcctca |
| | | tctataacatgaagtgctagcatcagatatttgagagctcttagctctgtacccgggtgcctggtttttggggagtc |
| | | atccgcagagtcactcacccactgtgtttctggtgccaaggctcttgagggccccactctcatccctcctttcccta |
| | | ccagggactcggaggaaggcataggagatatttccaggcttacgaccctgggctcacgggtacctatttatatg |
| | | ctcagtgcagagcactgtggatgtgccaggaggggtagccctgttcaagagcaatttctgccctttgtaaattattt |
| | | aagaaacctgctttgtcattttattagaaagaaaccagcgtgtgactttcctagataacactgctttc |
| 15 | 203147_S_AT | accaatcacgcctacagtgctttgaaggtttcctctcctaggctagtttcaaacaggccctaaacaa |
| | | gtctgctgctgccctctcatcagacctccgcaccctcaccccaccatcacttanactactttaatcca |
| | | gttccttcaaagtgatacccccacaggtaagccctcagcatcctgaatacatcatccgcagcctgg |
| | | gaaccttctccctcgtacagcacaggaacctgacacatagtaggcacacagtaaacgtttgtgaa |
| | | tgaatgggagtcatccagtcctgactcttctgtctcttgaggtcccttgaatcttccgcttcctccccac |
| | | cgatttcagcgtgtccacatcacagctccctccagaagctgcaagagcttcttagcagttcctggtc |
| | | tgaaccctctcccagtcctcatcttccaccctaaaactagagtgatcttcctaaaacttcacttaacc |
| | | cctcagctatgaaaaggcttccaggagtttccatgaa |
| 130 | 218881_S_AT | aggtcacagtatcctcgtttgaaagataattaagatcccccgtggagaaagcagtgacacattca |
| | | cacagctgttccctcgcatgttatttcatgaacatgacctgttttcgtgcactagacacacagagtgg |
| | | aacagccgtatgcttaaagtacatgggccagtgggactggaagtgacctgtacaagtgatgcag |
| | | aaaggagggtttcaaagaaaaaggattttgtttaaaatactttaaaaatgttatttcctgcatcccttg |
| | | gctgtgatgcccctctcccgatttcccaggggctctgggagggacccttctaagaagattgggcag |
| | | ttgggtttctggcttgagatgaatccaagcagcagaatgagccaggagtagcaggagatgggca |
| | | aagaaaactggggtgcactcagctctcacaggggtaatca |
| 85 | 210016_AT | ataacagcatatgcatttccccaccgcgttgtgtctgcagcttctttgccaatatagtaatgcttttagtagagtacta |
| | | gatagtatcagttttggattcttattgttatcacctatgtacaatggaaagggattttaagcacaaacctgctgctcat |
| | | ctaacgttggtacataatctcaaatcaaaagttatctgtgactattatatagggatcacaaaagtgtcacatattaga |
| | | atgctgacctttcatatggattattgtgagtcatcagagtttattataacttattgttcatattcatttctaagttaatttaa |
| | | gtaatcatttattaagacagaattttgtataaactatttattgtgctctctgtggaactgaagtttgatttatttttgtacta |
| | | cacggcatgggtttgttgacactttaattttgctataaatgtgtggaatcacaagttgctgtgatacttcatttttaaatt |
| | | gtgaactttgtacaaattttgtcatgctggatgttaacacat |
| 11 | 202490_AT | gaggatggcacaagcgattcacgtaggatctgcccctgtgaccaaaacacctcccattgggccccacttccaa |
| | | cactggtgatcacatttcaacatgaggtttagggaaacaaatgcctaaactacagcactgtacataaactaacag |
| | | gaaatgctgcttttgatcctcaaagaagtgatatagccaaaattgtaatttaagaagcctttgtcagtatagcaagat |
| | | gttaactatagaatcaatctaggagtattcactgtaaaattcaacttttctgtatgtttgaacattttcacaatctcatag |
| | | gagtttttaaaaagaagagaaagaagatatactttgctttggagaaatctactttttgacttacatgggtttgctgtaa |
| | | ttaagtgcccaatattgaaaggctgcaagtactttgtaatcactctttggcatgggtaaataagcatggtaacttata |
| | | ttgaaatatagtgctcttgctttggataactgtaaagggacccatgctgatagactggaaa |
| 12 | 202707_AT | aagttcattcttaagcttgctttttttgagactggtgtttgttagacagccacagtcctgtctgggttagg |
| | | gtcttccacatttgaggatccttcctatctctccatgggactagactgctttgttattctatttattttttaattt |
| | | ttttcgagacaggatctcactctgttgcccaggatggagtgcagtggtgagatcacggctcattgca |
| | | gcctcgacctcccaggtgatcctcccacctcagcttccagattagctggtgctataggcatgcacc |
| | | accacgtccatctaaatttctttattatttgtagagatgaggtcttgccatgttacccaggctggtctca |
| | | actcctgggctcaagcgatcctcctgcctcagtctctcaaagtgctgggattacaggtgtgagcca |
| | | ctgtgcccagcctaattgcagtaagacaa |
| 14 | 203001_S_AT | acctcgcaacatcaacatctatacttacgatgatatggaagtgaagcaaatcaacaaacgtgcct |
| | | ctggccaggcttttgagctgatcttgaagccaccatctcctatctcagaagccccacgaactttagc |
| | | ttctccaaagaagaaagacctgtccctggaggagatccagaagaaactggaggctgcagggg |
| | | aaagaagaaagtctcaggaggcccaggtgctgaaacaattggcagagaagagggaacacg |
| | | agcgagaagtccttcagaaggctttggaggagaacaacaacttcagcaagatggcggaggaa |
| | | aagctgatcctgaaaatggaacaaattaaggaaaaccgtgaggctaatctagctgctattattga |
| | | acgtctgcaggaaaaggagaggcatgctgcggaggtgcgcaggaacaaggaactccaggtt |
| | | gaactgtctggctgaagcaagggagggtctggcacgcc |
| 13 | 202814_S_AT | tgcctctcgcgcatggaggacgagaacaaccggctgcggctggagagcaagcggctgggtgg |
| | | cgacgacgcgcgtgtgcgggagctggagctggagctggaccggctgcgcgccgagaacctcc |
| | | agctgctgaccgagaacgaactgcaccggcagcaggagcgagcgccgctttccaagtttggag |
| | | actagactgaaacttttttgggggagggggcaaaggggactttttacagtgatggaatgtaacatt |
| | | atatacatgtgtatataagacagtggacctttttatgacacataatcagaagagaaatccccctggc |
| | | tttggttggtttcgtaaatttagctatatgtagcttgcgtgctttctcctgttcttttaattatgtgaaactgaa |
| | | gagttgcttttcttgttttcctttttagaagtttttttccttaatgtgaaagtaatttgaccaagttataatgcat |
| | | ttttgtttttaacaaatcccctccttaaacggagctataaggtggccaaatctga |
| 133 | 219171_S_AT | cttttgttcttgctgggttatttattttgattttagcattaaatgtcatctcaggatatctctaaaaggggttgt |
| | | ttaattcctaattgtatagaaagctagtttggtgaattgtattggttaattgactgtttaaggccttaaca |
| | | ggtgaatctagagcctacttttattttggttaaagaaaaagaaaatatcaataattcaattttgtgtcttt |
| | | tctcaatttattagcaaacacaagacattttatgtattatttcgatttacttcctaattataaaagctgcttt |
| | | tttgcagaacattccttgaaaatataaggttttgaaaagacataattttacttgaatctttgtggggtac |
| | | aggttgatctttatattttactggttgttttaaaaattctagaaaagagatttctaggcctcatgtataacc |
| | | agggttttgaggataaagaactgtatttttagaactatctcatcatagcatatctgctttggaataactat |
| 49 | 206426_AT | gtaaagatcctatagctctttttttttgagatggagtttcgcttttgttgcccaggctggagtgcaatggcgcgatctt |
| | | ggctcaccataacctccgcctcccaggttcaagcaattctcctgccttagcctcctgagtagctgggattacagg |
| | | cgtgcgccactatgcctgactaattttgtagttttagtagagacggggtttctccatgttggtcaggctggtctcaaa |
| | | ctcctgacctcaggtgatctgcccgcctcagcctcccaaagtgctggaattacaggcgtgagccaccacgcctg |
| | | gctggatcctatatcttaggtaagacatataacgcagtctaattacatttcacttcaaggctcaatgctattctaacta |
| | | atgacaagtattttctactaaaccagaaattggtagaaggatttaaataagtaaaagctactatgtactgccttagtg |
| | | ctgatgcctgtgtactgccttaaatgtacctatggcaatttagctctcttgggttcccaaatccctctcacaagaatgt |
| 26 | 204584_AT | cctccctatcgtctgaacagttgtcttcctcagcctcctcccgcccccaccttgggaatgtaaataca |
| | | ccgtgactttgaaagtttgtacccctgtccttccctttacgccactagtgtgtaggcagatgtctgagtc |
| | | cctaggtggtttctaggattgatagcaattagctttgatgaacccatcccaggaaaaataaaaaca |
| | | gacaaaaaaaaaggaaagattggttctcccagcactgctcagcagccacagcctccctgtatgc |
| | | ctgtgcttggtctactgataagccctctacaaaa |

TABLE 10
Coefficient of individual genes in 15-gene signature:
Principal Component values

| | Gene | | | | | |
|---|---|---|---|---|---|---|
| Gene | Symbol | Probe set | pc1 | pc2 | pc3 | pc4 |
| 1 | ATP1B1 | 201243_s_at | −0.189 | −0.423 | 0.229 | 0.059 |
| 2 | IKBKAP | 202490_at | 0.364 | 0.070 | −0.357 | −0.120 |
| 3 | UMPS | 202707_at | 0.353 | −0.009 | 0.136 | 0.011 |
| 4 | HEXIM1 | 202814_s_at | −0.108 | 0.504 | 0.265 | 0.279 |
| 5 | STMN2 | 203001_s_at | 0.326 | 0.044 | −0.100 | −0.122 |
| 6 | TRIM14 | 203147_s_at | −0.148 | 0.212 | 0.132 | −0.368 |
| 7 | MB | 204179_at | 0.197 | 0.028 | 0.548 | −0.161 |
| 8 | L1CAM | 204584_at | 0.042 | 0.510 | 0.077 | 0.276 |
| 9 | MDM2 | 205386_s_at | 0.180 | 0.081 | 0.325 | −0.500 |
| 10 | MLANA | 206426_at | 0.366 | −0.240 | 0.114 | 0.157 |
| 11 | EDN3 | 208399_s_at | 0.413 | 0.042 | −0.188 | −0.260 |
| 12 | MYT1L | 210016_at | 0.270 | 0.014 | 0.273 | 0.245 |
| 13 | FOSL2 | 218881_s_at | 0.036 | −0.209 | −0.225 | 0.190 |
| 14 | ZNF236 | 219171_s_at | 0.188 | −0.313 | 0.297 | 0.332 |
| 15 | FAM64A | 221591_s_at | 0.283 | 0.216 | −0.174 | 0.320 |

| | | | | |
|---|---|---|---|---|
| Eigenvalues of principal | 3.33 | 1.82 | 1.37 | 1.32 |
| components | | | | |
| Weight of each PC for risk score | 0.557 | 0.328 | 0.430 | 0.335 |

Risk score = 0.557 * PC1 + 0.328 * PC2 + 0.43 * PC3 + 0.335 * PC4
where
PC1 = Sum [pc1 * (expression data)]_{Gene 1-15}
PC2 = Sum [pc2 * (expression data)]_{Gene 1-15}
PC3 = Sum [pc3 * (expression data)]_{Gene 1-15}
PC4 = Sum [pc4 * (expression data)]_{Gene 1-15}
Patients classified as high risk or lower risk according to risk score ≧−0.1 or <−0.1.

TABLE 11
Probe set target sequences for 172 genes

| SEQ | | | |
|---|---|---|---|
| ID | Probe | Gene | |
| NO: | Set ID | Symbol | Target Sequence |
| 1 | 200878_at | EPAS1 | cactttgcaactccctgggtaagagggacgacacctctggtttttcaataccaattacatggaact |
| | | | tttctgtaatgggtacnaatgaagaagtttctaaaaacacacacaaagcacattgggccaactat |
| | | | ttagtaagcccggatagacttattgccaaaaacaaaaaatagctttcaaaagaaatttaagttctat |
| | | | gagaaattccttagtcatggtgttgcgtaaatcatattttagctgcacggcattaccccacacagg |
| | | | gtggcagaacttgaagggttactgacgtgtaaatgctggtatttgatttcctgtgtgtgttgccctg |
| | | | gcattaagggcattttacccttgcagttttactaaaacactgaaaaatattccaagcttcatattaac |
| | | | cctacctgtcaacgtaacgat |
| 2 | 201228_s_at | ARIH2 | cctacccacctcaaaatgtctgtactgcaagagggccctgggcctctgctttccatattcacgttt |
| | | | ggccagagttgtagtcccaaagaagagcatgggtggcagatggtagggaattgaactggcct |
| | | | gtgcaatgggcatggagcacaaggggtcacagcatgcctcctgccttaccgtggcagtacgg |
| | | | agacagtccagaacatggtcttcttgccacggggtgttgttgtctctggtggtgctgcatgtctgt |
| | | | ggctcacctttattcttgaaactgaggtttacctggatctggctactgaggctagagcccacagc |
| | | | agaatggggttgggcctgtggccccccaaactagggggtgtgggttcatcacagtgttgccttt |
| | | | tgtctcctaaagatagggatctacttttgaagggaattgttcctcccaaata |
| 3 | 201242_s_at | ATP1B1 | agagctgatcacaagcacaaatctttcccactagccatttaataagttaaaaaaagatacaaaaa |
| | | | caaaaacctactagtcttgaacaaactgtcatacgtatgggacctacacttaatctatatgctttac |
| | | | actagctttctgcatttaataggttagaa |
| 4 | 201243_s_at | ATP1B1 | ggtgatgggttgtgttatgcttgtattgaatgctgtcttgacatctcttgccttgtcctccggtatgtt |
| | | | ctaaagctgtgtctgagatctggatctgcccatcactttggcctagggacagggctaattaatttg |
| | | | ctttatacattttcttttactttccttttttcctttctggaggcatcacatgctggtgctgtgtctttatgaa |
| | | | tgttttaaccattttcatggtggaagaattttatatttatgcagttgtacaattttatttttttctgcaaga |
| | | | aaaagtgtaatgtatgaaataaaccaaagtcacttgtttgaaaataaatctttattttgaactttataa |
| | | | aagcaatgcagtaccccatagactggtgttaaatgttgtctacagtgcaaaatccatgttctaaca |
| | | | tatgtaataattgccaggagtacagtgctcttgttgatcttgtattcagtcaggttaaaa |
| 5 | 201301_s_at | ANXA4 | ggtgaaatttctaactgttctctgttcccggaaccgaaatcacctgttgcatgtgtttgatgaatac |
| | | | aaaaggatatcacagaaggatattgaacagagtattaaatctgaaacatctggtagctttgaaga |
| | | | tgctctgctggctatagtaaagtgcatgaggaacaaatctgcatattttgctgaaaagctctataa |
| | | | atcgatgaagggcttgggcaccgatgataacaccctcatcagagtgatggtttctcgagcagaa |
| | | | attgacatgttggatatccgggcacacttcaagagactctatggaaagtctctgtactcgttcatc |
| | | | aagggtgacacatctggagactacaggaaagtactgcttgttctctgtggaggagatgattaaa |
| | | | ataaaaatcccagaaggacaggaggattctcaacactttgaatttttttaacttcatttttctacact |
| | | | gctattatcattatctc |
| 6 | 201502_s_at | NFKBIA | ccaactacaatggccacacgtgtctacacttagcctctatccatggctacctgggcatcgtgga |
| | | | gcttttggtgtccttgggtgctgatgtcaatgctcaggagccctgtaatggccggactgcccttca |
| | | | cctcgcagtggacctgcaaaatcctgacctggtgtcactcctgttgaagtgtggggctgatgtca |
| | | | acagagttacctaccagggctattctccctaccagctcacctggggccgcccaagcacccgga |
| | | | tacagcagcagctgggccagctgacactagaaaaccttcagatgctgccagagagtgaggat |
| | | | gaggagagctatgacacagagtcagagttcacggagttcacagaggacgagctgccctatga |
| | | | tgactgtgtgtttggaggccagcgtctgacgttatgag |
| 7 | 202023_at | EFNA1 | ccaccttcacctcggagggacggagaaagaagtggagacagtcctttcccaccattcctgcctt |
| | | | taagccaaagaaacaagctgtgcaggcatggtcccttaaggcacagtgggagctgagctgga |
| | | | aggggccacgtggatgggcaaagcttgtcaaagatgccccctccaggagagagccaggatg |
| | | | cccagatgaactgactgaaggaaaagcaagaaacagtttcttgcttggaagccaggtacagga |
| | | | gaggcagcatgcttgggctgacccagcatctcccagcaagacctcatctgtggagctgccaca |
| | | | gagaagtttgtagccaggtactgcattctctcccatcctggggcagcactccccagagctgtgc |
| | | | cagcaggggggctgtgccaacctgttcttagagtgtagctgtaagggcagtgcccatgtgtac |
| | | | attctgcctagagtgtagcctaaagggcagggcccacgtgtatagtatctgta |
| 8 | 202035_s_at | SFRP1 | tcggccagcgagtacgactacgtgagcttccagtcggacatcggcccgtaccagagcgggc |
| | | | gcttctacaccaagccacctcagtgcgtggacatccccgcggacctgcggctgtgccacaac |
| | | | gtgggctacaagaagatggtgctgcccaacctgctggagcacgagaccatggcggaggtga |
| | | | agcagcaggccagcagctgggtgcccctgctcaacaagaactgccacgccggcacccaggt |
| | | | cttcctctgctcgctcttcgcgcccgtctgcctggaccggcccatctacccgtgtcgctggctct |
| | | | gcgaggccgtgcgcgactcgtgcgagccggtcatgcagttcttcggcttctactggcccgaga |
| | | | tgcttaagtgtgacaagttccccgagggggacgtctgcatcgccatgacgccgcccaatgcca |
| | | | ccgaagcctccaagccccaaggcacaacggtgtgtcctccctgtgacaacgagttgaaatctg |
| | | | aggccatcattgaacatctctgt |
| 9 | 202036_s_at | SFRP1 | gacaaaccatttccaacagcaacacagccactaaaacacaaaaagggggattgggcggaaa |
| | | | gtgagagccagcagcaaaaactacattttgcaacttgttggtgtggatctattggctgatctatgc |
| | | | ctttcaactagaaaattctaatgattggcaagtcacgttgttttcaggtccagagtagtttctttctgt |
| | | | ctgctttaaatggaaacagactcataccacacttacaattaaggtcaagcccagaaagtgataa |
| | | | gtgcagggaggaaaagtgcaagtccattatgtaatagtgacagcaaaggcccaggggagag |
| | | | gcattgccttctctgcccacagtctttccgtgtgattgtctttgaatctgaatcagccagtctcagat |
| | | | gccccaaagtttcggttcctatgagcccggggcatgatctgatccccaagacatg |
| 10 | 202037_s_at | SFRP1 | taacacttggctcttggtacctgtgggttagcatcaagttctccccagggtagaattcaatcagag |
| | | | ctccagtttgcatttggatgtgtaaattacagtaatcccatttcccaaacctaaaatctgtttttctcat |
| | | | cagactctgagtaactggttgctgtgtcataacttcatagatgcaggaggctcaggtgatctgttt |
| | | | gaggagagcaccctaggcagcctgcagggaataacatactggccgttctgacctgttgccag |
| | | | cagatacacaggacatggatgaaattcccgtttcctctagtttcttcctgtagtactcctcttttagat |
| | | | cc |
| 11 | 202490_at | IKBKAP | gaggatggcacaagcgattcacgtaggatctgcccctgtgaccaaaacacctcccattgggcc |
| | | | ccacttccaacactggtgatcacatttcaacatgaggtttagggaaacaaatgcctaaactacag |
| | | | cactgtacataaactaacaggaaatgctgcttttgatcctcaaagaagtgatatagccaaaattgt |
| | | | aatttaagaagcctttgtcagtatagcaagatgttaactatagaatcaatctaggagtattcactgt |
| | | | aaaattcaacttttctgtatgtttgaacattttcacaatctcataggagtttttaaaaagaagagaaa |
| | | | gaagatatactttgctttggagaaatctactttttgacttacatgggtttgctgtaattaagtgcccaa |
| | | | tattgaaaggctgcaagtactttgtaatcactctttggcatgggtaaataagcatggtaacttatatt |
| | | | gaaatatagtgctcttgctttggataactgtaaagggacccatgctgatagactggaaa |
| 12 | 202707_at | UMPS | aagttcattcttaagcttgctttttttgagactggtgtttgttagacagccacagtcctgtctgggtta |
| | | | gggtcttccacatttgaggatccttcctatctctccatgggactagactgctttgttattctatttatttt |
| | | | ttaatttttttcgagacaggatctcactctgttgcccaggatggagtgcagtggtgagatcacggc |
| | | | tcattgcagcctcgacctcccaggtgatcctcccacctcagcttccagattagctggtgctatag |
| | | | gcatgcaccaccacgtccatctaaatttctttattatttgtagagatgaggtcttgccatgttaccca |
| | | | ggctggtctcaactcctgggctcaagcgatcctcctgcctcagtctctcaaagtgctgggattac |
| | | | aggtgtgagccactgtgcccagcctaattgcagtaagacaa |
| 13 | 202814_s_at | HEXIM1 | tgcctctcgcgcatggaggacgagaacaaccggctgcggctggagagcaagcggctgggt |
| | | | ggcgacgacgcgcgtgtgcgggagctggagctggagctggaccggctgcgcgccgagaa |
| | | | cctccagctgctgaccgagaacgaactgcaccggcagcaggagcgagcgccgctttccaag |
| | | | tttggagactagactgaaacttttttgggggagggggcaaaggggactttttacagtgatggaat |
| | | | gtaacattatatacatgtgtatataagacagtggacctttttatgacacataatcagaagagaaatc |
| | | | cccctggctttggttggtttcgtaaatttagctatatgtagcttgcgtgctttctcctgttcttttaattat |
| | | | gtgaaactgaagagttgcttttcttgttttcctttttagaagtttttttccttaatgtgaaagtaatttgac |
| | | | caagttataatgcatttttgtttttaacaaatcccctccttaaacggagctataaggtggccaaatct |
| | | | ga |
| 14 | 203001_s_at | STMN2 | acctcgcaacatcaacatctatacttacgatgatatggaagtgaagcaaatcaacaaacgtgcct |
| | | | ctggccaggcttttgagctgatcttgaagccaccatctcctatctcagaagccccacgaacttta |
| | | | gcttctccaaagaagaaagacctgtccctggaggagatccagaagaaactggaggctgcagg |
| | | | ggaaagaagaaagtctcaggaggcccaggtgctgaaacaattggcagagaagagggaaca |
| | | | cgagcgagaagtccttcagaaggctttggaggagaacaacaacttcagcaagatggcggag |
| | | | gaaaagctgatcctgaaaatggaacaaattaaggaaaaccgtgaggctaatctagctgctatta |
| | | | ttgaacgtctgcaggaaaaggagaggcatgctgcggaggtgcgcaggaacaaggaactcca |
| | | | ggttgaactgtctggctgaagcaagggagggtctggcacgcc |
| 15 | 203147_s_at | TRIM14 | accaatcacgcctacagtgctttgaaggtttcctctcctaggctagtttcaaacaggccctaaaca |
| | | | agtctgctgctgccctctcatcagacctccgcaccctcaccccaccatcacttanactactttaat |
| | | | ccagttccttcaaagtgatacccccacaggtaagccctcagcatcctgaatacatcatccgcag |
| | | | cctgggaaccttctccctcgtacagcacaggaacctgacacatagtaggcacacagtaaacgt |
| | | | ttgtgaatgaatgggagtcatccagtcctgactcttctgtctcttgaggtcccttgaatcttccgctt |
| | | | cctccccaccgatttcagcgtgtccacatcacagctccctccagaagctgcaagagcttcttag |
| | | | cagttcctggtctgaaccctctcccagtcctcatcttccaccctaaaactagagtgatcttcctaaa |
| | | | acttcacttaacccctcagctatgaaaaggcttccaggagtttccatgaa |
| 16 | 203438_at | STC2 | gtccacattcctgcaagcattgattgagacatttgcacaatctaaaatgtaagcaaagtagtcatt |
| | | | aaaaatacaccctctacttgggctttatactgcatacaaatttactcatgagccttcctttgaggaa |
| | | | ggatgtggatctccaaataaagatttagtgtttattttgagctctgcatcttaacaagatgatctgaa |
| | | | cacctctcctttgtatcaataaatagccctgttattctgaagtgagaggaccaagtatagtaaaatg |
| | | | ctgacatctaaaactaaataaatagaaaacaccaggccagaactatagtcatactcacacaaag |
| | | | ggagaaatttaaactcgaaccaagcaaaaggcttcacggaaatagcatggaaaaacaatgctt |
| | | | ccagtggccacttcctaaggaggaacaaccccgtctgatctcagaattggcaccacgtgagctt |
| | | | gctaagtgataatatctgtttctactacggatttaggcaacaggacctgtacattgtcacattgcat |
| 17 | 203444_s_at | MTA2 | cacaaaggataccagggccctacggaaggctctgacccatctggaaatgcggcgagctgctc |
| | | | gccgacccaacttgcccctgaaggtgaagccaacgctgattgcagtgcggccccctgtccctc |
| | | | tacctgcaccctcacatcctgccagcaccaatgagcctattgtcctggaggactgagcacctgt |
| | | | ggggaagggaggtgggctgagaggtagagggtggatgcccagggcacccaaacctccctt |
| | | | ccctttcgtgtcgaagggagtgaggagtgaattaaggaagagagcaagtgagtgtgtgtccct |
| | | | ggaggggttgggcgccctctggtgttaccacctcgagacttgtctcatgcctccatgcttgccg |
| | | | atggaggacagactgcaggaacttggcccatgtgggaacctagcctgttttggggggtagga |
| | | | cccacagatgtcttggac |
| 18 | 203475_at | CYP19A1 | gaaattctttcccagtctgtcgatttatgcctcagccacttgcctgtgctacaattcattgtgttacct |
| | | | gtagattcaggtaatacaaaccatatataatcatcaagtaatacaaactaatttagtaatagcctgg |
| | | | gttaagtattattagggccctgtgtctgcatgtagaaaaaaaaattcacatgatgcacttcaaattc |
| | | | aaataaaaatccttttggcatgttcccatttttgcttagctcaattagtgtggctaaccaagagataa |
| | | | ctgtaaatgtgacattgatttgctcttactacagctacagtgattgggggaggaaaagtcccaac |
| | | | ccaatgggctcaaacttctaaggggtactcctctcatccccttatccttctccctcgacattttctcc |
| | | | ctctttcttcccatgaccccaaagccaagggcaacagatcagtaaagaacgtggtcagagtag |
| | | | aacccctg |
| 19 | 203509_at | SORL1 | gaatatcacagcttaccttgggaatactactgacaatttctttaaaatttccaacctgaagatgggt |
| | | | cataattacacgttcaccgtccaagcaagatgcctttttggcaaccagatctgtggggagcctgc |
| | | | catcctgctgtacgatgagctggggtctggtgcagatgcatctgcaacgcaggctgccagatct |
| | | | acggatgttgctgctgtggtggtgcccatcttattcctgatactgctgagcctgggggtggggttt |
| | | | gccatcctgtacacgaagcaccggaggctgcagagcagcttcaccgccttcgccaacagcca |
| | | | ctacagctccaggctggggtccgcaatcttctcctctggggatgacctgggggaagatgatga |
| | | | agatgcccctatgataactggattttcagatgacgtccccatggtgatagcctgaaagagctttc |
| | | | ctcactagaaacca |
| 20 | 203928_x_at | MAPT | gagtccagtcgaagattgggtccctggacaatatcacccacgtccctggcggaggaaataaaa |
| | | | agattgaaacccacaagctgaccttccgcgagaacgccaaagccaagacagaccacggggc |
| | | | ggagatcgtgtacaagtcgccagtggtgtctggggacacgtctccacggcatctcagcaatgt |
| | | | ctcctccaccggcagcatcgacatggtagactcgccccagctcgccacgctagctgacgagg |
| | | | tgtctgcctccctggccaagcagggtttgtgatcaggcccctggggcggtcaataatngtgga |
| | | | gaggagagaatgagagagtgtggaaaaaaaaagaataatgacccggcccccgccctctgcc |
| | | | cccagctgctcctcgcagttcggttaattggttaatcacttaacctgcttttgtcactc |
| 21 | 203973_s_at | CEBPD | aagcggcgcaaccaggagatgcagcagaagttggtggagctgtcggctgagaacgagaag |
| | | | ctgcaccagcgcgtggagcagctcacgcgggacctggccggcctccggcagttcttcaagc |
| | | | agctgcccagcccgcccttcctgccggccgccgggacagcagactgccggtaacgcgcgg |
| | | | ccggggcgggagagactcagcaacgacccatacctcagacccgacggcccggagcggag |
| | | | cgcgccctgccctggcgcagccagagccgccgggtgcccgctgcagtttcttgggacatagg |
| | | | agcgcaaagaagctacagcctggacttaccaccactaaactgcgagagaagctaaacgtgttt |
| | | | attttcccttaaattatttttgtaatggtagctttttctacatcttactcctgttgatgcagctaaggtac |
| | | | atttgtaaaaagaaaaaaaaccagacttttcagacaaaccctttgtattgtagataagaggaaaa |
| | | | gactgagcatgctcacttttttatattaa |
| 22 | 204179_at | MB | tgttccggaaggacatggcctccaactacaaggagctgggcttccagggctaggcccctgcc |
| | | | gctcccacccccacccatctgggccccgggttcaagagagagcggggtctgatctcgtgtag |
| | | | ccatatagagtttgcttctgagtgtctgctttgtttagtagaggtgggcaggaggagctgagggg |
| | | | ctggggctggggtgttgaagttggctttgcatgcccagcgatgcgcctccctgtgggatgtcat |
| | | | caccctgggaaccgggagtgcccttggctcactgtgttctgcatggtttggatctgaattaattgt |
| | | | cctttcttctaaatcccaaccgaacttcttccaacctccaaactggctgtaaccccaaatccaagc |
| | | | cattaactacacctgacagtagcaattgtctgattaatcactggccccttgaagacagcagaatg |
| | | | tccctttgcaatgaggaggagatctgggctgggcgggccagctggggaagcatttgactatct |
| | | | ggaacttgtgtgtgcctcctcaggtatggca |
| 23 | 204267_x_at | PKMYT1 | ctgtggtgcatggcagcggaggccctgagccgagggtgggccctgtggcaggccctgcttg |
| | | | ccctgctctgctggctctggcatgggctggctcaccctgccagctggctacagcccctgggcc |
| | | | cgccagccaccccgcctggctcaccaccctgcagtttgctcctggacagcagcctctccagca |
| | | | actgggatgacgacagcctagggccttcactctcccctgaggctgtcctggcccggactgtgg |
| | | | ggagcacctccaccccccggagcaggtgcacacccagggatgccctggacctaagtgacat |
| | | | caactcagagcctcctcggggctccttcccctcctttgagcctcggaacctcctcagcctgtttg |
| | | | aggacaccctagacccaacctgagccccagactctgcctctgcacttttaaccttttatcctgtgt |
| | | | ctctcccgtcgcccttgaaagctggggcccctcgggaactcccatggtcttctctgcctggccg |
| | | | tgtctaataa |
| 24 | 204338_s_at | RGS4 | gaaacatcggctaggtttcctgctgcaaaaatctgattcctgtgaacacaattcttcccacaacaa |
| | | | gaaggacaaagtggttatttgccagagagtgagccaagaggaagtcaagaaatgggctgaat |
| | | | cactggaaaacctgattagtcatgaatgtgggctggcagctttcaaagctttcttgaagtctgaat |
| | | | atagtgaggagaatattgacttctggatcagctgtgaagagtacaagaaaatcaaatcaccatct |
| | | | aaactaagtcccaaggccaaaaagatctataatgaattcatctcagtccaggcaaccaaagag |
| | | | gtgaacctggattcttgcaccagggaagagacaagccggaacatgctagagcctacaataac |
| | | | ctgctttgatgaggcccagaagaagattttcaacctgatggagaaggattcctaccgccgcttcc |
| | | | tcaagtctcgattctatcttgatttggtcaacccgtcca |
| 25 | 204531_s_at | BRCA1 | ttcaagaaccggtttccaaagacagtcttctaattcctcattagtaataagtaaaatgtttattgttgt |
| | | | agctctggtatataatccattcctcttaaaatataagacctctggcatgaatatttcatatctataaaa |
| | | | tgacagatcccaccaggaaggaagctgttgctttctttgaggtgatttttttcctttgctccctgttg |
| | | | ctgaaaccatacagcttcataaataattttgcttgctgaaggaagaaaaagtgtttttcataaaccc |
| | | | attatccaggactgtttatagctgttggaaggactaggtcttccctagcccccccagtgtgcaag |
| | | | ggcagtgaagacttgattgtaca |
| 26 | 204584_at | L1CAM | cctccctatcgtctgaacagttgtcttcctcagcctcctcccgcccccaccttgggaatgtaaata |
| | | | caccgtgactttgaaagtttgtacccctgtccttccctttacgccactagtgtgtaggcagatgtct |
| | | | gagtccctaggtggtttctaggattgatagcaattagctttgatgaacccatcccaggaaaaata |
| | | | aaaacagacaaaaaaaaaggaaagattggttctcccagcactgctcagcagccacagcctcc |
| | | | ctgtatgcctgtgcttggtctactgataagccctctacaaaa |
| 27 | 204684_at | NPTX1 | ttccttttgtagattcccagtttattttctaagactgcaaagatcactttgtcaccagccctgggacct |
| | | | gagaccaagggggtgtcttgtgggcagtgagggggtgaggagaggctggcatgaggttcag |
| | | | tcattccagtgagctccaaagaggggccacctgttctcaaaagcatgttggggaccaggaggt |
| | | | aaaactggccatttatggtgaacctgtgtcttggagctgacttactaagtggaatgagccgagga |
| | | | tttgaatatcagttctaaccttgatagaagaaccttgggttacatgtggttcacattaagaggatag |
| | | | aatcctttggaatcttatggcaaccaaatgtggcttgacgaagtcgtggtttcatctctt |
| 28 | 204810_s_at | CKM | gcaagcaccccaagttcgaggagatcctcacccgcctgcgtctgcagaagaggggtacaggt |
| | | | gcggtggacacagctgccgtgggctcagtatttgacgtgtccaacgctgatcggctgggctcg |
| | | | tccgaagtagaacaggtgcagctggtggtggatggtgtgaagctcatggtggaaatggagaa |
| | | | gaagttggagaaaggccagtccatcgacgacatgatccccgcccagaagtaggcgcctgcc |
| | | | cacctgccaccgactgctggaaccccagccagtgggagggcctggcccaccagagtcctgc |
| | | | tccctcactcctcgccccgccccctgtcccagagtccacctgggggctctctccacccttctca |
| | | | gagttccagtttcaaccagagttccaaccaatgggctccatcctctggattctggccaatgaaat |
| | | | atctccctggcagggtcctcttcttttcccagagctcctccccaaccaggagctctagttaatg |
| 29 | 204817_at | ESPL1 | tgtttggctgtagcagtgcggccctggctgtgcatggaaacctggagggggctggcatcgtgc |
| | | | tcaagtacatcatggctggttgccccttgtttctgggtaatctctgggatgtgactgaccgcgaca |
| | | | ttgaccgctacacggaagctctgctgcaaggctggcttggagcaggcccaggggcccccctt |
| | | | ctctactatgtaaaccaggcccgccaagctccccgactcaagtatcttattggggctgcacctat |
| | | | agcctatggcttgcctgtctctctgcggtaaccccatggagctgtcttattgatgctagaagcctc |
| | | | ataactgttctacctc |
| 30 | 204933_s_at | TNFRSF11B | gataaaacggcaacacagctcacaagaacagactttccagctgctgaagttatggaaacatca |
| | | | aaacaaagcccaagatatagtcaagaagatcatccaagatattgacctctgtgaaaacagcgtg |
| | | | cagcggcacattggacatgctaacctcaccttcgagcagcttcgtagcttgatggaaagcttac |
| | | | cgggaaagaaagtgggagcagaagacattgaaaaaacaataaaggcatgcaaacccagtga |
| | | | ccagatcctgaagctgctcagtttgtggcgaataaaaaatggcgaccaagacaccttgaaggg |
| | | | cctaatgcacgcactaaagcactcaaagacgtaccactttcccaaaactgtcactcagagtcta |
| | | | aagaagaccatcaggttccttcacagc |
| 31 | 204953_at | SNAP91 | agagaggtgctattcaagtgattctgaaggcaccccaaggtatatctgtaatttaaagattactgc |
| | | | aaatatctttactttactgtgggtttttagtacatctgttaatttagtgtttctttgtgtgttttgtagacta |
| | | | gtgttcttccatccttcaactgagctcaaagtaggttttgttgtaacattgtgattaggatttaaacta |
| | | | attcagagaattgtatcttttactgtacatactgtattctttaagttttaatttgttgtcatactgtctgtg |
| | | | ctgatggcttggcttaagattttgatgcataaatgaggtcactgttgatcagtgttgctagtagcttg |
| | | | gcagctcttcataaaagcatattgggttggaaaggtgtttgcctatttttca |
| 32 | 205046_at | CENPE | aatcagcatctttccaatgaggtcaaaacttggaaggaaagaacccttaaaagagaggctcac |
| | | | aaacaagtaacttgtgagaattctccaaagtctcctaaagtgactggaacagcttctaaaaagaa |
| | | | acaaattacaccctctcaatgcaaggaacggaatttacaagatcctgtgccaaaggaatcacca |
| | | | aaatcttgtttttttgatagccgatcaaagtctttaccatcacctcatccagttcgctattttgataact |
| | | | caagtttaggcctttgtccagaggtgcaaaatgcaggagcagagagtgtggattctcagccag |
| | | | gtccttggcacgcctcctcaggcaaggatgtgcctgagtgcaaaactcagtagactcctctttgt |
| | | | cacttctctggagatccagcattccttatttggaaatgactttgtttatgtgtctatccctggtaatga |
| | | | tgttgtagtgcagcttaatttcaattcagtctttactttgccactag |
| 33 | 205189_s_at | FANCC | ttccctccacctccaagacaggtggcggccgggcaggcactcttaagcccacctccccctctt |
| | | | gttgccttcgatttcggcaaagcctgggcaggtgccaccgggaaggaatggcatcgagatgct |
| | | | gggcggggacgcggcgtggcgagggggcttgacggcgttggcggggctgggcacaggg |
| | | | gcagccgcagggaggcagggatggcaaggcgtgaagccaccctggaaggaactggacca |
| | | | aggtcttcagaggtgcgacagggtctggaatctgaccttactctagcaggagtttttgtagactct |
| | | | ccctgatagtttagtttttgataaagcatgctggtaaaaccactaccctcagagagagccaaaaa |
| | | | tacagaagaggcggagagcgcccctccaaccaggctgttattcccctggactc |
| 34 | 205217_at | TIMM8A | gtacatgggactatgcttttctcaaagccccattaactgcttcctataattttgatagtgggaccac |
| | | | atacgtaaaaatctctcatttgtgtggagtcatttctgatttcaggggagatccttgtgtttatcaga |
| | | | aagggcagaagtaggggaagaataatttggtatccttatctagtgtttgattgtcaatgctggaga |
| | | | aaaatatctgtaagagtgtttatacagtacacttcagttatcttgatctccctttcctatatgatgattt |
| | | | gcttaaatatccatattaagtaagtctcaaggtagggtaggcagcctgagagtctagaggccttt |
| | | | agttataaaggaatctagccagtgaacataattcttattactagactgccacaaggaagaaattaa |
| | | | cttaccctgtatatcagggtacaaaaaattcagtgatgtgcctaaataagttataaagatttaggcc |
| | | | aatcagaagctaacagcagtttcaggtagaggtgcatgcctaatgttagttagtgtagattccatt |
| | | | tactgcattctt |
| 35 | 205386_s_at | MDM2 | tttcccctagttgacctgtctataagagaattatatatttctaactatataaccctaggaatttagaca |
| | | | acctgaaatttattcacatatatcaaagtgagaaaatgcctcaattcacatagatttcttctctttagt |
| | | | ataattgacctactttggtagtggaatagtgaatacttactataatttgacttgaatatgtagctcatc |
| | | | ctttacaccaactcctaattttaaataatttctactctgtcttaaatgagaagtacttggttttttttttctt |
| | | | aaatatgtatatgacatttaaatgtaacttattattttttttgagaccgagtcttgctctgttacccagg |
| | | | ctggagtgcagtgggtgatcttggctcactgcaagctctgccctccccgggttcgcaccattctc |
| | | | ctgcctcagcctcccaattagcttggcctacagtcatctgcc |
| 36 | 205433_at | BCHE | ggaaagcaggattccatcgctggaacaattacatgatggactggaaaaatcaatttaacgatta |
| | | | cactagcaagaaagaaagttgtgtgggtctctaattaatagatttaccctttatagaacatattttcc |
| | | | tttagatcaaggcaaaaatatcaggagcttttttacacacctactaaaaaagttattatgtagctga |
| | | | aacaaaaatgccagaaggataatattgattcctcacatctttaacttagtattttacctagcatttca |
| | | | aaacccaaatggctagaacatgtttaattaaatttcacaatataaagttctacagttaattatgtgca |
| | | | tattaaaacaatggcctggttcaatttctttctttccttaataaatttaagttttttccccccaaaattatc |
| | | | agtgctctgcttttagtcacgtgtattttcattaccactcgtaaaaaggtatcttttttaaatgaattaa |
| | | | atattgaaacactgtacaccatagtttaca |
| 37 | 205481_at | ADORA1 | gaggagaacactagacatgccaactcgggagcattctgcctgcctgggaacggggtggacg |
| | | | agggagtgtctgtaaggactcagtgttgactgtaggcgcccctggggtgggtttagcaggctg |
| | | | cagcaggcagaggaggagtacccccctgagagcatgtgggggaaggccttgctgtcatgtg |
| | | | aatccctcaatacccctagtatctggctgggttttcaggggctttggaagctctgttgcaggtgtc |
| | | | cgggggtctaggactttagggatctgggatctggggaaggaccaacccatgccctgccaagc |
| | | | ctggagcccctgtgttggggggcaaggtgggggagcctggagcccctgtgtgggagggcg |
| | | | aggcgggggagcctggagcccctgtgtgggagggcgaggcgggggatcctggagcccct |
| | | | gtgtcggggggcgagggaggggaggtggccgtcggttgaccttctgaacatgagtgtcaact |
| | | | ccaggacttgcttccaagcccttccctctgttggaaattgggtgtgccctggctcc |
| 38 | 205491_s_at | GJB3 | tgcttccagccttcgtaattagacttcaccctgagtacacacacaatcactgccactctcactata |
| | | | gacaaaccacactccctcctctgtcacccagtcactgccatctcaacacacatccccaccctgt |
| | | | gtacacacaatctctgttattcatactctcactccttatgcgcactctcaacagggcatgtagtctg |
| | | | cactcaagcatgccatcccagcctcaccctgcattttattcggctcatcccattttccctgaacattt |
| | | | tcgctgaactagggccctggcaggatgctgggactgtgcaaggaggtaggacctatgcccac |
| | | | ggagctaagagacaggaacacaggctcatctcccgcactaaccaacccctgggatggctcac |
| | | | agcctgctcccagtgctgtgtcatgacctgaa |
| 39 | 205501_at | PDE10A | atgcttgcccaacacactgtgaaatagttaccaaaatttgtacaaatgcagcatcttcattctttctg |
| | | | agaagacaagatggttttctttacatgaacaaatgaacaaaagagatcctagatccataacgtag |
| | | | ctaaggcatctaagagtttgctgttgataatcttgctgaccaaaaactactggagagtaacacag |
| | | | gttatatgccatcacaaatacaatgctcatgaagaactgatttgtagagtcaatgaacctgtgtcc |
| | | | agaattttaataggctctctattggaaggagaaagaatttcaagttaacagtatctaactttatcata |
| | | | gttgatgttagtaaattttaaaaaatgattttatatgtatgacaaaaatctttgtaaaatgcgcaagtg |
| | | | caataatttaaagaggtcttaactttgcatttataaattataaatattgtacatgtgtgtaattttttcat |
| | | | gtattcatttgcagtctttgtatttaaaa |
| 40 | 205825_at | PCSK1 | tttccattcccaatctagtgctagatgtataaatctttcttttgattcttcctaacaaaatattttctgggt |
| | | | taaaaccccagccaactcattgggttgtagccaaaggttcactctcaagaagctttaatatttaaa |
| | | | taaaatcatattgaatgtttccaacctggagtataatattcagatataaaacagttttgtcagtctttct |
| | | | tagtgcctgtgtggatttttgtgaaaatgtcaaagagaaaacttatatactatttcccttgaaatttta |
| | | | aactatattttctttacaggtatttataatataccaatgcttttatcaaacagaattttaaagagcataa |
| | | | taaattatattaaagaaccaaaagttttcctgagaataagaaagtttcacccaataaaatatttttga |
| | | | aaggcatgttcctctgtcaatgaaaaaaagtacatgtatgtgttgtgatattaaaagtgacatttgt |
| | | | ctaatagcctaatacaacatgtagctgagtttaacatgtgtggtcttg |
| 41 | 205893_at | NLGN1 | gaacctaggagagtcaacatctggaggattttagtctttcttacacatatgtgtgattttaaacgaa |
| | | | tattctcagaccacaggaaactcttcatccccctgttgtttaccagtaacagtatatcacagacctt |
| | | | tccaaatgtttgtatatgtaatcagatgtacatttatattgaaaaacaaatgagatggacttaaaga |
| | | | gcacatcctgataaatactttctctctcacctgtactatatttctattagactaaagttatgtgattttttt |
| | | | tttacattttttcagatgactagcaattttgatagtttataagataatgcaaagaactttctctgacaaa |
| | | | ctaactgcagtaacagaaacctttcttttcagttactctttttcaagaatgaaagattattatacaaaa |
| | | | aattgtatactacttgatggaaccaactttgtacatcttggccatgtcactggtcattg |
| 42 | 205938_at | PPM1E | catgctaggctttctcagtggggaaaaaaatggctggatagaactgggacaaacacagaccca |
| | | | tctttaggggtctggattttgtaggtccgactacacagcagtgttaactcatttctcatgccattagc |
| | | | tctctacaaaataaagcaaagtagttctagtgtggtcgttataaaccaatattgtgaaaaatagca |
| | | | actattcatttgttcacaacatgcgtatttatagagtagttaggtaccatttgtaaggtaaatccttta |
| | | | aaattctataatacatactaaaatagtggttattggtctgatatatgctgctcttggttctataaacta |
| | | | gataaaagcagtgctttgtgaaatgcagtgttctctcttaacgccactggtgataggaagtagttc |
| | | | ccttcagttcaaatc |
| 43 | 205946_at | VIPR2 | ttcctcccctgtagggtttggacagacccacccccagccttgcccagctttcaaaggacaaaag |
| | | | ggagcatcccccacctactctcaggtttttgaggaaacaaagatttgtggtaactgaaggtgttg |
| | | | ggtcagtggccaggtgccgacactgagctgtgacccagaggggacgctgaggaagtgggc |
| | | | gtgagtggacntgtcaggtggttaccaggcactggttgttgatggtcggtggttgggtgtgggc |
| | | | agtcatcagtcatcaggtgtgctcaggggacaatctcccctcaaccgcacatgtgccactgttc |
| | | | agcggagctgactggtttcncctggtagagggnccggctgtttcctgacagatgcctggtgag |
| | | | caggggaagcaggacccagtggtcancaggtgtctttaactgtcattgtgtgtggaatgtcgca |
| | | | gactcctccacgtggcgggaatgagct |
| 44 | 206043_s_at | ATP2C2 | gcaccacgacgatgacgttcacttgttttgtgtttttcgatctcttcaacgccttgacctgccgctct |
| | | | cagaccaagctgatatttgagatcggctttctcaggaaccacatgttcctctactccgtcctggg |
| | | | gtccatcctggggcagctggcggtcatttacatccccccgctgcagagggtcttccagacgga |
| | | | gaacctgggagcgcttgatttgctgtttttaactggattggcctcatccgtcttcattttgtcagagc |
| | | | tcctcaaactatgtgaaaaatactgttgcagccccaagagagtccagatgcaccctgaagatgt |
| | | | gtagtggaccgcactccgcggcaccttccctaatcatctcgatctggttgtgactgtggcccctg |
| | | | ccgtgtctcctcgtcaggggagacttttaggaggccgcagccttccatcaccggatcagtttttc |
| | | | ctcttaggaaagctgcaggaacctcgtgggc |
| 45 | 206096_at | ZNF35 | gtggctttcctaggaatgggtcgtacaaagctaagtggtaatgatgctatttggggaaaggtcttt |
| | | | tttgcttaantttgttttttaaaactctgatgattncttgagcaacaggcaggttatctgcctggttga |
| | | | attctggttgaaccgtgtattctaatatttctggttaagtggtgactgggtaaggaaaccacttggg |
| | | | gtagcagttcaacaattcacttacgaatgtttataagctttccatttcctaggtaattttttaaaagcc |
| | | | agtcaaaacaaaaactttactgaaaatggacagaaataggaaatggactttttccttactgtctat |
| | | | acctcctgaaccttggtattgtaaagatctggggacctctgggtctgttctgaccattccctagtct |
| | | | ccatggccaagcactcaaggattgatggacaccacacaccagctatattcatttgccaagatca |
| | | | acagctccttctccaaacaactcaagcccccaattccnatcgcattcnnttngggtgagatgca |
| | | | actaacagcccctt |
| 46 | 206228_at | PAX2 | gcaggctagatccgaggtggcagctccagcccccgggctcgccccctngcgggcgtgccc |
| | | | cgcgcgccccgggcggccgaaggccgggccgccccgtcccgccccgtagttgctctttcgg |
| | | | tagtggcgatgcgccctgcatgtctcctcacccgtggatcgtgacgactcgaaataacagaaa |
| | | | caaagtcaataaagtgaaaataaataaaaatccttgaacaaatccgaaaaggcttggagtcctc |
| | | | gcccagatctctctcccctgcgagccctttttatttgagaaggaaaaagagaaaagagaatcgtt |
| | | | taagggaacccggcgcccagccaggctccagtggcccgaacggggcggcgagggcggc |
| | | | gagggcgccgaggtccggcccatcccagtcctgtggggctggccgggcagagaccccgga |
| | | | cccaggcccaggcctaacctgctaaatgtccccggacggttctggtctcctcggccactttcag |
| | | | tgcgtcggttcgttttgattctttt |
| 47 | 206232_s_at | B4GALT6 | tgcagttttgcatgtaatcggttatacctttattggacttttatagacattttttatttgcatgaaaaaaa |
| | | | ctcactaaatttacatcactaaacaaaggttaacccttgtgtgaaatgaaggaactgtcaataatt |
| | | | gacagccaactaatacagtaaactgttatactagttttgagctttagacctcagccttttgtgtgga |
| | | | agaagtcacagctttcttaggctttaaaggaaaagaaggaaggacttaaatagcttttcttcctac |
| | | | cgggattacctatgtttttccttgcttgcaatctcatctgattttgctagaaatcacaaccatattgttt |
| | | | atgcatattgcatgagtattaccaagaaaaaaatctttaaaagttgtgatgtgacatgatataaag |
| | | | gatctctttatgttaaatgtctttccatgtacctctggtgtgtcagggattttgtgcctcaaaaaatgtt |
| | | | tccaaggttgtgtgtttatactgtgtattttttttaaattcacggtgaacagcacttttattatttcca |
| 48 | 206401_s_at | MAPT | aggtggcagtggtccgtactccacccaagtcgccgtcttccgccaagagccgcctgcagaca |
| | | | gcccccgtgcccatgccagacctgaagaatgtcaagtccaagatcggctccactgagaacct |
| | | | gaagcaccagccgggaggcgggaaggtgcaaatagtctacaaaccagttgacctgagcaag |
| | | | gtgacctccaagtgtggctcattaggcaacatccatcataaaccaggaggtggccaggtggaa |
| | | | gtaaaatctgagaagcttgacttcaaggacagagtccagtcgaagattgggtccctggacaata |
| | | | tcacccacgtccctggcggaggaaataaaaagattgaaacccacaagctgaccttccgcgag |
| | | | aacgccaaagccaagacagaccacggggcggagatcgtgtacaagtcgccagtggtgtctg |
| | | | gggacacgtctccacggcatctcagcaatgtctcctccaccggcagcatcgacatggtagact |
| | | | cgccccagctcgccacgctagctgacgaggtgtctgcctcc |
| 49 | 206426_at | MLANA | gtaaagatcctatagctctttttttttgagatggagtttcgcttttgttgcccaggctggagtgcaat |
| | | | ggcgcgatcttggctcaccataacctccgcctcccaggttcaagcaattctcctgccttagcctc |
| | | | ctgagtagctgggattacaggcgtgcgccactatgcctgactaattttgtagttttagtagagacg |
| | | | gggtttctccatgttggtcaggctggtctcaaactcctgacctcaggtgatctgcccgcctcagc |
| | | | ctcccaaagtgctggaattacaggcgtgagccaccacgcctggctggatcctatatcttaggta |
| | | | agacatataacgcagtctaattacatttcacttcaaggctcaatgctattctaactaatgacaagta |
| | | | ttttctactaaaccagaaattggtagaaggatttaaataagtaaaagctactatgtactgccttagt |
| | | | gctgatgcctgtgtactgccttaaatgtacctatggcaatttagctctcttgggttcccaaatccctc |
| | | | tcacaagaatgt |
| 50 | 206496_at | FMO3 | aaagcccaacatcccatggctgtttctcacagatcccaaattggccatggaagtttattttggccc |
| | | | ttgtagtccctaccagtttaggctggtgggcccagggcagtggccaggagccagaaatgccat |
| | | | gctgacccagtgggaccggtcgttgaaacccatgcagacacgagtggtcgggagacttcaga |
| | | | agccttgcttctttttccattggctgaagctctttgcaattcctattctgttaatcgctgttttccttgtgt |
| | | | tgacctaatcatcattttctctaggatttctgaaagttactgacaatacccagacaggggctttgc |
| 51 | 206505_at | UGT2B4 | taattacgtctgaggctggaagctgggaaacccaataaatgaactcctttagtttattacaacaag |
| | | | aagacgttgtgatacaagagattcctttcttcttgtgacaaaacatctttcaaaacttaccttgtcaa |
| | | | gtcaaaatttgttttagtacctgtttaaccattagaaatatttcatgtcaaggaggaaaacattaggg |
| | | | aaaacaaaaatgatataaagccatatgaggttatattgaaatgtattgagcttatattgaaatttatt |
| | | | gttccaattcacaggttacatgaaaaaaaatttactaagcttaactacatgtcacacattgtacatg |
| | | | gaaacaagaacattaagaagtccgactgacagtatcagtactgttttgcaaatactcagcatactt |
| | | | tggatccatttcatgcaggattgtgttgttttaac |
| 52 | 206524_at | T | agcagtggaggagcacacggacctttccccagagcccccagcatcccttgctcacacctgca |
| | | | gtagcggtgctgtccaggtggcttacagatgaacccaactgtggagatgatgcagttggccca |
| | | | acctcactgacggtgaaaaaatgtttgccagggtccagaaactttttttggtttatttctcatacagt |
| | | | gtattggcaactttggcacaccagaatttgtaaactccaccagtcctactttagtgagataaaaag |
| | | | cacactcttaatcttcttccttgttgctttcaagtagttagagttgagctgttaaggacagaataaaa |
| | | | tcatagttgaggacagcaggttttagttgaattgaaaatttgactgctctgccccctagaatgtgtg |
| | | | tattttaagcatatgtagctaatctcttgtgtt |
| 53 | 206552_s_at | TAC1 | ttcagcttcatttgtgtcaatgggcaatgacaggtaaattaagacatgcactatgaggaataattat |
| | | | ttatttaataacaattgtttggggttgaaaattcaaaaagtgtttatttttcatattgtgccaatatgtatt |
| | | | gtaaacatgtgttttaattccaatatgatgactcccttaaaatagaaataagtggttatttctcaaca |
| | | | aagcacagtgttaaatgaaattgtaaaacctgtcaatgatacagtccctaaagaaaaaaaatcat |
| | | | tgctttgaagcagttgtgtcagctactgcggaaaaggaaggaaactcctgacagtcttgtgctttt |
| | | | cctatttgttttcatggtgaaaatgtactgagattttggtattacactgtatttgtatctctgaagcatg |
| | | | tttcatgttttgtgactatatagagatgtttttaaaagtttcaatgtgattctaatgtcttcatttcattgta |
| | | | tgatg |
| 54 | 206619_at | DKK4 | ctgtctgacacggactgcaataccagaaagttctgcctccagccccgcgatgagaagccgttct |
| | | | gtgctacatgtcgtgggttgcggaggaggtgccagcgagatgccatgtgctgccctgggaca |
| | | | ctctgtgtgaacgatgtttgtactacgatggaagatgcaaccccaatattagaaaggcagcttga |
| | | | tgagcaagatggcacacatgcagaaggaacaactgggcacccagtccaggaaaaccaaccc |
| | | | aaaaggaagccaagtattaagaaatcacaaggcaggaagggacaagagggagaaagttgtc |
| | | | tgagaacttttgactgtggccctggactttgctgtgctcgtcatttttggacgaaaatttgtaagcc |
| | | | agtccttttggagggacaggtctgctccagaagagggcataaagacactgctcaagctccaga |
| | | | aatcttccagcgttgcgactgtggccctggactactgtgtcgaagccaattgaccagcaatcgg |
| | | | cagcatgctcgat |
| 55 | 206622_at | TRH | gccctcttcctttaggcatgtgagaaaatcagcctagcagtttaaaccccactttcctccacttag |
| | | | caccataggcaagggggcagatcccagagcccctctcaccccccccaccacaggcctgctc |
| | | | cttccttagccttggctaagatggtccttctgtgtcttgcaaagactccccaagtggacagggag |
| | | | cccctgggagggcagccagtgagggtggggtgggactgaagcgttgtgtgcaaatccagctt |
| | | | ccatcccctccccaacctggcaggattctccatgtgtaaacttcacccccaggacccaggatctt |
| | | | ctcctttctgggcatccctttgtgggtgggcagagccctgacccacagctgtgttactgcttgga |
| | | | gaagcatatgtaggggcataccctgtggtgttgtgctgtgtctggctgtgggataaatgtgtgtg |
| | | | ggaatattgaaacatcgcctaggaattgtggtttgtatataaccctctaagcccctatcccttgtcg |
| | | | atgacagtca |
| 56 | 206661_at | DBF4B | accaggagtgtcagcttttagaaggatcatggtcatgtgagcttctggtcaccggaagccagaa |
| | | | atactcagctgccatgttgatccacaaaggtgggaggatgtggggaagggggaaagcggtga |
| | | | ggacgcagagtgcaggctgtggcctcggcatcccgcaggaggtccctagaacatgccgtttc |
| | | | atgtcacctgctacagctctcccccagctagtatgatgatccgttttacaaatgcagaaatgatctt |
| | | | aatattcatgaccactggccaggcgaggtggctcacacctgtaatcccagcactttgggaggc |
| | | | caaggcgggtggatcacaaggtcaagagttcgagaccagcctgaccaacgtggtgaaaccc |
| | | | cgtctctactaaaaatagaagcattagccgagcctggtgg |
| 57 | 206672_at | AQP2 | gcgcagagtagctgcttcctggacgtgcgcgcccaggccagtgctgtgagcaggcggggag |
| | | | gaggctgccggaggagcctgagcctggcaggttcccctgccctgaggctgtgagcagctagt |
| | | | ggtggcttctcctgcctttttcagggaactgggaaacttaggggactgagctggggagggagg |
| | | | caggtgggtggtaagagggaaactctggagagcctgcacccaggtactgagtggggagtgt |
| | | | acagaccctgccttgggggttctgggaatgatgcaactggttttactagtgtgcaagtgtgttcat |
| | | | ccccaagttctcttttgtcctcacatgcagagttgtgcatgcccctgagtgtgaacaggtttgccta |
| | | | cgttggtgca |
| 58 | 206678_at | GABRA1 | tggtttattgccgtgtgctatgcctttgtgttctcagctctgattgagtttgccacagtaaactatttca |
| | | | ctaagagaggttatgcatgggatggcaaaagtgtggttccagaaaagccaaagaaagtaaag |
| | | | gatcctcttattaagaaaaacaacacttacgctccaacagcaaccagctacacccctaatttggc |
| | | | caggggcgacccgggcttagccaccattgctaaaagtgcaaccatagaacctaaagaggtca |
| | | | agcccgaaacaaaaccaccagaacccaagaaaacctttaacagtgtcagcaaaattgaccga |
| | | | ctgtcaagaatagccttcccgctgctatttggaatctttaacttagtctactgggctacgtatttaaa |
| | | | cagagagcctcagctaaaagcccccacaccacatcaatagatcttttactcacattctgttgttca |
| | | | gttcctctgcactgggaatttatttatgttctcaacgcagtaattccca |
| 59 | 206799_at | SCGB1D2 | tagaagtccaaatcactcattgtttgtgaaagctgagctcacagcaaaacaagccaccatgaag |
| | | | ctgtcggtgtgtctcctgctggtcacgctggccctctgctgctaccaggccaatgccgagttctg |
| | | | cccagctcttgtttctgagctgttagacttcttcttcattagtgaacctctgttcaagttaagtcttgcc |
| | | | aaatttgatgcccctccggaagctgttgcagccaagttaggagtgaagagatgcacggatcag |
| | | | atgtcccttcagaaacgaagcctcattgcggaagtcctggtgaaaatattgaagaaatgtagtgt |
| | | | gtgacatgtaaaaactttcatcctggtttccactgtctttcaatgacaccctgatctt |
| 60 | 206835_at | STATH | aagcttcacttcaacttcactacttctgtagtctcatcttgagtaaaagagaacccagccaactatg |
| | | | aagttccttgtctttgccttcatcttggctctcatggtttccatgattggagctgattcatctgaagag |
| | | | aaatttttgcgtagaattggaagattcggttatgggtatggcccttatcagccagttccagaacaa |
| | | | ccactatacccacaaccataccaaccacaataccaacaatataccttttaatatcatcagtaactg |
| | | | caggacatgattattgaggcttgattggcaaatacgacttctacatccatattctcatctttcatacc |
| | | | atatcacactactaccactttttgaagaatcatcaaagagcaatgcaaatgaaaaacactataattt |
| | | | actgtatactctttgtttcaggatacttgccttttcaattgtcacttgatgatataattgcaatttaaact |
| | | | gttaagctgtgttcagtactgtttc |
| 61 | 206940_s_at | LOC100131317 | ggtttgttaccatcctttaatcataactaaaacattgaaaacagaacaaatgagaaaagaaaaaa |
| | | /// | aacctgccgattaacaatgacgaaaatcatgcatgatctgaaaggtgtggaaagaaacacaatt |
| | | POU4F1 | aggtctcactctggttaggcattatttatttaattatgttgtatatcattgtttgcagggcaacattctat |
| | | | gcattgaactgagcactaactgggctagcttctggtagacgtttgtggctagtgcgattcacagt |
| | | | ctactgcctgttccactgaaacattttgtcatattcttgtattcaaagaaaaaaggaaaaaaagatt |
| | | | attgtaaatattttatttaatgcacacattcacacagtggtaacagactgccagtgttcatcctgaaa |
| | | | tgtctcacggattgatctacctgtccatgtatgtctgctgagctttctccttggttatgttttt |
| 62 | 206984_s_at | RIT2 | taaagagctcatttttcaggtccgccacacctatgaaattcccctggtgctggtgggtaacaaaat |
| | | | tgatctggaacagttccgccaggtttctacagaagaaggcttgagtcttgcccaagaatataatt |
| | | | gtggtttttttgagacctctgcagccctcagattctgtattgatgatgcttttcatggcttagtgagg |
| | | | gaaattcgcaagaaggagtccatgccatccttgatggaaaagaaactgaagagaaaagacag |
| | | | cctgtggaagaagctcaaaggttctttgaagaagaagagagaaaatatgacatgatatctttgct |
| | | | tttgagttcctcacgctctctgaattttattagttggacaattccatatgtagcattctgcttcaatatta |
| | | | tctctctatgtgtctctctctctttaaatatctgcctgtaggtaaaagcaagctctgcatatctgtacc |
| | | | tcttgagatagttttgttttgcctttaacagttggatgga |
| 63 | 207003_at | GUCA2A | gaggggtcaccgtgcaggatggaaatttctccttttctctggagtcagtgaagaagctcaaaga |
| | | | cctccaggagccccaggagcccagggttgggaaactcaggaactttgcacccatccctggtg |
| | | | aacctgtggttcccatcctctgtagcaacccgaactttccagaagaactcaagcctctctgcaag |
| | | | gagcccaatgcccaggagatacttcagaggctggaggaaatcgctgaggacccgggcacat |
| | | | gtgaaatctgtgcctacgctgcctgtaccggatgctaggggggcttgcccactgcctgcctccc |
| | | | ctccgcagcagggaagctcttttctcctgcagaaagggccacccatgatactccactcccagc |
| | | | agctcaacctaccctggtccagtcgggaggagcagcccggggaggaactgggtgact |
| 64 | 207028_at | LOC100129296 | ctccccccgagagaaggctgcaaagctgggaagcccagggtgtgctcctcccgcccttttgg |
| | | /// | acccccgggcttgcaccggctgcactctgagaaccagctgcgcgcggagcggtgcaatgca |
| | | MYCNOS | gcacccaccctgcgagcctggcaattgcttgtcattaaaagaaaaaaaaattacggagggctc |
| | | | cgggggtgtgtgttggggaggggagaccgatgcttctaacccagcccccgctttgactgcgtg |
| | | | ttgtgcagctgagcgcgaggccaacgttgagcaaggccttgcagggaggttgctcctgtgtaa |
| | | | ttacgaaagaaggctagtccgaaggtgcaaaatagcagggagaggacgcgcccccttagga |
| | | | acaagacctctggatgtttccagtttcaaattgaaagaagaggggcgccccccttg |
| 65 | 207208_at | RBMXL2 | acagcagcagttatggccggagcgaccgctactcgaggggccgacaccgggtgggcagac |
| | | | cagatcgtgggctctctctgtccatggaaaggggctgccctccccagcgtgattcttacagccg |
| | | | gtcaggctgcagggtgcccaggggcggaggccgtctaggaggccgcttggagagaggag |
| | | | gaggccggagcagatactaagcaggaacagacttgggaccaaaaatcccttttcaacgaaac |
| | | | taacaaaaagaagaacctgttgtatggtaactacccaaggactagtacaaggaagagttgttttt |
| | | | accttttaagaatttcctgttaagatcgtctccatttttatgcttttgggagaaaaaacttaaaattcgt |
| | | | ttagtttagttttggaattgttaacgtttctttcaacaagctcctgttaaaagtatatgaacctgagtac |
| | | | tagtcttcttacatttacaagtagaaattcgattaatggcttcttcccttgtaaattttcttg |
| 66 | 207219_at | ZNF643 | cagccagagcattggactgatccagcatttgagaactcatgttagagagaaaccttttacatgca |
| | | | aagactgtggaaaagcgtttttccagattagacaccttaggcaacatgagattattcatactggtg |
| | | | tgaaaccctatatttgtaatgtatgtagtaaaaccttcagccatagtacatacctaactcaacacca |
| | | | gagaactcatactggagaaagaccatataaatgtaaggaatgtgggaaagcctttagccagag |
| | | | aatacatctttctatccatcagagagtccatactggagtaaaaccttatgaatgcagtcattgtgg |
| | | | gaaagcctttaggcatgattcatcctttgctaaacatcagagaattcatactggagaaaaacctta |
| | | | tgattgtaatgagtgtggaaaagccttcagctgtagttcatcccttattagacactgcaaaacaca |
| | | | tttaagaaataccttcagcaatgttgtgtgaaatatactaaacatcaaagaatctatgttggagcac |
| | | | aagattctaaatcagtggttccctg |
| 67 | 207529_at | DEFA5 | gagtcactccaggaaagagctgatgaggctacaacccagaagcagtctggggaagacaacc |
| | | | aggaccttgctatctcctttgcaggaaatggactctctgctcttagaacctcaggttctcaggcaa |
| | | | gagccacctgctattgccgaaccggccgttgtgctacccgtgagtccctctccggggtgtgtga |
| | | | aatcagtggccgcctctacagactctgctgtcgctgagcttcctagatagaaaccaaagcagtg |
| | | | caagattcagttcaaggtcctgaaaaaagaaaaacattttactctgtgtaccttgtgtctt |
| 68 | 207597_at | ADAM18 | gtgacgctcaatctacagtttattcatatattcaagaccatgtatgtgtatctatagccactggttcct |
| | | | ccatgagatcagatggaacagacaatgcctatgtggctgatggcaccatgtgtggtccagaaat |
| | | | gtactgtgtaaataaaacctgcagaaaagttcatttaatgggatataactgtaatgccaccacaaa |
| | | | atgcaaagggaaagggatatgtaataattttggtaattgtcaatgcttccctggacatagacctcc |
| | | | agattgtaaattccagtttggttccccagggggtagtattgatgatggaaattttcagaaatctggt |
| | | | gacttttatactgaaaaaggctacaatacacactggaacaactggtttattctgagtttctgcattttt |
| | | | ctgccgtttttcatagttttcaccactgtgatctttaaaagaaatgaaataagtaaatcatgtaacag |
| | | | agagaatgcagagtataatcgtaattcatccgttgtatcag |
| 69 | 207814_at | DEFA6 | gagccactccaagctgaggatgatccactgcaggcaaaagcttatgaggctgatgcccagga |
| | | | gcagcgtggggcaaatgaccaggactttgccgtctcctttgcagaggatgcaagctcaagtctt |
| | | | agagctttgggctcaacaagggctttcacttgccattgcagaaggtcctgttattcaacagaatat |
| | | | tcctatgggacctgcactgtcatgggtattaaccacagattctgctgcctctgagggatgagaac |
| | | | agagagaaatatattcataatttactttatgacctagaaggaaactgtcgtgtgtcccatacattgc |
| | | | catcaactttgtttcctcat |
| 70 | 207843_x_at | CYB5A | gctggaggtgacgctactgagaactttgaggatgtcgggcactctacagatgccagggaaatg |
| | | | tccaaaacattcatcattggggagctccatccagatgacagaccaaagttaaacaagcctccag |
| | | | aaccttaaaggcggtgtttcaaggaaactcttatcactactattgattctagttccagttggtggac |
| | | | caactgggtgatccctgccatctctgcagtggccgtcgccttgatgtatcgcctatacatggcag |
| | | | aggactgaacacctcctcagaagtcagcgcaggaagagcctgctttggacacgggagaaaa |
| | | | gaagccattgctaactacttcaactgacagaaaccttcacttgaaaacaatgattttaatatatctct |
| | | | ttctttttcttccgacattagaaacaaaacaaaaagaactgtcctttctgcgctcaaatttttcgagt |
| | | | gtgcctttttattcatctacttt |
| 71 | 207878_at | KRT76 | gagctcaagccagcatagctccaccaagtgatctactgttccaaatctctataaccacctgcttc |
| | | | ccactcagcctgcaatagtgtttcccactctctgcttggcatcaatagatgcataagggtcaacc |
| | | | acatttttcctcaagttccctggagaagaagctgaactcctggtttctccatccccatgaccttccc |
| | | | agggccatggaggtcctgctgctggtctgggatgatgatgcccctggaaaccttcctgcaatg |
| | | | gccccttactttggacagcaacccctgagcccaagccagttttggccttcacagcctggccggt |
| | | | tcccactctggcccatctcccattcttactgggagttggagatttgaagccagtcatctcagcact |
| | | | gtctgaggagggcagagccatgggttctgtgctggagggtgcacggccaagatctccagact |
| | | | gctggttcccagggaaccctccctacatctgggcttcagatcctgactcccttctgtcccctaatt |
| | | | ccctgagctgtagatcctctggt |
| 72 | 207937_x_at | FGFR1 | cgcacccgcatcacaggggaggaggtggaggtgcaggactccgtgcccgcagactccggc |
| | | | ctctatgcttgcgtaaccagcagcccctcgggcagtgacaccacctacttctccgtcaatgtttc |
| | | | agcttgcccagatctccaggaggctaagtggtgctcggccagcttccactccatcactcccttg |
| | | | ccatttggacttggtactcggcttagtgattagaggccctgaacaggtggtggtatccctgctctg |
| | | | ctggagaggaacccagatgctctcccctcctcggaggatgatgatgatgatgatgactcctctt |
| | | | cagaggagaaagaaacagataacaccaaaccaaaccccgtagctccatattggacatcccca |
| | | | gaaaagatggaaaagaaattgcatgcagtgccggctgccaagacagtgaagttcaaatgccct |
| | | | tccagtgggaccccaaaccccacactgcgctggttgaaaaatggcaaagaattcaaacctgac |
| | | | cacagaattggaggctacaaggtccgttatgccacctgga |
| 73 | 208157_at | SIM2 | ctgccctgtacatgctagttcaacagaaaggaatggcctttcaccttctcctggtggcaggcaag |
| | | | cagatgtcctctgcggagataccgccagctccccaggacgcagactgactcctgtttgctcgct |
| | | | ggaccaaccccaggcagaaggtggaaggtgggaacagaggtttagctgcaggacatgtattc |
| | | | ccattgcaccgagacctaactgccgctcagagtgtagaccgagatggtgcagatgcctgcagt |
| | | | gccattaaaatgtgggtgaaggtgacatcaggattatgtgccccaggccgggctcagtggctc |
| | | | acacctgtaatcccagcactttgggaggccaaggtgggcggatcacctgaggtcaggagtttg |
| | | | cgacaagcctgccaacaagctgaaacc |
| 74 | 208233_at | PDPN | gaaatctctgatataagctgggtgtggtggctcgtgcctgtagtctcagctgctgggcaactgca |
| | | | gaccagcctgggcaacatagtaagaccctgtctcaaaaaaataatctctggtacaatggtcatgt |
| | | | tccaaagttccttacttgggcctcttgagtgcagtggctcacacctggaatcccagtgctttgaga |
| | | | ggctgaggaggcaggaggttcacttgtgcccaggaatttgaggctgcagtgagctatgattgt |
| | | | gccactgcactccagcctgggtgacagagcaagactgtgctctcttaaaaataagaaagagcc |
| | | | tcttcatcttcaaaaggactacatctgaagtttccccagaaggacaaatgtctacttagaccttata |
| | | | aatttccaaaataagagagtcagagccagaggtggcttgtaagttgacttctgttgagatctgac |
| | | | cacatttgatctcttgttttaattttccaactaactgaacttggaagaaaacccaaaccaagttttaat |
| | | | ctgatgccta |
| 75 | 208292_at | BMP10 | ccatgagcaacttccagagctggacaacttgggcctggatagcttttccagtggacctgggga |
| | | | agaggctttgttgcagatgagatcaaacatcatctatgactccactgcccgaatcagaaggaac |
| | | | gccaaaggaaactactgtaagaggaccccgctctacatcgacttcaaggagattgggtggga |
| | | | ctcctggatcatcgctccgcctggatacgaagcctatgaatgccgtggtgtttgtaactaccccc |
| | | | tggcagagcatctcacacccacaaagcatgcaattatccaggccttggtccacctcaagaattc |
| | | | ccagaaagcttccaaagcctgctgtgtgcccacaaagctagagcccatctccatcctctatttag |
| | | | acaaaggcgtcgtcacctacaagtttaaatacgaaggcatggccgtctccgaatgtggctgtag |
| | | | atagaagaagagtcctatggcttatttaataactgtaaatgtgtatatttggtgttcctatttaatgag |
| | | | attatttaataagggtgtacagtaatagaggcttgctgccttcaggaa |
| 76 | 208314_at | RRH | atgatctgcatgtttctggtggcatggtccccttattccatcgtgtgcttatgggcttcttttggtgac |
| | | | ccaaagaagattcctccccccatggccatcatagctccactgtttgcaaaatcttctacattctata |
| | | | acccctgcatttatgtggttgctaataaaaagtttcggagggcaatgcttgccatgttcaaatgtca |
| | | | gactcaccaaacaatgcctgtgacaagtattttacccatggatgtatctcaaaacccattggcttc |
| | | | tggaagaatctgaaataagagaaaaggacacgctatcaaaacactttagttttttgacaatgctttt |
| | | | cttttaaatatgagcccatttagatcaagtgcagacatggatcattgtcctatgagagtgtaagctc |
| | | | ctcaagcacagctcgtgcttccgtttgtgcactctggctgctgtagtgtatgcttctctgtgtcctg |
| | | | atatatcaacttattgctcatctcctttgatgaattaggcatcagaggttaaggtcccctttc |
| 77 | 208368_s_at | BRCA2 | gaacaggagagttcccaggccagtacggaagaatgtgagaaaaataagcaggacacaatta |
| | | | caactaaaaaatatatctaagcatttgcaaaggcgacaataaattattgacgcttaacctttccagt |
| | | | ttataagactggaatataatttcaaaccacacattagtacttatgttgcacaatgagaaaagaaatt |
| | | | agtttcaaatttacctcagcgtttgtgtatcgggcaaaaatcgttttgcccgattccgtattggtata |
| | | | cttttgcttcagttgcatatcttaaaactaaatgtaatttattaactaatcaagaaaaacatctttggct |
| | | | gagctcggtggctcatgcctgtaatcccaacactttgagaagctgaggtgggaggagtgcttg |
| | | | aggccaggagttcaagaccagcctgggcaacatagggagacccccatctttacgaagaaaaa |
| | | | aaaaaaggggaaaagaaaatcttttaaatctttggatttgatcactacaagt |
| 78 | 208399_s_at | EDN3 | ccgagccgagcttactgtgagtgtggagatgttatcccaccatgtaaagtcgcctgcgcaggg |
| | | | gagggctgcccatctccccaacccagtcacagagagataggaaacggcatttgagtgggtgt |
| | | | ccagggccccgtagagagacatttaagatggtgtatgacagagcattggccttgaccaaatgtt |
| | | | aaatcctctgtgtgtatttcataagttattacaggtataaaagtgatgacctatcatgaggaaatga |
| | | | aagtggctgatttgctggtaggattttgtacagtttagagaagcgattatttattgtgaaactgttct |
| | | | ccactccaactcctttatgtggatctgttcaaagtagtcactgtatatacgtatagagaggtagata |
| | | | ggtaggtagattttaaattgcattctgaatacaaactcatactccttagagcttgaattacatttttaa |
| | | | aatgcatatgtgctgtttggcaccgtggcaagatggtatcagagagaaacccatcaattgctcaa |
| | | | atactc |
| 79 | 208511_at | PTTG3 | ttgtggctacaaaggatgggctgaagctggggtctggaccttcaatcaaagccttagatggga |
| | | | gatctcaagtttcaatatcatgttttggcaaaacattcgatgctcccacatccttacctaaagctac |
| | | | cagaaaggctttgggaactgtcaacagagctacagaaaagtcagtaaagaccaatggacccc |
| | | | tcaaacaaaaacagccaagcttttctgccaaaaagatgactgagaagactgttaaagcaaaaa |
| | | | actctgttcctgcctcagatgatggctatccagaaatagaaaaattatttcccttcaatcctctagg |
| | | | cttcgagagttttgacctgcctgaagagcaccagattgcacatctccccttgagtgaagtgcctc |
| | | | tcatgatacttgatgaggagagagagcttgaaaagctgtttcagctgggccccccttcacctttg |
| | | | aagatgccctctccaccatggaaatccaatctgttgcagtctcctttaagcattctgttgaccctgg |
| | | | atg |
| 80 | 208684_at | COPA | ggtttaaggatcagtcctctgcagtttcgctaaggccccctttgtgtgcatgggtcagtcaccata |
| | | | tgttccccccagagaatgtgtctatatcctccttctaacagcaccttccccctgcagctactcttca |
| | | | gatctggctctctgtaccctaaaacctagtatctttttctcttctatggaaaatccgaaggtctaaac |
| | | | ttgacttttttgaggtcttctcaacttgactacagttgtgctcataattgtccttgcctttccagcttaat |
| | | | tattttaaggaacaaatgaaaactctgggctgggtggagtggctcatacctgtaatcccagcact |
| | | | ttgggaggctacggtgggcagatcatctgaggccaggagttcgagacctgcctggccaacat |
| | | | ggcaacaccccgtctctaataaaaatataaaaattagcctggcatggtagcatgcgcctatagtc |
| | | | ccagctgctcaggaggctgaggcatgagaatcgcttgaacctaggaggtggaggttgcattca |
| | | | actgagatcatacc |
| 81 | 208992_s_at | STAT3 | actggtctatctctatcctgacattcccaaggaggaggcattcggaaagtattgtcggccagag |
| | | | agccaggagcatcctgaagctgacccaggcgctgccccatacctgaagaccaagtttatctgt |
| | | | gtgacaccaacgacctgcagcaataccattgacctgccgatgtccccccgcactttagattcatt |
| | | | gatgcagtttggaaataatggtgaaggtgctgaaccctcagcaggagggcagtttgagtccctc |
| | | | acctttgacatggagttgacctcggagtgcgctacctcccccatgtgaggagctgagaacgga |
| | | | agctgcagaaagatacgactgaggcgcctacctgcattctgccacccctcacacagccaaac |
| | | | cccagatcatctgaaactactaactttgtggttccagattttttttaatctcctacttctgctatctttga |
| | | | gc |
| 82 | 209434_s_at | PPAT | ttgacagctctttaagcccacatgcagcagtgggtcagataaccctgtggcagtgacacgggc |
| | | | aaattggcatttgaataaagccctgggaccacctcaacatgcgtagcctcttgtcttaaatgtact |
| | | | ccccatggcagcatggaggaggcaagacctgtgggtcaattttgaactggccttactttgattttt |
| | | | aaaacaagagactcagggaaagtactaaaccaaaatctctgattttactttgcgttttctgtagtttt |
| | | | tgttttactgagatgcttttgtaaaggaaaataatactgtgacagtttagtaattctacagattcttaat |
| | | | atttctccatcatggccttttacttcacaattttctgaagtctgaattcaattacaattttttttttttacca |
| | | | atttaatctcaaatgttgtttaactgctttaaattcatatacgtagagtattataaactgcagagatga |
| | | | aaaatgtgttttcacgggatttatattgtgaactaaactaagcctactttttgtgact |
| 83 | 209839_at | DNM3 | gagacttctcacttctggttggaggtttcacatatggctcaactcaagtcattaatctctttttaatttt |
| | | | tactcttgaattccttaaacttcgctcattatgaaatgttttaaaattatgacaaaaattactctgtcta |
| | | | accacttgccttgtctgctaccagtttgttaaaaattattccccccaaccagtaattccaccagtact |
| | | | acttgatttgtgttatatttcctatgtacatgtacagcctttgttttgcttgcttgtctatttttactttccct |
| | | | tttttgggtcaaatttttcttttgctttgtttgaagaaggaatatacagaagtaaaatcttgtcttctctg |
| | | | ctgattctttaattaatatgagccggatactttccactgtcttcttggcactttcaggatttcttaatgc |
| | | | tgatatatggactcttagaatggaatttttgaagaaaaatctcaaagcctgtatcgttct |
| 84 | 209859_at | TRIM9 | ataggttacccttgaaattcattagtttgtcataaagttttaggaaaggtaggacccggaaagaag |
| | | | ttctaattagttgtctaaatatttttcagtgagccaagaaattcaccatgaaaaaacaagaataaca |
| | | | aatagaagggaagagataggatgggaaagctaacaaattaaagttttggcaaaaaggaatata |
| | | | tgtaaatagctaattatttacttttgtgcttactttatttagattatttctacagttacaatctttttctagtt |
| | | | aagtgtacctaatttatggaatgggtgctatcctgtttatgtgtgtcttggtttttcttggctacagaa |
| | | | aaactgttgcagggcaacactagtttgatatttgatttactctccaatgagactcaatggctgggc |
| | | | cgtggtagactcatagttcctcttgttctttattaaattcatcctgctaattagatttctagtgacttgta |
| | | | acatgtagtttacactgaattgcaattacagatgcatacaactactatacta |
| 85 | 210016_at | LOC100134606 | ataacagcatatgcatttccccaccgcgttgtgtctgcagcttctttgccaatatagtaatgctttta |
| | | /// | gtagagtactagatagtatcagttttggattcttattgttatcacctatgtacaatggaaagggatttt |
| | | MYT1L | aagcacaaacctgctgctcatctaacgttggtacataatctcaaatcaaaagttatctgtgactatt |
| | | | atatagggatcacaaaagtgtcacatattagaatgctgacctttcatatggattattgtgagtcatc |
| | | | agagtttattataacttattgttcatattcatttctaagttaatttaagtaatcatttattaagacagaatt |
| | | | ttgtataaactatttattgtgctctctgtggaactgaagtttgatttatttttgtactacacggcatggg |
| | | | tttgttgacactttaattttgctataaatgtgtggaatcacaagttgctgtgatacttcatttttaaattg |
| | | | tgaactttgtacaaattttgtcatgctggatgttaacacat |
| 86 | 210247_at | SYN2 | tcatgtcttattcttccctgtgaaaccaggattaatcgtggactcctggcagcttaacctagctcag |
| | | | ttgcagtgctaagcatgccccgcccccattcagtgatacctgtttgggaagtatatacttccccaa |
| | | | aagtactcttggccctaagttttaggaactttccccgacctggatcccttgtcatacctgtgttactg |
| | | | tttaaagcacacccacccaacttacaagatcttaggctgctgtggtggtgaagcaccttgagtct |
| | | | gctgatattcgggagaacaaggatctgcagtttccccttttctcccctctgaagagtggttcttatg |
| | | | tgcaatctgcagtaaccttgaactccagagctgcactatagaggagaatgcatgccactatgac |
| | | | agcagtatgccaagctttgtgttcatctcctaata |
| 87 | 210302_s_at | MAB21L2 | atttcgttttgcttttggttgcctgaatgttgtcaccaagtgaaaaaattatttaactatatgtaaaattt |
| | | | ctcttttaaaaaaaagttttactgatgttaaacgttctcagtgccaatgtcagactgtgctcctccct |
| | | | ctcctgaacctctaccctcaccctgagctgtcttgttgaaaacagt |
| 88 | 210315_at | SYN2 | tattctcgactgtaatggcattgcagtagggccaaaacaagtccaagcttcttaaaatgattggtg |
| | | | gttaatttttcaaagcagaaattttaagccaaaaacaaacgaaaggaaagcggggaggggaaa |
| | | | acagaccctcccactggtgccgttgctgcgttctttcaatgctgactggactgtgtttttcctatgc |
| | | | agtgtcagctcctctgtctggttgtttacctgttcctgttcgtgcttgtaatgctcacttatgttttctct |
| | | | gtataacttgtgattccagggctgtttgtcaacagtatacaaaagaattgtgcctctcccaagtcc |
| | | | agtgtgactttatcttctgggtggtttg |
| 89 | 210455_at | C10orf28 | gaaatcagcgaggctcaagttccaagcaaaccattccaaaatgtggaattctgtgacttcagta |
| | | | ggcatgaacctgatggggaagcatttgaagacaaagatttggaaggcagaattgaaactgata |
| | | | ccaaggttttggagatactatatgagtttcctagagtttttagttctgtcatgaaacctgagaatatg |
| | | | attgtaccaataaaactaagctctgattctgaaattgtacaacaaagcatgcaaacatcagatgg |
| | | | aatattgaatcccagcagcggaggcatcaccactacttctgttcctggaagtccagatggtgtct |
| | | | ttgatcaaacttgcgtagattttgaagttgagagtgtaggtggtatagccaatagtacaggtttcat |
| | | | cttagatcaaaagatacagattccattcctgcaactatgggtcacatctctctgtcagagagcaca |
| | | | aatgacactgttagtccagtaatgattagagaatgtgagaagaatgacagcactgctgatgagtt |
| | | | acatgtaaagcacgaacctcctgatacag |
| 90 | 210758_at | PSIP1 | gggctcaaagcattaatccagttactgaaaagagaatacaagtggagcaaacaagagatgaa |
| | | | gatcttgatacagactcattggactgaatttcccccttccccccatgatggaagaatgttcagattc |
| | | | taaattgaggacttcattattaatggcattactgtgttatgattaacaaatttcttgtaaggtacacac |
| | | | tacatactaaggtcggccatcattccgtttttttttttttttttttttttaaccaagcttaaaatgaagctta |
| | | | aaatgaagctttgtgtttgaaagtaataacaagctcagacgaagatggtggttgtacattattcatc |
| | | | tagaaaatataaaaattcattttgttttgaagctagttattaaactggaatagcagttatatccctgag |
| | | | aatggggccctt |
| 91 | 210918_at | — | gctgctgttttcttctaactgcagggaaaatgctgtctaaaagaaaataataaatttgtatctgctga |
| | | | gttctcttagcataaggcaccaacaaaacaaccttcaggaagggagaagaaaccatcctccca |
| | | | ctcatccttcagaggatttagataaagtgaagggaagaatcgttctccagctccttcggaatttac |
| | | | gccggcatcagggcaggcttgttactgctggatccattgtctgctcaaggttacttattccactaa |
| | | | gacgtacatcctaccacggaccacggctttgtagctagccaggctctgagtgtgtgtgtagatg |
| | | | aaccatttctctctccagtaaatgaatgacagtctttctagggctcttgtcttctgctgggaggcag |
| 92 | 211204_at | ME1 | agtcactctcccagatggacggactctgtttcctggccaaggcaacaattcctacgtgttccctg |
| | | | gagttgctcttggggtggtggcctgcggactgagacacatcgatgataaggtcttcctcaccac |
| | | | tgctgaggtcatatctcagcaagtgtcagataaacacctgcaagaaggccggctctatcctcctt |
| | | | tgaataccattcgagacgtttcgttgaaaattgcagtaaagattgtgcaagatgcatacaaagaa |
| | | | aagatggccactgtttatcctgaaccccaaaacaaagaagaatttgtctcctcccagatgtacag |
| | | | cactaattatgaccagatcctacctgattgttatccgtggcctgcagaagtccagaaaatacaga |
| | | | ccaaagtcaaccagtaacgcaacagcta |
| 93 | 211264_at | GAD2 | gttccacttctctaggtagacaattaagttgtcacaaactgtgtgaatgtatttgtagtttgttccaaa |
| | | | gtaaatctatttctatattgtggtgtcaaagtagagtttaaaaattaaacaaaaaagacattgctcct |
| | | | tttaaaagtcctttcttaagtttagaatacctctctaagaattcgtgacaaaaggctatgttctaatca |
| | | | ataaggaaaagcttaaaattgttataaatacttcccttacttttaatatagtgtgcaaagcaaacttta |
| | | | ttttcacttcagactagtaggactgaatagtgccaaattgcccctgaatcataaaaggttctttggg |
| | | | gtgcagtaaaaaggacaaagtaaatataaaatatatgttgacaataaaaactcttgcctttttcata |
| | | | gtattagaaaaaaatttctaatttacctatagcaacatttcaaat |
| 94 | 211341_at | LOC100131317 | gcatttgaaactgagcactaaactgggctagctttctggtagaccgttttgtggctagtgcgatttc |
| | | /// | acagtctactgcctgtttccactgaaaacatttttgtcatattcttgtattcaaagaaaacaggaaaa |
| | | POU4F1 | aagttattgtaaatattttatttaatgcacacattcacacagtggtaacagactgccagtgttcatcc |
| | | | tgaaatgtctcacggattgatctacctgtctatgtatgtctgctgagctttctccttggttatgttttttc |
| | | | tcttttacctttctcctcccttacttctatcagaaccaattctatgcgccaaatacaacagggggatg |
| | | | tgtcccagtacacttacaaaataaaacataactgaaagaagagcagttttatgatttgggtgcgtt |
| | | | tttgtgtttatactgggccaggtcctg |
| 95 | 211516_at | IL5RA | ggcagccttccttgtgatcaaaaaaggtaatcccagaaacgtacccgttcactcgtgggtcttaa |
| | | | aatggtttcatatctctattgtgactaattttctctcggtctactgccttttcaatcaggaatagatttg |
| | | | ccatgaagccagtgaagtttttaagtgtctaggcttctcattagtgccaactctcctagacctggtg |
| | | | cctgttttttttccaagttttgtttctacttctatccattttttaaattaaactttttattttgaaataattat |
| | | | cacactcacaagctgtgggaagaaataatagagatcctgtgtctctttcatccagttttcctcaaggg |
| | | | taacatct |
| 96 | 211772_x_at | CHRNA3 | tgctcaacgtgcactacagaaccccgacgacacacacaatgccctcatgggtgaagactgtat |
| | | | tcttgaacctgctccccagggtcatgttcatgaccaggccaacaagcaacgagggcaacgctc |
| | | | agaagccgaggcccctctacggtgccgagctctcaaatctgaattgcttcagccgcgcagagt |
| | | | ccaaaggctgcaaggagggctacccctgccaggacgggatgtgtggttactgccaccaccg |
| | | | caggataaaaatctccaatttcagtgctaacctcacgagaagctctagttctgaatctgttgatgct |
| | | | gtgctgtccctctctgctttgtcaccagaaatcaaagaagccatccaaagtgtcaagtatattgct |
| | | | gaaaatatgaaagcacaaaatgaagccaaagaggaacaaaaagcccaagagatccaacaat |
| | | | tgaaacgaaaagaaaagtccacagaaacatccgatcaagaacctgggctatgaatttccaatct |
| | | | tcaacaacctgtt |
| 97 | 212359_s_at | KIAA0913 | cagcgctgccagcaggcatacatgcagtacatccaccaccgcttgattcacctgactcctgcg |
| | | | gactacgacgactttgtgaatgcgatccggagtgcccgcagcgccttctgcctgacgcccatg |
| | | | ggcatgatgcagttcaacgacatcctacagaacctcaagcgcagcaaacagaccaaggagct |
| | | | gtggcagcgggtctcactcgagatggccaccttctccccctgagtctttcacccttagggtccta |
| | | | tacagggacccaggcctgtggctatgggggcccctcacacagggggagtgaaacttggctg |
| | | | gacagatcatcctcactcagttccctggtagcacagactgacagctgctcttgggctatagcttg |
| | | | gggccaagatgtctcacaccctagaagcctagggctgggggagacagccctgtctgggagg |
| | | | gggcgttgggtggcctctggtatttattt |
| 98 | 212528_at | — | gtcactcatttccttgaacagcacccccctttatactagcagccatttgtgccattgcctgtgccct |
| | | | agggtttgtggggagagagcgagggatcactgagcagttttcccagagctccatgggaaggc |
| | | | aagctctccctcccaatgggagccccactgtcactaactgtaaactcaggctcaggcttcaact |
| | | | gcctacccccatcctcatatttctgtctgtcccagcacctcaggagcattctcattgtggccggct |
| | | | aactccgcctggatgtgaacaggcaagcacagtgggaaatgagtcacgtacttgtattgcaca |
| | | | gtggacacctctagaggtccattggtttaaagggatagggaaggaggagggatgagaccatc |
| | | | accccctcccagaagtaaatctagtatctgagttttctttat |
| 99 | 212531_at | LCN2 | caagagctacaatgtcacctccgtcctgtttaggaaaaagaagtgtgactactggatcaggactt |
| | | | ttgttccaggttgccagcccggcgagttcacgctgggcaacattaagagttaccctggattaac |
| | | | gagttacctcgtccgagtggtgagcaccaactacaaccagcatgctatggtgttcttcaagaaa |
| | | | gtttctcaaaacagggagtacttcaagatcaccctctacgggagaaccaaggagctgacttcg |
| | | | gaactaaaggagaacttcatccgcttctccaaatctctgggcctccctgaaaaccacatcgtctt |
| | | | ccctgtcccaatcgaccagtgtatcgacggctgagtgcacaggtgccgccagntgccgcacc |
| | | | agcccgaacaccattgaggga |
| 100 | 213197_at | ASTN1 | tttccccttggaagacactattgatctcaacctgctgacttttcctaatgcttacctgaaggaaccc |
| | | | atcctggctagaaagggtgatggtactggaccggtattcaaccttgagttttcaagctgccaaac |
| | | | aggtcttaagggaggtgcttatatcccaccaacactctcccagctcccatgtccccaagacctct |
| | | | ggagtttcctcttgaatgtacatgaaccactgtaatagcattagacttttaattgagtgtgcaatcgt |
| | | | tttccatggagtttggtccgttcattattttttagttaactacacttcttgatattcaaatgttctattaaa |
| | | | aaaactgagtatgaagaaaaacactttactactgcagaa |
| 101 | 213260_at | FOXC1 | tcccccatttacaatccttcatgtattacatagaaggattgcttttttaaaaatatactgcgggttgga |
| | | | aagggatatttaatctttgngaaactattttagaaaatatgtttgtagaacaattatttttgaaaaaga |
| | | | tttaaagcaataacaagaaggaaggcgagaggagcagaacattttggtctagggtggtttctttt |
| | | | taaaccattttttcttgttaatttacagttaaacctaggggacaatccggattggccctcccccttttg |
| | | | taaataacccaggaaatgtaataaattcattatcttagggtgatctgccctgccaatcagactttgg |
| | | | ggagatggcgatttgattacagacgttcgggggggtggggggcttgcagtttgttttggagata |
| | | | atacagtttcctgctatctgccgctcctatctagaggcaacacttaagcagtaattgctgttgcttgt |
| | | | tgtca |
| 102 | 213458_at | FAM149B1 | agcctgaaacaggaactcacatgagactcagggccaccaggaaatgcttaaaatacatactctt |
| | | | tcccaaaagcaaatctataattctgtttcaattttatgaatatatgaatagacaaaatgaatcgaatt |
| | | | acataactatgtcattcattaaatggcaacaatgctgacagcaagcagtagatcctctgattccaa |
| | | | ttaccatttgttttttacccaattctatttgctagaggtagtaagtactctggcactcataaatcacat |
| | | | gatgataaaaaggaacatgaggccgggtatggtggctcacaactgtaatccccataccttggg |
| 103 | 213482_at | DOCK3 | tatgggtcagttacagcagccctcacctcaaagggctggcctgcttctcagcctacattcatttgc |
| | | | aagcttcaatctctggaccatctggtgttcacaggtgttagagggttaggggttaggggctagttt |
| | | | tggatttgattcataggtaggagggcttagattttaaggcacttctgaaagtcaatccctggacaa |
| | | | ggcagtcatcacataagaacagctaccttctccacttggtggcacaagaggtagggagggga |
| | | | gtatgggttcatttgncttcgcattatgcaaggtgaaaccgtttgttttccctctccattttccctaac |
| | | | taaatgaaaaggacacattctgaaatcccttttgttggagaataagtcagtctgaggggaaatgg |
| | | | gaggccagagatgagaaccctttgaaaagattgtaaaatactgattttcattctttcaagcttatttg |
| | | | taaatacctatttgaatgctgtgtatttgtacaggaatttgagcaaaaaatgtatagagtgtgatgtc |
| | | | caattggtattcagcactat |
| 104 | 213603_s_at | RAC2 | gagcttcgttgatggtcttttctgtactggaggcctcctgaggcnnnnnnagccccaggaccc |
| | | | attaagccacccccgtgttcctgccgtcagtgccaactnnnnnatgtggaagcatctacccgtt |
| | | | cactccagtcccaccccacgcctgactcccctctggaaactgcaggccagatggttgctgcca |
| | | | caacttgtgtaccttcagggatggggctcttactccctcctgaggccagctgctctaatatcgatg |
| | | | gtcctgcttgccagagagttcctctacccagcaaaaatgagtgtctcagaagtgtgctcctctgg |
| | | | cctcagttctcctcttttggaacaacataaaacaaatttaattttctacgcctctggggatatctgct |
| | | | cagccaatggaaaatctgggttcaaccagcccctgccatttcttaagactttctgctccactcaca |
| | | | ggatcctgagctgcacttacctgtgagagtcttcaaacttttaaaccttgccagtcaggacttttgc |
| | | | tattgcaaatagaaaacccaactcaacctgctt |
| 105 | 213917_at | PAX8 | ctgcctggttaccgtggcgatgtgcttaatgcagcgttgaaaatacagaatactgactcctctgtc |
| | | | cctcctggccccggactccctccctccctcccttcctcttctggagcgtgaaatgagattggtca |
| | | | agataaaaaaggaaaagattcggttatttttttaagagtgtggataatggggcctctcaatcaaaa |
| | | | tcccagtctccagtcggttccccccattccccttccaacccctccaccttcccctgccgcctgctt |
| | | | agaggaggaggaagaaacataaagcacaaggcttttctcttaattatgaatcattccctgaggg |
| | | | caggcccagggcaaggggttcctggggcccagagtctgacctgtgaggtagctagaaggctt |
| | | | gagcctctcatcaaagtcc |
| 106 | 214457_at | HOXA2 | ctttgcaggactttagcgttttctccacagattcctgcctgcagctttcagatgcagtttcacccagt |
| | | | ttgccaggttccctcgacagtcccgtagatatttcagctgacagcttagacttttttacagacacac |
| | | | tcaccacaatcgacttgcagcatctgaattactaaaaacattaaagcaaaacaaagcatcacca |
| | | | aacaaaaactcctttgaccaggtggttttgccttcttttatttgggagtttattttttattttcttcttgac |
| | | | ctaccccttccctcctttaagtgttgaggattttctgtttagtgattccctgacccagtttcaaacaga |
| | | | gccatcttttacagattattttggagttttagttgttttaaacctaactcaacaaccctttatgtgattcc |
| | | | tgagagc |
| 107 | 214608_s_at | EYA1 | gtcaccctgaggaaggttcattgccattgtcatcaccatggaaacaacgttcctctccacctgca |
| | | | ttatgtactacatgacaggcatcaatctggggaaataataaaattatcacctttgtcagaccataa |
| | | | gagtttctccaaaagtggtcagtttggctgggcaatatttnctctcatctaacaaacacaatccatt |
| | | | gtcatgaaattacccttaggatgagtcttctttaatcaatcatatattgggcggaaaaaacaccag |
| | | | ctttgacccgaagtagttgaagagctacttcattcttttctgaagttgtgtgttgctgctagaaatag |
| | | | tcatttgtgaattatccaaattgtttaaattcacaattgaattagttttttcttcctttttgcttgaagcaa |
| | | | acagttgacaatttttaaccttttcattttatgtttttgtactctgcagactgaaaagacaaagtttatct |
| | | | tggccttactgtataaaggtgtgctgtgtccaccgttgtgtacaga |
| 108 | 214665_s_at | CHP | gaggtctggcactagtagcacaacctaaggtggcattacagatctttgagcgagccacagcaa |
| | | | cttttctgccaagtcagcttnagttnagacttcagtgaatcaggntattgctatcctaatgtatgtct |
| | | | ctatgagtgtatntagccacanantctgcccttggttgantttctgactcattgcttgcttgcttgttt |
| | | | ccttgctttggaaaactatnnaagattgctaaaaaataccactgcaaagtgatggaaaagggtg |
| | | | gagaacaggggagtagccaggctggatggctcaaatataaatgaatgaggaattctttatgaa |
| | | | gtatcagtcagattttatgattaagtgatgtaatataggaattatgtaaaagggaagaatgtctgat |
| | | | actgatctattagagaggtactttagaggcttcttgattggcataaagttcctaaggttatagatttt |
| | | | ccccccttttggctgtatagcaaagtgttttaatccacggttgtgccttattgttccattaaaa |
| 109 | 214822_at | FAM5B | caatgggaggggtcggagctcttccttcccctctgtggagtcacttttgtattctttttaaccagatt |
| | | | tcttaaaatgttgttgttttgtgaatcctgacattggttcttacttttgtatgctgcctcctctgtgccct |
| | | | cccagacgctgactgggaaacacaagaagtacaaccaacaggaaccagcgccaagggcag |
| | | | gcagcggcctccttgctcccctcccttactcctccctctgctgcctcctccccccaccaagtttca |
| | | | gggccctggattgttcccagttcccattgtggtcccttcagagctcctttccaacagcatctctctg |
| | | | tcgaagaaagaagctctgtcaagttagagagagacaatgtgtaggaaatgttcttttttaaaaaa |
| | | | aaataacaaaaacaaaacaaaactatnnannntgtgattgttttccttgttaatctgctccaacca |
| | | | cctgaacatctaagta |
| 110 | 215102_at | DPY19L1P1 | gagacgggagtttaccccgatcacagaaaccataccaactgaaagacaaatcagcatcttgct |
| | | | ggacgacccctcacagagctcctagatccttgaagtgtgaacttcagcagctgagagagatgg |
| | | | ggtctcactatgttgcccaggctggtcttgaactcctggactcaagcaatcctctcacctcagcct |
| | | | cccaaagtgctgggattacagattttataaatattgttgatctttttgaaaaaccaactgttggcttc |
| | | | attttntttattgtgtaatactaccttagaggacagcagttcctaatacctacttttattatgagtctct |
| | | | gccatttataaagaactgtggacagcacagggaatgggggaagaaaactctggtgcagcttga |
| | | | atcttggtagcaaaacagtgacttcatcagaaaattttgtcactctctattagatataatggagtttg |
| | | | accatttggaatttggaatttttcaaatgaatatgacaaaaatttaaaaaactcttgtattactatgtg |
| | | | ataacacagatctttacaacttta |
| 111 | 215180_at | — | aagccttcaccagatggtcaagcagatgctggtgccatgcccttgancntcncnccaccatcc |
| | | | cccacctagccactatatgggttgttagatattttgaccacctcctcttdnctcactccactattcaa |
| | | | ctcactgcatcatcaatgtacttattacaaacctgtcacaagccaggtcttatgctaggtgctcctc |
| | | | tcaacaggttcttgagctggcaggggagagagagacattcaaacaccaaggattaatatacca |
| | | | ttacaggtttaaagacagaggcctataagggtcccctggcagtgccatggaggtagggcatgg |
| | | | tcggctgtacctgtagaggtgtctaaagggaggcttgcaagctgccccttgaaggacgagcag |
| | | | aaaattgtacatgaggacaagtaggaaaggaattccaggaggagggatcagcatgtgca |
| 112 | 215289_at | HLA- | ggactaaatcgagccttattatacatcagcagtctcacactggagaaagtccttttaagttaaggg |
| | | DRB1 /// | anngnnnnnnannntnnancaaatgtaatactggtcagcgccaaaaaactcacactggaga |
| | | HLA- | aaggtcttatgagtgtggtgaatccagcaaagtgtttaaatacaactccagcctcattaaacatca |
| | | DRB2 /// | gataattcatactggaaaaaggccttagtggagtgaatgcaggaaagtcaccaaaactgtcacc |
| | | HLA- | tcattcagcaccaaaaggttcacatcggaccaagaacctattaatatatgtaaatctaatgttgaa |
| | | DRB3 /// | agagttcagatggaaatctgcgaggatttcctgctgggaactacatta |
| | | HLA- | |
| | | DRB4 /// | |
| | | HLA- | |
| | | DRB5 /// | |
| | | LOC100133484 | |
| | | /// | |
| | | LOC100133661 | |
| | | /// | |
| | | LOC100133811 | |
| | | /// | |
| | | LOC730415 | |
| | | /// | |
| | | RNASE2 | |
| | | /// | |
| | | ZNF749 | |
| 113 | 215356_at | TDRD12 | aattgggcaggctcttgggaagtagaaagttctggtgtttttgctggtgaaggttttgactgtgga |
| | | | gctcttctaacacccatatcagtgtctgtttctctgcatgtggctgctgccctgttggtggagctct |
| | | | gggggcagagaccaggccgccgtccagtggcgcnccgtgcgcaccagctgcctgctgttta |
| | | | cacccaggtgcgccgagtctctttcatacagcacagcaaatgataatagctagtgacaatgtgtt |
| | | | tcctgtgcactcgtgaaaatgcagggaggacaactgcatgcttagatctgtttcttttttcagacat |
| | | | tcaaatgttctaatatctgaagctaacattttgtaggatataggatgctgattatgtgaacaattagt |
| | | | cattggttttctgtactgctatgaatatgtctgatttcaagttttggtcaaatatctaaaatgcaaggt |
| | | | gaaagtgcctttgtctctatgcttctaaaatcgctcatgcttagttgtggtatggatgtcttccgcag |
| | | | tg |
| 114 | 215476_at | — | cttggtaagccttgcctgtagcggctccgctgccgagtgctttgacaccaggcgctcccagag |
| | | | ctctgcccccactgccaagcggcagctgctccggagggcacggggggctggatttggctgtg |
| | | | gcttctccagctctgcacaagagccccccttccctggccctgctgcagcatgactgcctcctgg |
| | | | ctcgtgtcacccactctgtctctgtctctcttcatacgtttccagctgagctgggatccatagtctgt |
| | | | ttccctctccacgaccaatctatttatcttctctggaacttcttgtaatgccgggagtgcagagctta |
| | | | caagttggggcaggaagctttagaagcccaggnagccctgagaggctctttccttgtaagtgg |
| | | | gtctctccccaggagcctcttggaatatttagcagggacttttacccatgctgggtctagagacc |
| | | | ctcccgcccctctgtttcctgccctcctacttagactgggatctggtttccctcagctggttcccttg |
| | | | ctagcgtgtgactctgtgtgtct |
| 115 | 215705_at | PPP5C | gttcacagcagtgggtaggcccagcagtggttcttgacatcacacgatgaggcgngcatctcc |
| | | | cgtcatccagggagaccagaggacccttgtctcactcccagttggctnttagtcacagccccg |
| | | | ctttgtctttgacatggacgtttgtgatgatcacgttcctcccgctccccgtgtntgaagagtgctc |
| | | | cctgactggctgccgtctcctccctgtcgggtctggctgggttctccanagggagtgctgcgga |
| | | | ggggacacagcanaggccccatgctcgtgatgtatgttgcagatcattttcccccattctgtcctt |
| | | | ttttgttaaattgtggtaaaaagcacataacataaactgtaccnccttaaccatttgaaagtatatat |
| | | | cccagactgtcttttatctttagacttcacttgtggtttgttgcc |
| 116 | 215715_at | SLC6A2 | tcccctggaagttgtcctttctgatcctctcttcttttcccatttacaaatgatttcgtgactgtagttttt |
| | | | gttcaccttctgtgcatctggcctgggggctgttagctcagaggagaggagcaaacaggaaaa |
| | | | tgacttctgttctgtccccgctgttttgggggaagtctctcccactttgggatcctgctgaagctag |
| | | | gttcatgaggtcggaaatccccaccacatttgcctagactttgggcacaggagttcttagtccac |
| | | | caaatcaga |
| 117 | 215850_s_at | NDUFA5 | cattttctctaactttatctcctatgcatttccttatgtgtcctgtacagcagtatattccaaaatcccc |
| | | | agtggatgtctgaaaaccacatatagtaccaaactgtatatatgctatgttttgtttcatacatacct |
| | | | ataataaagtttaatttatgaattaggcacaataagagataagcaggctggacgtgctggctcac |
| | | | gcctgtaatcccagcactttgggaggctgaggcgggtggattgctttagcccaggagtttaaga |
| | | | ccagcctggccaacatggcaaaaccccgtctctataaaaaatgtggaaattaatcaggtgtggt |
| 118 | 215944_at | — | gagatgaccgaaaacttcaacccctgcagtcagcaatggtcaacagaaagggcccaattctcc |
| | | | acgacaatgcatgatcgcacattacacaactaaagcttcaaaagttgaactaactgggctacga |
| | | | agttttgcctcatccaccatattcacctgacctcccgccaaccgactaccacttcttcaatcatctc |
| | | | gacaactttttgcaaggaaaacacttccacaaccagtagaatgcaaaaagtgctttccaagagtt |
| | | | cactgaatcctgaagcacggatttttatgctacaggaataaacaaacttatttttcattggtaaaaat |
| | | | gtgttgattgtaatggatcctattttgattaatgaagatgtgtttgagcctagttataatgatttaaaat |
| | | | tcacgatccaaaaccgcaattacttttgcatcagcctaatatgaggaagtaatagttgaacagaat |
| | | | aattctttcctggaagtct |
| 119 | 215953_at | DKFZP564C196 | ttggtttggtctggtttggctacctgattcctgctgtctttttctacgccaggtgaagaggcactttc |
| | | | aagatccttctctgagacctgcaccaataagactataccaatgttcagttgaaacatcaggtataa |
| | | | gtttagcggaaacgaaagtacaacctgctttgaaataaattccaaggacagattgtcattaacga |
| | | | aatagaaagtggactatgcccctcatgctgccagcgcctggtatgatgcggcgtgacacgcag |
| | | | cgcttgcggcagtacaatgcccccaatcacccgccccgccccgacgcgccgcccactcacg |
| | | | gcaaagagagccacctagtgagggattattctcatttccgcggtggggttctgcttttctttctacc |
| | | | atgagcgcccaaggatagacactcctactacctattacctcaaatagcctacatttctttccgaa |
| 120 | 215973_at | HCG4P6 | agaacactgagcgaggctctgtagatggatgtaataaaaatctataaaacaatgtgtttaaacct |
| | | | aagaattctactgctttccaattccttccctctgctccttttcctaacctcctgcttctccagcccttcc |
| | | | ctctgtccctttcanccctcaggccctcctctccccttagtccccaccaccctgtcacttctaaatt |
| | | | gtggctctagcattgtcccattacctgctangtgactgttctctccacagtggtcctgctcctgtga |
| | | | gtcagagtgtgtcatttcctcacctaaaacactccagtggctccacctcggtcttgtgaagcttct |
| | | | agaatgtcaggcacgtgagcatatgagggcatacctggttcatcttaggcactaaattnnnnttt |
| | | | gttgactgaatgaatgaaatatgaatgtattaaattgcatcacagaaagttataaaatgtaaaaca |
| | | | ctgaaaaattaagaaatattttatnttatgtaactagtgtgcatatcaattcattccgagtctgttgag |
| | | | cctgtgtat |
| 121 | 216050_at | — | aatgattcaactcatgtgatccagtgttacattcagtgtggtaatgaagaacagtcaaaacaggct |
| | | | tttgaagaattgggagataatttggttgaattaagtaaagccaaatactccagaaatattttaaaga |
| | | | aatgtctcacgttgtgaacatgtaccctagaacttaaagtataataaaaaaaaaaaaaanngga |
| | | | aagtatcttgcacaagctcacgtagctggtaagttacatagttgggatctgaattcagttgtggctt |
| | | | catgcctgagcttttaactactactactaaactgagaaggcacttgcttgagtaaattatgtcatcc |
| | | | tcttaat |
| 122 | 216066_at | ABCA1 | gatgtggcatgtgatgacattgcacatggncagttaantgngccaagaagngcagcagtagc |
| | | | agcaacnggagatgcaaagcccaacatgatggggagagaaantnttctttcaatatgtgcttct |
| | | | gtaccaaaagtggaatttcacgagagacatattttggaacatttttccttttgtgtgtgcgtgagtgt |
| | | | ttccctgtttccagccaagggtattgtgagtttctcctgggcctccttcagaatctgggtgctctgg |
| | | | aaagcagtgttttggcaacatggggaaagtatggcagtgtgggagggtcagctgggtctgggt |
| | | | ttgaatattgcatttgaatattttaccagcattgatgtcggataaattatttagtccctgtaagcctca |
| | | | gttttntcttnttctacatacacataatatatttgactctttgttgtgat |
| 123 | 216240_at | PVT1 | tttcctaactttctgatcccttggaggtgataatcaaatattctagtctgaggcattgggatacatgg |
| | | | tgctaggttctgagactctgcgtcaggcctgaaccctgcattttgtggaggtgggtgggagaat |
| | | | gtncccctggggaacatgcctagacacgggggacaacagttgccctcatggggaggtacctg |
| | | | tttactcgctgttatgggaccgctttcacaaaaccactgcaggtgagtgagttcctgctgaatatc |
| | | | aggcctggtgtctctagactcattattncccccacccaacccctatgttagttcatctcgagccac |
| | | | atttttattgccataatccaggcctggacaggccaagatcttttaacaattttaattactgaaaataa |
| | | | taactgcattttttttnaaagcccaacttttnggtanagtcagcccaaaatacagtctttgtgttgcc |
| | | | atctgggaactggatttggaattgttcttccatgagactgcagagcag |
| 124 | 216881_x_at | PRB1 /// | ccacctcctccaggaaagccagaaagaccacccccacaaggaggtaaccagtcccaaggtc |
| | | PRB4 /// | ccccacctcatccaggaaagccagaaggaccacccccacaggaaggaaacaagtcccgaa |
| | | PRH1 /// | gtgcccgatctcctccaggaaagccacaaggaccaccccaacaagaaggcaacaagcctca |
| | | PRH2 /// | aggtcccccacctcctggaaagccacaaggcccacccccagcaggaggcaatccccagca |
| | | PRR4 | gcctcaggcacctcctgctggaaagccccaggggccacctccacctcctcaagggggcagg |
| | | | ccacccagacctgcccagggacaacagcctccccagtaatctaggattcaatgacaggaagt |
| | | | gaataagaagatatcagtgaattcaaataattcaattgctacaaatgccgtgacattggaacaag |
| | | | gtcatcatagctctaac |
| 125 | 216989_at | SPAM1 | gtttgatgtctattatctcacttcatcctcaccaggaccccatccgagccttaatttcagttgacagt |
| | | | aactattggatccccaggaatatgtttgcatatttggggagaaaatactattggaggggaacaga |
| | | | aatgctactaagggtctcactgtgtcacccaggctggagtccatcaaagctcactgcagcctta |
| | | | accttctgtgctcaagggatcctcccacttaagcctcctgagtagctggaactacaggcatatgc |
| | | | caccgagcctggctaatctttgatttttttgtacagattgtgtctccttatgttgctcaggctggactc |
| | | | aaacttctggtctcaagcgatctttccatcttagcttcccaaattgttggaattatggacatgagcc |
| | | | agtgtgcttggcctgattttttttttttttttaatgagaaaaacgttccttaagaaaagtttcattgtaag |
| | | | acgaggacttgctatgttgccagtttggtcttgaactcggtctcaagtgattctcctgccttgggtt |
| | | | cccaaagcgtttgggccggcagatgt |
| 126 | 217004_s_at | MCF2 | ctgaattggaacacaccagcactgtggtggaggtctgtgaggcaattgcgtcagttcaggcag |
| | | | aagcaaatacagtttggactgaggcatcacaatctgcagaaatctctgaagaacctgcggaatg |
| | | | gtcaagcaactatttctaccctacttatgatgaaaatgaagaagaaaataggcccctcatgagac |
| | | | ctgtgtcggagatggctctcctatattgatgaagctactatgtcaaatggcaagtagctctttcctg |
| | | | cctgcttctcagctcatttggaaaaatactgcgcaaaagacattgagctcaaatgatgcagatgtt |
| | | | gttttcaggttaatggacacgcaaagaaaccacagcacatacttcttttctttcatttaataaagctt |
| | | | ttaattatggtacgctgtctttttaaaatcatgtatttaatgtgtcagatattgtgcttgaaagattctca |
| | | | tctcagaatacttttggact |
| 127 | 217253_at | SH3BP2 | gagtgtcttgactattctggctctttgtattttcatgtaaggtttttctcccatataagttttaaaatcag |
| | | | cttgtcaattccaacaacaatgatgcacttgatagtttgggaatttattatagctatcaatcagttttg |
| | | | ggaaaattgacgtctttacaatattgagttttctgattcatgaacatggtttacctctcttcccatggg |
| | | | ggtctcctttaaggtttaccaataggattttatatttggggccattgnggtcttgcttatcttaagtnn |
| | | | nnnnnnnnnnnnnaaatctcttgaccncatgatctgcccgccttgtcctcccaaagtgctgg |
| | | | gattacaggcgtgagccaccgcacctggcctgcaatacagtattgttaaccgtcttcaccatgtt |
| | | | gtacgttagagctccagaaattatttancatgcataactgaaactttatactctttgaacaccacct |
| | | | ccccatttccctctcccggcagccatttgtgcctctcggttctctttattagcttccattttgtgggtc |
| | | | agt |
| 128 | 217995_at | SQRDL | tacgtcaaagaccgctgctgcagtagctgcccagtcaggaatacttgataggacaatttctgtaa |
| | | | ttatgaagaatcaaacaccaacaaagaagtatgatggctacacatcatgtccactggtgaccgg |
| | | | ctacaaccgtgtgattcttgctgagtttgactacaaagcagagccgctagaaaccttcccctttga |
| | | | tcaaagcaaagagcgcctttccatgtatctcatgaaagctgacctgatgcctttcctgtattggaa |
| | | | tatgatgctaaggggttactggggaggaccagcgtttctgcgcaagttgtttcatctaggtatga |
| | | | gttaaggatggctcagcacttgctcatcttggatggcttctgggccaaaactgcagtcactgaat |
| | | | gaccaagagcagcacgaaggacttggaacctatccttgtaaagagttccttgatgggtaatggt |
| | | | gaccaaatgcctcccttttcagtacctttgaacagcaaccatgtgggctactcatgatgggcttgat |
| 129 | 218768_at | NUP107 | ttggatgccctaactgctgatgtgaaggagaaaatgtataacgtcttgttgtttgttgatggagggt |
| | | | ggatggtggatgttagagaggatgccaaagaagaccatgaaagaacacatcaaatggtcttac |
| | | | tgagaaagctttgtctgccaatgttgtgttttctgcttcatacgatattgcacagtactggtcagtat |
| | | | caggaatgcctacagttagcagatatggtatcctctgagcgccacaaactgtacctggtattttct |
| | | | aaggaagagctaaggaagttgctgcagaagctcagagagtcctctctaatgctcctagaccag |
| | | | ggacttgacccattagggtatgaaattcagttatagtttaatctttgtaatctcactaattttcatgata |
| | | | aatgaagtttttaataaaatatacttgttattagtaattttttcttttgcattaccatgtaaaatttagaca |
| | | | tttgaattttgtacttttcagaatattatcgtgacactttcaacatgtagggatatcagcgtttctctgt |
| | | | gtgct |
| 130 | 218881_s_at | FOSL2 | aggtcacagtatcctcgtttgaaagataattaagatcccccgtggagaaagcagtgacacattc |
| | | | acacagctgttccctcgcatgttatttcatgaacatgacctgttttcgtgcactagacacacagagt |
| | | | ggaacagccgtatgcttaaagtacatgggccagtgggactggaagtgacctgtacaagtgatg |
| | | | cagaaaggagggtttcaaagaaaaaggattttgtttaaaatactttaaaaatgttatttcctgcatc |
| | | | ccttggctgtgatgcccctctcccgatttcccaggggctctgggagggacccttctaagaagatt |
| | | | gggcagttgggtttctggcttgagatgaatccaagcagcagaatgagccaggagtagcagga |
| | | | gatgggcaaagaaaactggggtgcactcagctctcacaggggtaatca |
| 131 | 218980_at | FHOD3 | gcacctcggagttgcagctgtgacactcataggttactcccaggagtgtgctgagcagaaggc |
| | | | aagctcttgctggatgaaacccctccaggtggggttggggagacttgatattcacatccaacag |
| | | | tttgaaaagggagagctcaattcccagcgtcaccccatggcttgtgttgcctgctacgcattgac |
| | | | ttggatctccaggagtcccctgcacataccttctccatcgtgtcagctgtgtttctcttgattccgtg |
| | | | acacccggtttattagttcaaaagtgtgacaccttttctgggcaaggaacagcccctttaaggag |
| | | | caaatcacttctgtcacagttattatggtaatatgaggcaatctgattagcttcacagactgagtct |
| | | | ccacaacacc |
| 132 | 219000_s_at | DSCC1 | tcaagtgagtgagttcccctctacttttagccttccacccaaactggaagcctctaggtgctatca |
| | | | attatttatatccatcgtttacatccatgaaattggctgaataattactcctctgcctggcgtagacat |
| | | | gtgctttgggaaaaaaacgagtttataatcctataatgaagaatactggcacaggcaatgctcac |
| | | | tcgaaaacttcaagtaatttctagttggttttggaatgcttgataaagttcctttacagctttattttcct |
| | | | gatttgttttggtttagatcaaagttcaaattaattttaacttagctaatgaactcatcaccaggacag |
| | | | ttggagggggtaggccgaggttaaatggtccacgtttcaaaaatgttaat |
| 133 | 219171_s_at | ZNF236 | cttttgttcttgctgggttatttattttgattttagcattaaatgtcatctcaggatatctctaaaagggg |
| | | | ttgtttaattcctaattgtatagaaagctagtttggtgaattgtattggttaattgactgtttaaggcctt |
| | | | aacaggtgaatctagagcctacttttattttggttaaagaaaaagaaaatatcaataattcaattttg |
| | | | tgtcttttctcaatttattagcaaacacaagacattttatgtattatttcgatttacttcctaattataaaa |
| | | | gctgcttttttgcagaacattccttgaaaatataaggttttgaaaagacataattttacttgaatctttg |
| | | | tggggtacaggttgatctttatattttactggttgttttaaaaattctagaaaagagatttctaggcct |
| | | | catgtataaccagggttttgaggataaagaactgtatttttagaactatctcatcatagcatatctgc |
| | | | tttggaataactat |
| 134 | 219182_at | FLJ22167 | ttaccctcgtggctaagcaagtgtctgcaggagcagagatggctggaaggggcctctgcaca |
| | | | cggaagatggcttgttcagcccattcacctcctgaggatgtgggcagtctcctccaagaacaca |
| | | | tggagctgcttcctgatcccaagcaggtcattgccactggaaggacatggccccggtgatccat |
| | | | gcttcatgcccacccagaaacacacccctcagtgtgtgcctcagtttactttggagatcagttgtc |
| | | | gtttttagtgctcctttaggcttactaaaacagttttggaaacaaagctattttgaagtattcaagca |
| | | | gaggaattccctaacactgacc |
| 135 | 219425_at | SULT4A1 | gaccattttgcgagtgtagccctgtttcactcggatcaggttggcacggccgcctgcgtgtctgt |
| | | | ccacctcatccctccgtgtatctgagggagtaaaggtgaggtctttattgcttcactgcctaatttt |
| | | | ctcacccacattcgctgaagcgatggagagtcgggggccagtagccagccaaccccgtggg |
| | | | gaccggggttgtctgtcatttatgtggctggaaagcacccaaagtggtggtcaggagggtcgct |
| | | | gctgtggaaggggtctccgttcttggtgctgtatttgaaacgggtgtagagagaagcttgtgttttt |
| | | | gtttgtaatggggagaagcgtggccaggcagtggcacgtggcatcgcatggtgggctcggca |
| | | | gcaccttgcctgtgtttctgtgagggaggctgctttctgtgaaatttctttatatttttctatttttagta |
| | | | ctgtatggatgttactgagcactacacatgatccttctgtgcttgcttg |
| 136 | 219520_s_at | WWC3 | aaggaaggccagagagccgcgcagttctctgcaggtgcagatgcaggcagtggaggtggc |
| | | | ctgagcaggcagaaggacaccaagcgccctatgttgcttgtcattcatgacgtggtcttggagc |
| | | | ttctgactagttcagactgccacgccaaccccagaaaataccccacatgccagaaaagtgaag |
| | | | tcctaggtgtttccatctatgtttcaatctgtccatctaccaggcctcgcgataaaaacaaaacaaa |
| | | | aaaacgctgccaggttttagaagcagttctggtctcaaaaccatcaggatcctgccaccagggt |
| | | | tcttttgaaatagtaccacatgtaaaagggaatttggctttcacttcatctaatcactga |
| 137 | 219537_x_at | DLL3 | tcccggctacatgggagcgcggtgtgagttcccagtgcaccccgacggcgcaagcgccttgc |
| | | | ccgcggccccgccgggcctcaggcccggggaccctcagcgctaccttttgcctccggctctg |
| | | | ggactgctcgtggccgcgggcgtggccggcgctgcgctcttgctggtccacgtgcgccgcc |
| | | | gtggccactcccaggatgctgggtctcgcttgctggctgggaccccggagccgtcagtccac |
| | | | gcactcccggatgcactcaacaacctaaggacgcaggagggttccggggatggtccgagct |
| | | | cgtccgtagattggaatcgccctgaagatgtagaccctcaagggatttatgtcatatctgctcctt |
| | | | ccatctacgctcgggaggtagcgacgccccttttccccccgctacacactgggcgcgctggg |
| | | | cagaggcagcacctgctttttccctacccttcctcgattctgtccgtgaaatgaattgggtagagt |
| | | | ctctggaaggttttaagcccattttcagttctaacttactttcatcctattttgcatccc |
| 138 | 219617_at | C2orf34 | tgaagaaaaccttcattacccgcttctgcttattttgaccaaacatggatagaagattaagcttctc |
| | | | aaagacgaagaaacgtatcaagtgcatagggaatatttttacaaaaacggaaatctgtaaggg |
| | | | gtataatcgcctgcctgcgccctttgcagcatttcacgtgtgggctatggactccacctgtcctca |
| | | | cccacgttattccccagctgccctctccagctccctccccgcctctttttacactctgcttgttgctc |
| | | | gtcctgccctaaacctttgtttgtctttaaatgtgtataagctgcctgtctgtgacttgaatttgactg |
| | | | gtgaacaaactaaatatttttccctgtaattgagacagaatttcttttgatgatacccatccctccttc |
| | | | attttttttttttttttggtctttgttctgttttggtggtggtagtttttaatcagtaaacccagcaaatatca |
| | | | tgattctttcctggttagaaaaataaataaagtgtatctttttatctccctc |
| 139 | 219643_at | LRP1B | tattcacaagttttggagggctttttgttcctctgatagacatgactgacttttagctgtcataatgtat |
| | | | taacctaacagatgaaatatgttaaatatgtggttgctctttatccctttgtacaagcattaaaaaaa |
| | | | ctgctgttttataagaagactttttgttgtactatgtgcatgcatactacctatttctaaactttgccata |
| | | | ttgaggcctttataaactattgatttatgtaatactagtgcaattttgcttgaacaatgttatgcatatc |
| | | | ataaactttttcaggttcttgtttaagtacattttttaaattgaacagtatttttcattttggttataatata |
| | | | gtcattttgcctatgtttc |
| 140 | 219704_at | YBX2 | ctcagcccctgtcaacagtggggaccccaccaccaccatcctggagtgattccaactcaactc |
| | | | aaaggacacccagagctgccatctggtatctgccagtttttccaaatgacctgtaccctacccag |
| | | | taccctgctccccctttcccataattcatgacatcaaaacaccagcttttcaccttttccttgagact |
| | | | caggaggaccaaagcagcagccttttgctttttcttttttcttccctccccttatcaagggttgaag |
| | | | gaagggagccatccttactgttcagagacagcaactccctcccgtaactcaggctgagaag |
| 141 | 219882_at | TTLL7 | gtttctgtgattcaggatcctcttgggagagtatattcaataaaagcccggaggtggtgactccttt |
| | | | gcagctccagtgttgccagcgcctagtggagctttgtaaacagtgcctgctagtggtttacaaat |
| | | | atgcaactgacaaaagaggatcactttcaggcattggtcctgactggggtaattccaggtattta |
| | | | ctaccagggagcacccaattcttcttgagaacaccaacctacaacttgaagtacaattcacctgg |
| | | | aatgactcgctccaatgttttgtttacatccagatatggccatctgtgaaacagaagggaagatc |
| | | | gccattggttat |
| 142 | 219937_at | TRHDE | ggaggtcccaaatatgtggtctatcaccactgaattcatgtaatagataagaaaaaaattagagg |
| | | | tggatgtcttgttttgtgtcatgaattactaaaatctcttagtagttgtggtatatttttgagtaaaatta |
| | | | ccatttccagatttgagtttgaagggcttttatagttgtattttcctcctcactgttaataatcataatcc |
| | | | tttttcagtattttagtggccttgaacaactggtttatctacaatctcaaatcctaagtgtataattatgt |
| | | | gcaatgttcaatacctcatataatacttgctcaacagtatagtggtaccaatggcattaagatggt |
| | | | gtttttgttctacatatttttcaataatttattctttctaatgttgaaattatatcaggctttaccggtt |
| 143 | 219955_at | L1TD1 | gaagttgcaacattcgtttgataggaattccagaaaaggagagttatgagaatagggcagagg |
| | | | acataattaaagaaataattgatgaaaactttgcagaactaaagaaaggttcaagtcttgagattg |
| | | | tcagtgcttgtcgagtacctagtaaaattgatgaaaagagactgactcctagacacatcttggtg |
| | | | aaattttggaattctagtgataaagagaaaataataagggcttctagagagagaagagaaattac |
| | | | ctaccaaggaacaagaatcaggttgacagcagacttatcactggacacactggatgctagaag |
| | | | taaatggagcaatgtcttcaaagttctgctggaaaaaggctttaatcctagaatcctatatccagc |
| | | | caaaatggcatttgattttaggggcaaaacaaaggtatttcttagtattgaagaatttagagattat |
| | | | gttttgcatatgcccaccttgagagaattactggggaataatataccttagcacgccagggtgac |
| | | | taca |
| 144 | 220029_at | ELOVL2 | gttatacagatgccatgctccacaccacgagcagtgtacaaatctggctgcccgtttactttctga |
| | | | gcaagcactggagtccactccgacctttttctttgaacatgcatgctgctggaatatgtataaatc |
| | | | agaactagcagaagtagcagagtgatgggagcaaaataggcactgaattcgtcaactctttttt |
| | | | gtgagcctacttgtgaatattacctcagatacctgttgtcactcttcacaggttatttaagttcttgaa |
| | | | gctgggaggaaaaagatggagtagcttggaaagattccagcactgagccgtgagccggtcat |
| | | | gagccacgataaaaaatgccagtttggcaaactcagcactcctgttccctgctcaggtatatgc |
| | | | gatctctactgagaagcaagcacaaaagtagaccaaagtattaatgagtatttcctttctccataa |
| | | | gtgcaggactgttactcactactaaactct |
| 145 | 220076_at | ANKH | gaacgtcgtatgagatcctacaatggaagaataaaatcacctcattcttcatttcagatctgaaca |
| | | | ttagcagtgatctagatttttttttttttaaacaaaattaagtgtgcttagagtcatccctctacatggg |
| | | | ctgtggctgtcagcccataggtttgtcagtttcacatcaaaactgtgggtataaactgttgaaacc |
| | | | aatcacattaaaatatttagctgggcacagtggtgtgcatctgtagtcccagctacttgggaggct |
| | | | gaggcaggaggatcgcttaagcacaggagttggaatccagcctgagcaacagagcaaaacc |
| | | | ccgtctctaaaatacaaataaaatatttgtgtagtttttgattaaaattgactacagcggtcagtata |
| | | | aaatacatgtcgcttttaaggaagtgctctttatgtatctaacagatggaagtttttgcattggtaag |
| | | | agcatttatatatgctttgtttcagggtttatggatttgtattcatatattgtcaaataggtttcatactct |
| | | | aattttactt |
| 146 | 220294_at | KCNV1 | agattatatccctatcttctttttcatgtaaaccactggtcacaaatgaactgatctctgtatcccatt |
| | | | attactataagaggtgggaatcccaaaactgcttagattgcagtacatgagtttacacaaagactt |
| | | | caacaattgcacatcttcattctcccaactgagtgtagtatgtggagcataaaacagcatattctta |
| | | | gtatttcatgaatatcagatggtctttaaatgtctctttatggatgtattgttcacattatggctttaaaa |
| | | | taatgaatatgtaaaagtgaggtagtgaacatcctaaatttctacactggaattactaaataatctta |
| | | | tttcataaaatgggaaatatatgttaaatgacatcactggatgaacttgaagatcttttacttgttaac |
| | | | aaaaaaatactatggacagctttctgattgttggggtaaatagcaaatgttcaaactttgcaggca |
| | | | ttttgacattcatcataacaacacaattcctagacatt |
| 147 | 220366_at | ELSPBP1 | ttaggcagtctgtggtgctcagtcacctctgtcttcgatgagaaacagcagtggaaattctgtga |
| | | | aacgaatgagtatgggggaaattctctcaggaagccctgcatcttcccctccatctacagaaata |
| | | | atgtggtctctgattgcatggaggatgaaagcaacaagctctggtgcccaaccacagagaaca |
| | | | tggataaggatggaaagtggagtttctgtgccgacaccagaatttccgcgttggtccctggcttt |
| | | | ccttgtcactttccgttcaactataaaaacaagaattattttaactgcactaacaaaggatcaaagg |
| | | | agaaccttgtgtggtgtgcaacttcttacaactacgaccaagaccacacctgggtgtattgctga |
| | | | tgctgaggaaaggagaaatatcttcagaggaagactgccgccatactgaggctgagcacaga |
| | | | tttgtctttttcattgcatctgtcaa |
| 148 | 220394_at | FGF20 | gtgtggcagtgggactggtcagtattagaggtgtggacagtggtctctatcttggaatgaatgac |
| | | | aaaggagaactctatggatcagagaaacttacttccgaatgcatctttagggagcagtttgaaga |
| | | | gaactggtataacacctattcatctaacatatataaacatggagacactggccgcaggtattttgt |
| | | | ggcacttaacaaagacggaactccaagagatggcgccaggtccaagaggcatcagaaattta |
| | | | cacatttcttacctagaccagtggatccagaaagagttccagaattgtacaaggacctactgatg |
| | | | tacacttgaagtgcgatagtgacattatggaagagtcaaaccacaaccattctttcttgtcatagtt |
| | | | cccatcataaaataatgacccaagcagacgttcaaa |
| 149 | 220397_at | MDM1 | tatgcattttttaccacaatttttaaaaagtttgaatagaaatttttaatgtctttgagtggattttgtttttt |
| | | | gaacagttggatagacttctgcgtaagaaagctggattgactgttgttccttcatataatgccttga |
| | | | gaaattctgaatatcaaaggcagtttgtttggaagacttctaaagaaactgctccagcttttgcag |
| | | | ccaatcaggtagcttaatggatgtaatacatttctgagtaccattatcttatctagtaatgtagattta |
| | | | catagaattaagagttgaaagaaattaagtacttaagtagcctggaggtaggttctagaaaacca |
| | | | aaatgagagttttgctaaaatcatcctattacttatgatttatggtagtaatattatactgtcctaggct |
| | | | tctgatgatcattgttgccagatgcagcacatatactaaatatgagacagggtaatgaaaacttg |
| | | | gggaactggtaagtttttgcatgctac |
| 150 | 220541_at | MMP26 | tgacccctttgatattccagcaagtgcagaatggagatgcagacatcaaggtttctttctggcagt |
| | | | gggcccatgaagatggttggccctttgatgggccaggtggtatcttaggccatgcctttttacca |
| | | | aattctggaaatcctggagttgtccattttgacaagaatgaacactggtcagcttcagacactgg |
| | | | atataatctgttcctggttgcaactcatgagattgggcattctttgggcctgcagcactctgggaat |
| | | | cagagctccataatgtaccccacttactggtatcacgaccctagaaccttccagctcagtgccg |
| | | | atgatatccaaaggatccagcatttgtatggagaaaaatgttcatctgacataccttaatgttagca |
| | | | cagaggacttattcaacctgtcctttcagggagtttattggaggatcaaagaactgaaagcacta |
| | | | gagcagccttggggactgctaggatgaagccctaaagaatgcaacctagtcaggttagctgaa |
| | | | ccgacactcaaaacgctac |
| 151 | 220653_at | PBG3 /// | aaggtagaaagccttccgtccagtgtgcgaatctctgtgaacgtgtaagaattcacagtcagga |
| | | ZIM2 | ggactactttgaatgttttcagtgcggcaaagcttttctccagaatgtgcatcttcttcaacatctca |
| | | | aagcccatgaggcagcaagagtccttcctcctgggttgtcccacagcaagacatacttaattcg |
| | | | ttatcagcggaaacatgactacgttggagagagagcctgccagtgttgtgactgtggcagagtc |
| | | | ttcagtcggaattcatatctcattcagcattatagaactcacactcaagagaggccttaccagtgt |
| | | | cagctatgtgggaaatgtttcggccgaccctcatacctcactcaacattatcaactccattctcaa |
| | | | gagaaaactgttgagtgcgatcactgttgagaaacctttagtcacagcacacacttttctcaacat |
| | | | tattggcttcctcctagagtgttgtgagtgtgagaaggcctttcactagcccc |
| 152 | 220700_at | — | atgttactacaaacttgattaaacttctggtggaaattccatcacattttatgcaattttcaatttatttc |
| | | | tccaatttatttttaatgccacatggacattatattccttaaccattcttttgcatgtgattaacatttgtg |
| | | | aaattaaccacttaagcaagtgtttttgctttgatgaaagaaaaatgtttaaaatcctactggatatg |
| | | | aaactgaaagtaatgttttgtgttttttgtttcaaatgaaagtgtaaattaagaatttgttggcagggc |
| | | | gtggtggctcatgcctgtaatcccagcactttgggaggccgaggtgggcagatcacctgaggt |
| | | | cagcagtccaagaccaccctggccaacatggtgaagtcccgtctctactaaaaatacaaaaat |
| | | | cagctgggcatggtggcgggcacttgtagtcccagctactcaggaggctgaagcaggagaat |
| | | | cacttgaactcaggaggcagaagttgcggttagccga |
| 153 | 220703_at | C10orf110 | cctctctccactctctagaaatattaaggctaggctgctgctgtatgtcagggctagtcccctcttc |
| | | | tatgaatccagaataactctgaagaagccgagtaacaggcatgaagtgaagagaaatcgctgt |
| | | | aacaggaagacagcaaagcagatgctaatgaccacactatttaacgaactggaaccaacgag |
| | | | aaaatacggtattactgaagactgcacttccttgaacagagtgctcttctcagcaaatcggaaat |
| | | | gcctacacaaatcgctttacaagaaagactgtttcaaagcagcacctttctcaatgttctcgttca |
| | | | ggtgacaattcttcttggtctcagctccaattttattgtcattttcatcaataaggatacacatctctg |
| | | | ccaggagttgaacctgttgcttgtcgaggtggttagtgtttatttcaggcatcattacaaaatgtct |
| | | | gatctgttctagaaccct |
| 154 | 220771_at | LOC51152 | aagtatctccatacaaaatacggttgaattacaaaaagaaaattgtaacattagcatggacaaac |
| | | | ctggcaggtactccttaactctcctaagtaataaaaactgtaaaatgcaaataagccttcgatgac |
| | | | atttactaacctttactaaagtatcaatgatgacttggttgtttaaacagctgacatttgggcaatttg |
| | | | agtatgtcaaactcaataatactggttttcatttgcaagatccacttaaaacttaaggaggccaaa |
| | | | aaacatcatttaaaataccctataaattataatcatacatatgatacgaaaaatatcctacttcag |
| 155 | 220817_at | TRPC4 | catacacatacgtattttccgtagtgctctgggtgggggaaaatgtttaaattgtattagcaaatgc |
| | | | taacttacactttatagcatttatcagctgtggcatattacctgtaacatgtttaaattaaggcaaag |
| | | | gcaatcaaaaacctttttgttttgtagcctgcttttgctttcacaatttgtcttacaatt |
| 156 | 220834_at | MS4A12 | gctggccaagactactgggccgtgctttctggaaaaggcatttcagccacgctgatgatcttctc |
| | | | cctcttggagttcttcgtagcttgtgccacagcccattttgccaaccaagcaaacaccacaacca |
| | | | atatgtctgtcctggttattccaaatatgtatgaaagcaaccctgtgacaccagcgtcttcttcagc |
| | | | tcctcccagatgcaacaactactcagctaatgcccctaaatagtaaaagaaaaaggggtatcag |
| | | | tctaatctcatggagaaaaactacttgcaaaaacttcttaagaagatgtcttttattgtctacaatga |
| | | | tttctagtctttaaaaactgtgtttgagatttgtttttaggttggtcgctaatgatggctgtatctccctt |
| | | | cactgtctcttcctacattaccactactacatgctggcaaaggtgaaggatcagaggactgaaaa |
| | | | atgattctgcaactctcttaaa |
| 157 | 220847_x_at | ZNF221 | tgacatgcaccagagggtccacaggggagagcgaccctataattgtaaggaatgtggaaaga |
| | | | gctttggctgggcttcatgtcttttgaaacatcagagactccacagtggagaaaagccattgaaa |
| | | | tctggagtgtgggaagagatctactcagaattcacagcttcatttacatcagtaagtctatgtggg |
| | | | agaaaagccatataaatgtgagaagtgtgggaagggctttggctgggcctcaactcatctgac |
| | | | ccatcaattctccacagcagagaaaaaccattcaaatatgagaactgtgggaagagctttgtac |
| | | | atagatcatatctttttttttttttttgagacagagtctcactctttcacccaagcctgactgcagtggcg |
| 158 | 220852_at | PRO1768 | gaaaagcgccctgtgctgagtaaagcagccagtcttctcttgtcacagtaaaaggctgggagta |
| | | | aaatttcccataaacacaggggaaacctacatttactcacatgccaaggaaaatggcacggaa |
| | | | gacccacgtgtagccacagcagagtctatgcagagggcctgcaaatgcctggggtgcgagtg |
| | | | aatgcctggaggggcggagtttccaagataacagctattgtgttttctttttcacacttcagaaga |
| | | | gaatcctaaggactagactccgctcagtgcattcctttttcatacactgatctcaagtacaatcaca |
| | | | taattttgaaaatccatgtagtcctccctaaataaaattataaggataggtttctatttccttccgatta |
| | | | cctagatacctccgtcttctggaaaaccccaaaaagaccagtagacgaatcaggaaggtccta |
| | | | ggagtgattcctccaat |
| 159 | 220970_s_at | KAP2.1 | tgcccccacagagcaatacactgaagcctaaacatctatctggtgtttttaaaaagttaaaagaa |
| | | B /// | aaatagattttttttcacaaggtgacaatagtgatttttaccatctggatacagcctggtgtaagca |
| | | KRTAP2- | gacgtccattaccaccctcacccacattttcaggtgtctacatcagccttagtcattatggatagta |
| | | 4 /// | aatcgacctttaagaattcctggggtggactttgcaaacacattctacaacctgatggtttttactg |
| | | LOC644350 | ctcaaactgtcaccatcatcttttgcaatgtgttgctcactgttgtcaata |
| | | /// | |
| | | LOC728285 | |
| | | /// | |
| | | LOC728934 | |
| | | /// | |
| | | LOC730755 | |
| 160 | 220981_x_at | LOC650686 | ggacagtctcagggttctgttctcgccttcacccggaccttcattgctacccctggcagcagttc |
| | | /// | cagtctgtgcatcgtgaatgacgagctgtttgtgagggatgccagcccccaagagactcagag |
| | | NXF2 /// | tgccttctccatcccagtgtccacactctcctccagctctgagccctccctctcccaggagcagc |
| | | NXF2B | aggaaatggtgcaggctttctctgcccagtctgggatgaaactggagtggtctcagaagtgcct |
| | | | tcaggacaatgagtggaactacactagagctggccaggccttcactatgctccagaccgagg |
| | | | gcaagatccccgcagaggccttcaagcaaatctcctaaaaggagccctccgatgtcttctttgtc |
| | | | ttcgttcacatcctctttgtttcctcttttcaccagcctaaggcctggctgaccaggaagccaacgt |
| | | | taacttgcaggccacgtgacataac |
| 161 | 220993_s_at | GPR63 | aagtctgcattgaatccgctgatctactactggaggattaagaaattccatgatgcttgcctggac |
| | | | atgatgcctaagtccttcaagtttttgccgcagctccctggtcacacaaagcgacggatacgtcc |
| | | | tagtgctgtctatgtgtgtggggaacatcggacggtggtgtgaatattggaactggctgacatttt |
| | | | gggtgatgcttgttctttattgacattgaattctctttctcatagcctctccactttatttttttttataggg |
| | | | tttgtgtatgtatgtgtgtgagcagtgtaaagaaagaatggtaattatagttctgttaccaagaata |
| | | | aataataggaaagtgattacaaatattacctccagggttcaatagaaatcctcaatttagggtgag |
| | | | gagacttttttttggttttggggtttttccttgattgattttgttttcatagtgggaatcaggattgtgctt |
| | | | tattgagcctgcagttacattgaattgtaggtgtttcgtgtgctgctaaggta |
| 162 | 221018_s_at | TDRD1 | gggactgtcgatgtagctgataagctagtgacatttggtctggcaaaaaacatcacacctcaaa |
| | | | ggcagagtgctttaaatacagaaaagatgtataggacgaattgctgctgcacagagttacagaa |
| | | | acaagttgaaaaacatgaacatattcttctcttcctcttaaacaattcaaccaatcaaaataaattta |
| | | | ttgaaatgaaaaaactggtaaaaagttaagtaagttaaatcgtatgttttcgcctcttctgtgatcac |
| | | | caataggacatcttcaggcatattggcaggatagagctaatggagtgaaacctattgtaaggct |
| | | | gtactttcgtgatttaatgacctgaggtttggtcataatgcttctgctgtttttgtaggtttatctgatc |
| | | | gttttcctttgctactgctaatggaactgaacccccaggggtattccagttgtaatagcctttcctta |
| | | | ctgttgtttgg |
| 163 | 221077_at | ARMC4 | gttgagttgaaattctgccgcttactcaatggccttgggtgatgatgctgtaccctaattctaaagg |
| | | | aagcaatgaacccccttttcagctaccttactgataagcacttatgttctgccttctgctatcctgat |
| | | | ggttcgggttgtctgtcttactatctacttcttgagtagagagaccacattaaatttattgctgtatct |
| | | | cacagggcatcttgctagtgtgcacaggctcgcctccctacctctgccccgatggtgtgaagg |
| | | | ggagagggcgaggttccttagtggcagggctttgctgttcttcactctcagccccctgaaagca |
| | | | gttcttcctgcctctgagcctgtctttccttctgctgttaacttctttcctacttttcttgcatccctctcc |
| | | | cttccttttcctgccgtctttcttgtagacat |
| 164 | 221137_at | — | aaaaggactaactcacatggctgcagtaagtgctggctgttagctggaagcacaaccaaggct |
| | | | gttaacaggtgtgccttggttctcttccatatggcttctcttttgttttcagtactctgcagtttaattat |
| | | | gatgcatgcaggtgtgaatttctgtttattctgcttgggatgtgttttccttctgggatctgtgaatcg |
| | | | gtttctcattatttttgtaaaacctgaagccagttatctcttaaaataccagctctccttg |
| 165 | 221168_at | PRDM13 | ctggacttcttggatgagctcaccctgaaccgcccaggcggtctgctcttggtgttcagaatcac |
| | | | atcaatgcgaacgtcacagcgccttcgagggcgcagatttaactgccacgtatttttaagttgta |
| | | | cttttctgtggaggaaattgtgccttttgaaacgacgttttgtgtgtgtatttcacgttagcatttcatt |
| | | | gcataggcaaaacactagtcacaattgggtagatgtgacatccatatacttgtttacattttatctgt |
| | | | tctcatgtcaaagactactccttgccccattgaatatatagtggtagcaggtgtacaaattggtca |
| | | | agttgcaattatttatgagagaataatgataaatgtaaaatatctaaagcatgaatctaagagcac |
| | | | gcaatatataattttaaagaaaatattctatttggtagaatacaaatgtggtgtgtgttgttttataatg |
| | | | actgctgtacagtgggtatagtattttggttttggttccagattgtgcaatc |
| 166 | 221258_s_at | KIF18A | gtgaagacatcaagagctcgaagtgtaaattacccgaacaagaatcactaccaaatgataaca |
| | | | aagacattttacaacggcttgatccttcttcattctcaactaagcattctatgcctgtaccaagcatg |
| | | | gtgccatcctacatggcaatgactactgctgccaaaaggaaacggaaattaacaagttctacat |
| | | | caaacagttcgttaactgcagacgtaaattctggatttgccaaacgtgttcgacaagataattcaa |
| | | | gtgagaagcacttacaagaaaacaaaccaacaatggaacataaaagaaacatctgtaaaataa |
| | | | atccaagcatggttagaaaatttggaagaaatatttcaaaaggaaatctaagataaatcacttcaa |
| | | | aaccaagcaaaatgaagttgatcaaatctgcttttcaaagtttatcaataccctttcaaaaatatatt |
| | | | taaaatctttgaaagaagacccatcttaaagctaagtttacccaagtactttcagcaagc |
| 167 | 221319_at | PCDHB8 | cgggagcctgtctcagaactatcagtacgaggtgtgcctggcaggaggctcagggacgaatg |
| | | | agttccagttcctgaaaccagtattacctaatattcagggccattcttttgggccagaaatggaac |
| | | | aaaactctaactttaggaatggctttggtttcagccttcagttaaagta |
| 168 | 221393_at | TAAR3 | gaactccaccataaagcaactgctggcattttgctggtcagttcctgctcttttttcttttggtttagtt |
| | | | ctatctgaggccgatgtttccggtatgcagagctataagatacttgttgcttgcttcaatttctgtgc |
| | | | ccttactttcaacaaattctgggggacaatattgttcactacatgtttctttacccctggctccatcat |
| | | | ggttggtatttatggcaaaatctttatcgtttccaaacagcatgctcgagtcatcagccatgtgcct |
| | | | gaaaacacaaagggggcagtgaaaaaacacctatccaagaaaaaggacaggaaagcagcg |
| | | | aagacactgggtatagtaatgggggtgtttctggcttgctggttgccttgttttcttgctgttctgatt |
| | | | gacccatacctagactactccactcccatactaatattggatcttttagtgtggctccggtacttca |
| | | | actctacttgcaaccctcttattcatggcttttttaatccatggtttcagaaagcattcaagtacatag |
| | | | tgtcaggaaaaatatttagctcccattcagaaactgc |
| 169 | 221591_s_at | FAM64A | cacatctggacccatcagtgactgcctgccatagcctgagagtgtcttggggagaccttgcaga |
| | | | gggggagaattgttccttctgctttcctaggggactcttgagcttagaaactcatcgtacacttga |
| | | | ccttgagccttctatttgcctcatctataacatgaagtgctagcatcagatatttgagagctcttagc |
| | | | tctgtacccgggtgcctggtttttggggagtcatccgcagagtcactcacccactgtgtttctggt |
| | | | gccaaggctcttgagggccccactctcatccctcctttccctaccagggactcggaggaaggc |
| | | | ataggagatatttccaggcttacgaccctgggctcacgggtacctatttatatgctcagtgcaga |
| | | | gcactgtggatgtgccaggaggggtagccctgttcaagagcaatttctgccctttgtaaattattt |
| | | | aagaaacctgctttgtcattttattagaaagaaaccagcgtgtgactttcctagataacactgctttc |
| 170 | 221609_s_at | WNT6 | ccgccaggagagcgtgcagctcgaagagaactgcctgtgccgcttccactggtgctgcgtag |
| | | | tacagtgccaccgttgccgtgtgcgcaaggagctcagcctctgcctgtgacccgccgcccgg |
| | | | ccgctagactgacttcgcgcagcggtggctcgcacctgtgggacctcagggcaccggcacc |
| | | | gggcgcctctcgccgctcgagcccagcctctccctgccaaagcccaactcccagggctctgg |
| | | | aaatggtgaggcgaggggcttgagaggaacgcccacccacgaaggcccagggcgccaga |
| | | | cggccccgaaaaggcgctcggggagcgtttaaaggacactgtacaggccctccctccccttg |
| | | | gcctctaggaggaaacagttttttagactggaaaaaagccagtctaaaggcctctggatactgg |
| | | | gctccccagaactgc |
| 171 | 221718_s_at | AKAP13 | gcgatgcagaaatgaaccaccggagttcaatgcgagttcttggggatgttgtcaggagacctc |
| | | | ccattcataggagaagtttcagtctagaaggcttgacaggaggagctggtgtcggaaacaagc |
| | | | catcctcatctctagaagtaagctctgcaaatgccgaagagctcagacacccattcagtggtga |
| | | | ggaacgggttgactctttggtgtcactttcagaagaggatctggagtcagaccagagagaacat |
| | | | aggatgtttgatcagcagatatgtcacagatctaagcagcagggatttaattactgtacatcagc |
| | | | catttcctctccattgacaaaatccatctcattaatgacaatcagccatcctggattggacaattca |
| | | | cggccctt |
| 172 | 221950_at | EMX2 | gtaggctcagcgatagtggtcctcttacagagaaacggggagcaggacgacgggggngctg |
| | | | gggntggcgggggagggtgcccacaaaaagaatcaggacttgtactgggaaaaaaacccct |
| | | | aaattaattatatttcttggacattccctttcctaacatcctgaggcttaaaaccctgatgcaaacttc |
| | | | tcctttcagtggttggagaaattggccgagttcaaccattcactgcaatgcctattccaaactttaa |
| | | | atctatctattgcaaaacctgaaggactgtagttagcggggatgatgttaagtgtggccaagcgc |
| | | | acggcggcaagttttcaagcactgagtttctattccaagatcatagacttactaaagagagtgac |
| | | | aaatgcttccttaatgtcttctataccagaatgtaaatatttttgtgttttgtgttaatttgttagaattct |
| | | | aacacactatatacttccaa |

TABLE 12
Validation of the independent prognostic value of
the 15-gene signature in four other separate stage
IB-II patient cohorts who received no adjuvant treatment

| | Tumour | | Hazard | | |
|---|---|---|---|---|---|
| Trial/Source | Type | n | Ratio | 95% CI | p value |
| JBR.10 | All NSCLC | 62 | 18.00 | 5.78-56.05 | <0.0001 |
| DCC | ADC | 96 | 2.26 | 1.02-4.97 | 0.044 |
| NLCI | All NSCLC | 133 | 2.27 | 1.18-4.35 | 0.014 |
| Duke | All NSCLC | 48 | 1.96 | 0.87-4.42 | 0.11 |
| UM-SQ | SQC | 79 | 3.57 | 1.48-8.58 | 0.005 |

HR: hazard ratio;
OBS: observation;
NSCLC: non-small cell lung cancer;
ADC: adenocarcinoma;
SQC: squamous cell carcinoma;
DCC: Director's Challenge Consortium adenocarcinoma dataset;
NLCI: Netherlands Cancer Institute;
Duke: Duke University;
UM-SQ: University of Michigan, squamous cell carcinoma dataset.

TABLE 13
Demographic features of patients in the four validation sets of stage IB and II patients.

| | Director's Challenge (DCC) | | | | | | |
|---|---|---|---|---|---|---|---|
| | All | UM | HLM | MSK | NLCI | Duke | UM-SQ |
| Clinical Factors | n = 96 (%) | n = 27 (%) | n = 38 (%) | n = 31 (%) | n = 133 (%) | n = 48 (%) | n = 79 (%) |

Pathologic subtype

| | | | | | | | |
|---|---|---|---|---|---|---|---|
| Adeno | 96 (100) | 27 (100) | 38 (100) | 31 (100) | 39 (29) | 18 (38) | 0 |
| Non-Adeno | 0 (0) | 0 (0) | 0 (0) | 0 (0) | 94 (71) | 30 (62) | 79 (100) |

Stage

| | | | | | | | |
|---|---|---|---|---|---|---|---|
| IB | 68 (71) | 17 (63) | 29 (76) | 22 (71) | 78 (59) | 30 (63) | 46 (59) |
| II | 28 (29) | 10 (37) | 9 (24) | 9 (29) | 55 (41) | 18 (37) | 33 (41) |

Age (years)

| | | | | | | | |
|---|---|---|---|---|---|---|---|
| <65 | 40 (42) | 14 (52) | 14 (37) | 12 (39) | 68 (51) | 20 (42) | 26 (33) |
| ≧65 | 56 (58) | 13 (48) | 24 (63) | 19 (61) | 65 (49) | 28 (58) | 53 (67) |

Sex

| | | | | | | | |
|---|---|---|---|---|---|---|---|
| Male | 49 (51) | 16 (59) | 21 (55) | 12 (39) | NA | 32 (67) | 49 (62) |
| Female | 47 (49) | 11 (41) | 17 (45) | 19 (61) | NA | 16 (33) | 30 (38) |

DCC: Director's Challenge Consortium;
UM: University of Michigan;
HLM: H. Lee Moffitt Cancer Center;
MSK: Memorial Sloan-Kettering Cancer Center;
NLCI: Netherlands Cancer Institute.
*Only Stage IB-II patients who did not receive adjuvant therapy of any type (chemotherapy or radiotherapy);
NA: not available.

TABLE 14
Demographic features of patients in UHN183 validation set
(stage I and II) and the training set (BR10-OBS).
Clinical factors - A comparative table of the 2 datasets (training and
current validation)

| | UHN | BR10-OBS |
|---|---|---|
| | N = 183 | N = 62 |
| | N (%) | N (%) |
| Age | | |
| Median (range) | 70 | 61.2 |
| | (40-88) | (35.4-76.7) |
| 65 | 60 (33) | 44 (69) |
| 65 | 123 (67) | 19 (31) |
| Sex | | |
| Women | 84 (46) | 18 (29) |
| Men | 99 (54) | 44 (71) |
| Stage | | |
| 1A | 49 (27) | |
| 1B | 80 (44) | 34 (55) |
| 2A | 9 (5) | 28* (45) |
| 2B | 45 (25) | |
| 3A | | |
| Histology | | |
| Adenocarcinoma (ADE) | 130 (71) | 32 (52) |
| Squamous (SQC) | 43 (24) | 26 (42) |
| Adenosuamous (ASQ) | 2 (1) | |
| Large Cell (LC) | 8 (4) | |
| Other | | 4 (6) |
| 15 gene signature | | |
| Low Risk | 90 (49) | 29 (47) |
| High Risk | 93 (51) | 33 (53) |

*Stage 2 or higher.

### REFERENCES

1. Jemal A, Siegel R, Ward E, Murray T, Xu J, Thun M J. Cancer Statistics, 2007. CA Cancer J Clin 2007;57:43-66.
2. Arriagada R, Bergman B, Dunant A, Le Chevalier T, Pignon J P, Vansteenkiste J. Cisplatin-based adjuvant chemotherapy in patients with completely resected non-small-cell lung cancer. N Engl J Med 2004;350:351-60.
3. Winton T, Livingston R, Johnson D, et al. Vinorelbine plus cisplatin vs. observation in resected non-small-cell lung cancer. N Engl J Med 2005;352:2589-97.
4. Douillard J Y, Rosell R, De Lena M, et al. Adjuvant vinorelbine plus cisplatin versus observation in patients with completely resected stage IB-IIIA non-small-cell lung cancer (Adjuvant Navelbine International Trialist Association [ANITA]): a randomised controlled trial. Lancet Oncol 2006;7:719-27.
5. Strauss G M, Herndon J E, II, Maddaus M A, et al. Adjuvant chemotherapy in stage IB non-small cell lung cancer (NSCLC): Update of Cancer and Leukemia Group B (CALGB) protocol 9633. ASCO Meeting Abstracts 2006;24:7007-.
6. Pignon J P, Tribodet H, Scagliotti G V, et al. Lung Adjuvant Cisplatin Evaluation (LACE): A pooled analysis of five randomized clinical trials including 4,584 patients. ASCO Meeting Abstracts 2006;24 :7008- .
7. Scagliotti G V, Fossati R, Torri V, et al. Randomized study of adjuvant chemotherapy for completely resected stage I, II, or IIIA non-small-cell Lung cancer. J Natl Cancer Inst 2003;95:1453-61.
8. Waller D, Peake M D, Stephens R J, et al. Chemotherapy for patients with non-small cell lung cancer: the surgical setting of the Big Lung Trial. Eur J Cardiothorac Surg 2004;26:173-82.
9. Douillard J Y, Rosell R, Delena M, Legroumellec A, Torres A, Carpagnano F. ANITA: Phase III adjuvant vinorelbine (N) and cisplatin (P) versus observation (OBS) in completely resected (stage I-III) non-small-cell lung cancer (NSCLC) patients (pts): Final results after 70-month median follow-up. On behalf of the Adjuvant Navelbine International Trialist Association. ASCO Meeting Abstracts 2005;23:7013-.
10. Hoffman P C, Mauer A M, Vokes E E. Lung cancer. Lancet 2000;355:479-85.
11. Nesbitt J C, Putnam J B, Jr., Walsh G L, Roth J A, Mountain C F. Survival in early-stage non-small cell lung cancer. Ann Thorac Surg 1995;60:466-72.
12. Beer D G, Kardia S L, Huang C C, et al. Gene-expression profiles predict survival of patients with lung adenocarcinoma. Nat Med 2002;8:816-24.
13. Chen H Y, Yu S L, Chen C H, et al. A five-gene signature and clinical outcome in non-small-cell lung cancer. N Engl J Med 2007;356:11-20.
14. Lu Y, Lemon W, Liu P Y, et al. A gene expression signature predicts survival of patients with stage I non-small cell lung cancer. PLoS Med 2006;3:e467.
15. Potti A, Mukherjee S, Petersen R, et al. A genomic strategy to refine prognosis in early-stage non-small-cell lung cancer. N Engl J Med 2006;355:570-80.
16. Raponi M, Zhang Y, Yu J, et al. Gene expression signatures for predicting prognosis of squamous cell and adenocarcinomas of the lung. Cancer Res 2006;66:7466-72.
17. Wigle D A, Jurisica I, Radulovich N, et al. Molecular profiling of non-small cell lung cancer and correlation with disease-free survival. Cancer Res 2002;62:3005-8.
18. Bianchi F, Nuciforo P, Vecchi M, et al. Survival prediction of stage I lung adenocarcinomas by expression of 10 genes. J Clin Invest 2007;117:3436-44.
19. Sun Z, Wigle D A, Yang P. Non-overlapping and non-cell-type-specific gene expression signatures predict lung cancer survival. J Clin Oncol 2008;26:877-83.
20. Lau S K, Boutros P C, Pintilie M, et al. Three-gene prognostic classifier for early-stage non small-cell lung cancer. J Clin Oncol 2007;25:5562-9.
21. Oshita F, Ikehara M, Sekiyama A, et al. Genomic-wide cDNA microarray screening to correlate gene expression profile with chemoresistance in patients with advanced lung cancer. J Exp Ther Oncol 2004;4:155-60.
22. Bolstad B M, Irizarry R A, Astrand M, Speed T P. A comparison of normalization methods for high density oligonucleotide array data based on variance and bias. Bioinformatics 2003;19:185-93.
23. Affymetrix, ed. Transcript assignment for NetAffxTM annotation; 2006.
24. Dworakowska D, Jassem E, Jassem J, et al. Clinical significance of apoptotic index in non-small cell lung cancer: correlation with p53, mdm2, pRb and p21WAF1/CIP1 protein expression. J Cancer Res Clin Oncol 2005;131:617-23.
25. Allory Y, Matsuoka Y, Bazille C, Christensen E I, Ronco P, Debiec H. The L1 cell adhesion molecule is induced in renal cancer cells and correlates with metastasis in clear cell carcinomas. Cancer Res 2005;11:1190-7.
26. Boo Y J, Park J M, Kim J, et al. L1 expression as a marker for poor prognosis, tumor progression, and short survival in patients with colorectal cancer. Ann Surg Oncol 2007;14:1703-11.
27. Gast D, Riedle S, Schabath H, et al. L1 augments cell migration and tumor growth but not beta3 integrin expression in ovarian carcinomas. Int J Cancer 2005;115:658-65.
28. Thies A, Schachner M, Moll I, et al. Overexpression of the cell adhesion molecule L1 is associated with metastasis in cutaneous malignant melanoma. Eur J Cancer 2002;38:1708-16.
29. Ouellet V, Provencher D M, Maugard C M, et al. Discrimination between serous low malignant potential and invasive epithelial ovarian tumors using molecular profiling. Oncogene 2005;24:4672-87.

1-28. (canceled)

29. A method for prognosing or classifying a subject with NSCLC comprising:
a. calculating a combined score from relative expression levels of at least 15 different biomarkers in the subject, wherein the at least 15 biomarkers comprise FAM64A, MB, EDN3, ZNF236, FOSL2, MYT1L, MLANA, L1CAM, TRIM14, STMN2, UMPS, ATP1B1, HEXIM1, IKBKAP, and MDM2, and
b. classifying the subject into a high or low risk group based on the combined score.

30. The method of claim 29 wherein the combined score is calculated from the relative expression levels of FAM64A, MB, EDN3, ZNF236, FOSL2, MYT1L, MLANA, L1 CAM, TRIM14, STMN2, UMPS, ATP1B1, HEXIM1, IKBKAP, and MDM2.

31. The method of claim 29, wherein the combined score is calculated from the relative expression levels of 16, 17, or 18 different biomarkers, wherein the one, two, or three additional biomarkers are selected from the genes listed in Table 3.

32. The method of claim 31, wherein the additional one, two, or three biomarkers are selected from the group consisting of RGS4, UGT2B4, and MCF2.

33. A method for prognosing or classifying a subject with NSCLC comprising:
a. determining relative expression levels of at least 15 different biomarkers, wherein the biomarkers comprise FAM64A, MB, EDN3, ZNF236, FOSL2, MYT1L, MLANA, L1 CAM, TRIM14, STMN2, UMPS, ATP1B1, HEXIM1, IKBKAP, and MDM2,
b. calculating a combined score from the relative expression levels of at least 15 different biomarkers in the subject, and
c. classifying the subject into a high or low risk group based on the combined score.

34. The method according to claim 33, wherein the relative expression levels of fifteen, sixteen, seventeen, or eighteen different biomarkers selected from the group consisting of FAM64A, MB, EDN3, ZNF236, FOSL2, MYT1L, MLANA, L1CAM, TRIM14, STMN2, UMPS, ATP1B1, HEXIM1, IKBKAP, MDM2, RGS4, UGT2B4, and MCF2 are determined.

35. The method according to claim 29, wherein the combined score is calculated according to Formula I.

36. (canceled)

37. A method for selecting therapy comprising the steps of claim 29, and further comprising selecting adjuvant chemotherapy for a subject in the high risk group or no adjuvant chemotherapy for a subject in the low risk group, wherein the subject is a human.

38. A kit to prognose or classify a subject with NSCLC comprising detection agents capable of detecting the expression product of at least 15 different biomarkers wherein the at least 15 different biomarkers comprise FAM64A, MB, EDN3, ZNF236, FOSL2, MYT1L, MLANA, L1CAM, TRIM14, STMN2, UMPS, ATP1B1, HEXIM1, IKBKAP, and MDM2.

39. The kit of claim 38, comprising detection agents capable of detecting the expression product of 16, 17, or 18 different biomarkers, wherein the additional one, two, or three biomarkers are selected from the genes listed in Table 3.

40. The kit of claim 38, comprising detection agents capable of detecting the expression products of 15, 16, 17, or 18 different biomarkers, selected from the group consisting of FAM64A, MB, EDN3, ZNF236, FOSL2, MYT1L, MLANA, L1CAM, TRIM14, STMN2, UMPS, ATP1B1, HEXIM1, IKBKAP, MDM2, RGS4, UGT2B4, and MCF2.

41. The kit of claim 38, further comprising an addressable array comprising probes for the expression products of the at least 15 biomarkers.

42. The kit of claim 38, wherein the detection agents comprise primers capable of hybridizing to the expression products of at least 15 biomarkers.

43. The kit of claim 38, wherein the detection agents comprise primers capable of hybridizing to the expression products of 16, 17, or 18 biomarkers.

44. A kit according to claim 38, further comprising a computer implemented product for calculating a combined score for a subject.

45. The method according to claim 33, wherein the combined score is calculated according to Formula I.

46. A method for selecting therapy comprising the steps of claim 33, and further comprising selecting adjuvant chemotherapy for a subject in the high risk group or no adjuvant chemotherapy for a subject in the low risk group, wherein the subject is a human.
